(19) United States
(12) Patent Application Publication
(10) Pub. No.: US 2026/0240959 A1
(43) Pub. Date: Aug. 20, 2026

(54) METHODS FOR IN VIVO EDITING OF KLKB1

(71) Applicant: INTELLIA THERAPEUTICS, INC., Cambridge, MA (US)

(72) Inventors: David MAAG, Cambridge, MA (US); David LEBWOHL, Cambridge, MA (US); James BUTLER, Cambridge, MA (US); Michael MAITLAND, Cambridge, MA (US); Jonathan PHILLIPS, Cambridge, MA (US); Yuanxin XU, Cambridge, MA (US); Ahmed ABDELHADY ABOZEID, Cambridge, MA (US)

(21) Appl. No.: 18/992,548

(22) PCT Filed: Jul. 7, 2023
(86) PCT No.: PCT/US2023/069753

§ 371 Date: Jan. 8, 2025

Related U.S. Application Data
(60) Provisional application No. 63/359,656, filed on Jul. 8, 2022.
(60) Provisional application No. 63/375,497, filed on Sep. 13, 2022.
(60) Provisional application No. 63/422,561, filed on Nov. 4, 2022.
(60) Provisional application No. 63/499,794, filed on May 3, 2023.

(51) Int. Cl. A61K 38/46; A61K 9/51; A61K 31/415; A61K 31/58; A61K 31/7105; A61P 7/10

(52) U.S. Cl. CPC A61K 38/465; A61K 9/5123; A61K 31/415; A61K 31/58; A61K 31/7105; A61P 7/10

(57) ABSTRACT

The first systemic administration of a CRISPR/Cas9-based therapeutic for in vivo editing of a liver KLKB1 gene in a clinical trial is described. Described herein are methods for in vivo editing of a liver KLKB1 gene by systemically administering a lipid nanoparticle composition comprising an mRNA encoding a Cas9 nuclease and a guide RNA that targets the gene. Assessment of biosafety metrics and clinical efficacy metrics, as well as methods of treatment, are also described herein.

### RELATED APPLICATIONS

The instant application is a U.S. national stage entry under 35 U.S.C. § 371 of international application number PCT/US2023/069753, filed Jul. 7, 2023, which designates the U.S. and claims the benefit of and priority to U.S. Provisional Application No. 63/499,794, filed on May 3, 2023; U.S. Provisional Application No. 63/422,561, filed on Nov. 4, 2022; U.S. Provisional Application No. 63/375,497, filed on Sep. 13, 2022; and U.S. Provisional Application No. 63/359,656, filed on Jul. 8, 2022, the entire contents of each of which are expressly incorporated herein by reference in their entireties.

### SEQUENCE LISTING

The instant application contains a Sequence Listing which has been submitted electronically in XML format and is hereby incorporated by reference in its entirety. Said XML copy, created on Jul. 7, 2023, is named 12793_0037-00000_SL and is 367,935 bytes in size.

### BACKGROUND

Hereditary angioedema (HAE) is a rare, autosomal dominant genetic disorder characterized by severe recurring and unpredictable inflammatory attacks in various organs and tissues of the body which can be painful, debilitating, and life-threatening. Hereditary angioedema stems from excess bradykinin in the blood promoting vascular permeability and episodes of swelling. Most patients with HAE have a C1 inhibitor (also called C1 esterase inhibitor or C1-INH) protein deficiency (either reduced levels of functional C1-INH or normal levels of a variant with reduced activity). In the absence of sufficient C1-INH function, bradykinin levels can rise, initiate vascular leakage, and cause swelling attacks.

Bradykinin production is controlled via the kallikrein-kinin (contact) pathway which is endogenously inhibited by C1-INH. Bradykinin peptide is formed when high-molecular weight kininogen (HMWK) is cleaved by plasma kallikrein (pKal), an activated form of the protein prekallikrein. Prekallikrein, also called KLKB1 protein, is encoded by KLKB1. Prekallikrein is produced in the liver and secreted into plasma where it can be activated by factor XIIa. Once KLKB1 is activated to become kallikrein, pKal can increase bradykinin levels. An excess of bradykinin in the blood leads to fluid leakage through the walls of blood vessels into body tissues. Excessive accumulation of fluids in body tissues causes the episodes of swelling seen in individuals with HAE.

Medications are used either to prevent attacks (prophylaxis) or treat attacks on demand. Whereas on-demand treatments are administered at the onset of an HAE attack, prophylactic agents require chronic intravenous (IV) or subcutaneous (SC) administration as often as twice per week or daily oral administration to ensure constant pathway suppression for disease control. Despite chronic administration, breakthrough attacks still occur.

Multiple new therapeutic strategies have improved outcomes for patients with HAE (Busse and Christiansen, 2020). The strategies are based on detailed characterization of the biochemical pathways regulating aberrant production of bradykinin and designed to restore normal function of the contact activation pathway. These strategies include C1 esterase inhibitor (C1-INH) replacement (recombinant or plasma-derived), inhibition of the B2 bradykinin receptor (B2R) (icatibant), and kallikrein inhibition (ecallantide, lanadelumab, berotralstat). However, injection-site reactions (with lanadelumab) and gastrointestinal side effects (with berotralstat) occurred in a substantial proportion of the patients (Cohn et al., 2020).

Enhancements in regulation of aberrant production of bradykinin by rebalancing the function of the contact activation pathway, including sustained knockdown of prekallikrein, may translate to improved outcomes for subjects with HAE. Thus, there remains an unmet need for gene editing therapies that are capable of producing long-lasting effects in gene expression, e.g., knockdown of prekallikrein, without requiring chronic administration.

### SUMMARY

The present disclosure describes systemic administration of a CRISPR/Cas9-based therapeutic for in vivo editing of KLKB1. In some embodiments, the present invention provides methods using a single guide RNA with a Cas9 nuclease such as the CRISPR/Cas9 system to substantially reduce or knockdown expression of the KLKB1 gene, thereby substantially reducing or eliminating the production of prekallikrein—associated with signaling through the contact activation pathway. The substantial reduction or elimination of the production of prekallikrein protein through alteration of the KLKB1 gene can be a long-term reduction or elimination of plasma kallikrein levels, such as a durable reduction of plasma kallikrein. Additional embodiments include a lipid nanoparticle system for use in in vivo liver-targeted LNP delivery of a CRISPR/Cas9 RNA components to a human subject, such as single guide RNA targeted to KLKB1 gene and mRNA encoding a Cas9 nuclease, and methods of using the same.

The following non-limiting embodiments are provided:
1. In a first embodiment, disclosed herein is a method of treating hereditary angioedema (HAE) in a human subject comprising systemically administering to the human subject a LNP composition comprising a therapeutically effective amount of: i. an mRNA encoding a Cas nuclease, and ii. a guide RNA that targets the KLKB1 gene in the liver.
2. In a second embodiment, disclosed herein is a method of preventing HAE attacks in a human subject comprising systemically administering to the human subject a LNP composition comprising a therapeutically effective amount of: i. an mRNA encoding a Cas nuclease, and ii. a guide RNA that targets the KLKB1 gene in the liver.
3. In a third embodiment, disclosed herein is a method of reducing the frequency of angioedema attacks in a human subject with HAE comprising systemically administering to the human subject a LNP composition comprising a therapeutically effective amount of: i. an mRNA encoding a Cas nuclease, and ii. a guide RNA that targets the KLKB1 gene in the liver.
4. In a fourth embodiment, disclosed herein is a method for in vivo editing of the kallikrein (KLKB1) gene in a human subject having HAE, comprising: a. systemically administering to the human subject a LNP composition comprising an therapeutically effective amount of: i. an mRNA encoding a Cas nuclease, and ii. a guide RNA that targets the KLKB1 gene; and b. editing the KLKB1 gene at the site targeted by the guide RNA in a hepatocyte of the subject; wherein the administration of the composition results in a clinically significant improvement in a level of a clinical metric in the subject as compared to a baseline level.
5. In a fifth embodiment, disclosed herein is a method for treating hereditary angioedema (HAE) in a human subject, comprising: a. systemically administering to the human subject a LNP composition comprising a therapeutically effective amount of: i. an mRNA encoding a Cas nuclease, and ii. a guide RNA that targets the KLKB1 gene wherein the guide RNA comprises a targeting sequence comprising the nucleotide sequence GGAUUGCGUAUGGGACACAA (SEQ ID NO: 15), wherein the administration of the composition reduces total plasma kallikrein protein level relative to baseline total plasma kallikrein protein level.
6. In a sixth embodiment, disclosed herein is method of treating hereditary angioedema (HAE) in a human subject comprising: a. systemically administering to the human subject a LNP composition comprising a therapeutically effective amount of: i. an mRNA encoding a Cas nuclease, and ii. a guide RNA that targets the KLKB1 gene in the liver; b. determining a first level of a clinical metric in the subject prior to administration; c. determining a second level of the clinical metric in the subject a period of time after administration; and d. assessing the change between the first and the second levels of the clinical metric, wherein the administration of the composition results in a change in the level of the clinical metric in the subject that is improved as compared to a baseline level, thereby treating HAE.
7. In a seventh embodiment, disclosed herein is a method of treating hereditary angioedema (HAE) in a human subject comprising: a. systemically administering to the human subject a LNP composition comprising a therapeutically effective amount of: i. an mRNA encoding a Cas nuclease, and ii. a guide RNA that targets the KLKB1 gene in the liver; b. determining a first level of a biosafety metric in the subject prior to administration; c. determining a second level of the biosafety metric in the subject a period of time after administration; and d. assessing the change between the first and the second levels of the biosafety metric, wherein the administration of the composition results in a change in a level of a biosafety metric in the subject that is acceptable as compared to a baseline level.
8. In an eighth embodiment, disclosed herein is a method of treating hereditary angioedema (HAE) in a human subject comprising: a. systemically administering to the human subject a LNP composition comprising an therapeutically effective amount of: i. an mRNA encoding a Cas nuclease, and ii. a guide RNA that targets the KLKB1 gene, wherein the administration of the composition results in a clinically significant improvement in a level of a clinical metric in the subject as compared to a baseline level of the clinical metric.
9. In a ninth embodiment, disclosed herein is a method for treating HAE in a human subject, comprising: a. systemically administering to the human subject a LNP composition comprising a therapeutically effective amount of: i. an mRNA encoding a Cas nuclease, and ii. a guide RNA that targets the KLKB1 gene, wherein the mRNA encoding a Cas nuclease and the guide RNA that targets the KLKB1 gene are administered at a combined dose of about 25 mg to about 75 mg.
10. In a tenth embodiment, disclosed herein is a method for treating HAE in a human subject, comprising: a. systemically administering to the human subject a LNP composition comprising a therapeutically effective amount of: i. an mRNA encoding a Cas nuclease, and ii. a guide RNA that targets the KLKB1 gene, wherein the mRNA encoding a Cas nuclease and the guide RNA that targets the KLKB1 gene are administered at a combined dose of about 50 mg to about 75 mg.
11. Provided herein is a method of any one of embodiments 1-10, wherein kallikrein protein level is reduced by at least 60% following administration of the composition. 12 Provided herein is a method of any one of embodiments 1-11, further comprising reducing kallikrein activity level by at least 60% following administration of the composition.
13. Provided herein is a method of any one of embodiments 1-4 or 6-12, wherein the guide RNA comprises a targeting sequence comprising the nucleotide sequence

(SEQ ID NO: 15)
GGAUUGCGUAUGGGACACAA.

14. Provided herein is a method of any one of embodiments 1-13, wherein the guide RNA further comprises a scaffold sequence comprising the nucleotide sequence

(SEQ ID NO: 387)
GUUUUAGAGCUAGAAAUAGCAAGUUAAAAUAAGGCUAGUCCGU
UAUCAACUUGAAAAAGUGGCACCGAGUCGGUGCUUUU.

15. Provided herein is a method of any one of embodiments 1-14, wherein the guide RNA is a modified guide RNA comprising or consisting of the nucleotide sequence mG*mG*mA*UUGCGUAUGGGACACAAGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 391), wherein m indicates a 2′-O-methyl modified nucleotide and * indicates a phosphorthioate linkage between nucleotides.
16. Provided herein is a method of any one of embodiments 1-15, wherein the subject is concurrently treated with HAE prophylaxis at the time of systemically administering to the human subject the LNP composition.
17. Provided herein is a method of embodiment 16, wherein the HAE prophylaxis comprises an agent selected from a C1 esterase inhibitor (C1-INH) replacement (e.g., recombinant or plasma-derived), an inhibitor of the B2 bradykinin receptor (B2R) (e.g., icatibant), a kallikrein inhibitor (e.g., ecallantide, lanadelumab, berotralstat), an attenuated androgen (e.g., danazol, oxandrolone, stanozolol), or an anti-fibrinolytic agent. In certain embodiments, the HAE prophylaxis comprises berotralstat. In certain embodiments, the HAE prophylaxis comprises danazol. In certain embodiments, the HAE prophylaxis comprises a recombinant or plasma-derived C1 esterase inhibitor (C1-INH) replacement. In certain embodiments, the HAE prophylaxis comprises icatibant. In certain embodiments, the HAE prophylaxis comprises ecallantide. In certain embodiments, the HAE prophylaxis comprises lanadelumab. In certain embodiments, the HAE prophylaxis comprises oxandrolone. In certain embodiments, the HAE prophylaxis comprises stanozolol.
18. Provided herein is a method of any one of embodiments 1-17, wherein the subject has an attack frequency of at least 2 confirmed HAE attacks in 90 days immediately prior to systemically administering to the human subject the LNP composition.
19. Provided herein is a method of any one of embodiments 1-17, wherein the subject has an attack frequency of at least 3 confirmed HAE attacks per 90 days immediately prior to systemically administering to the human subject the LNP composition.
20. Provided herein is a method of any one of embodiments 1-17, wherein the subject has an average attack frequency of at least 6 confirmed HAE attacks per 90 days immediately prior to systemically administering to the human subject the LNP composition.
21. Provided herein is a method of any one of embodiments 18-20, wherein the subject confirmed attack frequency is reduced by at least 50% for at least 90 days immediately after systemically administering to the human subject the LNP composition, optionally for at least 6 months immediately after systemically administering to the human subject the LNP composition.
22. Provided herein is a method of any one of embodiments 18-20, wherein the subject confirmed attack frequency is reduced by at least 80% for at least 90 days immediately after systemically administering to the human subject the LNP composition, optionally for 6 months immediately after systemically administering to the human subject the LNP composition.
23. Provided herein is a method of any one of embodiments 18-20, wherein the subject has no confirmed attacks for 90 days immediately after systemically administering to the human subject the LNP composition, optionally for 6 months immediately after systemically administering to the human subject the LNP composition.
24. Provided herein is a method of any one of embodiments 18-20, wherein the subject confirmed attack frequency is reduced by at least 80% on days 29-112 after systemically administering to the human subject the LNP composition, optionally on days 29-216 after systemically administering to the human subject the LNP composition.
25. Provided herein is a method of any one of embodiments 18-20, wherein the subject confirmed attack frequency is reduced by at least 90% on days 29-112 after systemically administering to the human subject the LNP composition, optionally on days 29-216 after systemically administering to the human subject the LNP composition.
26. Provided herein is a method of any one of embodiments 18-20, wherein the subject has no confirmed attacks on days 29-112 after systemically administering to the human subject the LNP composition, optionally on days 29-216 after systemically administering to the human subject the LNP composition.
27. Provided herein is a method of any one of embodiments 16-26, wherein prophylaxis is withdrawn concurrently with systemically administering to the human subject the LNP composition.
28. Provided herein is a method of any one of embodiments 16-27, wherein prophylaxis is withdrawn on or after day 36 after systemically administering to the human subject the LNP composition.
29. Provided herein is a method of any one of embodiments 1-28, wherein the subject has a reduced frequency of use of acute therapies for treatment of an HAE attack.
30. Provided herein is a method of any one of embodiments 1-29, wherein the subject has a reduced frequency of use of hospitalization related to an HAE attack as compared to control, e.g., the subject prior to treatment with the composition as a control.
31. Provided herein is a method of any one of embodiments 1-30, wherein the subject has a reduced severity of confirmed HAE attacks.
32. Provided herein is a method of any one of embodiments 1-31, wherein the subject has a reduced frequency of confirmed severe HAE attacks.
33. Provided herein is a method of any one of embodiments 1-31, wherein the subject has a reduced frequency of confirmed HAE attacks with laryngeal edema.
34. Provided herein is a method of any one of embodiments 1-33, wherein the subject has an improved quality of life, optionally as determined by the MOXIE Angioedema Quality of Life assessment.
35. Provided herein is a method of any one of embodiments 1-34, wherein the subject does not tolerate long term treatment with an attenuated androgen or has an average attack frequency of at least 1 per 90 days while on treatment with an attenuated androgen.
36. Provided herein is a method of any one of embodiments 1-35, wherein the subject is a child, a pregnant woman, a subject with liver disease, a subject with breast cancer, a subject with prostate cancer, a subject with cardiovascular risk factors, and a subject with hepatocellular carcinoma.
37. Provided herein is a method of embodiment 36, wherein the subject is a pregnant woman.
38. Provided herein is a method of any one of embodiments 1-37, wherein the subject is a woman of child-bearing potential.
39. Provided herein is a method of any one of embodiments 1-38, wherein the subject has had at least one confirmed HAE attack with laryngeal edema.
40. Provided herein is a method of any one of embodiments 1-28 wherein the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate.
41. Provided herein is a method of any one of embodiments 1-40, wherein the LNP comprises a PEG lipid.
42. Provided herein is a method of embodiment 41, wherein the PEG lipid comprises 1,2-dimyristoyl-rac-glycero-3-methylpolyoxyethylene glycol 2000.
43. Provided herein is a method of any one of embodiments 1-42, wherein the LNP comprises a neutral lipid.
44. Provided herein is a method of embodiment 43, wherein the neutral lipid comprises 1,2-distearoyl-sn-glycero-3-phosphocholine (DSPC).
45. Provided herein is a method of any one of embodiments 1-44, wherein the LNP composition has an N/P ratio of about 5-7.
46. Provided herein is a method of any one of embodiments 1-45, wherein the guide RNA and the mRNA encoding the Cas nuclease are present in a ratio ranging from about 5:1 to about 1:5 by weight.
47. Provided herein is a method of any one of embodiments 1-46, wherein the mRNA encodes a Cas9 nuclease.
48. Provided herein is a method of any one of embodiments 1-47, wherein the nRNA encodes S. pyogenes Cas9.
49. Provided herein is a method of any one of embodiments 1-48, wherein the mRNA encoding the Cas nuclease is codon-optimized.
50. Provided herein is a method of any one of embodiments 1-49, wherein the effective amount of mRNA encoding a Cas nuclease and the guide RNA that targets the KLKB1 gene is a combined dose of about 25-75 mg total RNA.
51. Provided herein is a method of any one of embodiments 1-50, wherein the effective amount of mRNA encoding a Cas nuclease and the guide RNA that targets the KLKB1 gene is a combined dose of about 25 mg total RNA.
52. Provided herein is a method of any one of embodiments 1-50, wherein the effective amount of mRNA encoding a Cas nuclease and the guide RNA that targets the KLKB1 gene is a combined dose of about 50 mg total RNA.
53. Provided herein is a method of any one of embodiments 1-50, wherein the effective amount of mRNA encoding a Cas nuclease and the guide RNA that targets the KLKB1 gene is a combined dose of about 75 mg total RNA.
54. Provided herein is a method of any one of embodiments 1-53, wherein administration of the composition reduces total plasma kallikrein protein level by 60%-95%, 60%-90%, 70%-95%, 70%-90%, or 80%-95% as compared to baseline total plasma kallikrein level before administration of the composition.
55. Provided herein is a method of any one of embodiments 1-54, wherein administration of the composition reduces plasma kallikrein activity by 60%-95%, 60%-90%, 70%-95%, 70%-90%, or 80%-95% as compared to baseline plasma kallikrein activity level before administration of the composition.
56. Provided herein is a method of any one of embodiments 54-55, wherein the plasma kallikrein level is determined at least 28 days after administration of the LNP composition.
57. Provided herein is a method of any one of embodiments 54-56, wherein the plasma kallikrein level is determined at 56 days after administration of the LNP composition.
58. Provided herein is a method of any one of embodiments 1-57, wherein administration of the composition results in a change in a level of a biosafety metric in the subject that is acceptable as compared to a baseline level of the biosafety metric.
59. Provided herein is a method of embodiment 58, wherein the biosafety metric is activated partial thromboplastin time (aPTT).
60. Provided herein is a method of embodiment 58 or 59, wherein the biosafety metric is alanine aminotransferase (ALT).
61. Provided herein is a method of any one of embodiments 58-60, wherein the biosafety metric is aspartate aminotransferase (AST).
62. Provided herein is a method of any one of embodiments 58-61, wherein the change in the biosafety metric resolves within 14 days.
63. Provided herein is a method of any one of embodiments 58-62, wherein the biosafety metric is grade 3 or less.
64. Provided herein is a method of any one of embodiments 1-63, wherein the composition is administered with a second therapeutic for treatment of HAE.
65. Provided herein is a method of embodiment 64, where in the second therapeutic is an HAE prophylaxis treatment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Schema showing pathways inhibited by C1-INH. C1-INH deficiency results in up-regulated release and buildup of bradykinin, activating endothelial cells, and leading angioedema.

FIG. 2: NTLA-2002 validated off-target and on-target editing in primary human hepatocytes (PHH).

FIG. 3: NTLA-2002, MAPKI targeting LNP-G018729, and non-targeting LNP-G000739 MAPK1 intronic locus-editing dose response in primary human hepatocytes.

FIG. 4: Percent editing of KLKB1, KLKB1 mRNA level, and serum prekallikrein levels as a percent of TSS vehicle control treated mice.

FIG. 5: Percent liver editing of KLKB1 and serum prekallikrein levels in humanized mice as a function of NTLA-2002 dosage.

FIG. 6: Vascular permeability assessed with the Evans blue vascular permeability assay in humanized mice treated with the LNP formulation comprising gRNA (G013901) and control.

FIG. 7: Plasma kallikrein activity levels in non-human primates treated with the LNP formulation comprising gRNA (G013901) vs control.

FIG. 8: Plasma kallikrein protein levels in non-human primates treated with the LNP formulation comprising gRNA (G013901) vs control.

FIG. 9: Plasma kallikrein protein levels in non-human primates treated with the LNP formulation comprising gRNA (G012267) vs control.

FIG. 10: Plasma kallikrein activity levels in non-human primates treated with the LNP formulation comprising gRNA (G012267) vs control.

FIG. 11: Diagram of clinical trial design describing dose escalation protocol.

FIG. 12: Mean percent change in plasma kallikrein levels in subjects following treatment with a single, 25 mg and 75 mg dose of NTLA-2002.

FIG. 13: Mean absolute kallikrein reduction in subjects following treatment with a single, 25 mg and 75 mg dose of NTLA-2002.

FIG. 14: Results from Cohort 1 and Cohort 2 of mean percent reduction of kallikrein in subjects following treatment with a single, 25 mg or 75 mg dose of NTLA-2002.

FIG. 15: Swim plot illustrating attack frequency and severity for individual patients following treatment with a single, 25 mg (Patients 1-3) or 75 mg (Patients 4-6) dose of NTLA-2002. The use of prophylaxis is indicated. Danazol was withdrawn at Day 151 from Patient 2. Berotralstat was withdrawn at day 112 from Patient 3.

FIGS. 16A and B: Results from (A) Cohort 1 (n=3) and (B) Cohort 2 (n=3) HAE attack rate percent change from baseline in subjects following treatment with a single, 25 mg or 75 mg dose of NTLA-2002.

FIG. 17: Mean absolute kallikrein reduction in subjects following treatment with a single, 25 mg, 50 mg, or 75 mg dose of NTLA-2002.

FIG. 18: Results from Cohorts 1, 2, and 3 of mean percent reduction of kallikrein in subjects following treatment with a single, 25 mg, 50 mg, or 75 mg dose of NTLA-2002.

FIG. 19: Swim plot illustrating attack frequency and severity for individual patients following treatment with a single, 25 mg (Patients 1-3), 50 mg (Patients 7-10), or 75 mg (Patients 4-6) dose of NTLA-2002. Withdrawal of prophylaxis is indicated by arrowhead.

FIGS. 20A and B: Updated results from (A) Cohort 1 (n=3) and (B) Cohort 2 (n=3) HAE attack rate percent change from baseline in subjects following treatment with a single, 25 mg or 75 mg dose of NTLA-2002.

FIG. 21: Investigator-confirmed monthly attack rate at baseline and during the 16-week primary observation period following treatment with a single 25 mg or 50 mg dose of NTLA-2002 and the mean % change from baseline from weeks 1-16 and weeks 5-16 in each cohort.

FIG. 22: Updated results of mean absolute kallikrein reduction in subjects following treatment with a single, 25 mg, 50 mg, or 75 mg dose of NTLA-2002.

FIG. 23: Updated results from Cohorts 1, 2, and 3 of mean percent reduction of kallikrein in subjects following treatment with a single, 25 mg, 50 mg, or 75 mg dose of NTLA-2002.

FIG. 24: Updated swim plot illustrating attack frequency and severity for individual patients following treatment with a single, 25 mg (Patients 1-3), 50 mg (Patients 7-10), or 75 mg (Patients 4-6) dose of NTLA-2002. Withdrawal of prophylaxis is indicated by arrowhead.

FIG. 25: Updated results from Cohort 1 (n=3) HAE attack rate percent change from baseline in subjects following treatment with a single, 25 mg dose of NTLA-2002.

FIG. 26: Updated results from Cohort 1 (n=4) HAE attack rate percent change from baseline in subjects following treatment with a single, 50 mg dose of NTLA-2002.

FIG. 27: Updated results from Cohort 1 (n=3) HAE attack rate percent change from baseline in subjects following treatment with a single, 75 mg dose of NTLA-2002.

FIG. 28: Mean absolute kallikrein activity values over study period to date for patients in Cohorts 1, 2, and 3.

FIG. 29: Mean percentage change from baseline of kallikrein activity values over study period to date for patients in Cohorts 1, 2, and 3.

FIG. 30: Spaghetti plot of kallikrein protein level percentage change from baseline for patients in Cohort 1 (25 mg).

FIG. 31: Spaghetti plot of kallikrein protein level percentage change from baseline for patients in Cohort 3 (50 mg).

FIG. 32: Spaghetti plot of kallikrein protein level percentage change from baseline for patients in Cohort 2 (75 mg).

FIG. 33: Spaghetti plot of kallikrein activity level percentage change from baseline for patients in Cohort 1 (25 mg).

FIG. 34: Spaghetti plot of kallikrein activity level percentage change from baseline for patients in Cohort 3 (50 mg).

FIG. 35: Spaghetti plot of kallikrein activity level percentage change from baseline for patients in Cohort 2 (75 mg).

FIG. 36: Correlation plot of plasma kallikrein protein concentration (percentage change from baseline) and kallikrein activity (percentage change from baseline) in HAE patients following administration of a single dose (25, 50 or 75 mg) of NTLA-2002.

FIG. 37: Geometric mean plasma concentration-time profiles of LP01 following single dose IV infusion of NTLA-2002 at the indicated doses. Available LP01 (Lipid A) pharmacokinetic data are depicted up through up to 76 hours from start of NTLA-2002 infusion.

FIG. 38: Angioedema attacks during screening, primary observation, and post-primary observation periods.

FIG. 39: Change over time in angioedema monthly attack rate.

### DETAILED DESCRIPTION

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended embodiments.

Before describing the present teachings in detail, it is to be understood that the disclosure is not limited to specific compositions or process steps, as such may vary. It should be noted that, as used in this specification and the appended embodiments, the singular form “a” “an” and “the” include plural references unless the context clearly dictates otherwise. Thus, for example, reference to “a conjugate” includes a plurality of conjugates and reference to “a cell” includes a plurality or population of cells and the like. As used herein, the term “include” and its grammatical variants are intended to be non-limiting, such that recitation of items in a list is not to the exclusion of other like items that can be substituted or added to the listed items.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

The term “about”, when used before a list or range, modifies each member of the list or range. The term “about” or “approximately” means an acceptable error for a particular value as determined by one of ordinary skill in the art, which depends in part on how the value is measured or determined. For example, in some embodiments, about includes within 2 standard deviations of the mean. In some embodiments of the invention, “about” includes ±10%, or optionally ±5% of the stated value. It is understood that some values are inherently variable, for example, the number of days in a month varies, and that for the purpose of monitoring, there are accepted tolerances within the art for monitoring at less frequent intervals.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 20 nucleotide nucleic acid molecule” means that 18, 19, or 20 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless stated otherwise, the following terms and phrases as used herein are intended to have the following meanings.

“mRNA” is used herein to refer to a polynucleotide comprising RNA or modified RNA that includes an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of a nucleic acid phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. In some embodiments, a polynucleotide is chemically synthesized or in vitro transcribed. A polynucleotide may be an mRNA, such as in vitro transcribed RNA comprising modified uridine. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. An RNA may comprise DNA or one or more deoxynucleosides or deoxynucleoside analogs.

“Guide RNA”, “gRNA”, and “guide” are used herein interchangeably to refer to an RNA such as a crRNA (also known as CRISPR RNA), or the combination of a crRNA and a trRNA (also known as tracrRNA), which targets a Cas nuclease to a genomic location. Cognate guide RNA structures for Cas nucleases such as Cas9 nucleases are known in the art. The crRNA and trRNA sequences of a guide RNA may be associated as a single RNA molecule (single guide RNA, sgRNA) or as, e.g. separate RNA molecules (dual guide RNA, dgRNA). The trRNA may be a naturally-occurring sequence, or a trRNA sequence with modifications or variations compared to naturally-occurring sequences. Guide RNAs can include modified RNAs as described herein.

As used herein, a “guide sequence” refers to a sequence within a guide RNA that is complementary to a target sequence and functions to direct a guide RNA to a target sequence for binding or modification (e.g., cleavage) by a Cas nuclease. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs, e.g., 18, 19, or 20 contiguous nucleotides in length of the guide target sequence. In some embodiments, the guide sequence and the target region may be 100% complementary or identical. In some embodiments, the guide sequence and the target region may contain 1 or 2 mismatches where the guide sequence comprises 20 nucleotides of the target region.

Target sequences for Cas proteins include both the positive and negative strands of genomic DNA (i.e., the sequence given and the sequence's reverse compliment), as a nucleic acid substrate for a Cas protein is a double-stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a “Cas nuclease” means a polypeptide or complex of polypeptides having RNA and DNA binding activity, such as a Cas9 nuclease, wherein the DNA binding activity is sequence-specific and depends on the sequence of a guide RNA. Exemplary Cas nucleases (and also “Cas protein”) include Cas cleavases, i.e., the Cas nuclease is a dsDNA cleavase that cleaves two strands of the DNA. As used herein, a “Cas9 nuclease” is a single-chain polypeptide, e.g. with dsDNA cleavase activity. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015). A Spy Cas9 dsDNA cleavase is specifically included herein. As used herein, delivery of a Cas nuclease (e.g., a Cas9 nuclease, or an S. pyogenes Cas9 nuclease) includes delivery of the polypeptide or mRNA. For example, the LNP composition described herein may comprise an mRNA encoding a Cas nuclease.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton, e.g., N1-methyl pseudouridine. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in, or accompanying, this disclosure can be a uridine or a modified uridine.

As used herein, “treatment” refers to any administration or application of a therapeutic for disease or disorder in a subject, and includes inhibiting the disease, relieving one or more symptoms of the disease, curing the disease, reducing HAE attack frequency, reducing HAE attack severity, improving one or more clinical metrics described herein (e.g., a suitable Quality of Life (QoL) questionnaire, e.g., MOXIE Angioedema Quality of Life (AE-QoL) questionnaire), or preventing reoccurrence of one or more symptoms of the disease. In some embodiments, treatment of HAE comprises reducing HAE attack frequency. In some embodiments, treatment of HAE may comprise a substantial reduction or knockdown in expression of the KLKB1 gene, e.g., a durable reduction by at least 60% of total plasma kallikrein protein level, thereby substantially reducing or eliminating the production of kallikrein protein associated with HAE. In some embodiments, treatment of HAE may comprise a substantial reduction or knockdown of expression of the KLKB1 gene, e.g., a durable reduction by at least 80% of total kallikrein protein level, thereby substantially reducing or eliminating the production of kallikrein protein associated with HAE.

As used herein, “concurrently treated” and the like, is understood as treatment with two or more agents that, based on pharmacokinetic properties such as half-life, are likely to have pharmacodynamic effect. As used herein, “concurrently treated” at the time of systemically administering to the human subject the LNP composition in regard to treatment with an HAE propylaxis agent is understood as being within five half-lives from the time of dosing of the HAE prophylaxis agent. Half-lives for such agents are known in the art and exemplary values are provided herein for various agents. For especially long-acting agents, washout periods may be defined during which the long-acting agent is likely to have pharmacodynamic effect. Systemically administering to the human subject the LNP composition during the washout period in regard to treatment with an HAE prophylaxis agent would be understood as concurrent treatment.

As used herein, “knockdown” refers to a decrease in expression of a particular gene product (e.g., KLKB1 or kallikrein), in a sample, e.g., in a cell, population of cells, tissue, organ, or fluid; wherein the sample is optionally a subject sample; by gene editing. In some embodiments, gene editing can be assessed by sequence, e.g., next generation sequencing (NGS). Expression may be decreased by at least 60%, 65%, 70%, 75%, 80%, 85%, 90%, 95%, or to below the level of detection of the assay as compared to a suitable control, e.g., the subject's baseline or prior to treatment. Methods for measuring knockdown of mRNA are known and include sequencing of mRNA isolated from a tissue or cell population of interest, e.g., in liver hepatocytes. Knockdown of a protein can be measured by detecting the amount of the protein from a tissue, cell population, or fluid of interest, e.g., plasma in non-human primates, plasma in humans, and serum in mouse. Flow cytometry analysis is a known method for measuring knockdown of protein expression. For secreted proteins, knockdown may be assessed in a fluid such as tissue culture media or blood, or serum or plasma derived therefrom. Plasma protein levels can be measured by quantitative assay, e.g., ELISA, and used to detect knockdown. In some embodiments, “knockdown” may refer to some loss of expression of a particular gene product, for example a decrease in the amount of full-length, wild-type mRNA transcribed or translated into full-length protein, or a decrease in the amount of protein expressed by a population of cells. In certain embodiments, the sample may be treated, e.g., to activate the product in the sample prior to performing the enzymatic assay. In some embodiments, “knockdown” may refer to some loss of expression of a particular gene product, e.g., kallikrein.

As used herein, “durable” in the context of a kallikrein knockdown (e.g., a durable knockdown) or “durably” reducing expression of the gene (e.g. the KLKB1 gene) refers to a lasting effect, such as a lasting knockdown or a lasting reduction in gene expression. In some embodiments, a durable knockdown in plasma kallikrein refers to a reduction (level relative to baseline) that is maintained, as measured at post-dose time points to reach maximal reduction at ≥~28 days e.g., at about 56 days after administration of the LNP composition, e.g., for at least 6 months, 9 months, 1 year, 2 years, 3 years, 4 years, 5 years, or more. In some embodiments, the level maintained can vary. In some embodiments, the reduction correlates to a desired clinical efficacy for the disorder being treated. The level of reduction to achieve a desired clinical efficacy for a given disorder, e.g., HAE, is known in the art. For example, a reduction by at least 60%, 65%, 70%, 75%, 80%, 85%, 90%, 95%, or more correlates to a desired clinical efficacy for a specific disorder. For example, a reduction by at least 60%, 65%, 70%, 75%, 80%, 85%, 90%, 95%, or more correlates to a desired clinical efficacy for HAE.

As used herein, an “effective amount” or a “therapeutically effective amount” refers to an amount of mRNA encoding a Cas nuclease and a guide RNA that is capable of producing a therapeutic effect, including reducing HAE attack frequency, reducing plasma kallikrein activity, reducing total plasma kallikrein level, e.g., by at least 60% in a subject relative to a baseline total plasma kallikrein level or reduces total plasma kallikrein to less than about 20 μg/mL after administration of the mRNA encoding the Cas nuclease and the guide RNA. For instance, an LNP composition may comprise an effective amount of the mRNA encoding the Cas nuclease and the guide RNA, e.g., a guide RNA that targets KLKB1 (the combined or total RNA). In some embodiments, an LNP composition delivers the mRNA encoding the Cas9 nuclease and the single guide RNA, which can comprise an “effective amount” of RNA measured as total RNA. In some embodiments, an effective amount of the mRNA encoding the Cas9 nuclease and the single guide RNA reduces serum kallikrein level by at least 60%, or by 60%-95%, 60%-90%, 70%-95%, 70%-90%, or 80%-95% in a subject relative to a baseline plasma kallikrein level, e.g., total plasma kallikrein level. In some embodiments, an effective amount of the mRNA encoding the Cas9 nuclease and the single guide RNA reduces total plasma kallikrein to less than about 20 μg/mL, less than about 15 μg/mL, or less than about 10 μg/mL after administration of the mRNA encoding the Cas9 nuclease and the single guide RNA that targets KLKB1.

As used herein, a “biosafety metric” refers to a clinical metric used to monitor for safety events associated with administration of the LNP composition described herein to a human subject. A biosafety metric may allow for a determination of a safety event, including an adverse event (NCI-CTCAE Grade 3 or higher), a serious adverse event, an adverse event of special interest, or a treatment-emergent adverse event (CTCAE Grade 3 or higher), as described herein. Guidelines for defining the severity of a safety event (e.g., adverse event) are known in the art (e.g., Common Terminology Criteria for Adverse Events (CTCAE) including National Cancer Institute (NCI)-CTCAE, version 5.0). In some embodiments, the biosafety metric is a liver enzyme level, e.g., ALT, AST. In some embodiments, the biosafety metric is aPTT. In some embodiments, an adverse event is an infusion-related reaction (IRR), e.g., flushing, dyspnea, chest pain, syncope, rash, increased heart rate, and/or facial edema. In some embodiments, a level of a biosafety metric is measured prior to administration of the LNP composition. In some embodiments, a level of a biosafety metric is measured following administration of the LNP composition. In some embodiments, a level of a biosafety metric is measured prior to and following the administration of the LNP composition, thereby allowing for a comparison of the levels of the biosafety metric before and after treatment with the LNP composition to determine a change, e.g., an acceptable change. As used herein, an “acceptable” change refers to a change in biosafety metric level, wherein the resulting change does not constitute a safety event (e.g., an adverse event (NCI-CTCAE Grade 3 or higher), a serious adverse event, an adverse event of special interest, a treatment-emergent adverse event (CTCAE Grade 3 or higher)), or an event that otherwise requires discontinuation of the study drug as determined by a clinician. In some embodiments, a level of a biosafety metric measured prior to administration of the LNP composition can serve as a baseline for comparison against one or more levels of the biosafety metric measured following administration of the LNP composition (e.g., measurements taken at specific intervals following administration can be compared against baseline level). In some embodiments, a baseline is the last available measurement taken prior to administration of the LNP composition. In some embodiments, the biosafety metric is not compared against a baseline if the value alone can be used determine a safety event.

As used herein, “safe and well-tolerated” refers to the absence of a safety event as described herein, e.g., the absence of: an adverse event (NCI-CTCAE Grade 3 or higher), a serious adverse event, a treatment-emergent adverse event (CTCAE Grade 3 or higher), or an event that otherwise requires discontinuation of the study drug as determined by a clinician.

In some embodiments, “safe and well-tolerated” includes patients who experience an adverse event of NCI-CTCAE Grade 3 or higher that is unrelated to administration of the composition described herein, e.g., LNP composition comprising an effective amount of an mRNA encoding Cas nuclease and a guide RNA targeting KLKB1, or resolves, e.g., with or without intervention, after an acceptable period of time for said event.

In some embodiments, an adverse event of special interest includes, e.g., infusion-related reaction (IRR) (e.g., requiring treatment or discontinuation of infusion, or Grade 3 or higher); incidence of cytokine release syndrome; abnormal coagulation findings defined by clinically relevant abnormal bleeding or thrombotic or hemorrhagic incidence or CTCAE ≥Grade 2 abnormal blood test results; acute liver injury evidenced by CTCAE ≥Grade 2 elevation in ALT, CTCAE ≥Grade 2 elevation in AST, CTCAE ≥Grade 2 elevation in total bilirubin, CTCAE ≥Grade 2.

In some embodiments, an adverse event is any untoward medical occurrence in a subject administered a study drug or has undergone study procedures and which does not necessarily have a causal relationship with the treatment. In some embodiments, an adverse event is an unintended sign (including an abnormal laboratory finding), symptom, or disease temporally associated with the treatment, whether or not related to the medicinal (investigational) product. In some embodiments, an adverse event that induces clinical signs or symptoms. In some embodiments, an adverse event requires active intervention. In some embodiments, an adverse event requires interruption or discontinuation of the treatment. In some embodiments, an adverse event is an abnormality that is clinically significant in the opinion of the investigator. Grading criteria for adverse events are known in the art, such as, e.g., Common Terminology Criteria for Adverse Events (CTCAE), including National Cancer Institute (NCI)-CTCAE.

Biosafety metrics include known laboratory assessments relating generally to, e.g., coagulation, hematology, clinical chemistry, urinalysis, and other bioanalytical assessments (e.g., cytokines, complement). Particular biosafety metrics include, but are not limited to: liver enzyme levels (e.g., an elevation in alanine aminotransferase (ALT) or aspartate aminotransferase (AST) >5×ULN for more than 4 weeks after administration of a treatment, ALT or AST >3×ULN and total bilirubin >2×ULN (Hy's law) after administration of a treatment), levels of activated partial thromboplastin time (aPTT) (e.g., an elevation in aPTT) >5×ULN for more than 4 weeks after administration of a treatment), levels of prothrombin time (PT), levels of thrombin generation time (TGT) (e.g., peak height, lag time, or endogenous thrombin potential), levels of fibrinogen, prothrombin international normalized (INR) ratio, levels, levels of d-dimer, laboratory parameters consistent with disseminated intravascular coagulation, changes in hematology values (e.g. a CTCAE ≥Grade 2 abnormal blood test results after administration of a treatment), changes in chemistry values, changes in coagulation, changes in urinalysis, levels of Glutamate Dehydrogenase, levels of C-reactive protein, levels of complement (C3a, C5a, Bb), levels of cytokines (GM-CSF, INF-γ, IL-1β, IL-4, IL-5, IL-6, IL-8, IL-10, IL-13, IL-23, TNF-α, IL-17, MCP-1), acute liver injury (e.g. a CTCAE >Grade 2 elevations in ALT, AST, total bilirubin or clinically relevant symptoms/signs of liver injury after administration of a treatment), and changes in a 12-Lead Electrocardiogram, Additional biosafety metrics, including those associated with administration of an LNP composition, are known in the art. Similarly, acceptable levels or changes in the biosafety metrics are known in the art and may be assessed by routine methods, e.g., by a clinician or laboratory.

As used herein, a “clinical efficacy metric” refers to a metric used to assess amelioration of disease in a human subject treated with the LNP composition described herein. In some embodiments, a level of a clinical efficacy metric is measured following administration of the LNP composition. In some embodiments, a level of a clinical efficacy metric is measured prior to and following the administration of the LNP composition, thereby allowing for a comparison of the levels of the clinical efficacy metric before and after treatment with the LNP composition. In some embodiments, the efficacy metric is determined over time, e.g., attack frequency per month, e.g., average attack frequency per month over a given period of time, e.g., three-month period (e.g., reduction in HAE attack number, breakthrough attack, number or proportion of patients who are attack-free over a given period of time, frequency of hospitalization related to an HAE attack, or frequency of use of acute therapies related to an HAE attack). In some embodiments, a level of a clinical metric measured prior to administration of the LNP composition can serve as a baseline or control for comparison against one or more levels of the clinical metric measured following administration of the LNP composition, either immediately after administration of the LNP, or after an interval to allow for the onset of action of the LNP composition, e.g., measured starting at 29 days following the administration of the LNP composition. In some embodiments, a baseline is the last available measurement taken prior to administration of the LNP composition.

For disorders characterized by HAE attacks, clinical efficacy metrics include, but are not limited to: a decrease in total plasma kallikrein protein level (e.g. a 60% decrease of total plasma kallikrein protein as measured, e.g., by ELISA after administration of a treatment), a decrease in plasma total kallikrein activity (e.g., at least a 60% decrease in plasma kallikrein activity). Additional clinical efficacy metrics, including HAE attack frequency (e.g., reduction in HAE attack number, breakthrough attack, number or proportion of patients who are attack-free over a given period of time, frequency of hospitalization related to an HAE attack, or frequency of use of acute therapies related to an HAE attack), and HAE attack severity. Methods for confirming an HAE attack and grading of HAE attacks are provided herein. Criteria for a “confirmed” HAE attack are provided below. In some embodiments, the “confirmed” HAE attack is one that a physician determines.

Similarly, “clinically significant improvement” in a clinical efficacy metric, i.e., levels or changes in clinical efficacy metric(s) indicative of amelioration of disease, including HAE attack frequency, e.g., confirmed HAE attack frequency, and HAE attack severity, are known in the art and may be assessed by routine methods, e.g., by a health care professional or laboratory. For example, plasma kallikrein protein level, e.g., total plasma kallikrein protein level, is a clinical efficacy metric for HAE. A “clinically significant improvement” in this clinical efficacy metric for the treatment of HAE includes at least 60%, 70%, 80%, 85%, 90%, or 95% reduction of total plasma kallikrein protein level, e.g., after treatment, e.g., immediately after treatment or starting at a predefined period after treatment, as compared to baseline, e.g., prior to treatment, with the LNP composition described here. For example, reduction in HAE attack frequency, e.g., confirmed attack frequency per month, e.g., average confirmed attack frequency per month over a 3-month or 6-month period, is also a clinical efficacy metric for HAE. A “clinically significant improvement” in this clinical efficacy metric for the treatment of HAE includes at least 40%, 50%, 60%, 70%, 80%, 85%, 90%, or 95% reduction of attack frequency after treatment as compared to baseline, e.g., prior to treatment, e.g., with the LNP composition described herein.

As used herein, the term “lipid nanoparticle” (LNP) refers to a particle that comprises a plurality of (i.e., more than one) lipid molecules physically associated with each other by intermolecular forces and encapsulating RNA. See, e.g., WO2017173054 and WO2019067992, the contents of which are hereby incorporated by reference in their entirety.

As used herein, the phrase “pharmaceutically acceptable” means that which is useful in preparing a pharmaceutical composition that is generally non-toxic and is not biologically undesirable and that are not otherwise unacceptable for pharmaceutical use. In certain embodiments, pharmaceutically acceptable composition is sterile. In certain embodiments, pharmaceutically acceptable composition is non-pyrogenic.

As used herein, systemic administration may be by intravenous infusion. “Infusion” refers to an active administration of one or more agents with an infusion time of, for example, approximately 2 hours. In some embodiments, the infusion time is completed within 6 hours of opening of the vial(s) containing the LNP composition. In some embodiments, an LNP, e.g., comprising an mRNA encoding a Cas9 nuclease described herein and a sgRNA described herein is systemically administered to a human subject.

As used herein, “infusion prophylaxis” refers to a regimen administered to a subject before treatment (e.g., comprising administration of an LNP) comprising, for example, oral dexamethasone 8 mg or equivalent 8 to 24 hours prior to the LNP composition; and intravenous steroid (e.g., dexamethasone 10 mg); intravenous H1 blocker (e.g., diphenhydramine 50 mg) or oral H1 blocker (e.g., cetirizine 10 mg); and intravenous or oral H2 blocker (e.g., famotidine 20 mg) administration about 1-2 hours prior to administration of the LNP composition.

### I. Compositions Targeting a Gene

Disclosed herein are methods for editing a gene of interest (e.g., KLKB1) in the liver of a human subject, modifying the gene in a hepatocyte of the subject, or treating a disease, as well as related compositions, including compositions for use in such methods. In general, disclosed herein are LNP compositions comprising an mRNA encoding a Cas nuclease, e.g., Cas9, and a guide RNA that targets a gene, e.g., a guide RNA that targets the KLKB1 gene. The subjects treated with such methods and compositions may have wild-type or non-wild type gene of interest sequences, such as, for example, subjects with HAE.

In some embodiments, methods disclosed herein comprise systemic administration of a lipid nanoparticle system for in vivo liver delivery of a guide RNA and an mRNA encoding a Cas nuclease.

1. Guide RNA (gRNAs)

The single guide RNA used in the disclosed methods and compositions comprises a guide sequence targeting a gene of interest (e.g., the KLKB1 gene) comprising at least 18 contiguous nucleotides, preferably 20 nucleotides of the nucleotide sequence

(SEQ ID NO: 15)
GGAUUGCGUAUGGGACACAA.

In the case of a sgRNA, the above Guide Sequences may further comprise additional nucleotides to form a sgRNA, e.g., with the following exemplary nucleotide sequence 3′ of the 3′ end of the Guide Sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 387) in 5′ to 3′ orientation, providing a sgRNA with the nucleotide sequence GGAUUGCGUAUGGGACACAAGUUUUAGAGCUAGAAAUAGCAAGUUAAAAUAA GGCUAGUCCGUUAUCAACUUGAAAAAGUGGCACCGAGUCGGUGCUUUU (SEQ ID NO: 388). In alternative embodiments, the sgRNA can include the following exemplary nucleotide sequence 3′ of the 3′ end of the Guide Sequence GUUUUAGAGCUAGAAAUAGCAAGUUAAAAUAAGGCUAGUCCGUUAUCACGAA AGGGCACCGAGUCGGUGC (SEQ ID NO: 389). In alternative embodiments, the sgRNA can include the following exemplary nucleotide sequence 3′ of the 3′ end of the Guide Sequence

(SEQ ID NO: 390)
GUUUUAGAGCUAGAAAUAGCAAGUUAAAAUAAGGCUAGUCCGU
UAUCAAAAUGGCACCGAGUCGGUGC.

In some embodiments, the sgRNA is modified. In some embodiments, the sgRNA comprises 5′ end modification, 3′ end modification, or 5′ and 3′ end modification. In some embodiments, the sgRNA comprises the modification pattern shown below in SEQ ID NO: 391, wherein the modified sgRNA comprises the following sequence: mG*mG*mA*UUGCGUAUGGGACACAAGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 391), wherein A, C, G, and U indicate RNA nucleosides (2′-OH) adenine, cytosine, guanine, and uridine, respectively; mA, mC, mG, and mU indicate 2′-O-methyl modified adenine, cytosine, guanine, and uridine, respectively; and * indicates a phosphorothioate linkage.

In some embodiments, the gRNA comprises a guide sequence that directs a Cas9 nuclease, e.g., a SpyCas9 nuclease, to a target DNA sequence. The sgRNA comprising 18, 19, or 20 contiguous nucleotides of the targeting sequence

(SEQ ID NO: 15)
GGAUUGCGUAUGGGACACAA.

The single guide RNAs provided herein is useful for recognizing a target sequence in the KLKB1 genomic locus by hybridization of the targeting sequence to the target sequence. For example, the gene of interest target sequence is recognized and cleaved by a provided Cas9 cleavase comprising a guide RNA. Thus, a Cas9 cleavase, such as a Spy Cas9 cleavase, is directed by a guide RNA to a target sequence in the KLKB1 genomic locus, where the guide sequence of the guide RNA hybridizes with the target sequence and the Cas9 cleavase, such as a Spy Cas9 cleavase, cleaves within the target sequence in the genomic locus.

2. Modification of gRNAs

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.”

A modified guide RNA may comprise nucleosides or nucleoside analogs which have nitrogenous heterocyclic bases or base analogs linked together along a backbone, including conventional RNA, DNA, mixed RNA-DNA, and polymers that are analogs thereof. A guide RNA “backbone” can be made up of a variety of linkages, including one or more of sugar-phosphodiester linkages, phosphorothioate linkages, or combinations thereof. Sugar moieties of a guide RNA can be ribose, deoxyribose, or similar compounds with substitutions, e.g., 2′ methoxy. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion of chemical modifications for a guide RNA, see The Biochemistry of the Nucleic Acids 5-36, Adams et al., ed., 11^{th} ed., 1992). Guide RNAs can comprise only conventional RNA or DNA sugars, bases and linkages, or can include both conventional components and substitutions (e.g., conventional bases with 2′ methoxy linkages, or polymers containing both conventional bases and one or more base analogs). RNA and DNA have different sugar moieties and can differ by the presence of uracil or analogs thereof in RNA and thymine or analogs thereof in DNA.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a nonbridging phosphate oxygen, generating a PS bond in lieu of a phosphodiester bond:

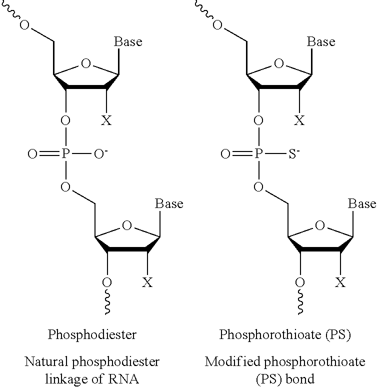

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide, for example.

In some embodiments, the guide RNA comprises a modified sgRNA. In some embodiments, the sgRNA comprises the modification pattern shown in SEQ ID NO: 391, where N is any natural or non-natural nucleotide, and where the totality of the N's comprise a guide sequence that directs a nuclease to a target sequence.

In some embodiments, the guide RNA comprises a sgRNA shown in any one of Table 2 of WO2021158858A1, the contents of which are hereby incorporated in their entirety. In some embodiments, the guide RNA comprises a sgRNA comprising any one of the guide sequences of Table 1 of WO2021158858A1, the contents of which are hereby incorporated in their entirety, and wherein the guide sequence may be modified as shown in SEQ ID NO: 391. In some embodiments, the guide RNA comprises a sgRNA shown in any one of Table 24 or Table 25.

### 3. RNA Comprising an Open Reading Frame Encoding a Cas9 Nuclease

Any RNA comprising an ORF encoding a Cas9 nuclease such as an S. pyogenes Cas9, disclosed herein may be combined in a composition or method with the sgRNAs disclosed herein. In any of the embodiments set forth herein, the nucleic acid comprising an open reading frame encoding a Cas9 nuclease may be an mRNA.

Codons that Improve Protein Expression or that Correspond to Highly Expressed tRNAs; Exemplary Codon Sets

In some embodiments, the nucleic acid comprises an ORF having codons that improve protein expression in a mammal, such as a human. In further embodiments, the nucleic acid comprises an ORF having codons that improve protein expression in an organ, such as the liver, of a human. In further embodiments, the nucleic acid comprises an ORF having codons that improve protein expression in a cell type, such as a hepatocyte, of a human. An improvement in protein expression in a hepatocyte, liver, or human, etc., can be determined relative to the extent of translation wild-type sequence of the ORF, or relative to an ORF having a codon distribution matching the codon distribution of the organism from which the ORF was derived or the organism that contains the most similar ORF at the amino acid level, such as S. pyogenes, S. aureus, or another prokaryote as the case may be for prokaryotically-derived Cas nucleases, such as the Cas nucleases from other prokaryotes described below. Alternatively, in some embodiments, an improvement in protein expression for a Cas9 sequence in a mammal, cell type, organ of a mammal, human, organ of a human, etc., is determined relative to translation of an ORF with the sequence of SEQ ID NO: 393 (Table 23) with all else equal, including any applicable point mutations, heterologous domains, and the like. Codons useful for increasing expression in a human, including the human liver and human hepatocytes, can be codons corresponding to highly expressed tRNAs in the human liver/hepatocytes, which are discussed in Dittmar KA, PLos Genetics 2(12): e221 (2006). In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian liver, such as a human liver. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian hepatocyte, such as a human hepatocyte.

Alternatively, codons corresponding to highly expressed tRNAs in an organism (e.g., human) in general may be used. Various codon usage schemes are known in the art (see, e.g., WO2019067910, WO2020198641) and can be applied to ORF provided herein.

### Exemplary Sequences

In some embodiments, the ORF encoding the Cas nuclease comprises a sequence with at least 95%, 96%, 97%, 98%, 99%, 99.5%, or 100% identity to any of SEQ ID NOs: 393-407, 392, and 408.

In some embodiments, the mRNA comprises an ORF encoding a Cas nuclease, wherein the Cas nuclease comprises an amino acid sequence with at least 95%, 96%, 97%, 98%, 99%, 99.5%, or 100% identity to any of SEQ ID NOs: 406-407.

In some embodiments, the ORF encoding the Cas nuclease comprises a sequence that is codon optimized according to the sequences provided in Table 3 from any of SEQ ID NOs: 393-407, 392, and 408 or a sequence with at least 95%, 96%, 97%, 98%, 99%, 99.5%, or 100% identity to any of SEQ ID NOs: 393-407, 392, and 408.

As used herein, a first sequence is considered to “comprise a sequence with at least X % identity to” a second sequence if an alignment of the first sequence to the second sequence shows that X % or more of the positions of the second sequence in its entirety are matched by the first sequence. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

Additional Features of RNA, mRNAs, and ORFs

Any of the additional features described herein may be combined to the extent feasible with any of the embodiments described above.

### Encoded Cas Nuclease

In some embodiments, the Cas9 nuclease has cleavase activity, which can also be referred to as double-strand endonuclease activity. Examples of Cas9 nucleases include those of the type II CRISPR systems of S. pyogenes, S. aureus, and other prokaryotes, and modified (e.g., engineered or mutant) versions thereof. See, e.g., US20160312198; US 20160312199.

### Poly-A Tail

In some embodiments, the RNA (e.g., mRNA) further comprises a poly-adenylated (poly-A) tail. In some instances, the poly-A tail is “interrupted” with one or more non-adenine nucleotide “anchors” at one or more locations within the poly-A tail, e.g., when it is encoded by a plasmid. The poly-A tails may comprise at least 8 consecutive adenine nucleotides, and in some embodiments, the poly-A tail also comprises one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the polynucleotide (e.g., mRNA) described herein may comprise consecutive adenine nucleotides located 3′ to nucleotides encoding a Cas nuclease or a sequence of interest. In some instances, the poly-A tails on mRNA comprise non-consecutive adenine nucleotides located 3′ to nucleotides encoding a Cas nuclease or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence (SEQ ID NO: 410) in a plasmid may result in up to 100 poly-A sequence (SEQ ID NO: 410) in the transcribed mRNA. In some embodiments, an encoded poly-A tail is about 90 or 95 nucleotides in length. In some embodiments, the poly-A tail is not encoded in the plasmid, and is added by PCR tailing or enzymatic tailing, e.g., using E. coli poly(A) polymerase.

### UTRs; Kozak Sequences

In some embodiments, the RNA encoding a Cas nuclease (e.g., mRNA) comprises a 5′ UTR, a 3′ UTR, or 5′ and 3′ UTRs. In some embodiments, the RNA (e.g., mRNA) comprises at least one UTR from Hydroxysteroid 17-Beta Dehydrogenase 4 (HSD17B4 or HSD), e.g., a 5′ UTR from HSD. In some embodiments, the RNA (e.g., mRNA) comprises at least one UTR from a globin mRNA, for example, human alpha globin (HBA) mRNA, human beta globin (HBB) mRNA, or Xenopus laevis beta globin (XBG) mRNA. In some embodiments, the polynucleotide (e.g., mRNA) comprises a 5′ UTR, 3′ UTR, or 5′ and 3′ UTRs from a globin mRNA, such as HBA, HBB, or XBG. In some embodiments, the polynucleotide (e.g., mRNA) comprises a 5′ UTR from bovine growth hormone, cytomegalovirus (CMV), mouse Hba-al, HSD, an albumin gene, HBA, HBB, or XBG. In some embodiments, the polynucleotide (e.g. mRNA) comprises a 3′ UTR from bovine growth hormone, cytomegalovirus, mouse Hba-al, HSD, an albumin gene, HBA, HBB, or XBG. In some embodiments, the polynucleotide (e.g., mRNA) comprises 5′ and 3′ UTRs from bovine growth hormone, cytomegalovirus, mouse Hba-al, HSD, an albumin gene, HBA, HBB, XBG, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the polynucleotide (e.g., mRNA) comprises 5′ and 3′ UTRs that are from the same source, e.g., a constitutively expressed mRNA such as actin, albumin, or a globin such as HBA, HBB, or XBG.

In some embodiments, the polynucleotide (e.g., mRNA) comprises a Kozak sequence. Kozak sequences are known in the art. The Kozak sequence can affect translation initiation and the overall yield of a polypeptide translated from a nucleic acid. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is gccgccRccAUGG (SEQ ID NO: 392) with zero mismatches or with up to one, two, three, or four mismatches to positions in lowercase.

### Modified Nucleotides

In some embodiments, the mRNA comprising an ORF encoding a Cas9 nuclease comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof. In some embodiments the modified uridine is 5-methoxyuridine. In some embodiments the modified uridine is 5-iodouridine. In some embodiments the modified uridine is pseudouridine. In some embodiments the modified uridine is N1-methyl-pseudouridine. In some embodiments, the modified uridine is a combination of pseudouridine and Ni-methyl-pseudouridine. In certain embodiments, the mRNA comprises Ni-methyl-pseudouridine at all uridine positions.

In some embodiments, at least 80%, 85%, 90%, 95%, 98%, or 99%; or 100% of the uridine positions in the nucleic acid are modified uridines. In some embodiments, 80-95% or 90-100% of the uridine positions in the nucleic acid are modified uridines, e.g., N1-methyl pseudouridine, pseudouridine, or a combination thereof. In some embodiments, 80-95% or 90-100% of the uridine positions in the nucleic acid are pseudouridine. In some embodiments, 80-95% or 90-100% of the uridine positions in the nucleic acid are N1-methyl pseudouridine.

### 5′ Cap

In some embodiments, mRNA comprising an ORF encoding a Cas nuclease (e.g., Cas9) comprises a 5′ cap, such as a Cap0, Cap1, or Cap2. A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115.

A cap can be included in an RNA co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’cap analogs 7-methyl(3′-O-methyl)GpppG and 7-methyl(3′deoxy)GpppG,” RNA 7: 1486-1495. The ARCA structure is shown below.

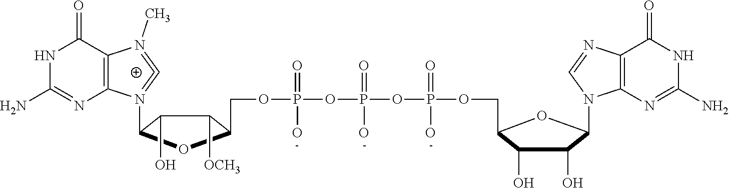

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

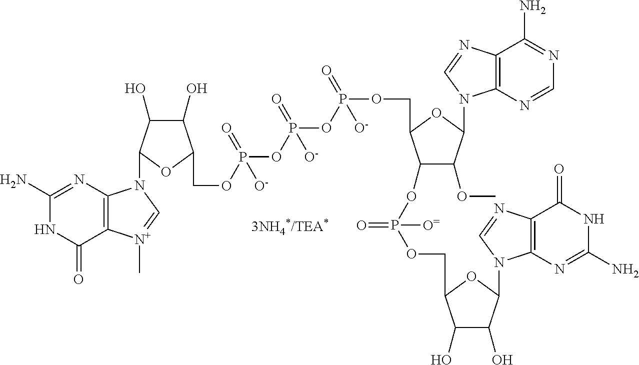

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give CapO, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### 4. Delivery of Nucleic Acid Compositions

In some embodiments, a method of inducing a double-stranded break (DSB) or gene editing within KLKB1 is provided comprising systemically administering a composition comprising a guide RNA as described herein. In some embodiments, the guide RNA is systemically administered to induce a DSB in KLKB1. The guide RNA is systemically administered together with an mRNA encoding a Cas9 nuclease, e.g., S. pyogenes Cas9. In particular embodiments, the guide RNA is chemically modified. In some embodiments, the guide RNA and the RNA encoding a Cas9 nuclease are systemically administered in an LNP described herein, such as an LNP comprising an ionizable lipid, referred to herein as Lipid A (which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). In further embodiments, the LNP comprises a lipid component that includes Lipid A, a helper lipid (e.g., cholesterol), a stealth lipid (e.g., a PEG lipid, such as PEG2k-DMG), and a neutral lipid (e.g., DSPC).

In some embodiments, a method of inducing a double-stranded break (DSB) within the KLKB1 gene is provided comprising systemically administering a LNP composition comprising a single guide RNA, such as a chemically modified single guide RNA. The guide RNA is systemically administered together with an mRNA described herein encoding a Cas9 nuclease e.g., an S. pyogenes Cas9. In particular embodiments, the guide RNA is chemically modified. In some embodiments, the guide RNA and the RNA encoding a Cas nuclease are systemically administered in an LNP described herein, such as an LNP comprising a Lipid A, a helper lipid (e.g., cholesterol), a stealth lipid (e.g., a PEG lipid, such as PEG2k-DMG), and a neutral lipid (e.g., DSPC).

In some embodiments, a method of modifying the KLKB1 gene is provided comprising systemically administering a composition comprising a single guide RNA, such as a chemically modified single guide RNA. The guide RNA is systemically administered together with an mRNA described herein encoding a Cas9 nuclease, e.g., an S. pyogenes Cas9. In particular embodiments, the guide RNA is chemically modified. In some embodiments, the guide RNA and the RNA encoding a Cas nuclease are systemically administered in an LNP described herein, such as an LNP comprising a Lipid A, a helper lipid (e.g., cholesterol), a stealth lipid (e.g., a PEG lipid, such as PEG2k-DMG), and optionally a neutral lipid (e.g., DSPC).

In some embodiments, a method of treating HAE is provided comprising systemically administering a composition comprising a single guide RNA targeting the KLKB1 gene. The guide RNA is systemically administered together with an mRNA described herein encoding a Cas9 nuclease, e.g., an S. pyogenes Cas9. In particular embodiments, the guide RNA is chemically modified. In some embodiments, the guide RNA and the nucleic acid encoding a Cas nuclease are systemically administered in an LNP described herein, such as an LNP comprising Lipid A, a helper lipid (e.g., cholesterol), a stealth lipid (e.g., a PEG lipid, such as PEG2k-DMG), and a neutral lipid (e.g., DSPC).

In some embodiments, a method of reducing plasma kallikrein protein level or activity is provided comprising systemically administering a guide RNA targeting KLKB1 gene. In some embodiments, the gRNA is systemically administered to reduce plasma kallikrein protein level or activity. In some embodiments, plasma kallikrein protein level is total plasma kallikrein protein level. The gRNA is systemically administered together with a nucleic acid encoding a Cas9 nuclease e.g., an S. pyogenes Cas9. In particular embodiments, the guide RNA is chemically modified. In some embodiments, the guide RNA and the mRNA encoding a Cas nuclease are systemically administered in an LNP described herein, such as an LNP comprising Lipid A, a helper lipid (e.g., cholesterol), a stealth lipid (e.g., a PEG lipid, such as PEG2k-DMG), and a neutral lipid (e.g., DSPC).

In some embodiments, the gRNA comprising a guide sequence together with a Cas9 nuclease translated from the nucleic acid induce DSBs, and non-homologous ending joining (NHEJ) during repair leads to a mutation in the KLKB1 gene. In some embodiments, NHEJ leads to a deletion or insertion of a nucleotide(s), which induces a frame shift or nonsense mutation in the KLKB1 gene.

### 5. Lipid Compositions

In some embodiments, the nucleic acid compositions described herein, comprising a gRNA and a nucleic acid described herein encoding a Cas nuclease e.g. Cas9, are formulated in or systemically administered via a lipid nanoparticle; see e.g., WO2017173054A1 entitled “LIPID NANOPARTICLE FORMULATIONS FOR CRISPR/CAS COMPONENTS,” and WO2019067992A1 entitled “FORMULATIONS,” the contents of which, in particular the LNP compositions disclosed therein, are hereby incorporated by reference in their entirety. Lipid nanoparticles (LNPs) known to those of skill in the art to be capable of delivering therapeutic RNAs to subjects may be utilized with the guide RNAs described herein and the nucleic acid encoding a Cas nuclease.

Compositions comprising LNPs may include two active substances, a guide RNA and an RNA encoding a Cas nuclease, e.g. a Cas9 nuclease such as a Spy. Cas9 nuclease, together with a lipid component comprising an ionizable lipid. By lipid nanoparticle is meant a particle that comprises a plurality of (i.e. more than one) lipid molecules physically associated with each other by intermolecular forces.

### Ionizable Lipids

Lipid compositions for delivery of CRISPR/Cas mRNA and guide RNA components to a liver cell may comprise Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

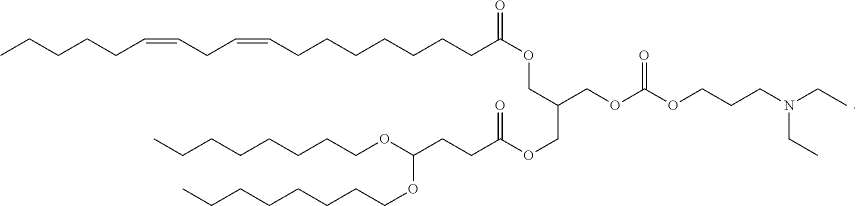 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86).

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol.

“Stealth lipids” are lipids that alter the length of time the nanoparticles can exist in vivo (e.g., in the blood), and a stealth lipid may be a PEG lipid. Stealth lipids may assist in the formulation process by, for example, reducing particle aggregation and controlling particle size. Stealth lipids used herein may modulate pharmacokinetic properties of the LNP. Stealth lipids suitable for use in a lipid composition of the disclosure include, but are not Typically, the PEG lipid comprises a lipid moiety and a polymer moiety based on PEG. PEG lipids known in the art are contemplated, including lipids comprising a “PEG-2K,” also termed “PEG 2000,” which has an average molecular weight of about 2,000 daltons. PEG-2K is represented herein by the following formula (I), wherein n is 45, meaning that the number averaged degree of polymerization comprises about 45 subunits. However, other PEG embodiments known in the art may be used.

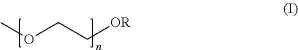

In any of the embodiments described herein, the PEG lipid may be selected from PEG-dilauroylglycerol, PEG-dimyristoylglycerol (PEG-DMG) (catalog #GM-020 from NOF, Tokyo, Japan), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE), or 1,2-dimyristoyl-rac-glycero-3-methoxypolyethylene glycol-2000 (PEG2k-DMG), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-2000](PEG2k-DSA). In one embodiment, the PEG lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

### LNP Formulations

The LNP composition may comprise a lipid component and an RNA component that includes a Cas nuclease mRNA (e.g. a Cas9 mRNA, such as a Spy. Cas9 mRNA), and a gRNA targeting KLKB1. In some embodiments, an LNP composition includes an mRNA encoding a Cas9 nuclease and a gRNA as the RNA component. In certain embodiments, an LNP composition may comprise the RNA component, Lipid A, a helper lipid, a neutral lipid, and a stealth lipid. In certain LNP compositions, the helper lipid is cholesterol. In certain compositions, the neutral lipid is DSPC. In additional embodiments, the stealth lipid is PEG2k-DMG.

In certain embodiments, lipid compositions are described according to the respective molar ratios of the component lipids in the formulation. Embodiments of the present disclosure provide lipid compositions described according to the respective molar ratios of the component lipids in the formulation. In one embodiment, the mol-% of the ionizable lipid such as Lipid A is from about 40 mol-% to 60 mol-%, optionally about 50 mol-%. In one embodiment, the mol-% of the ionizable lipid is about 55 mol-%. In some embodiments, the ionizable lipid mol-% of the LNP batch will be ±30%, ±25%, ±20%, ±15%, ±10%, ±5%, or ±2.5% of the target mol-%. In some embodiments, the ionizable lipid mol-% of the LNP batch is ±4 mol-%, ±3 mol-%, ±2 mol-%, ±1.5 mol-%, ±1 mol-%, ±0.5 mol-%, or 0.25 mol-% of the target mol-%. All mol-% numbers are given as a fraction of the lipid components of the LNP composition.

In one embodiment, the mol-% of the neutral lipid, e.g., neutral phospholipid, is from about 5 mol-% to 15 mol-%, optionally about 9 mol-%. In some embodiments, the neutral lipid mol-% of the LNP batch is +30%, +25%, +20%, +15%, +10%, +5%, or +2.5% of the target neutral lipid mol-%.

In one embodiment, the mol-% of the helper lipid is from about 20 mol-% to 60 mol-%. In one embodiment, the mol-% of the helper lipid is from about 25 mol-% to 55 mol-%, optionally, the mol-% of the helper lipid is from about 30 mol-% to 40 mol-%. In one embodiment, the mol-% of the helper lipid is adjusted based on ionizable lipid, neutral lipid, and PEG lipid concentrations to bring the lipid component to 100 mol-%. In one embodiment, the mol-% of the helper lipid is adjusted based on ionizable lipid and PEG lipid concentrations to bring the lipid component to at least 99 mol-%. In some embodiments, the helper mol-% of the LNP batch is ±30%, +25%, +20%, +15%, +10%, ±5%, or ±2.5% of the target mol-%.

In one embodiment, the mol-% of the PEG lipid is from about 1 mol-% to 10 mol-%. In one embodiment, the mol-% of the PEG lipid is from about 2 mol-% to 4 mol-%. In one embodiment, the mol-% of the PEG lipid is from about 2.5 mol-% to 4 mol-%. In one embodiment, the mol-% of the PEG lipid is about 3 mol-%. In some embodiments, the PEG lipid mol-% of the LNP batch is ±30%, +25%, +20%, +15%, +10%, ±5%, or ±2.5% of the target PEG lipid mol-%.

In certain embodiments, the cargo includes a nucleic acid (e.g., mRNA) encoding a Cas9 nuclease and a sgRNA. In some embodiments, the ionizable lipid is Lipid A. In some embodiments, an LNP composition comprises an ionizable lipid (e.g. Lipid A), a neutral lipid, a helper lipid, and a PEG lipid. In certain embodiments, the helper lipid is cholesterol. In certain embodiments, the neutral lipid is DSPC. In specific embodiments, PEG lipid is PEG2k-DMG. In some embodiments, an LNP composition may comprise a Lipid A, a helper lipid, a neutral lipid, and a PEG lipid. In additional embodiments, an LNP composition comprises Lipid A, cholesterol, DSPC, and PEG2k-DMG.

Embodiments of the present disclosure also provide lipid compositions described according to the molar ratio between the positively charged ionizable groups of the ionizable lipid (N) and the negatively charged phosphate groups (P) of the nucleic acid to be encapsulated. This may be mathematically represented by the ratio N/P. In some embodiments, an LNP composition may comprise a lipid component that comprises an ionizable lipid, a helper lipid, a neutral lipid, and a PEG lipid; and a nucleic acid component, wherein the N/P ratio is about 3 to 10. In some embodiments, the N/P ratio is about 5 to 7, optionally the N/P ratio is about 6. In some embodiments, the N/P ratio is 6±1. In some embodiments, the N/P ratio is 6±0.5. In some embodiments, the N/P ratio is ±30%, ±25%, ±20%, ±15%, ±10%, ±5%, or ±2.5% of the target N/P ratio.

In some embodiments, the RNA component comprises an mRNA, such as a nucleic acid disclosed herein, encoding a Cas9 nuclease, e.g., a Spy Cas9 nuclease, mRNA described herein, and a sgRNA described herein. In any of the foregoing embodiments, the sgRNA is a chemically modified sgRNA described herein.

In certain embodiments, the LNP compositions include a Cas9 nuclease mRNA (such as a Spy Cas9 mRNA) described herein and a sgRNA described herein. In certain embodiments, the LNP composition includes a ratio of gRNA to Cas9 nuclease mRNA, such as a Spy Cas9 nuclease mRNA from about 10:1 to 1:10, e.g., about 1:1, 1:2, or 1:3.

In some embodiments, LNPs are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of LNPs, may be used.

In some embodiments, microfluidic mixing, T-mixing, or cross-mixing is used. In certain aspects, flow rates, junction size, junction geometry, junction shape, tube diameter, solutions, or RNA and lipid concentrations may be varied. LNPs or LNP compositions may be concentrated or purified, e.g., via dialysis, tangential flow filtration, or chromatography. The LNPs may be composed of 4 lipids including Lipid A; DSPC; cholesterol; and DMG-PEG2k. In some embodiments, the LNP is suspended and formulated in an aqueous buffer of 50 mM Tris, 45 mM NaCl, and 5% (w/v) sucrose, pH 7.4.

Dynamic Light Scattering (“DLS”) can be used to characterize the polydispersity index (“pdi”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, LNPs disclosed herein have a size of 50 to 100 nm. In some embodiments, the LNPs have a size of 85 to 90 nm. Unless indicated otherwise, all sizes referred to herein are the average sizes (diameters) of the fully formed nanoparticles, as measured by dynamic light scattering on a Malvern Zetasizer. The nanoparticle sample is diluted in phosphate buffered saline (PBS) so that the count rate is approximately 200-400 kcts. The data are presented as a weighted-average of the intensity measure.

In some embodiments, LNPs associated with the gRNAs disclosed herein and mRNA encoding a Cas9 nuclease, e.g., Spy Cas9 nuclease disclosed herein are for use in preparing a medicament for treating HAE. In some embodiments, LNPs associated with the gRNAs disclosed herein and mRNA encoding a Cas9 nuclease (e.g. Spy Cas9 nuclease) disclosed herein are for use in preparing a medicament for reducing plasma kallikrein in subjects having HAE. In some embodiments, LNPs associated with the gRNAs disclosed herein and mRNA encoding a Cas9 nuclease (e.g. Spy Cas9) disclosed herein are for use in preparing a medicament for reducing plasma kallikrein concentration. In some embodiments, LNPs associated with the gRNAs disclosed herein and mRNA encoding a Cas9 nuclease (e.g. Spy Cas9) disclosed herein are for use in preparing a medicament for reducing plasma kallikrein activity. In some embodiments, LNPs associated with the gRNAs disclosed herein and mRNA encoding an Cas9 nuclease (e.g. Spy Cas9) disclosed herein are for use in treating HAE in a human subject. In some embodiments, LNPs associated with the gRNAs disclosed herein and mRNA encoding a Cas9 nuclease (e.g. Spy Cas9) disclosed herein are for use in reducing plasma kallikrein protein level or plasma kallikrein activity level in a human subject. In some embodiments, LNPs associated with the gRNAs disclosed herein and mRNA encoding a Cas9 nuclease (e.g. Spy Cas9) disclosed herein are for use in reducing total plasma kallikrein protein level in a human subject. In some embodiments, LNPs associated with the gRNAs disclosed herein and mRNA encoding a Cas9 nuclease (e.g. Spy Cas9) disclosed herein are for use in reducing HAE attack frequency in a human subject.

In some embodiments, the LNP comprises a lipid component and the lipid component comprises, consists essentially of, or consists of: about 50 mol-% ionizable lipid such as Lipid A; about 9 mol-% neutral lipid such as DSPC; about 3 mol-% of a stealth lipid such as a PEG lipid, such as PEG2k-DMG, and the remainder of the lipid component is helper lipid such as cholesterol, wherein the N/P ratio of the LNP composition is about 6. In some embodiments, the ionizable lipid is Lipid A. In some embodiments, the neutral lipid is DSPC. In some embodiments, the stealth lipid is a PEG lipid. In some embodiments, the stealth lipid is a PEG2k-DMG. In some embodiments, the helper lipid is cholesterol. In some embodiments, the LNP comprises a lipid component and the lipid component comprises: about 50 mol-% Lipid A; about 9 mol-% DSPC; about 3 mol-% of PEG2k-DMG, and the remainder of the lipid component is cholesterol wherein the N/P ratio of the LNP composition is about 6.

### II. Methods of Systemic Delivery

In some embodiments, the LNP composition described herein, comprising an mRNA encoding a Cas9 nuclease, e.g., Spy Cas9; and a guide RNA that targets that targets the KLKB1 gene is administered systemically. As used herein, systemic administration refers to broad biodistribution within an organism, e.g., intravenous administration.

In some embodiments, a single administration of the LNP composition described herein is sufficient to knockdown expression of the target protein. In some embodiments, a single administration of the LNP composition is sufficient to knockdown expression of the target protein in a population of cells. In other embodiments, more than one administration of the LNP composition may be beneficial to maximize editing via cumulative effects. For example, the LNP composition can be administered a second or third time (a “follow-on dose”), e.g., a second dose or a third dose. The dose of the second dose or third dose can be determined by a clinician to provide, e.g., about or greater than 60%, 70%, 80%, or 90% reduction in plasma kallikrein total protein or activity level as compared to baseline levels (e.g., the level prior to first LNP administration). The more than one administration (second dose or third dose) can be administered as flat dose, e.g., 25 mg, 50 mg, 75 mg, 100 mg, 125 mg, or 150 mg; or as a weight-based dose.

In some embodiments, the LNP composition described herein is administered by infusion with an infusion time of about 2-4 hours. In some embodiments, the LNP composition described herein is administered by infusion with an infusion time of about 2-5 hours. In some embodiments, the LNP composition described herein is administered by infusion with an infusion time of at least 2 hours.

### III. Dose

In some embodiments, the LNP composition described herein (e.g., comprising an effective amount of mRNA encoding a Cas9 nuclease and a guide RNA that targets a KLKB1 gene, e.g., a guide RNA that targets the KLKB1 gene (the total or combined dose)) is administered using a fixed dose. The fixed dose may be about 25-75 mg in a human subject. In some embodiments, the effective amount of the mRNA encoding a Cas nuclease and the guide RNA that targets the KLKB1 gene is a combined dose of about 25-100 mg. In some embodiments, the effective amount of the mRNA encoding a Cas nuclease and the guide RNA that targets the KLKB1 gene is a combined dose of about 50-75 mg. In some embodiments, the effective amount of the mRNA encoding a Cas nuclease and the guide RNA that targets the KLKB1 gene is a combined dose of about 25 mg. In some embodiments, the effective amount of the mRNA encoding a Cas nuclease and the guide RNA that targets the KLKB1 gene is a combined dose of about 50 mg. In some embodiments, the effective amount of the mRNA encoding a Cas nuclease and the guide RNA that targets the KLKB1 gene is a combined dose of about 75 mg. The LNP composition may be administered in an effective amount, in that the LNP composition, when dosed based on total RNA, administers an effective amount of an mRNA encoding a Cas9 nuclease and a guide RNA that targets the KLKB1 gene.

In some embodiments of the invention, “about” means within +5% of the stated value, e.g., a range of 23.75 mg to 26.25 mg for a value that is about 25 mg or a range of 71.25 mg to 78.75 mg for a value that is about 75 mg. In some embodiments of the invention, “about” means within ±10% of the stated value. In some embodiments of the invention, “about” means within ±20% of the stated value, e.g., a range of 20 mg to 30 mg for a value that is about 25 mg or a range of 60 mg to 90 mg for a value that is about 75 mg. Acceptable tolerances within various arts are understood.

In some embodiments, the LNP composition described herein (e.g., comprising an effective amount of mRNA encoding a Cas9 nuclease and a guide RNA that targets a KLKB1 gene (the total or combined RNA)) is administered using a weight-based dose, e.g., to a pediatric patient.

In other embodiments, subjects who receive a dose not sufficient to provide a sufficient decrease in the level of total plasma kallikrein protein or total plasma kallikrein activity, may receive more than one administration of the LNP composition to maximize editing via cumulative effects. For example, the LNP composition can be administered 2, 3, 4, 5, or more times, such as 2 times e.g., a second administration, a third administration, a fourth administration, or a fifth administration. In some embodiments, the LNP composition is administered to a human subject that has previously been administered the LNP composition. In some embodiments, the LNP composition is administered to a human subject that has previously been administered the LNP composition and has not achieved a greater than 60%, 70%, or 80% reduction in total plasma kallikrein protein level (e.g., a less than 60%, 70%, or 80% decrease of total plasma kallikrein protein level after administration of the LNP composition) as measured by, e.g., ELISA (e.g., Prekallikrein and Kallikrein Human ELISA kit Catalog no. ab171015) as determined at, e.g., at least 28, e.g., at 56 days after the first LNP administration. In some embodiments, the LNP composition is administered to a human subject that has previously been administered the LNP composition and has not achieved a greater than 60%, 70%, or 80% reduction in plasma kallikrein activity level (e.g., a less than 60%, 70%, or 80% decrease of plasma kallikrein activity level after administration of the LNP composition) as measured by, e.g., an activity assay kit (e.g., SensoLyte® Rh110Plasma Kallikrein Activity Assay Kit *Fluorimetric*, Catalog no. AS-72255) as determined at, e.g., at least 28 days, e.g., at 56 days, after the first LNP administration. The percent decrease is compared to a baseline sample obtained prior to administration of the LNP composition. Commercial kits are available to determine total kallikrein protein level and total kallikrein activity level. Selection of appropriate controls and methods of validation of such kits are well known to those of skill in the art.

### IV. Methods of Use

Hereditary angioedema disease pathology and therapeutic interventions HAE is a rare, autosomal dominant genetic disorder characterized by recurring, painful, unpredictable, and potentially fatal inflammatory attacks of cutaneous and submucosal swelling in various organs and tissues of the body. These symptoms are caused by dysregulated production of bradykinin, a peptide which leads to increased vascular permeability and resultant swelling. Attacks may occur as frequently as every 7 to 14 days and last for 48 to 72 hours, with laryngeal edema being particularly life-threatening as it may result in airway obstruction and death by asphyxiation (Zuraw 2008; Busse 2020, The US HAEA Medical Advisory Board 2020 Guidelines for the Management of Hereditary Angioedema (doi.org/10.1016/j.jaip.2020.08.046)).

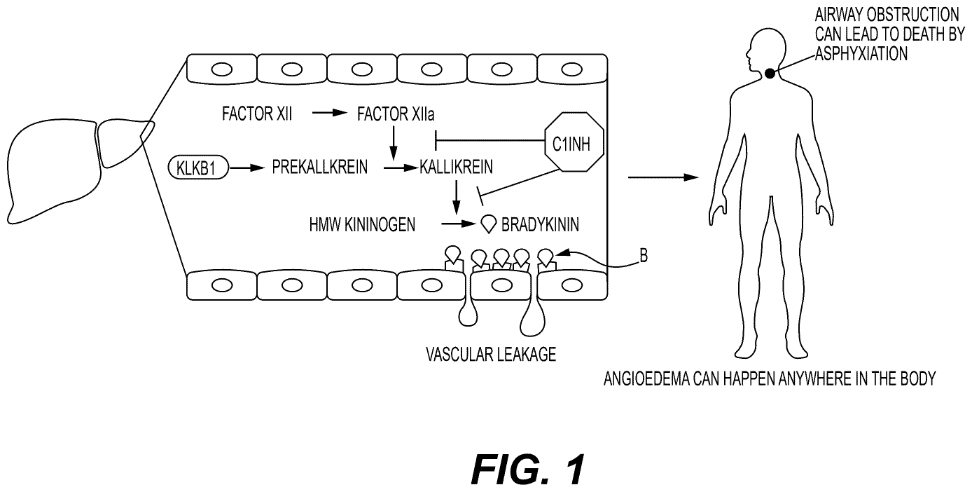

The most common forms of HAE are caused by a deficiency of C1 esterase inhibitor (C1-INH), a key regulator of the contact activation pathway. Deficiency is either due to low levels of functionally active C1-INH protein (Type I) or normal levels of functionally inactive C1-INH (Type II). See, e.g., FIG. 1. Factor XII (FXII) is a plasma protein at the start of the contact activation pathway where, upon activation to Factor XIIa (FXIIa), it converts plasma prekallikrein (PKK) into active kallikrein. Kallikrein catalyzes the conversion of high molecular weight kininogen to bradykinin. In a healthy subject, homeostasis of FXIIa and kallikrein activity is negatively controlled by C1-INH, which is a direct inhibitor of FXIIa and kallikrein. However, in HAE Types I and II, C1-INH deficiency or dysfunction leads to dysregulated FXIIa and kallikrein activity, local accumulation of bradykinin, and increased vascular permeability and vasodilation, subsequently leading to angioedema (Busse 2020). The US HAEA Medical Advisory Board 2020 Guidelines for the Management of Hereditary Angioedema recommended that HAE should be broadly divided into HAE due to C1INH deficiency (HAE-C1INH) and HAE-nl-C1INH (HAE with normal C1INH). HAE-C1INH is further subdivided into type I and type II, which appear to be clinically similar. HAE-nl-C1INH is further subdivided based on the underlying mutation or unknown in cases where the mutation has not been found.

The majority of currently approved treatments for HAE are designed to restore normal function of the contact activation pathway. These strategies include C1-INH replacement (recombinant or plasma-derived), inhibition of the B2 bradykinin receptor (icatibant), and kallikrein inhibition (ecallantide, lanadelumab, berotralstat). Depending on the agent, these medications are used either to prevent attacks (prophylaxis) or treat attacks on demand. They are administered IV, by subcutaneous (SC) injection, or orally. Whereas on-demand treatments are administered at the onset of an HAE attack, prophylactic agents require chronic IV or SC administration as often as twice per week or daily oral administration to ensure constant pathway suppression for disease control. However, despite chronic administration, breakthrough attacks still occur.

Agents for HAE attack prophylaxis can be used in conjunction with NTLA-2002. Such agents may be useful particularly in an initial period after dosing with NTLA-2002. The time for withdrawal of HAE attack prophylaxis can be determined by the health care provider. Although some agents for HAE attack prophylaxis were not permitted in the context of the study, such agents are not necessarily contraindicated for use with NTLA-2002. Instead, certain agents were not permitted for use in conjunction with the study, for example, as they could potentially interfere with certain diagnostic measures in the study.

Agents for HAE attack prophylaxis can be withdrawn prior to dosing with NTLA-2002. Half-lives and washout periods for such agents are known in the art and provided here. Dosing with NTLA-2002 can be performed during the washout period or after completion of the washout period.

### 1. Methods of In Vivo Editing

Methods of in vivo editing of a KLKB1 gene in the liver of a human subject, e.g., a human subject having HAE, are provided herein. In some embodiments, the method of in vivo editing of the gene comprises systemically administering to the human subject the LNP composition described herein comprising an mRNA encoding a Cas9 nuclease, e.g., Spy Cas9; and a guide RNA that targets the KLKB1 gene. In some embodiments, the in vivo editing occurs at the site targeted by the guide RNA in a hepatocyte of the subject.

In these embodiments, administration of the LNP composition to the subject may be associated with a change in a biosafety metric. In some embodiments, the subject is assessed to determine whether the change in the biosafety metric is an acceptable change. In some embodiments, an acceptable change can be determined by a clinician or laboratory. In some embodiments, an acceptable change can be one that does not qualify as a safety event, including an adverse event (NCI-CTCAE Grade not greater than or equal to 3), a serious adverse event, an adverse event of special interest, or a treatment-emergent adverse event (CTCAE Grade not greater than or equal to 3), as described herein. Biosafety metrics, including those associated with administration of an LNP composition, are known in the art. Acceptable levels or changes in the biosafety metrics are known in the art and may be assessed by routine methods.

In some embodiments, an acceptable biosafety metric level is one that falls within the subject inclusion criteria or does not fall within the subject exclusion criteria described herein.

In some embodiments, an acceptable change in a biosafety metric level is a change that is acceptable after a period of time, e.g., initially falls outside of acceptable levels but stabilizes to an acceptable level by, e.g., day 2, 3, 4, 5, 6, 7, 14, or 28 after administration. Acceptable changes in a biosafety metric levels and acceptable times to resolution are provided for various measures herein. Methods to measure and grade biosafety metric levels are known in the art.

In some embodiments, an acceptable biosafety metric level (or an acceptable change in a biosafety metric level) is one that does not constitute an adverse event of Grade 3 or higher according to CTCAE guidelines, including National Cancer Institute (NCI)-CTCAE guidelines, version 5.0. In some embodiments, a change in a biosafety metric level (e.g., one or more levels associated with a laboratory parameter, vital sign, ECG data, physical exam, etc., as described herein) constitutes as an adverse event if the change, e.g., induces clinical signs or symptoms; requires active intervention; requires interruption or discontinuation of the LNP composition; or the change in the biosafety metric is clinically significant, as determined by a clinician.

In some embodiments, an adverse event is any untoward medical occurrence in a subject administered a study drug or has undergone study procedures and which does not necessarily have a causal relationship with the treatment. In some embodiments, an adverse event is an unintended sign (including an abnormal laboratory finding), symptom, or disease temporally associated with the treatment, whether or not related to the medicinal (investigational) product. In some embodiments, an adverse event that induces clinical signs or symptoms. In some embodiments, an adverse event requires active intervention. In some embodiments, an adverse event requires interruption or discontinuation of the treatment. In some embodiments, an adverse event is an abnormality that is clinically significant in the opinion of the investigator. Grading criteria for adverse events are known in the art, such as, e.g., Common Terminology Criteria for Adverse Events (CTCAE), including National Cancer Institute (NCI)-CTCAE.

In some embodiments, an acceptable biosafety metric level (or an acceptable change in a biosafety metric level) is one that does not constitute a serious adverse event. In some embodiments, a serious adverse event results in death. In some embodiments, a serious adverse event is life threatening (e.g., places the subject at immediate risk of death as determined by a clinician). In some embodiments, a serious adverse event results in persistent or significant disability. In some embodiments, a serious adverse event results in incapacity or substantial disruption of the ability to conduct normal life functions. In some embodiments, a serious adverse event results in congenital anomaly or birth defect. In some embodiments, a serious adverse event requires inpatient hospitalization or leads to prolongation of hospitalization.

In some embodiments, an acceptable biosafety metric level (or an acceptable change in a biosafety metric level) is one that does not constitute a Common Terminology Criteria for Adverse Events (CTCAE) grade equal to or greater than 3 for a treatment-emergent adverse event.

The method of in vivo editing may comprise measuring known laboratory assessments relating generally to, e.g., coagulation, hematology, clinical chemistry, urinalysis, and other bioanalytical assessments (e.g., cytokines, complement). Particular biosafety metrics include, but are not limited to one or more of the following non-limiting biosafety metrics: liver enzyme, levels of activated partial thromboplastin time (aPTT), levels of prothrombin time (PT), levels of thrombin generation time (TGT) (e.g., peak height, lag time, or endogenous thrombin potential), levels of fibrinogen, prothrombin international normalized (INR) ratio, level of d-dimer, HBV, HBsAg, HCV Ab, laboratory parameters consistent with disseminated intravascular coagulation, changes in hematology values, changes in chemistry values, changes in coagulation, changes in urinalysis, levels of C-reactive protein, levels of complement (C3a, C5a, Bb), levels of cytokines (GM-CSF, INF-γ, IL-1p, IL-4, IL-5, IL-6, IL-8, IL-10, IL-13, IL-23, TNF-α, IL-17, MCP-1), acute liver injury (e.g. a CTCAE >Grade 2 elevations in ALT, AST, total bilirubin or clinically relevant symptoms/signs of liver injury after administration of a treatment), and changes in a 12-Lead Electrocardiogram. In certain embodiments, the laboratory assessment is an activated partial thromboplastin time (aPTT) level. In some embodiments, biosafety metrics include injection-site reaction(s) and gastrointestinal symptoms.

Other biosafety metrics relating to, e.g., hematology, coagulation, clinical chemistry, and urinalysis are known in the art. For example, biosafety metrics relating to hematology include, but are not limited to, platelet count, RBC count, hemoglobin, hematocrit, RBC indices (MCV, MCH, MCHC, RDW), precent reticulocytes, white blood cell (WBC) count with differential (neutrophils, lymphocytes, monocytes, eosinophils, basophils). For example, biosafety metrics relating to coagulation include, but are not limited to, aPTT, PT, INR, fibrinogen, d-dimer, and TGT. For example, biosafety metrics relating to clinical chemistry include, but are not limited to, albumin, blood urea nitrogen, creatinine, glucose non-fasting, potassium, sodium, chloride, carbon dioxide, calcium, AST, ALT, alkaline phosphatase, total and direct bilirubin, total protein, creatine kinase, lactose dehydrogenase, total cholesterol, and LDL cholesterol. For example, biosafety metrics relating to urinalysis include, but are not limited to, specific gravity, pH, glucose, protein, blood, ketones, bilirubin, urobilinogen, nitrite, and leukocyte esterase. In certain embodiments, the biosafety metric is an aPTT level.

In some embodiments, a level of a biosafety metric is measured following administration of the LNP composition. In some embodiments, a level of a biosafety metric is measured prior to and following the administration of the LNP composition, thereby allowing for a comparison of the levels of the biosafety metric before and after treatment with the LNP composition. In some embodiments, a level of a biosafety metric measured prior to administration of the LNP composition can serve as a baseline for comparison against one or more levels of the biosafety metric measured following administration of the LNP composition. In some embodiments, a baseline is the last available measurement taken prior to administration of the LNP composition. In these embodiments, the administration of the LNP composition results in an acceptable change in liver enzyme levels (e.g., no more than an elevation in ALT or AST >5×ULN for more than 4 weeks after administration of a treatment, ALT or AST >3×ULN and total bilirubin >2×ULN (Hy's law) after administration of a treatment). In these embodiments, the administration of the composition results in an acceptable change in levels of activated partial thromboplastin time (aPTT) (e.g., an elevation in aPTT) >5×ULN for more than 4 weeks after administration of a treatment). In these embodiments, the administration of the composition results in an acceptable change in levels of prothrombin time (PT). In these embodiments, the administration of the composition results in an acceptable change in levels of thrombin generation time (TGT) (e.g., peak height, lag time, or endogenous thrombin potential). In these embodiments, the administration of the composition results in an acceptable change in levels of fibrinogen. In some embodiments, the administration of the composition results in an acceptable change in the prothrombin international normalized (INR) ratio. In these embodiments, the administration of the composition results in an acceptable change in level of d-dimer. In these embodiments, the administration of the composition results in an acceptable change in laboratory parameters consistent with disseminated intravascular coagulation. In these embodiments, the administration of the composition results in an acceptable change in hematology values (e.g. a CTCAE >Grade 2 abnormal blood test results after administration of a treatment). In these embodiments, the administration of the composition results in an acceptable change in chemistry values. In these embodiments, the administration of the composition results in an acceptable change in abnormal coagulation findings defined by clinically relevant abnormal bleeding. In these embodiments, the administration of the composition results in an acceptable change in urinalysis. In these embodiments, the administration of the composition results in an acceptable change in levels of C-reactive protein. In these embodiments, the administration of the composition results in an acceptable change in levels of complement. In these embodiments, the administration of the composition results in an acceptable change in levels of cytokines.

In these embodiments, the administration of the composition results in an acceptable change in a biosafety metric level that does not constitute a treatment-emergent adverse event of Grade 3 or higher according to CTCAE guidelines. In these embodiments, the administration of the composition results in an acceptable change in a biosafety metric level that does not constitute an incidence of thrombosis. In these embodiments, the administration of the composition results in an acceptable change in a biosafety metric level that does not constitute an incidence of hemorrhage. In these embodiments, the administration of the composition results in an acceptable change in a biosafety metric level that does not constitute an incidence of disseminated intravascular coagulation. In these embodiments, the administration of the composition results in an acceptable change in a biosafety metric level that does not constitute an incidence of cytokine release syndrome. In these embodiments, the administration of the composition results in an acceptable change in a biosafety metric level that does not constitute an acute liver injury (e.g. a CTCAE >Grade 2 elevations in ALT, AST, total bilirubin or clinically relevant symptoms/signs of liver injury after administration of a treatment). In these embodiments, the administration of the composition results in an acceptable change in a 12-Lead Electrocardiogram as determined by a clinician.

In some embodiments, the method of in vivo editing of the gene comprises systemically administering to the human subject the LNP composition comprising an effective amount of an mRNA encoding Cas9 and guide RNA that targets the KLKB1 gene, and the administration results in an acceptable change in levels of anti-Cas9 antibodies.

In some embodiments, the administration of the LNP composition results in an acceptable change in the pharmacokinetics of Lipid A. In some embodiments, the administration of the LNP composition results in an acceptable change in the pharmacokinetics of DMG-PEG2k. In some embodiments, the administration of the LNP composition results in an acceptable change in the pharmacokinetics of Cas9 mRNA. In some embodiments, the administration of the LNP composition results in an acceptable change in the pharmacokinetics of sgRNA.

### 2. Methods of Treatment

Methods of treating a human subject by in vivo editing of a KLKB1 gene in the liver are provided herein. In some embodiments, the method of in vivo editing of the KLKB1 gene comprises systemically administering to the human subject a LNP composition described herein (e.g., comprising an effective amount of an mRNA encoding a Cas9 nuclease, e.g., Spy Cas9; and a guide RNA that targets the KLKB1 gene) and in vivo editing the KLKB1 gene occurs at the site targeted by the guide RNA in a hepatocyte of the subject. In certain embodiments, the guide RNA comprises a targeting sequence comprising least 18 contiguous nucleotides, preferably 20 nucleotides of GGAUUGCGUAUGGGACACAA (SEQ ID NO: 15). In some embodiments, the guide RNA comprises the nucleotide sequence of GGAUUGCGUAUGGGACACAAGUUUUAGAGCUAGAAAUAGCAAGUUAAAAUAA GGCUAGUCCGUUAUCAACUUGAAAAAGUGGCACCGAGUCGGUGCUUUU (SEQ ID NO: 388). In some embodiments, the sgRNA is modified. In some embodiments, the sgRNA comprises the modification pattern shown below in SEQ ID NO: 391, wherein the modified sgRNA comprises the following sequence: mG*mG*mA*UUGCGUAUGGGACACAAGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 391), wherein A, C, G, and U indicate RNA nucleosides (2′-OH) adenine, cytosine, guanine, and uridine, respectively; mA, mC, mG, and mU indicate 2′-O-methyl modified adenine, cytosine, guanine, and uridine, respectively; and * indicates a phosphorothioate linkage.

In some embodiments, provided herein are methods for treating a human subject suffering from HAE, as described herein (e.g., reduction in HAE attack number, breakthrough attack, or number or proportion of patients who are attack-free over a given period of time, frequency of hospitalization related to an HAE attack, or frequency of use of acute therapies related to an HAE attack). In some embodiments, HAE is Type I. In some embodiments, the HAE is Type II.

In some embodiments, provided herein is a method for treating HAE in a human subject, comprising systemically administering to the human subject a LNP composition described herein (e.g., comprising an effective amount of an mRNA encoding a Cas9 nuclease, e.g., Spy Cas9; and a guide RNA that targets a KLKB1 gene, e.g., a guide with a targeting sequence of SEQ ID NO: 15 (GGAUUGCGUAUGGGACACAA)), thereby treating HAE, wherein the administration of the composition results in a clinically significant improvement in a level of a clinical metric in the subject as compared to a baseline level of the clinical metric.

In some embodiments, provided herein is a method for treating HAE in a human subject, comprising systemically administering to the human subject a LNP composition described herein (e.g., comprising an effective amount of an mRNA encoding a Cas9 nuclease, e.g., Spy Cas9; and a guide RNA that targets a KLKB1 gene e.g., a guide with a targeting sequence of SEQ ID NO: 15 (GGAUUGCGUAUGGGACACAA)), thereby treating HAE, wherein the mRNA encoding a Cas9 nuclease and the guide RNA that targets the KLKB1 gene are administered at a combined dose of about 25-150 mg, e.g. about 25-100 mg.

In some embodiments, provided herein is a method for treating HAE in a human subject, comprising systemically administering to the human subject a LNP composition described herein (e.g., comprising an effective amount of an mRNA encoding a Cas9 nuclease, e.g., Spy Cas9; and a guide RNA that targets a KLKB1 gene e.g., a guide with a targeting sequence of SEQ ID NO: 15 (GGAUUGCGUAUGGGACACAA)), thereby treating HAE, wherein the mRNA encoding a Cas9 nuclease and the guide RNA that targets the KLKB1 gene are administered at a combined dose of about 25-75 mg.

In some embodiments, provided herein is a method for treating HAE in a human subject, comprising systemically administering to the human subject a LNP composition described herein (e.g., comprising an effective amount of an mRNA encoding a Cas9 nuclease, e.g., Spy Cas9; and a guide RNA that targets a KLKB1 gene e.g., a guide with a targeting sequence of SEQ ID NO: 15 (GGAUUGCGUAUGGGACACAA)), thereby treating HAE, wherein the mRNA encoding a Cas9 nuclease and the guide RNA that targets the KLKB1 gene are administered at a combined dose of about 50-75 mg.

In some embodiments, provided herein is a method for treating HAE in a human subject, comprising systemically administering to the human subject a LNP composition described herein (e.g., comprising an effective amount of an mRNA encoding a Cas9 nuclease, e.g., Spy Cas9; and a guide RNA that targets a gene, e.g., a guide RNA that targets the KLKB1 gene e.g., a guide with a targeting sequence of SEQ ID NO: 15 (GGAUUGCGUAUGGGACACAA)), thereby treating HAE, wherein the administration of the composition reduces total plasma kallikrein protein level or kallikrein activity level relative to baseline plasma. In some embodiments, the HAE is Type I. In some embodiments, the HAE is Type II. In some embodiments, the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate. In some embodiments, the LNP comprises a PEG lipid. In embodiments where the LNP comprises a PEG lipid, the PEG lipid comprises dimyristoylglycerol (DMG). In embodiments where the PEG lipid comprises dimyristoylglycerol ( )MG), the PEG lipid comprises PEG-2k. In some embodiments, the LNP composition has an N/P ratio of about 5-7. In some embodiments, the guide RNA and Cas nuclease are present in a ratio ranging from about 5:1-1:5 by weight. In some embodiments, the mRNA encodes a Cas9 nuclease. In some embodiments, the mRNA encodes S. pyogenes Cas9. In some embodiments, the Cas nuclease is codon-optimized. In some embodiments, the guide RNA comprises at least one modification. In embodiments where the guide RNA comprises at least one modification, the guide RNA includes a 2′-O-methyl modified nucleotide or a phosphorothioate bond between nucleotides. In some embodiments, effective amount of the mRNA encoding a Cas nuclease and the guide RNA that targets the KLKB1 gene is a combined dose of about 25-150 mg of total RNA. In some embodiments, the effective amount of mRNA encoding a Cas nuclease and the guide RNA that targets the KLKB1 gene is a combined dose of about 25-100 mg of total RNA. In some embodiments, the effective amount of mRNA encoding a Cas nuclease and the guide RNA that targets the KLKB1 gene is a combined dose of about, e.g., 25 mg, 50 mg, 75 mg, 100 mg, 125 mg, or 150 mg. In some embodiments, the effective amount of mRNA encoding a Cas nuclease and the guide RNA that targets the KLKB1 gene is a combined dose of about 50-75 mg of total RNA. In some embodiments, the effective amount of mRNA encoding a Cas nuclease and the guide RNA that targets the KLKB1 gene is a combined dose of about 25 mg of total RNA. In some embodiments, the effective amount of mRNA encoding a Cas nuclease and the guide RNA that targets the KLKB1 gene is a combined dose of about 75 mg of total RNA. In some embodiments, the effective amount of mRNA encoding a Cas nuclease and the guide RNA that targets the KLKB1 gene is a combined dose of about 50 mg of total RNA. In some embodiments, the effective amount of mRNA encoding a Cas nuclease and the guide RNA that targets the KLKB1 gene is a combined dose of about 0.3-2 mg/kg. In some embodiments, the effective amount of mRNA encoding a Cas nuclease and the guide RNA that targets the KLKB1 gene is a combined dose of about 0.5-1 mg/kg. In some embodiments, the effective amount of mRNA encoding a Cas nuclease and the guide RNA that targets the KLKB1 gene is a combined dose of about 0.3 mg/kg. In some embodiments, the effective amount of mRNA encoding a Cas nuclease and the guide RNA that targets the KLKB1 gene is a combined dose of about 0.5 mg/kg. In some embodiments, the effective amount of mRNA encoding a Cas nuclease and the guide RNA that targets the KLKB1 gene is a combined dose of about 0.7 mg/kg. In some embodiments, the effective amount of mRNA encoding a Cas nuclease and the guide RNA that targets the KLKB1 gene is a combined dose of about 0.9 mg/kg. In some embodiments, the effective amount of mRNA encoding a Cas nuclease and the guide RNA that targets the KLKB1 gene is a combined dose of about 1 mg/kg. In some embodiments, administration of the composition reduces total plasma kallikrein protein by at least 60%, or by 60%-95%, 60%-90%, 70%-95%, 70%-90%, or 80%-95% as compared to baseline total plasma kallikrein protein. before administration of the composition. In some embodiments, the total plasma kallikrein protein level is less than about 20 μg/mL after administration of the composition. In some embodiments, the total plasma kallikrein protein level is less than about 15 μg/mL after administration of the composition. In some embodiments, the total plasma kallikrein protein level is less than about 10 μg/mL after administration of the composition.

In certain embodiments, the methods provided herein are for reducing the frequency of HAE attacks in a subject relative to the historical frequency of attacks for that subject.

In certain embodiments, the methods provided herein are for improving one or more clinical metrics described herein for HAE, e.g., according to a suitable Quality of Life (QoL) questionnaire, e.g., MOXIE Angioedema Quality of Life (AE-QoL) questionnaire, or preventing reoccurrence of one or more symptoms.

In certain embodiments, the methods provided herein are for reducing the severity of HAE attacks in a subject relative to the historical severity of attacks for that subject. In certain embodiments, the methods provided herein are for reducing the frequency of attacks with laryngeal edema, which is typically associated with an overall attack severity of severe, characterized by a marked limitation in activity, and the subject requiring assistance.

In certain embodiments, the methods provided herein are for reducing the frequency and severity of HAE attacks in a subject relative to the historical frequency and severity of attacks for that subject.

In certain embodiments, the subject has a reduced frequency of use of hospitalization related to an HAE attack. In certain embodiments, the subject has a reduced frequency of use of acute therapies related to an HAE attack.

In certain embodiments, due to the severity of an attack, the methods provided herein can be used for treatment of a subject with rare or infrequent attacks, or even after a single attack. In certain embodiments, the methods provided herein are used in a subject with a single attack wherein the attack includes laryngeal edema, as such edema can be life-threatening.

In some embodiments, the method of treatment comprises systemically administering to the human subject a LNP composition described herein e.g., comprising an effective amount of an mRNA encoding a Cas9 nuclease and a guide RNA that targets a KLKB1 gene, wherein the subject has an average of at least two HAE attacks, e.g., confirmed HAE attacks, per month, as averaged over a three-month (90-day) period, prior to administration of the LNP composition. In some embodiments, the subject has an average of at least one HAE attack, e.g., confirmed HAE attack, per month, as averaged over a three-month (90-day) period, prior to administration of the LNP composition.

Breakthrough attacks with HAE are common, particularly in those with more severe disease, despite HAE prophylaxis. In some embodiments, provided herein are methods for treating a subject receiving HAE prophylaxis, including methods for treating a subject who has breakthrough attacks while receiving HAE prophylaxis. In some embodiments, the method of treatment comprises systemically administering to the human subject a LNP composition described herein e.g., comprising an effective amount of an mRNA encoding a Cas9 nuclease and a guide RNA that targets a KLKB1 gene, wherein the subject has been or is currently being treated with a different HAE therapy, e.g., a non-acute HAE therapy, i.e., HAE prophylaxis. In some embodiments, the subject has an average of at least two HAE attacks, e.g., confirmed HAE attacks, per month, as averaged over a three-month (90-day) period, while on HAE prophylaxis, prior to administration of the LNP composition. In some embodiments, the subject has an average of at least one HAE attack, e.g., confirmed HAE attack, per month, as averaged over a three-month (90-day) period while on HAE prophylaxis, prior to administration of the LNP composition.

In some embodiments, the subject does not tolerate long term treatment, e.g., a treatment emergent event after ongoing non-acute treatment or unsatisfactory treatment with chronic treatment (e.g., needle fatigue), with at least one type of HAE prophylaxis. In certain embodiments, the subject does not tolerate long term treatment with at least one type of HAE prophylaxis due to clinically unacceptable adverse events, e.g., a grade 3 or higher adverse event. In certain embodiments, the adverse event is determined by the subject to be unacceptable, although clinically acceptable, e.g., weight gain, acne, such that the subject has chosen to discontinue HAE prophylaxis with the specific agent. In certain embodiments, the subject does not tolerate long term treatment with at least one type of HAE prophylaxis because the treatment is contraindicated in the subject, e.g., women who are pregnant, trying to become pregnant, or lactating, children. In certain embodiments, the subject does not tolerate long term treatment with at least one type of HAE prophylaxis because the treatment is contraindicated in the subject due to existing or prior co-morbidities, e.g., certain types of liver disease, breast cancer, prostate cancer, hepatocellular carcinoma, and certain cardiovascular risk factors. In certain embodiments, the HAE prophylaxis is prophylaxis with an attenuated androgen, e.g., danazol, oxandrolone, stanozolol.

It is understood that subjects who do not tolerate treatment (e.g., a treatment emergent event after ongoing non-acute treatment or unsatisfactory treatment with chronic treatment (e.g., needle fatigue)), such as long-term treatment with certain agents that can be used for HAE prophylaxis may tolerate the agents for acute therapy, i.e., short or intermittent treatment, rather than long term treatment.

In some embodiments, the method of in vivo editing of the KLKB1 gene comprises systemically administering to the human subject a LNP composition described herein, e.g., comprising an effective amount of an mRNA encoding a Cas9 nuclease and a guide RNA that targets the KLKB1 gene, and results in a clinically significant improvement in a level of a clinical metric, e.g., attack frequency, attack severity. In certain embodiments, the attack frequency is measured while the subject is maintained on HAE prophylaxis. In certain embodiments, the attack frequency is measured after HAE prophylaxis has been withdrawn from the subject. In certain embodiments, the attack severity is measured while the subject is maintained on HAE prophylaxis. In certain embodiments, the attack severity is measured after HAE prophylaxis has been withdrawn from the subject.

In certain embodiments, the confirmed HAE attack frequency is reduced by at least 60%, 70%, preferably at least 80%, 85%, 90%, or 95% as compared to an appropriate historical period, e.g., confirmed HAE attacks, per month, as averaged over a period of time, three-month (90-day) period; for at least 6 months after administration of the LNP composition, optionally for at least one year after the administration of the LNP composition. In certain embodiments, the period for determining the attack frequency after administration of the LNP composition begins immediately after (e.g., within one day) administration of the LNP composition. In certain embodiments, the period for determining the attack frequency after administration of the LNP composition begins after 4 weeks, i.e., at day 29 after administration of the LNP composition.

In some embodiments, the method of in vivo editing of the KLKB1 gene comprises systemically administering to the human subject a LNP composition described herein, e.g., comprising an effective amount of an mRNA encoding a Cas9 nuclease and a guide RNA that targets the KLKB1 gene, and results in a significant improvement in a level of an aspect in a quality-of-life indicator assessment, e.g., MOXIE Angioedema Quality of Life (AE-QoL) assessment. In some embodiments, the method of in vivo editing of the KLKB1 gene comprises systemically administering to the human subject a LNP composition described herein, e.g., comprising an effective amount of an mRNA encoding a Cas9 nuclease and a guide RNA that targets the KLKB1 gene, and results in a significant improvement in a level of a quality-of-life assessment, e.g., MOXIE Angioedema Quality of Life (AE-QoL) assessment.

Additional clinical efficacy metrics, including metrics for assessing efficacy for HAE treatment are known in the art. Similarly, levels or changes in clinical efficacy metrics indicative of amelioration of HAE, are known in the art and may be assessed by routine methods, e.g., by a clinician or laboratory.

In some embodiments, a method of treating HAE comprises administering the LNP composition described herein and measuring a clinical efficacy metric following administration of the LNP composition. In some embodiments, a method of treating HAE comprises administering the LNP composition described herein and measuring a clinical efficacy metric prior to and following the administration of the LNP composition, thereby allowing for a comparison of the levels of the clinical efficacy metric before and after treatment with the LNP composition.

For example, plasma kallikrein level, e.g., total plasma kallikrein protein level, is a clinical efficacy metric for HAE. In some embodiments, the method of treating HAE comprises administering the LNP composition described herein and reducing kallikrein level, e.g., total plasma kallikrein protein level in the subject. In some embodiments, the method of treating HAE comprises administering the LNP composition described herein and reducing total plasma kallikrein protein level in the subject by at least after treatment (e.g., after 28 days or about 56 days after administration of the LNP composition) as compared to baseline, e.g., prior to treatment. In some embodiments, the method of treating HAE described herein yields at least 60% reduction in total plasma kallikrein protein level after treatment, e.g., at least 28 days, or about 56 days after administration of the LNP composition as compared to baseline. In some embodiments, the method of treating HAE described herein yields 60%-95%, 60%-90%, 70%-95%, 70%-90%, or 80%-95% reduction in kallikrein level, e.g., total plasma kallikrein protein level, after treatment, e.g., at least 28 days, or about 56 days after administration of the LNP composition as compared to baseline.

In other embodiments, administration of the LNP composition reduces total plasma kallikrein protein level in a subject to less than about 20 μg/mL. In some embodiments, administration of the LNP composition reduces total plasma kallikrein protein level to less than about 15 μg/mL. In some embodiments, administration of the LNP composition reduces total plasma kallikrein protein level to less than about 10 μg/mL. In certain embodiments, the level is measured at least 28 days, e.g., about 56 days after treatment.

a. Subject Inclusion Criteria

In some embodiments, a subject having HAE (HAE Type I or HAE Type II) to whom the LNP composition described herein e.g., comprising an mRNA encoding a Cas9 nuclease, e.g., Spy Cas9; and a guide RNA that targets a KLKB1 gene, e.g., a guide RNA that targets the KLKB1 gene, is assessed for one or more of the following subject inclusion criteria.

i. HAE Subject Inclusion Criteria

In some embodiments, a human subject has been or concurrently is diagnosed with HAE prior to treatment. In some embodiments, a human subject is diagnosed with HAE based on genetic testing. In some embodiments, a human subject is diagnosed with HAE based on a confirmed by assessment of functional C1-INH level, C1-INH antigenic level, and C4 level. Further, the subject must have confirmed HAE attacks. Subjects in Phase 1 must have an Investigator-confirmed and documented historical HAE attack number of at least 3 during the previous 3 months (90 days) from the start of screening. Subjects in the Phase 1 portion of the trial may be treated with certain types of prophylaxis during the observation period, i.e., 3 months (90 days) from the start of screening, with various exclusions discussed below. Subjects in Phase 2 must have an Investigator-confirmed and documented historical HAE attack number of at least 3 during the previous 3 months (90 days) to enter the run-in period, and must have an Investigator-confirmed and documented historical HAE attack number of at least 2 during the 8-week (56-day) run-in-period to be eligible for enrollment and randomization. In the Phase 2 portion of the study, subjects must agree to refrain from the use of prophylactic therapies from the start of the 8-week run-in period through the end of the 16-week primary observation period, and the Investigator must confirm that this is medically appropriate and does not place the subject at an undue safety risk. Other prophylactic agent exclusions are provided below.

In both the Phase 1 and Phase 2 trials, subjects must have access to, and the ability to use, ≥1 acute medication(s) to treat angioedema attacks.

In some embodiments, the human subject is at least 18 years of age at the time of administration. In some embodiments, the human subject has a diagnosis of HAE with the attack frequency, either with or without prophylaxis, as provided above. In some embodiments, the human subject has HAE, Type 1. In some embodiments, the human subject has HAE, Type II. In some embodiments, the human subject has an aspartate aminotransferase (AST) level ≤upper limit of normal (ULN) range at screening. In some embodiments, the human subject has an alanine aminotransferase (ALT) level ≤upper limit of normal (ULN) range at screening. In some embodiments, the human subject has a total bilirubin level ≤upper limit of normal (ULN) range at screening, with subjects with a history of Gilbert's Syndrome being allowed in the trials with a total bilirubin ≤2×ULN on screening evaluation. In some embodiments, the human subject has an aPTT, an international normalized ratio (INR), fibrinogen, and d-dimer levels within a reference range or Principal Investigator (PI)-determined clinically non-significant at screening. In some embodiments, the human subject has an estimated glomerular filtration rate (GFR) >45 mL/min/1.73m\hat{ }2, (e.g., as measured by the Modification of Diet in Renal Disease equation) at screening. In some embodiments, the human subject has a platelet count ≥100,000 cells/mm3 at screening. Although body weight is not specified as an inclusion criterion, historically, subjects in clinical trials for treatment of HAE had an average body weight of about 80 kg. Such an exemplary body weight can be used to approximate dose conversions between flat dosing and body weight-based dosing.

In some embodiments, the human subject meets all of the laboratory and other criteria described above at screening for the relevant phase of the study. In some embodiments, a human subject limits alcohol consumption to 1 alcoholic drink per day during screening and through 28-days after treatment with the composition described herein.

In some embodiments, a human subject is a male or female subject who is 18 to 90 years of age (inclusive), e.g., at the time of signing informed consent. In some embodiments, a high follicle-stimulating hormone (FSH) level in the postmenopausal range may be used to confirm a post-menopausal state in women not using hormonal contraception or hormonal replacement therapy. In some embodiments, in the absence of 12 months of amenorrhea, a single FSH measurement is insufficient.). In some embodiments, a female subject is surgically sterile (e.g., hysterectomy, bilateral salpingectomy, and bilateral oophorectomy) at least 1 month prior to screening. In some embodiments, a male subject with partner(s) of child-bearing potential or who are pregnant agree to using a condom prior to screening and for 84 days after study drug administration. In some embodiments, a male subject agrees not to donate sperm for 84 days after study drug administration. The timeframe may be extended beyond the 84 days, if sperm donation is contraindicated based on country-specific guidelines.

In some embodiments, a human subject is assessed for risk of transmission or contraction of SARS-CoV-2 determined acceptable to proceed with an elective procedure at the health care facility (e.g., document that vaccination series completed, recent PCR test negative, or such testing no longer required, etc.).

In some embodiments, a human subject agrees not to participate in another interventional study during the duration of the trial.

b. Subject Exclusion Criteria

In some embodiments, a subject having HAE (HAE Type I or HAE Type II) to whom the LNP composition described herein, e.g., comprising an mRNA encoding a Cas9 nuclease, e.g., Spy Cas9; and a guide RNA that targets a KLKB1 gene, is administered is assessed for one or more of the following subject exclusion criteria.

i. HAE Subject Exclusion Criteria

In some embodiments, the human subject meets the following criteria:

In the phase 2 study, a subject has not used of long-term prophylaxis for HAE within 5 half-lives prior to the start of screening; as provided in the list of prophylaxis agents, half-lives, and recommended washout period in the table below.

TABLE 1
HAE prophylactic products and estimated washout duration
HAE Prophylactic Products and Estimated Washout Duration

| | | Half Life | | | Estimated washout for |
|---|---|---|---|---|---|
| Brand Name | Generic Name | (t_{1/2}) | | Source | (5 half-lives) |
| Danocrine | Danazol | 9.7 | hours | https://www.accessdata.fda.gov | 48.5 hours = ~2 days |
| Cinryze | C1 Esterase | 56-62 | hours | www.cinryze.com | 310 hours = ~13 days |
| | Inhibitor [Human] | | | | |
| Haegarda | C1 Esterase | 69 | hours | www.haegarda.com | 345 hours = ~14.5 days |
| | Inhibitor [Human] | | | | |
| Orladeyo | Berotralstat | 93 | hours | www.orladeyo com | 465 hours = ~19.5 days |
| Oxandrin | Oxandrolone | 13.5 | hours | https://www.rxlist.com/oxandrin | 66.5 hours = ~3 days |

In certain embodiments, the subject has not used of C1-INH for HAE within 5 half-lives of the agent before initiation of the Phase 2 run-in period; i.e., 24-hour washout is required before starting the run-in period after the use of rabbit purified C1-INH (ruconest), and 4-day washout is required before starting the run-in period after the use of human plasma-purified C1-INH (berinert). An exception is made during the run-in period, C1-INH may be used to treat an acute HAE attack.

In certain embodiments, the subject does not have concurrent diagnosis of any other type of recurrent angioedema, including acquired or idiopathic angioedema.

In some embodiments, the human subject does not have a hypersensitivity to any lipid nanoparticles (LNP) component or has previously received LNP and experienced any treatment-related laboratory abnormalities or adverse events (e.g., an ALT or AST >3×ULN if baseline was normal or >3× baseline if baseline was above normal after receiving an LNP containing product, an INR, aPTT or d-dimer >1.5×ULN if baseline was normal or >1.5× Baseline if baseline was above normal after receiving an LNP containing product, a LNP treatment-related adverse event classified as CTCAE Grade 3 or higher, an infusion-related reaction (IRR) to an LNP containing product requiring treatment or discontinuation of infusion).

In certain embodiments, the subject has not been exposed to angiotensin-converting enzyme (ACE) inhibitors or any estrogen-containing medications with systemic absorption within 90 days prior to study drug administration. In certain embodiments, the subject has not used an antithrombotic therapy other than aspirin (e.g., warfarin, dabigatran, apixaban) within 14 days prior to study drug administration.

In certain embodiments, the subject does not have a history of thrombophilia, or positive genetic test for Factor V Leiden or prothrombin 20210. In some embodiments, the human subject does not have a history of cirrhosis. In some embodiments, the human subject does not have known or suspected systemic viral, parasitic, or fungal infection or received antibiotics for bacterial infection. In some embodiments, the human subject does not have a history of Hepatitis B or C infection or positive Hepatitis B surface antigen (HBsAg) or Hepatitis C Virus antibody (HCV Ab) test. In some embodiments, the human subject does not have a history of positive human immunodeficiency virus (HIV) status. In some embodiments, the human subject has not had prior liver, heart or other solid organ transplant or bone marrow transplant or anticipated transplant within 1 year of administration. In some embodiments, the human subject does not have a history of alcohol or drug abuse within 3 years prior to screening. In some embodiments, the human subject is not female and of child-bearing potential or is breastfeeding. In some embodiments, the human subject does not have a positive Severe Acute Respiratory Syndrome Coronavirus-2 (SARS-CoV-2) polymerase change reaction (PCR) test within 7 days of administration.

The study protocol was amended to include women of child-bearing potential, who were initially excluded out of caution as nonclinical studies investigating the potential for oocyte editing were ongoing at the time that the study commenced. While the study was ongoing, a definitive GLP breeding study in transgenic mice containing the human KLKB1 gene was completed. This study revealed no evidence of germline transmission of KLKB1 editing, suggesting that the overall benefit/risk assessment in women of child-bearing potential is comparable to that in the overall patient population. The benefit/risk assessment has been updated to reflect this, and additional changes related to the inclusion of women of child-bearing potential, including pregnancy testing, contraception, and pregnancy reporting requirements have been added.

Assessment of these and other exclusion criteria are known in the art.

In certain embodiments, the subject meets all of the exclusion criteria set forth above.

### 3. Infusion Prophylaxis

In some embodiments, a method described herein, e.g., comprising administering to a subject the LNP composition described herein (e.g., comprising an mRNA encoding a Cas nuclease, e.g., Cas9; and a guide RNA that targets a gene, e.g., a guide RNA that targets the KLKB1 gene), further comprises infusion prophylaxis. In some embodiments, an infusion prophylaxis is administered to a subject before administration of the gene editing composition. In some embodiments, the infusion prophylaxis regimen administered to a subject before administering the LNP composition comprises administering intravenous steroid; intravenous H1 blocker or oral H1 blocker; and intravenous or oral H2 blocker. The intravenous steroid may be dexamethasone, e.g., 10 mg. The intravenous H1 blocker may be diphenhydramine, e.g., 50 mg. The oral H1 blocker may be cetirizine, e.g., 10 mg. The intravenous or oral H2 blocker may be famotidine, e.g., 20 mg.

### Clinical Trial Assessment Protocols and Criteria

The following exemplary kits and methods used in the clinical trial are provided. For assessment of standard laboratory values, e.g., ALT, AST, no methods are provided as such methods are routine and well established in the art.

### Plasma Kallikrein Protein and Activity Levels

Kits for detection of total plasma kallikrein (pre-kallikrein+kallikrein) and for kallikrein activity level are known in the art, and methods to validate kits and assays are known in the art.

The Prekallikrein and Kallikrein Human ELISA kit (Abcam, catalog number ab171015) provides instructions for use of the kit for the quantitative measurement of Human Prekallikrein (Fletcher Factor) concentrations in plasma, serum, saliva, milk, cerebrospinal fluid and cell culture, and cell lysates.

The kit provides a prekallikrein specific antibody precoated onto 96-well plates and blocked for use in the method. Standards, provided with the kit, or test samples are added to the wells and subsequently a Prekallikrein specific biotinylated detection antibody is added and then followed by washing with wash buffer. Streptavidin-Peroxidase Conjugate is added and unbound conjugates are washed away with wash buffer. TMB is then used to visualize Streptavidin-Peroxidase enzymatic reaction. TMB is catalyzed by Streptavidin-Peroxidase to produce a blue color product that changes into yellow after adding acidic stop solution. The density of yellow coloration is directly proportional to the amount of Prekallikrein and Kallikrein captured in plate, and the assay is reported to be about 0.77 ng/ml. Protein assay is a quantitative method with a reference standard curve and concentrations are reported. Validation results demonstrated assay to be specific, accurate, precise, and sensitive to its intended use. Berotralstat in the sample does not interfere with detection. Method does not detect tissue kallikrein.

The SensoLyte® Rh 110 Plasma Kallikrein Activity Assay Kit *Fluorimetric* (AnaSpec, catalog number AS-72255) provides instruction for use of the kit for detection of plasma kallikrein activity using a 96-well plate format. The SensoLyte® Rh110 Plasma Kallikrein Activity Assay Kit employs a fluorescence peptide substrate for the detection of enzyme activity. This substrate contains rhodamine 110 fluorophore (Rh110). Plasma kallikrein cleaves this Rh110 substrate and results in the release of a bright green fluorescence and can be detected at excitation/emission=496 nm/520 nm. The longer wavelength spectra and higher extinction coefficient of Rh110 provide greater sensitivity and less interference from other reaction components. The assay can reportedly detect as low as 1 ng/mL active plasma kallikrein. The kit is can reportedly be used to detect enzyme activity in purified enzyme preparations, biological samples and can also be applied for compound screening. Activity assay is a qualitative method and reported as % inhibition directly based on slope (signal vs. time as a kinetic read out). For use in the clinical trial methods provided herein, per the kit instructions, samples were first activated to convert all pre-kallikrein to kallikrein, and kallikrein activity was subsequently measured. This fluorescent method is a qualitative assay (without a reference standard curve) based on kinetic measurement (fluorescent signal over time) of the enzymatic reaction. Assay slope (signal over time) at the linear range was determined, with post-dose activity (slope) reported as relative signal in comparison to the pre-dose slope.

For both total kallikrein protein levels and kallikrein activity levels, percent inhibition at post-dose time points is calculated for each patient based on their pre-dose levels (average of 3 pre-dose samples, 2 from screening period and 1 from dosing day before dose). The pre-dose samples are only tested once and reported as individual concentrations (protein assay) or % inhibition based on slope of post-dose vs. pre-dose (activity assay). Positive controls are from commercial Kallikrein protein, purified from pooled human plasma.

Normal human plasma (multiple pools) was used to confirm detection for both protein and activity. No patient samples were used for method development and validation. The normal plasma samples are from CRL employee donation program, and samples were processed using identical procedures as in the clinical trials.

### Hereditary Angioedema (HAE) Attack Data Collection and Confirmation

Investigator and the study site teams were provided with the following guidance related to the documentation and review of attack history, along with the collection, review, and assessment of events deemed potential angioedema attacks. The guidance was provided to help to:
Provide a definition of a Hereditary Angioedema (HAE) attack
Define best principles for the review of potential attack(s)
Provide approaches to determine whether those potential events reported by the subject are true HAE attacks

### HAE Attack

To be considered an HAE attack, the signs or symptoms reported as part of the event must include at least 1 of the following:
Peripheral angioedema: cutaneous swelling involving an extremity, the face, neck, torso, or genitourinary region
Abdominal angioedema: abdominal pain, with or without abdominal distention, nausea, vomiting, or diarrhea
Laryngeal angioedema: stridor, dyspnea, difficulty speaking, difficulty swallowing, throat tightening, or swelling of the tongue, palate, uvula, or larynx

Despite the presence of these signs or symptoms, clinically, the event may still not represent an HAE attack when:
There is a diagnosis that refutes the event
The reported event persists well beyond the typical time course of an attack for that subject
There is an alternate etiology

To be considered as a unique HAE attack distinct from a previously confirmed attack, the signs or symptoms must be determined to have commenced greater than 24 hours after resolution of the prior attack's signs or symptoms.

Prodromal symptoms by themselves are not considered an attack. Report of use of acute HAE attack treatment for a potential attack by itself is not confirmation of an HAE attack.

### Subject HAE Attack History

Per the study protocol, each subject's HAE attack history is documented at the study site and entered into the eCRF. Information is to include:
Typical attack frequency
Typical attack location(s) and symptoms
Whether subject ever experiences signs and symptoms of laryngeal attacks
Whether subject experiences prodromal signs and symptoms
Typical attack severity
Typical attack duration
Acute attack therapy use
Any history of prophylaxis therapy
Number of attacks in prior 3 months (90 days)
Complete history of HAE-related medications used in last 90 days

### Subject Training on Diary and Potential HAE Attack Recording

During screening, site personnel train subjects (and caregivers) on identifying symptoms of an attack, the requirements for diary completion and for reporting attacks in the diary. Per the trial protocol, the site personnel assess the subject's compliance with the reporting requirements throughout the study and will retrain the subject, if necessary, in order to maintain compliance with the protocol.

### Potential HAE Attack Information as Reported by the Subject

The following information should be recorded by the subject (or caregiver) in the diary at the time they are reporting a potential HAE attack (or as soon as possible), or when holding discussion with the study personnel:
Date and time signs or symptoms of an attack were first experienced
Description of signs and symptoms experienced, including location(s)
Any medications used to treat the attack
Severity of the attack
Was assistance, medical intervention, clinic visit, emergency room visit, or inpatient hospitalization required
Date and time the subject was no longer experiencing symptoms

Subjects do not have to wait for their symptoms to completely resolve to report a potential HAE attack in the diary.

Information about ongoing symptoms can be obtained by the site during a planned study visit or planned telemedicine visit or phone contact.

Subjects should not withhold or delay any treatment they would normally receive to treat their attack in order to enter potential attack information in the diary.

### Study Site Personnel and Investigator Review of Potential HAE Attack Information

As detailed in the trial protocol, site personnel review the diary information for overall diary compliance. In addition, during a planned study visit or planned telemedicine visit or phone contact the site personnel review the diary information with the subject or caregiver and collect additional information as necessary to further document any potential HAE attack. This additional information is considered study source documentation.

As detailed in the study protocol, the Investigator or designee review the attack information and evaluate if the event represents a confirmed HAE attack. If necessary, for the evaluation, the Investigator or designee may also contact the subject or caregiver to review and collect additional information. This additional information is considered study source documentation.

### Reporting Multiple Attacks

If a subject experiences signs and symptoms that they attribute to more than 1 unique attack they can report this as multiple attacks in the e-dairy. Based on the Investigator or designee review of the reported potential HAE attacks, a determination is to be made as to whether events reported as being separate are confirmed as separate attacks or not.

Site Contact with the Subject or Caregiver

Site personnel establish a recommended method and time to contact the subjects or caregiver per the Schedule of Activities. At Screening and periodically throughout the duration of the study (104 weeks), the site will confirm the primary contact person and, if possible, a back-up person, with contact information (e.g., email, mobile phone, home phone)

### Study Site Personnel and Investigator Review of Diary and Potential HAE Attack Information

Complete and accurate documentation of each reported potential HAE attack benefits in the Investigator or designee's clinical assessment of the attack. The site should review the diary information and ensure the diary contains or they also document and enter into the eCRF the following:
Date and time of contact with the subject
Date and time the subject first experienced signs and symptoms
Overall potential attack severity
Whether potential attack involved laryngeal site
Medications used to treat the potential attack including HAE acute therapy
Required assistance, medical intervention, clinic visit, emergency room visit, or
inpatient hospitalization
Date and time the subject was no longer experiencing any symptoms of the potential
attack
Whether the potential attack is considered to be a confirmed attack
If potential attack is not confirmed, the reason why
Whether the potential attack is considered to be an adverse event

All Investigator assessments for potential and confirmed attacks will documented at the site and also will be entered by site personnel into the eCRF.

### Attack Severity

The overall severity of the subject's potential HAE attack will be determined by the subject and site using the following guidelines:
Mild: Transient or mild discomfort
Moderate: Mild to moderate limitation in activity—some assistance needed
Severe: Marked limitation in activity, assistance required

### HAE Attacks and Reporting of Adverse Events

As detailed in the study protocol, site personnel review the diary information for overall diary compliance. In addition, during a planned study visit or planned telemedicine visit or phone contact the site personnel review the diary information with the subject or caregiver and collect additional information as necessary to further document any potential HAE attack. This additional information is considered study source documentation.

At the time of each contact and scheduled study visits, site personnel will ask if the subject experienced any attacks, adverse events, or changes to the medications they are taking.

Potential HAE attacks will be captured in the diary and eCRF, and assessed by the Investigator or designee. If after Investigator or designee review of a potential HAE attack, an alternate diagnosis is made and the event is deemed not to be an HAE attack, then the event is to be also entered by the site in the eCRF as an adverse event (AE). All AEs, regardless of seriousness, severity, or causal relationship to study drug, is recorded on the AE page of the eCRF. Additional AE reporting details are provided in the study protocol.

If a potential HAE attack is deemed by the Investigator to meet any of the following criteria, it is recorded as an attack in the diary and eCRF AND an AE eCRF or SAE case report form (CRF) must also be completed:
The HAE attack is reported and deemed by the Investigator as clinically more severe than those attacks reported by the subject as part of their HAE attack history
The location of the attack is novel or never previously experienced by the subject per their HAE attack history
The HAE attack is reported and deemed by the Investigator as clinically more prolonged in duration than those attacks reported by the subject as part of their HAE attack history
The HAE attack requires different or significantly greater medical or clinical intervention before attack resolution that those interventions reported by the subject as part of their HAE attack history
The HAE attack leads to hospitalization
Any other clinical assessment or judgment by the Principal Investigator (PI) that deems
that the HAE attack is different and clinically significant than those attacks reported by
the subject as part of their HAE attack history

### Angioedema Quality of Life

The MOXIE Angioedema Quality of Life (AE-QoL) questionnaire is a patient-reported outcome measure developed for the assessment of QoL impairment in subjects with recurrent angioedema, independent of its underlying causes (Weller et al, 2012). To develop the assessment, 110 angioedema patients took part in the validation of AE-QoL. AE-QoL was found to have a four-dimensional structure as well as a valid total score. The questionnaire includes four domains (Functioning, Fatigue/Mood, Fears/Shame, Food) and comprises 17 questions. Test-retesting has demonstrated a good reliability of the instruments total score and domain scores. Gender as well as the patients' self-rated disease activity was found to be predictors of the AE-QoL total score. It is a short assessment that can be used in clinical studies and in routine patient care. The assessment tool has a recall period of 4 weeks. The assessment can be performed at desired intervals, and takes less than 5 minutes to complete. It may be useful to help better characterize affected patients, aid in treatment decisions, and monitor treatment burdens, which can be substantial in HAE. In the trials provided herein, the assessment was used in the primary observation period and at a single post-baseline time point (Week 16) in the primary observation period. Further assessments were later added, specifically EuroQol Group EQ-5D-5L, a brief, multiattribute, generic, health status measure composed of 5 questions; and the Work Productivity and Activity Impairment Questionnaire: General Health (WPAI:GH) assessment.

### EXAMPLES

### Example 1. LNP-Particle Based Composition for KLKB1 Gene Editing

In Vitro Transcription (“IVT”) of Nuclease mRNA

Capped and polyadenylated mRNA containing N1-methyl pseudo-U was generated by in vitro transcription using routine methods. Briefly, a linearized plasmid DNA template and T7 RNA polymerase. Plasmid DNA containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01U/μL, and the reaction was incubated at 37° C. to remove the DNA template.

The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to Sequence Table. When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence e.g., up to 100 nts and are identified in Table 3. Guide RNAs are chemically synthesized by methods known in the art.

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), also called herein Lipid A, cholesterol, DSPC, and PEG2k-DMG in a 50:38:9:3 molar ratio, respectively. The LNPs were formulated with a lipid amine to RNA phosphate (N:P) molar ratio of about 6, and a ratio of gRNA to mRNA of 1:2 by weight. The LNPs used comprise a Cas9 mRNA and an sgRNA.

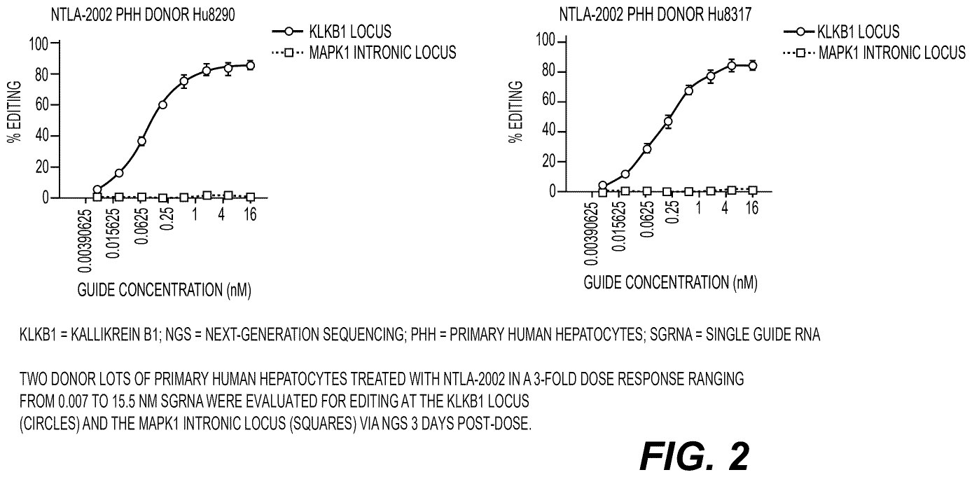

The LNPs were prepared using a cross-flow technique utilizing impinging jet mixing of the lipid in ethanol with two volumes of RNA solutions and one volume of water. The lipid in ethanol was mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). Diluted LNPs were concentrated using tangential flow filtration on a flat sheet cartridge (Sartorius, 100kD MWCO) and then buffer exchanged using PD-10 desalting columns (GE) into 50 mM Tris, 45 mM NaCl, 5% (w/v) sucrose, pH 7.5 (TSS). The resulting mixture was then filtered using a 0.2 m sterile filter. The final LNPs were characterized to determine the encapsulation efficiency, polydispersity index, and average particle size. The final LNP was stored at 4° C. or −80° C. until further use. Reversed phase Ion Pairing high performance liquid chromatography (IP-RP-HPLC) was used to determine the total RNA content in LNP. The lipid nanoparticles were de-formulated to release the RNA prior to IP-RP Chromatography with UV detection. The concentration of each RNA was calculated by comparing the absorbance signal from the sgRNA or cas9 mRNA peak to the corresponding standard curve generated using a reference standard for release testing. The sum of the sgRNA and cas9 mRNA concentrations were reported as total RNA concentration in mg/mL.

### Next-Generation Sequencing (“NGS”) and Analysis for Editing Efficiency

Genomic DNA was extracted from cells or tissue according to methods known in the art, for example using QuickExtract DNA Extraction solution (Epicentre, Cat. QE09050) or Quick Extract (Lucigen, Cat. SS000035-D2). To quantitatively determine the efficiency of editing at the target location in the genome, sequencing was utilized to identify the presence of insertions and deletions introduced by gene editing. PCR primers were designed around the target site within the gene of interest (e.g., KLKB1), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency” or “percent editing”) is defined as the total number of sequence reads with insertions or deletions (“indels”) over the total number of sequence reads, including wild type.

### Example 2. Selection of sgRNA Targeting KLKB1 Gene Including Off-Target Analysis

A sgRNA targeting KLKB1 gene sequence GGAUUGCGUAUGGGACACAA (SEQ ID No: 15; human genome build hg38 within chromosome locus chr4:186251793-186251812) was selected for efficient knockout and specificity after a comprehensive off-target characterization workflow that applied a combination of both in silico and empirical approaches. To select for a high therapeutic index (ratio of on- versus off-target editing), genome-wide assays and targeted sequencing, to identify and verify candidate sgRNA off-target sites were performed for G012267, the guide RNA in NTLA-2002, using the empirical off-target discovery assay, SITE-Seq.

The biochemical, empirical CRISPR/Cas9 off-target discovery assay SITE-Seq is a cell-free biochemical method that is among the most sensitive to potential off-target editing discovery and relies on isolated gDNA (Cameron 2017). SITE-Seq was executed on human gDNA derived from peripheral blood mononuclear cells from 2 unique male blood donors. Each gDNA sample was digested in vitro with assembled RNP of Cas9 and the human KLKB1 sgRNA (hu-G012267) used in NTLA-2002, to induce DNA cleavage at the on-target site and potential off-target sites with homology to the sgRNA sequence. After gDNA digestion, the free gDNA fragment ends were ligated with adapters to facilitate edited fragment enrichment and NGS library construction. The NGS libraries were sequenced, and through bioinformatic analysis, the reads were analyzed to determine the genomic coordinates of the free DNA ends. Locations in the human genome with an accumulation of reads were then annotated as potential off-target sites.

SITE-Seq discovered 61 potential off-target loci for the KLKB1 targeting guide hu-G012267. The potential off-target editing loci identified by SITE-Seq (Cameron 2017) for hu-G012267 were used to complement the computational off-target editing discovery approach Cas-OFFinder (Bae 2014) as an orthogonal discovery technology. SITE-Seq discovered 61 off-target sites, and Cas-OFFinder nominated 47 sites. As an added measure of precaution, potential off-target loci discovered by SITE-Seq from 2 additional experiments using alternate hu-G012267 sgRNA synthesis chemistry were also included for off-target editing validation, which increased the number of potential off-target loci from 108 (47 Cas-OFFinder plus 61 SITE-Seq loci) to 196.

The curated 196 potential off-target editing loci identified by Cas-OFFinder and SITE-Seq were subsequently characterized for off-target indel detection through targeted amplicon sequencing (rhAmpSeq or AMP-Seq) using PHH treated with NTLA-2002 at a supersaturating dose. Targeted amplicon sequencing can determine whether off-target editing at each potential site validates in the relevant cell type, PHH. To better contextualize off-target editing at a therapeutically relevant dose, editing frequency in a dose response curve was (DRC) determined for each validated off-target site.

To maximize the sensitivity of off-target editing detection, Intellia executed genome editing in vitro at supersaturating doses of guide RNA (6 nM) 50-fold higher than empirically determined therapeutically relevant dose (0.12 nM) where 80% kallikrein protein reduction is achieved in PHH. Purified gDNA from 2 PHH donors edited with the supersaturating dose of NTLA-2002 LNPs were then characterized with rhAMPSeq, a multiplexed amplicon PCR method. Due to limitations in PCR primer compatibility and technical failures of amplicon enrichment, rhAMPSeq is not capable of characterizing every potential off-target editing site discovered by Cas-OFFinder and SITE-Seq. Potential off-target loci were successfully characterized by rhAMPSeq or were alternatively characterized with single plex AMP-Seq to complete characterization of all potential off-target editing loci. rhAmpSeq and AMP-Seq were empirically determined to have a 0.3% and 0.5% lower limit of indel detection, respectively (TREP-0079 and TREP-0120, respectively).

The validated off-target locus was further characterized in DRCs of NTLA-2002 to put off-target editing validation and indel frequencies in context with a therapeutically relevant dose of NTLA-2002 which achieves >80% kallikrein protein reduction in PHH. Two donor lots of PHH (Hu8290 and Hu8317) were treated with NTLA-2002 in a DRC and editing at the on-target and off-target sites were assessed. rhAmpSeq detected 1 validated off--target editing site in the first intron of the gene MAPKI (chr22:21837431-21837451) at a frequency of 0.90% and 0.60% in PHH lots Hu8290 and Hu8317, respectively (Table 2) at concentrations 50-fold higher than empirically determined therapeutically relevant dose where 80% kallikrein protein reduction is achieved. AMP-Seq did not detect any validated off-target sites.

In the DRC experiment, saturating levels of >85% on-target editing at the KLKB1 locus were achieved in PHH from both donors at high doses (FIG. 2). The maximum MAPKI intronic locus editing observed was 1.5% at doses >50-fold above the therapeutically relevant dose where 80% kallikrein protein reduction was achieved. At a therapeutically relevant dose (0.12 nM guide RNA), no detectable MAPKI editing was observed in either lot of PHH (lower limit of detection=0.5%).

Of the 196 potential off-target sites nominated by Cas-OFFinder and SITE-Seq, only 1 site demonstrated valid CRISPR/Cas9 mediated editing in treated PHH. This site was located in intron 1 of MAPKI. Editing at the MAPKI off-target site was not detected at a therapeutically relevant dose of NTLA-2002, suggesting low risk associated with editing at this site.

TABLE 2
Validated Off Target Indel Detection after Genomic Editing with NTLA 2002

| | | Hu8290 | Hu8290 | Hu8317 | Hu8317 | |
|---|---|---|---|---|---|---|
| Identification | Location | ΔIndel % | p-value | ΔIndel % | p-value | Annotation |
| G012267 | chr4:186251792- | 95.37% | 9.65 × 10⁻⁶ | 95.57% | 7.91 × 10⁻⁶ | KLKB1 |
| On-target | 186251812(+) | | | | | (exon) |
| G012267 | chr22:21837431- | 0.90% | 2.04 × 10⁻³ | 0.60% | 5.89 × 10⁻³ | MAPK1 |
| Off-target | 21837451(−) | | | | | (intron 1) |

KLKB1 = kallikrein B1

To further characterize potential effects from off-target editing in the MAPKI intron, MAPKI mRNA transcripts were quantified with Droplet Digital™ PCR (ddPCR).

A guide with perfect homology to the MAPKI intronic site, hu-G018729, was designed to maximize any potential phenotype associated with editing at this locus. This guide, along with hu-G012267 (the KLBK1 targeting guide in NTLA-2002) and ctl-G000739 (a non-targeting control sgRNA) were formulated with Cas9 mRNA000042 for in vitro studies into individual LNPs: LNP-G018729 (LNP containing MAPKI intronic targeting hu-G018729), NTLA-2002, and ctl-LNP-G000739 (LNP containing non-targeting ctl-G000739), respectively. PHH were treated with each of the 3 LNPs in dose response analyses. gDNA was extracted from these cells and sequenced to determine indel rates at edited loci 3 days post-treatment. RNA was also extracted from these cells for MAPK1 expression analysis 10 days post-treatment. Paired 2-tailed t-tests were performed to estimate the true difference between treatment group and non-targeting control (LNP-G000739) group means using the ratio of the difference in group means over the pooled standard error of both groups. The Benjamini-Hochberg (B-H) procedure was used as a tool to reduce the false discovery rate.

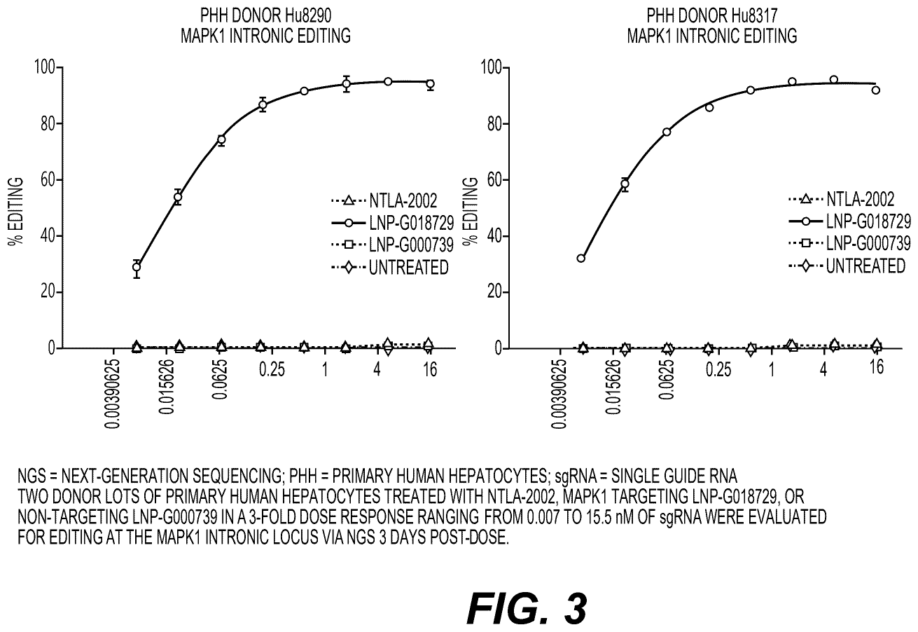

PHH from both donors achieved saturating levels of editing at the MAPK1 locus when treated withMAPK1 targeting LNP-G018729 (FIG. 3). SubsequentMAPK1 mRNA quantification 10 days post-treatment across all doses of both NTLA-2002 treated PHH and MAPKI targeting LNP-G018729 treated PHH resulted in no significant difference in MAPKI transcript when compared to control samples using the paired t-tests with the B—H procedure (Table 3).

These results demonstrating no change in MAPKI mRNA transcript levels are expected due to the location of the off-target site within the non-coding intronic region of the MAPKI gene. The intronic location of the validated off-target site combined with the observation of no impact on MAPKI mRNA levels even with LNP-G018729 achieving >90% editing at this locus, and no detectable editing at this locus at therapeutically relevant doses of NTLA-2002, suggest low risk associated with off-target editing.

TABLE 3
MAPK1 mRNA Expression In Primary Human Hepatocytes Treated with
NTLA-2002 or MAPK1 Targeting LNP1961

| | | | Percent | MAPK1 | | | | | |
|---|---|---|---|---|---|---|---|---|---|
| | | | MAPK1 | mRNA | | | Total | | |
| | PHH | Guide | intronic | Relative | p- | Rank | Tests | (i/m) | Significance |
| Treatmeat | Donor | (nM) | Editing | Expression | value | (i) | (m) | Q | P < (i/m)Q |
| MAPK1 | Hu8290 | 1.722 | 94.13 | 1.05 | 0.454 | 4 | 6 | 0.033 | FALSE |
| Targeting | | [symbol] | 86.77 | 0.83 | 0.780 | 10 | 6 | 0.083 | FALSE |
| LNP-G018729 | Hu8317 | 1.722 | 94.10 | 0.75 | 0.394 | 13 | 6 | 0.108 | FALSE |
| | | 0.191 | 85.67 | 0.96 | 0.283 | 10 | 6 | 0.083 | FALSE |
| NTLA-2002 | Hu8290 | 1.722 | 0.23 | 0.91 | 0.437 | 8 | 6 | 0.025 | FALSE |
| | | 0.191 | 0.23 | 0.95 | 0.814 | 11 | 6 | 0.092 | FALSE |
| | Hu8317 | 1.722 | 0.23 | 0.93 | 0.020 | 2 | 6 | 0.017 | FALSE |
| | Hu8290 | 0.191 | 0.13 | 0.86 | 0.162 | 6 | 6 | 0.050 | FALSE |

mRNA = messenger RNA; PHH = primary human hepatocytes; i = the individual p-value rank; m = number of tests; Q = false discovery rate [symbol]
indicates data missing or illegible when filed

### Example 3. DNA Structural Variant Characterization in Primary Human Hepatocytes

To characterize potential DNA structural variants (SVs), 2 complementary technologies were applied to characterize genomic stability and potential DNA structural variants that may occur as a result of genome editing with NTLA-2002: a qualified unique identifier tagmentation (UnIT) NGS assay and long-range PCR followed by long-read sequencing with Pacific Biosciences technology. Targeted PCR-based amplicon sequencing with Illumina-based NGS is limited in its capacity to characterize and quantify DNA structural variants (SVs) such as deletions >100 bp, duplications, inversions, and translocations.

The DNA SV discovery technology called UnIT, was developed based on published reports using Illumina NGS (Giannoukos 2018; Klein 2011). This method enabled the simultaneous measurement of small indels (<100 bp) at the on-target site and potential structural variants due to rearrangements after DNA repair such as inversions, duplications, and inter-chromosomal translocations.

DNA SVs were detected using split and discordant NGS alignments. When an NGS read or read pair aligned to >1 locus in the genome, the 2 fragments involved (2 segments from the same read in a split alignment or 2 reads from the same pair in a discordant alignment) were used to classify the SV.

A complementary long-range sequencing approach was implemented, which can capture large deletions around the edit site that short-read NGS sequencing may miss. Long-range PCR followed by long read sequencing with Pacific Biosciences technology characterized the potential for large deletions near the on-target site which may result from the DNA repair process after genome editing with NTLA-2002.

The UnIT DNA SV characterization assay was applied to gDNA purified from 2 PHH donors in triplicate after genome editing with NTLA-2002. No DNA SV above the empirically determined 0.5% limit of detection were detected.

The characterization of potential large deletions with long-range PCR and long read sequencing was executed on 2 unique donors of PHH. No large deletions above the limit of detection (0.5%) were observed in either PHH lot.

No DNA structural variants above the empirically determined 0.5% limit of detection were identified and no identified translocations were associated with any known oncogenic risk. Therefore, genomic rearrangements to the on-target locus do not represent a known safety risk.

### Example 4. In Vitro Evaluations of the Potency of NTLA-2002

In vitro. In vitro pharmacology studies have been performed for NTLA-2002 and cyn-LNP-G013901 in primary hepatocytes of human and cynomolgus monkey, respectively. In vitro activity (gene editing, KLKB1 mRNA reduction, and kallikrein protein reduction) were tested in human hepatocytes. Saturating levels of NTLA-2002 resulted in ≥85% indel formation (editing) in the KLKB1 gene, with subsequent ≥85% reduction in KLKB1 mRNA, and >99% reduction in kallikrein protein. Comparable in vitro activity for cyn-LNP-G013901 was observed in primary monkey hepatocytes. Relative in vitro activity of cyn-LNP-G013901 and NTLA-2002 was considered when pharmacokinetic (PK)/pharmacodynamic (PD) data from the monkey studies was used to predict in vivo human PD effect (kallikrein protein reduction).

### Example 5. In Vivo Evaluations of the Potency of NTLA-2002 in Transgenic Mice

Functional validation of in vivo pharmacology for NTLA-2002 was performed in a transgenic murine model expressing the human KLKB1 gene (huKLKB1 mouse). The Hu KLKB1 mouse model comprises a humanized KLKB1 locus in which the region from start codon to stop codon of mouse KLKB1 was replaced with the corresponding human genomic sequence. Animals were weighed and dosed at volumes specific to individual body weight. NTLA-2002 was dosed at 0.3, 0.1, 0.03 and 0.01 mg total RNA per kg bodyweight.

At day 13 post-LNP administration, mice were euthanized. lysed using a Zymo Research Bashing Bead Lysis Rack, and RNA was extracted using the Qiagen RNeasy Mini Kit (Qiagen, Cat. 74106) according to the manufacturer's protocol. RNA was quantified using a Nanodrop 8000 (ThermoFisher Scientific, Cat. ND-8000-GL). RNA samples were stored at −20° C. prior to use.

The SuperScript III Platinum One-Step qRT-PCR Kit (Invitrogen, Cat. 11732-088) was used to create the PCR reactions. Quantitative PCR probes targeting Hu KLKB1 and internal control Ms PPIB were used in the reactions. The quantitative PCR assay was performed according to the manufacturer's specifications, scaled to the appropriate reaction volume, as well as using the Hu KLKB1 and Ms PPIB probes specified above. The StepOnePlus Real-Time PCR System (Thermo Fisher Scientific, Cat. 4376600) was used to perform the real-time PCR reaction and transcript quantification according to the manufacturer's protocol.

Hu KLKB1 mRNA was quantified using a standard curve starting at 20 ng/uL of pooled mRNA from the vehicle control group, with five further 3-fold dilutions ending at 0.06ng/uL. Ct values were determined from the StepOnePlus Real-Time PCR System. Reduction of total secreted human prekallikrein protein for cells treated with KLKB1 reagents was determined by ELISA as described in Example 1.

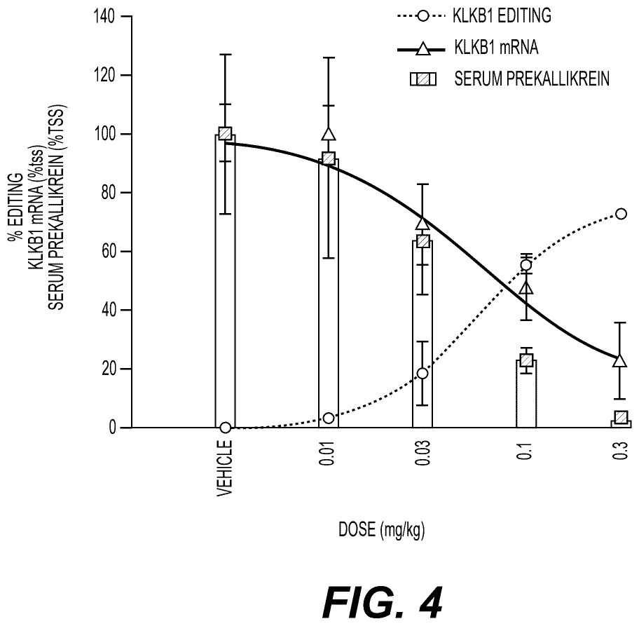

Table 4 and FIG. 4 show percent editing, serum prekallikrein levels as a percent of TSS vehicle control treated mice, and mRNA transcript levels as a percent of TSS vehicle control treated animals.

TABLE 4
Percent Editing, KLKB1 mRNA (% of basal level) and
Serum Prekallikrein Protein Levels (% of basal level)
in Hu KLKB1 Mouse Model

| | | | % | % | % | |
|---|---|---|---|---|---|---|
| | Dose | | Edit- | TSS | TSS | |
| Guide | (mpk) | | ing | protein | mRNA | SD |
| TSS | 0 | Mean | 0.1 | 100 | 100.5 | 9.7 |
| | | Animal 1 | 0.1 | 75.8 | | |
| | | Animal 2 | 0.1 | 93.8 | | |
| | | Animal 3 | 0.1 | 76.0 | | |
| | | Animal 4 | 0.1 | 137.6 | | |
| | | Animal 5 | 0.1 | 116.8 | | |
| NTLA- | 0.01 | Mean | 3.9 | 91.9 | 100.1 | 9.5 |
| 2002 | | Animal 1 | 4.4 | 55.3 | | |
| | | Animal 2 | 3.8 | 57.3 | | |
| | | Animal 3 | 4.5 | 126.2 | | |
| | | Animal 4 | 4.2 | 122.2 | | |
| | | Animal 5 | 2.6 | 98.6 | | |
| | 0.03 | Mean | 19.0 | 64.2 | 69.3 | 13.8 |
| | | Animal 1 | 22.1 | 38.6 | | |
| | | Animal 2 | 0.3 | 51.0 | | |
| | | Animal 3 | 26.9 | 78.9 | | |
| | | Animal 4 | 21.3 | 80.5 | | |
| | | Animal 5 | 24.3 | 72.2 | | |
| | 0.1 | Mean | 55.4 | 23.3 | 48 | 11.4 |
| | | Animal 1 | 52.1 | 17.6 | | |
| | | Animal 2 | 52.7 | 19.6 | | |
| | | Animal 3 | 56.5 | 25.3 | | |
| | | Animal 4 | 57.5 | 25.6 | | |
| | | Animal 5 | 58.0 | 28.3 | | |
| | 0.3 | Mean | 72.9 | 3.1 | 23 | 13 |
| | | Animal 1 | 73.9 | 2.7 | | |
| | | Animal 2 | 70.4 | 2.9 | | |
| | | Animal 3 | 72.7 | 3.1 | | |
| | | Animal 4 | 73.1 | 3.4 | | |
| | | Animal 5 | 74.3 | 3.4 | | |

A proof-of-concept study was performed to evaluate KLKB1 gene editing, total kallikrein protein expression, and vascular leakage in humanized mice. There were 6 groups (N=5 with 2 male, 3 female mice per group). Animals were weighed and dosed at volumes specific to individual body weight. NTLA-2002 was dosed via the lateral tail vein at 0.03 mg/kg, 0.1 mg/kg, or 0.3 mg/kg based on total RNA cargo in a volume of 10 ml per kilogram body weight or vehicle control (TSS).

At one day prior to the vascular leakage study, blood was collected and processed and secreted human prekallikrein was measured via an ELISA to detect total kallikrein. The Evans Blue vascular permeability assay is an established model of edema and vascular leakage that can be used as a model in the study of HAE (see, e.g., Bhattacharjee et al., 2013). Briefly, thirteen days post-dose, vascular permeability was induced using a 2.5 mg/kg intraperitoneal injection of the angiotensin converting enzyme (ACE) inhibitor captopril. After 15 minutes, a mixture of Evans Blue Dye (30 mg/kg) and dextran sulfate (0.3 mg/kg) was administered by intravenous tail injection. The animals were euthanized 15 minutes after this injection and evaluated for dye extravasation into the colon by optical density (OD) at the absorbance of 600 nm via the Clariostar plate reader (BMG LabTech). Liver and serum were collected to quantify huKLKB1 gene editing and kallikrein protein, respectively.

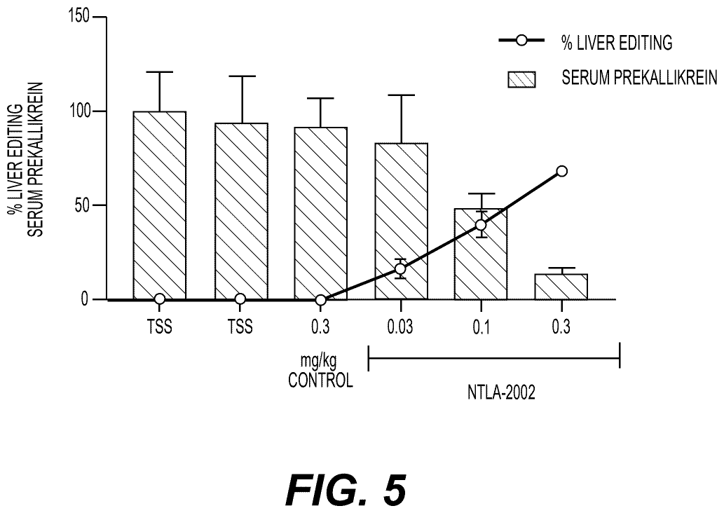

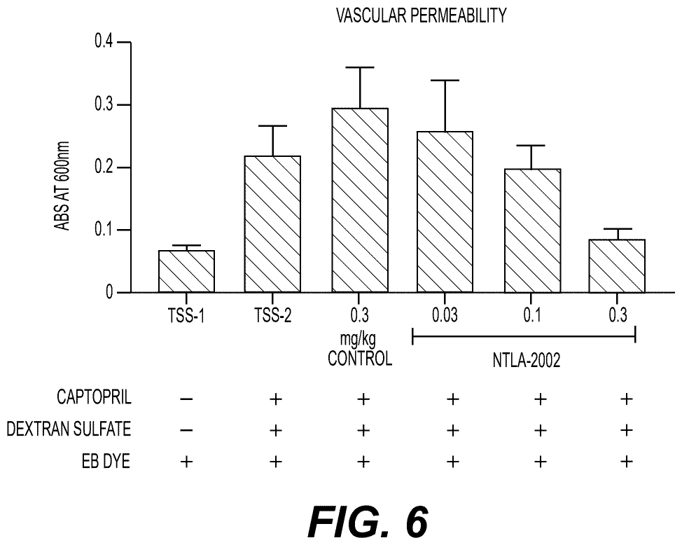

The results for percent editing, serum hu Prekallikrein levels, and vascular leakage are shown in Table 5 and FIGS. 5 and 6.

TABLE 5
Percent editing, Prekallikrein levels,
and vascular leakage in huKLKB1 mice

| | | | Serum | |
|---|---|---|---|---|
| | Dose | % | Kallikrein | Colon |
| | (mg/kg) | Editing | (ug/ml) | (OD) |
| TSS-1 | 0 | 0.02 | 100.00 | 0.07 |
| TSS-2 | 0 | 0.06 | 94.92 | 0.22 |
| Control | 0.3 | 0.1 | 92.99 | 0.30 |
| NTLA-2002 | 0.03 | 16.48 | 82.97 | 0.26 |
| NTLA-2002 | 0.1 | 40.46 | 48.97 | 0.20 |
| NTLA-2002 | 0.3 | 68.66 | 13.88 | 0.09 |

Dose dependent increases in KLKB1 gene editing and serum kallikrein reduction led to a decrease in captopril-induced vascular permeability. At the highest dose tested, induced permeability was similar to baseline levels.

### Example 7. LNP-Mediated Editing in Non-Human Primates

Cynomolgus monkeys were treated in cohorts of n=3. This study was conducted with LNP formulations according to Example 1. Each LNP formulation contained a polyadenylated Cas9 mRNA (comprising SEQ ID NO: 408) and gRNA (G013901, a cynomolgus specific KLKB1 guide RNA, mG*mG*mA*UUACAUAUGGGACACAAGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 409)) with an mRNA:gRNA ratio of 2:1 by weight. Animals were dosed at 1.5, 3, or 6 mg per kg doses based on total RNA cargo. Indel formation (percent editing) was measured by NGS. Total kallikrein activity and plasma kallikrein protein level were measured as described above.

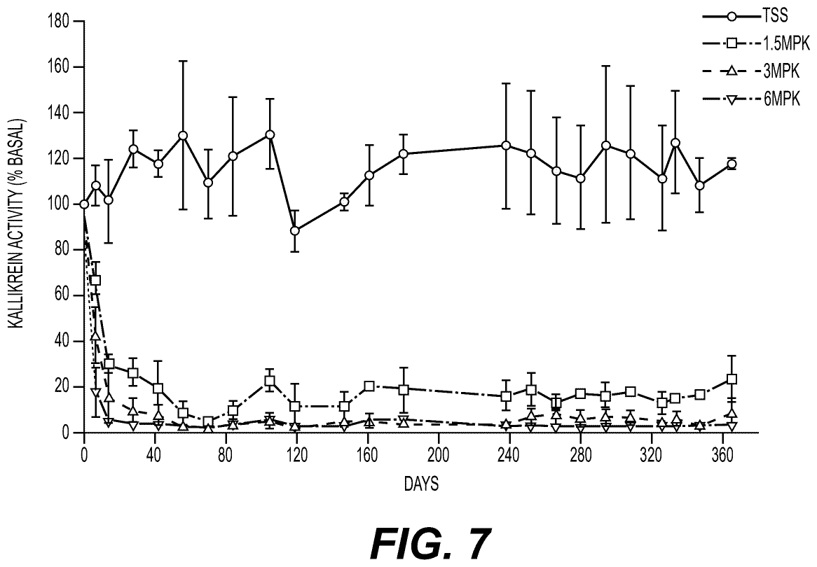

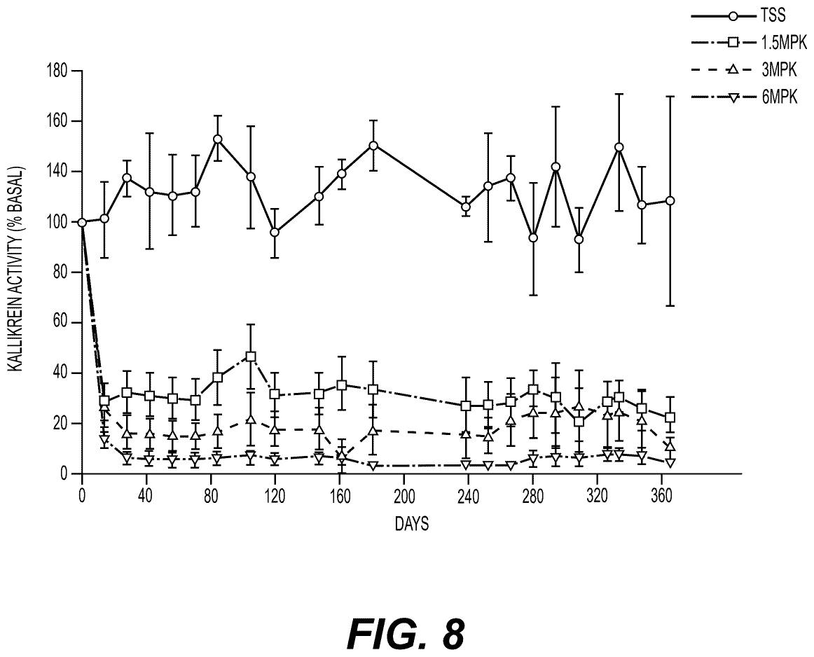

The study showed that knockout of KLKB1, which is part of a biological pathway that results in release of bradykinin, with G013901 produced up to a 9000 reduction in kallikrein activity in NHP groups, or more, a robust response that exceeds the target activity shown to achieve a therapeutically meaningful impact on HAL attack rates (600% kallikrein activity reduction; Banerji, 2017). This study showed a dose-dependent correlation between increased editing rates, reduced plasma kallikrein levels, and reduced kallikrein activity. The response has been durable through one year in NUPs. Circulating kallikrein protein and activity levels are provided in Tables 6 and 7; and FIGS. 7 and 8.

TABLE 6
Kallikrein Activity (% of basal activity)

| | TSS | | 1.5 mpk | | 3 mpk | | 6 mpk | |
|---|---|---|---|---|---|---|---|---|
| | (n = 3) | | (n = 3) | | (n = 3) | | (n = 3) | |
| Day | Mean | SD | Mean | SD | Mean | SD | Mean | SD |
| 0 | 100 | 0 | 100 | 0 | 100 | 0 | 100 | 0 |
| 7 | 108.4 | 9 | 67.8 | 7 | 42.8 | 12.4 | 18.2 | 10.6 |
| 14 | 101.8 | 18.3 | 31.1 | 3.2 | 15.9 | 11 | 5.3 | 1.6 |
| 28 | 124.4 | 7.9 | 26.8 | 6.5 | 10.6 | 5.4 | 3.9 | 1.4 |
| 42 | 117.8 | 5.8 | 19.8 | 11.9 | 8.6 | 4.4 | 3.6 | 0.4 |
| 56 | 130.3 | 32.5 | 8.8 | 5 | 3.4 | 0.6 | 4.2 | 2.2 |
| 70 | 109.2 | 15 | 6 | 1.7 | 3 | 0.4 | 3.9 | 1.6 |
| 84 | 121.3 | 25.7 | 10.6 | 3.8 | 4.2 | 2.1 | 4.7 | 2.5 |
| 105 | 130.8 | 15.5 | 23.3 | 4.9 | 6 | 3.6 | 4.7 | 2.5 |
| 119 | 88.2 | 9.1 | 12.2 | 10.2 | 3.4 | 0.5 | 3 | 0.5 |
| 147 | 101.1 | 3.3 | 11.9 | 6.6 | 4.5 | 1.4 | 3 | 0.5 |
| 161 | 113.2 | 13.4 | 21 | 1.8 | 6 | 2.8 | 5.6 | 1.8 |
| 180 | 122.3 | 8.7 | 19.2 | 9.5 | 4.7 | 1 | 5 | 1.9 |
| 238 | 125.5 | 27.3 | 16.6 | 6.5 | 4.7 | 1.4 | 3.2 | 0.7 |
| 252 | 122.7 | 27 | 19.1 | 7.3 | 8.2 | 4.4 | 3.1 | 0.7 |
| 266 | 115 | 23.3 | 13.9 | 3.3 | 8.9 | 5.2 | 2.7 | 0.6 |
| 280 | 112.2 | 22.7 | 17.5 | 2.3 | 6.8 | 3.6 | 3 | 1 |
| 294 | 126.3 | 34.1 | 17 | 5.8 | 7.9 | 4.1 | 2.9 | 0.6 |
| 308 | 122.9 | 28.9 | 18.2 | 2.3 | 7.4 | 3.3 | 3 | 0.8 |
| 326 | 111.6 | 23 | 13.3 | 5.2 | 5.5 | 2.9 | 3.5 | 0.4 |
| 333 | 127.3 | 22.4 | 15.9 | 1.8 | 6.8 | 2.8 | 3.3 | 0.4 |
| 347 | 108.4 | 11.7 | 16.9 | 1 | 4.1 | 1.8 | 2.9 | 0.3 |
| 365 | 118 | 2.2 | 24.1 | 10.2 | 10 | 5.7 | 3.4 | 0.7 |

TABLE 7
Plasma Kallikrein Protein Levels (% of basal level)

| | TSS | | 1.5 mpk | | 3 mpk | | 6 mpk | |
|---|---|---|---|---|---|---|---|---|
| | (n = 3) | | (n = 3) | | (n = 3) | | (n = 3) | |
| Day | Mean | SD | Mean | SD | Mean | SD | Mean | SD |
| 0 | 100 | 0 | 100 | 0 | 100 | 0 | 100 | 0 |
| 14 | 101.3 | 14.9 | 28.8 | 7.4 | 27.1 | 8.2 | 13.7 | 3.3 |
| 28 | 117.7 | 7 | 32.7 | 8.5 | 16.9 | 6.8 | 6.6 | 2.3 |
| 42 | 112.6 | 23.5 | 31.5 | 9.1 | 16 | 7 | 6.3 | 2.8 |
| 56 | 111 | 16 | 30.1 | 8.8 | 15.4 | 6 | 5.8 | 2.5 |
| 70 | 112.5 | 14.1 | 29.8 | 8 | 15.4 | 5.8 | 5.8 | 2.8 |
| 84 | 133.6 | 9 | 38.7 | 11.1 | 17.1 | 6.7 | 6.7 | 2.5 |
| 105 | 118.2 | 20.1 | 47 | 12.7 | 22.3 | 10.3 | 7.6 | 3.8 |
| 119 | 96 | 9.8 | 31.6 | 8.7 | 18.1 | 7.2 | 6.4 | 2.2 |
| 147 | 110.7 | 12 | 32.1 | 8.3 | 18.3 | 8.1 | 6.7 | 2.2 |
| 161 | 119.3 | 6.1 | 35.8 | 10.5 | 7.5 | 6.8 | 7.4 | 3.5 |
| 180 | 131 | 10.1 | 33.8 | 11.4 | 18 | 10.1 | 2.9 | 1 |
| 238 | 106.2 | 3.7 | 27.4 | 10.8 | 16.4 | 9.8 | 3.7 | 0.9 |
| 252 | 114.3 | 21.4 | 27.9 | 9.1 | 15.6 | 7.1 | 3.9 | 0.8 |
| 266 | 117.6 | 8.7 | 28.7 | 9.7 | 21.8 | 10.4 | 3.7 | 0.5 |
| 280 | 93.4 | 22.1 | 33.7 | 7.1 | 24.6 | 9.8 | 6.3 | 2.9 |
| 294 | 122.1 | 23.7 | 30.4 | 13.8 | 24.6 | 14 | 7.4 | 4 |
| 308 | 93.2 | 12.8 | 20.6 | 13.8 | 27.6 | 14.2 | 6.2 | 3.1 |
| 326 | N/A | | 29.7 | 7.3 | 23.8 | 12.5 | 8 | 2.8 |
| 333 | 130.2 | 25.5 | 30.9 | 6.4 | 25.3 | 12.4 | 7.8 | 2.3 |
| 347 | 106.9 | 15 | 26 | 8.2 | 22.1 | 11.2 | 7.3 | 3.2 |
| 365 | 108.5 | 41.3 | 22.7 | 8 | 11.4 | 5.3 | 4.3 | 1.6 |

Tests of select NUP samples found no observed impact on coagulation pathway biomarkers with KLKB1 knockout in NHPs at weeks 10 or 15 (based on measuring prothrombin, APTT, and fibrinogen (all at week 10), and Factor XII (at week 15)) when comparing TSS buffer control groups to treated groups.

The NUP study was repeated to evaluate total kallikrein protein expression, and total kallikrein activity levels in cynomolgus monkeys using guide G012267 which includes a guide sequence fully complementary to human KLKBL1. The guide sequence of G012267 has one nucleotide difference when compared to the G013901 which has a guide sequence fully complementary to cynomolgus KLKB1. The experimental protocol and LNP formulations in this study were essentially the same as described in the above experiment, except animals (n=3) were only dosed at 3 mg per kg based on total RNA cargo. Total kallikrein activity and kallikrein protein levels were measured using the methods described in Example 1.

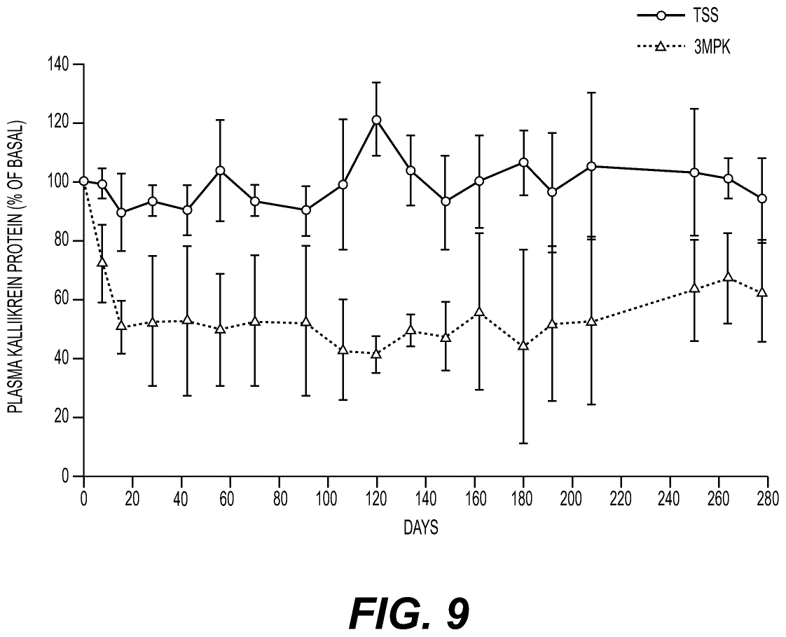

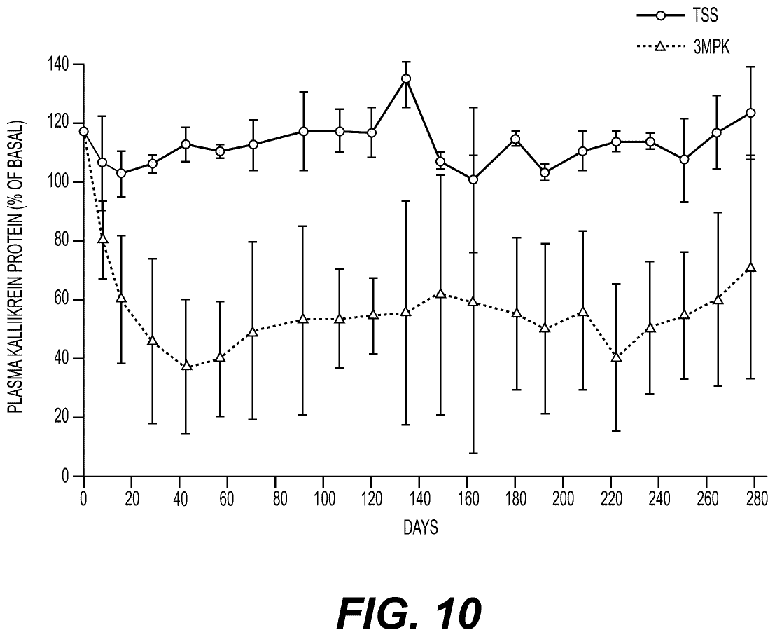

The study showed that knockdown of KLKB1 with G012267 produced up to a 65% reduction in kallikrein activity in NHP groups. The response was durable through 9 months in NHPs. Circulating kallikrein protein and activity levels are provided in Tables 8 and 9, and FIGS. 9 and 10.

TABLE 8
Kallikrein Activity (% of basal activity)

| | |
|---|---|
| TSS (n = 3) | 3 mpk (n = 3) |

| | | Std. | | Std. |
|---|---|---|---|---|
| Day | Mean | Dev | Mean | Dev |
| 0 | 100 | N/A | 100 | N/A |
| 7 | 90.9 | 13.7 | 68.7 | 11.3 |
| 15 | 87.7 | 6.6 | 51.3 | 18.7 |
| 28 | 90.5 | 2.5 | 39.3 | 24 |
| 42 | 96.5 | 4.8 | 31.9 | 19.5 |
| 56 | 94.5 | 1.6 | 34.1 | 16.7 |
| 70 | 96.2 | 7.2 | 42.3 | 25.7 |
| 91 | 100.2 | 11.7 | 45.3 | 27.6 |
| 106 | 100.2 | 6.3 | 45.7 | 14.3 |
| 120 | 99.8 | 7.4 | 46.5 | 11.1 |
| 134 | 115.2 | 8.5 | 47.5 | 32.4 |
| 148 | 91.5 | 2 | 52.9 | 34.7 |
| 162 | 86.1 | 21.1 | 50.1 | 43.2 |
| 180 | 97.9 | 1.8 | 47.3 | 22.1 |
| 192 | 87.9 | 1.9 | 43 | 24.6 |
| 208 | 94.4 | 5.8 | 48 | 23 |
| 222 | 97 | 2.9 | 34.6 | 21.1 |
| 236 | 97.2 | 2.2 | 43.5 | 19.1 |
| 250 | 91.8 | 12 | 46.5 | 18.3 |
| 264 | 99.9 | 10.6 | 51.3 | 25.2 |
| 278 | 105.3 | 13.4 | 60.8 | 32.3 |

TABLE 9
Plasma Kallikrein Protein Levels (% of basal level)

| | |
|---|---|
| TSS (n = 3) | 3 mpk (n = 3) |

| | | Std. | | Std. |
|---|---|---|---|---|
| Day | Mean | Dev | Mean | Dev |
| 0 | 100 | N/A | 100 | N/A |
| 7 | 99.6 | 5.1 | 72.3 | 13.3 |
| 15 | 89.7 | 13.1 | 50.6 | 9 |
| 28 | 93.9 | 5.2 | 52.8 | 22.3 |
| 42 | 90.3 | 8.6 | 52.7 | 25.5 |
| 56 | 104.2 | 17.3 | 49.9 | 19.1 |
| 70 | 93.9 | 5.2 | 52.8 | 22.3 |
| 91 | 90.3 | 8.6 | 52.7 | 25.5 |
| 106 | 99.3 | 21.9 | 43.1 | 17.3 |
| 120 | 121.7 | 12.6 | 41.1 | 6.2 |
| 134 | 104.2 | 12 | 49.7 | 5.5 |
| 148 | 93 | 15.8 | 47.5 | 11.8 |
| 162 | 100.3 | 15.7 | 56.2 | 26.7 |
| 180 | 106.8 | 10.9 | 44.1 | 33.2 |
| 192 | 96.5 | 20.5 | 52 | 26.6 |
| 208 | 105.6 | 25 | 52.9 | 28.5 |
| 250 | 103.7 | 21.5 | 63.3 | 17.4 |
| 264 | 101.4 | 7 | 67.3 | 15.4 |
| 278 | 94.3 | 14.4 | 62.6 | 16.7 |

TABLE 10
Predicted kallikrein protein reduction percent in humans
based on modeling from non-human primate studies

Predicted kallikrein % reduction

| Total | | | 7.9-fold more |
|---|---|---|---|
| RNA | | | potent in |
| dose | BW* | | human than |
| (mg) | (kg) | Equipotent | cyno |
| 25 | 81.2 | 29.4 | 80.7 |
| 75 | 81.2 | 58.4 | 95.1 |
| 150 | 81.2 | 75.2 | 99.4 |

*Mean Body Weight in HAE patients as reported by Wang et al., Clinical and Translational Science. 2020; 13 (6): 1208-16.

### Example 8. Biodistribution and Tissue Editing Studies

Pharmacokinetic studies using a cynomolgus surrogate LNP formulation containing sgRNA G013901 described in the example above, were performed in NHP. Exposure of LNP components was nearly or dose proportional in males, and nearly or greater than dose proportional in females over a 3-fold increase in dose level. Exposures to components were generally similar in male and female monkeys. In studies using the surrogate agent cyn-LNP-G013901, the components were cleared from the circulation in <5 days.

Tissue editing in NHP liver (target tissue) and biodistribution to other tissues was evaluated using both NTLA-2002 and its surrogate cyn-LNP-G013901. Dose-dependent liver editing was observed as described in the pharmacology summary. In sexually mature male NHP treated with either 1.5 or 4.5 mg/kg cyn-LNP-G013901, average editing in the adrenal was 2.4% and 18.7%, and average editing in the spleen was 12.2%, and 18% at those dose levels respectively. No editing was detected in semen or testis. In animals treated with 3, 6, or 9 mg/kg NTLA-2002, average editing in the adrenal was 1.2%, 4.0%, and 5.1%, and spleen was 2.4%, 3.1%, and 6.0% at those dose levels respectively. Similar to males treated with cyn-LNP-G013901, NTLA-2002-treated males exhibited no detectable editing in testis tissue. In females, low levels of editing ranging from 0.8-1.8% were detected in bulk ovary tissue from some, but not all, females.

Similar findings were observed in transgenic mice containing the human KLKB1 gene (huKLKB1). These findings in bulk ovary tissue warranted further investigation of the potential for inadvertent germline transmission of genome editing events. This risk was evaluated in a GLP breeding study in transgenic huKLKB1 mice. Female huKLKB1 mice (n=70) were treated with NTLA-2002 at doses 17-fold above an expected efficacious dose (3 mg/kg) and crossed with naïve male huKLKB1 mice. Average liver editing in treated females was 78%, indicating maximal activity in the target organ. Among 382 progeny, zero demonstrated a KLKB1 edited genotype. This study thus revealed no evidence of germline transmission of NTLA-2002-mediated editing events, supporting inclusion of women of childbearing potential (WOCBP) in clinical studies.

### Example 9. Clinical Trial

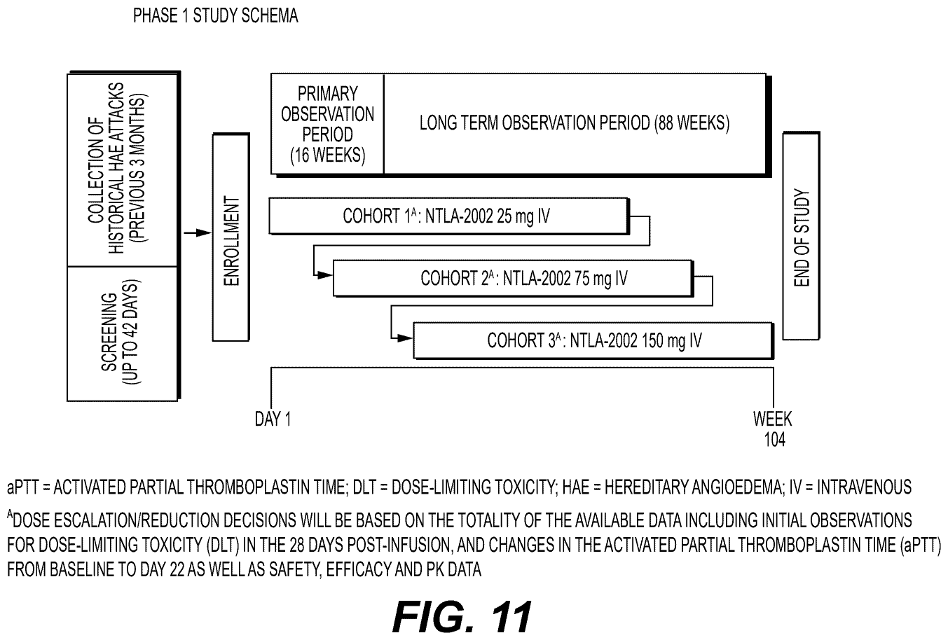

The overall original treatment design is summarized in FIG. 11. The treatment design was revised to reduce the dose for the third cohort from 150 mg to 50 mg.

### A. HAE Dose Escalation Study

Introduction: Hereditary angioedema (HAE) is a rare genetic disorder characterized by recurrent, debilitating and potentially fatal swelling attacks. Prophylactic treatments targeting kallikrein, a protease encoded by the KLKB1 gene, significantly reduce the frequency of attacks. NTLA-2002 is an investigational CRISPR/Cas9-based therapy targeting KLKB1 in hepatocytes, with the goal of achieving life-long control of HAE attacks after a single administration.

Methods: NCT05120830 is a first-in-human phase 1/2 study of NTLA-2002 in patients with HAE. The phase 1 is a single-ascending dose design with the primary objective of evaluating safety and identifying up to two doses to advance to the randomized phase 2 for further evaluation of efficacy and safety.

Results: Cohort 1 (25 mg; n=3) completed the 16-week primary observation period. No DLTs or clinically significant laboratory abnormalities were observed. Treatment emergent adverse events (TEAEs) were non-serious and spontaneously resolving, with the most common being infusion-related reactions (n=2; CTCAE G1). All subjects demonstrated clinically significant, durable reduction in plasma kallikrein levels (mean 62±27% at week 16) and HAE attack frequency from baseline, with 2 subjects remaining attack-free since infusion. Three subjects have been treated in Cohort 2 (75 mg). Follow-up is ongoing for all subjects in both cohorts.

Conclusion: A single 25 mg dose of NTLA-2002 has been well-tolerated to date, meeting pre-specified criteria for advancement to phase 2 with reduction in plasma kallikrein levels and HAE attack rate maintained throughout the 16-week period following infusion. Safety, pharmacodynamic and HAE attack rate data for both cohorts will be presented.

### Cohorts 1, 2, and 3

### Enrollment

Subjects were screened per eligibility criteria. Per protocol, subjects were enrolled in dose escalation cohorts 1 and 2 as shown in FIG. 11. Cohort 3 was enrolled with the same inclusion and exclusion criteria as cohorts 1 and 2. The dosing regimen was amended, and subjects were administered a 50 mg dose of NTLA-2002 rather than a 150 mg dose as in the original protocol.

### Clinical Trial Design and Eligibility

Data from two initial cohorts (Cohorts 1 and 2) from a global, phase 1, open-label, multi-center study are presented herein. Patients were treated with a single dose of NTLA-2002, at 25 mg total RNA (3 subjects) or 75 mg total RNA (3 subjects), administered intravenously. Also reported herein are data from these patients subsequently treated. Key eligibility criteria for Part 1 included at least 18 years of age, documented diagnosis of HAE (Type I or II) confirmed by assessment of functional C1-INH level, C1-INH antigenic level, and C4 level OR genetic testing, and an Investigator-confirmed and documented historical HAE attack number of at least 3 during the previous 3 months (90 days) from the start of screening. Subjects were also required to have access to, and the ability to use, ≥1 acute medication(s) to treat angioedema attacks; and meet laboratory criteria during screening including:
a. Aspartate aminotransferase (AST), alanine aminotransferase (ALT) and total bilirubin
(see exception for Gilbert's Syndrome below)≤upper limit of normal (ULN) range at Screening.
b. For subjects with a history of Gilbert's Syndrome, total bilirubin ≤2×ULN on screening evaluation.
c. Serum creatinine is ≤ULN, or, for subjects in whom serum creatinine is above the ULN, they can be included if the estimated glomerular filtration rate (eGFR) is >45 mL/min/1.73 m\hat{ }2 as measured by the Modification of Diet in Renal Disease equation at Screening. The lower limit was later changed to >60 mL/min/1.73 m^{A}2.
d. Platelet count ≥100,000 cells/mm\hat{ }3 at Screening.
e. Within reference range or Principal Investigator (PI)-determined clinically non-significant aPTT, international normalized ratio (INR), fibrinogen and d-dimer levels at Screening.

Subjects having been treated with certain therapeutic agents were excluded from the Phase 1 study as follows:
a. Use of ecallantide or lanadelumab within 6 months prior to the start of Screening.
b. Use of C1-INH for HAE within 5 half-lives of the agent before initiation of the Phase 2
run-in period; i.e., 24-hour washout is required before starting the run-in period after the
use of rabbit purified C1-INH (ruconest), and 4-day washout is required before starting
the run-in period after the use of human plasma-purified C1-INH (berinert). Note: during
the run-in period, C1-INH may be used to treat an acute HAE attack.
c. Exposure to angiotensin-converting enzyme (ACE) inhibitors or any estrogen-containing medications with systemic absorption within 90 days prior to study drug administration.
d. Antithrombotic therapy other than aspirin (e.g., warfarin, dabigatran, apixaban) within
14 days prior to study drug administration.

Subjects were excluded for the presence of certain infections or medical conditions including:
a. Concurrent diagnosis of any other type of recurrent angioedema, including acquired or
idiopathic angioedema.
b. History of thrombophilia, or positive genetic test for Factor V Leiden or prothrombin 20210.
c. History of cirrhosis.
d. Known or suspected systemic viral, parasitic, or fungal infection including coronavirus
disease (COVID)-19 or received antibiotics for bacterial infection within 14 days prior to
Screening.
e. History of Hepatitis B or C infection or positive Hepatitis B surface antigen (HBsAg) or Hepatitis C virus antibody (HCVAb) test at Screening.
f. History of positive human immunodeficiency virus (HIV) status.

Subjects were required to be willing and able to comply with other criteria including, but not limited to, use of effective contraception, and willingness to comply with study procedures including follow-up and cooperation with the investigator.

The Screening period for the Phase 1 part of the trial was up to 28 days (+14 days when required). A subject who meets all inclusion and none of the exclusion criteria can proceed to dosing. Angioedema attacks are recorded during screening and used in addition to 3 months of historical attack data when evaluating attack rate reduction following administration of NTLA-2002.

### Hereditary Angioedema (HAE) Attack Data Collection and Confirmation

Detailed guidance was provided to the Investigator and study site team to provide consistency related to the documentation and review of attack history, along with the collection, review, and assessment of events deemed potential angioedema attacks. A detailed discussion of the definitions and methods are provided above.

### Dose Escalation and Dose Limiting Toxicities

The Phase 1 clinical trial was designed to demonstrate safety of NTLA-2002 and to identify appropriate dosing for the Phase 2 study. As shown in FIG. 11, Phase 1 consists of up to 3 dose escalation cohorts, with Cohorts 1 through 3 consisting of a minimum of 3 dose limiting toxicity (DLT) evaluable subjects and maximum of 6 DLT evaluable subjects. The study also allows for up to 2 optional dose reduction cohorts, each consisting of a minimum of 3 DLT evaluable subjects and maximum of 6 DLT evaluable subjects. In total, up to 30 DLT evaluable subjects may be enrolled in Phase 1. NTLA-2002 will be dosed as shown in the table Cohort Dosing Overview as shown in FIG. 11, with possible modifications of escalation/reduction as described.

A minimum of 3 DLT evaluable subjects are required per cohort. DLT evaluable subjects are those that have received the full assigned dose of NTLA-2002 and complete Day 29 assessments, and any subjects who experience a dose-limiting toxicity (DLT) regardless of whether a full dose was administered.

### Cohort Dosing

During the single ascending dose phase, each cohort doses a sentinel subject to evaluate potential acute toxicities for 14 days. If the sentinel subject does not experience a DLT, then 2 additional subjects in the cohort may be dosed in parallel. If any subject experiences a DLT between dosing and up to and including Day 15 visit assessments, the next subject in that dose cohort is dosed after any DLT has resolved to at least Grade 1 and after all active subjects complete Day 15 visit assessments. If any subject experiences a DLT after Day 14 visit assessments and up to and including Day 29 visit assessments, the next subject in that dose cohort may be dosed after any DLT has resolved to at least Grade 1.

The last subject dosed in each cohort is evaluated for 28 days, and after that time dose escalation is considered based on the criteria provided below. The Sponsor, in conjunction with the Investigators and the Data Monitoring Committee (DMC), determines if an event meets the definition of DLT and occurred in the first 28 days. The Sponsor may upgrade adverse event (AE) severity, seriousness, or relatedness as reported by the Investigators; downgrading of AEs by the Sponsor is prohibited.

If 1 of 3 subjects has a DLT in any cohort, an additional 3 subjects may be enrolled sequentially with a minimum of 14 days between each subject at that dose level. If 2 or 3 of 3 subjects experience a DLT, enrollment to that cohort is discontinued and the next lower dose cohort may (re)open to enroll 3 subjects. In any cohort, if 3 of 3 subjects have an aPTT >125 seconds at Day 22, that dose level will be declared the maximum evaluated dose. If these cohort-closing events occur in the initial dose level 1 cohort (25 mg), a dose reduction cohort of 3 subjects is investigated to assess whether lower doses are safe and tolerable with evidence of pharmacologic activity. Up to 6 subjects may be enrolled in the final dose cohort to confirm safety.

DLTs include the following (event occurring in the first 28 days after dosing):
Any Common Terminology Criteria for Adverse Events (CTCAE) Grade 3 or higher adverse event that is possibly, probably, or definitely related to study drug excluding AEs associated with angioedema, aPTT prolongation, or a patient's other comorbid (underlying) conditions
Any CTCAE Grade 3 laboratory abnormality (excluding aPTT prolongation) that persists for ≥7 days and is possibly, probably, or definitely related to study drug
Any CTCAE Grade 4 laboratory abnormality that is possibly, probably, or definitely related to study drug
Any AE that meets protocol-defined criteria for pausing enrollment including fatal or life-threatening adverse event; elevation of ALT or AST >5×ULN for more than 4 weeks or ALT or AST >3×ULN and total bilirubin >2×ULN (without initial findings of cholestasis [elevated serum alkaline phosphatase]; Hy's law); or thrombosis, hemorrhage, or laboratory parameters consistent with disseminated intravascular coagulation wherein the protocol-defined criteria for pausing enrollment is determined by the Investigator to be possibly, probably, or definitely related to study drug.
Any other adverse event or laboratory abnormality that, in the opinion of the Sponsor in consultation with the Investigators and the DMC, would preclude further dosing.

### Dose Escalation, Dose Reduction, or Completion of Enrollment

After completion of enrollment in a dose-level cohort and follow-up of at least 3 subjects through the Day 29 visit assessments, the Sponsor reviews all available safety and tolerability data to determine, in consultation with the Investigators and the Data Monitoring Committee (DMC) (per the DMC charter), the next steps. Dose escalation/reduction decisions are based on the totality of the available data including initial observation for DLT in the 28 days post-infusion, changes in the aPTT from baseline to Day 22 as well as safety, efficacy, and PD data.
Dose escalation to the next planned dose level may be proposed by the Sponsor if both of the following criteria are met: administered dose level is found to be safe and well tolerated and review of individual aPTT reveals at least 1/3 subjects in the cohort has an aPTT <125 seconds at the Day 22 visit assessment. Based on review of all available data, the dose escalation may be modified such that the next planned dose level is a dose between the current dose and the next protocol-specified dose. Alternatively, the Sponsor may elect to open a cohort such that the next planned dose level is a dose reduction.
Dose reduction to a dose below the current dose level will be proposed by the Sponsor if either of the following criteria are met: administered dose in a cohort is not found to be safe and well tolerated; or review of a cohort's aPTT indicates that maximum intended pharmacologic effect (≥125 seconds) was achieved at Day 22 visit in all subjects in the cohort.

Dose escalation, dose reduction, or completion of study Phase 1 enrollment and initiation of Phase 2 will be decided by the Sponsor with Investigators and the DMC providing a recommendation (per the DMC charter). In addition to the criteria described above, at the discretion of the Sponsor, study enrollment may be stopped and the DMC convened for any reason to protect subject safety.

Completion of study enrollment in Phase 1 is declared after all 3-5 planned dose cohorts complete the Day 29 assessments; or if maximum evaluated dose is identified; or early termination of the study based on Sponsor assessment in consultation with the Investigators and the DMC.

To inform the selection of doses to test in the Phase 2 study, the Sponsor performs collective analysis of all Phase 1 safety, tolerability, PK, PD (reduction in prekallikrein/kallikrein concentrations), and therapeutic activity (analysis of HAE attack rate Weeks 5-16). Results are be reviewed with the Investigators and DMC to determine whether a dose is to be tested in the Phase 2 study.

Using an appropriate data cutoff point, a cumulative data summary is provided per national requirement to regulatory authorities or Institutional Review Board (TRB)/Ethics Committee (EC) prior to commencing the Phase 2 study.

### Phase 2

Phase 2 involves up to 25 subjects and 3 arms, including up to 2 arms receiving different doses of NTLA-2002 and 1 arm receiving saline placebo. If 2 dose levels of NTLA-2002 are used in Phase 2, subjects are randomized 2:2:1, NTLA-2002 dose level 1:NTLA-2002 dose level 2:placebo. If only 1 dose level of NTLA-2002 is used in Phase 2, up to 15 subjects are randomized 2:1, NTLA-2002 dose level 1:placebo. NTLA-2002 doses selected for Phase 2 are doses determined in Phase 1 to be safe and well tolerated, with at least 60% reduction in mean total plasma prekallikrein/kallikrein level from baseline to nadir, and evidence of reduction in HAE attacks.

Phase 2 involves up to 25 subjects and 3 arms, including up to 2 arms receiving different doses of NTLA-2002, 25 mg (n=10) and 50 mg (n=10), and 1 arm receiving saline placebo (n=5). Primary objectives are clinical efficacy against attacks through week 16, with other pharmacodynamic, safety, tolerability, pharmacokinetic, and quality of life assessments.

### Pharmacokinetics

Interim pharmacokinetic data suggest that following intravenous (IV) infusion, NTLA-2002 ionizable lipid exhibited a rapid decline from peak levels followed by a secondary peak and then a log-linear phase.

### Example 10. In Vivo CRISPR/-Cas9 Editing of KLKB1 in Patients with Hereditary Angioedema: a First-In-Human Study

### Cohort 1:

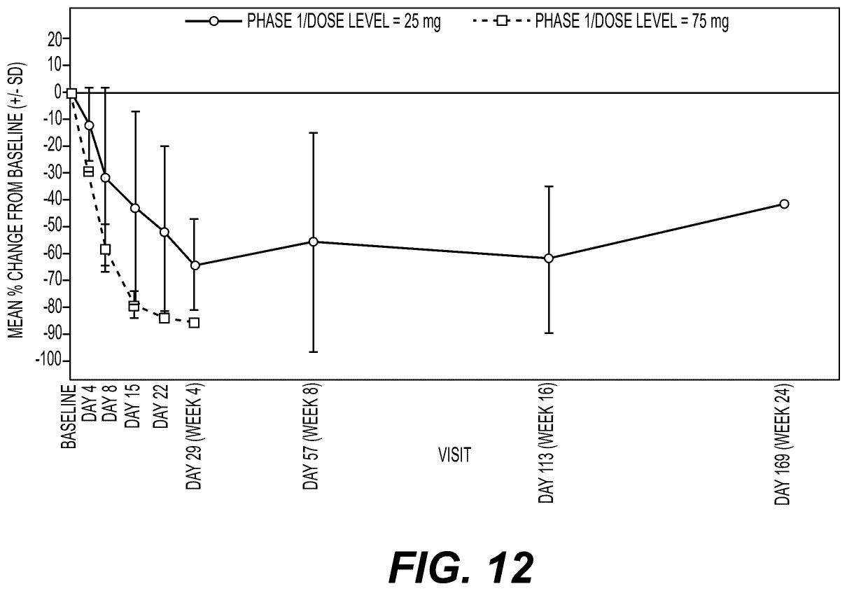

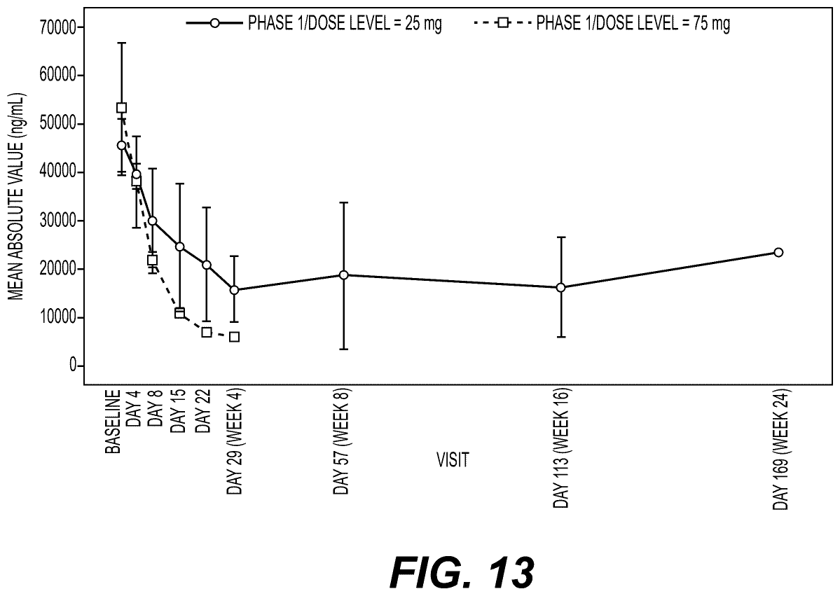

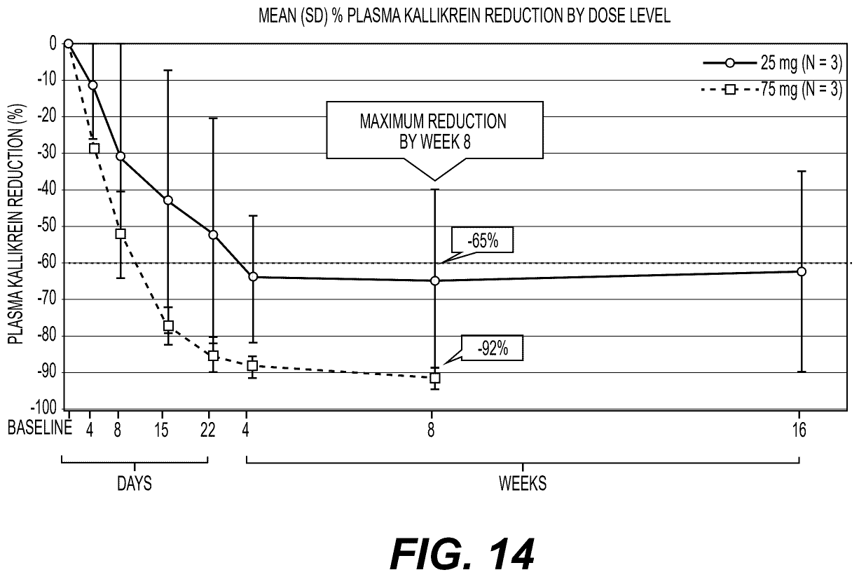

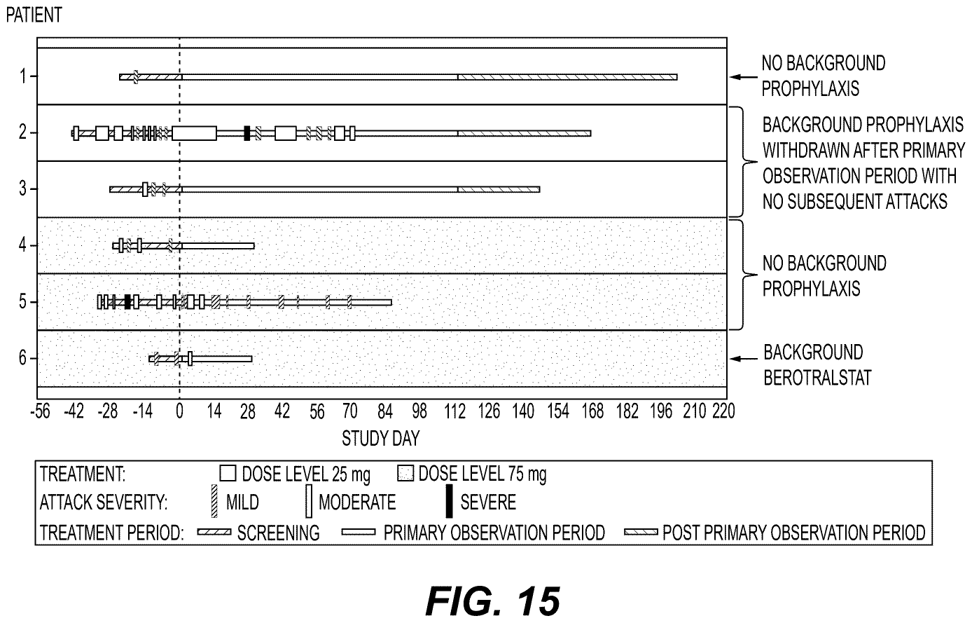

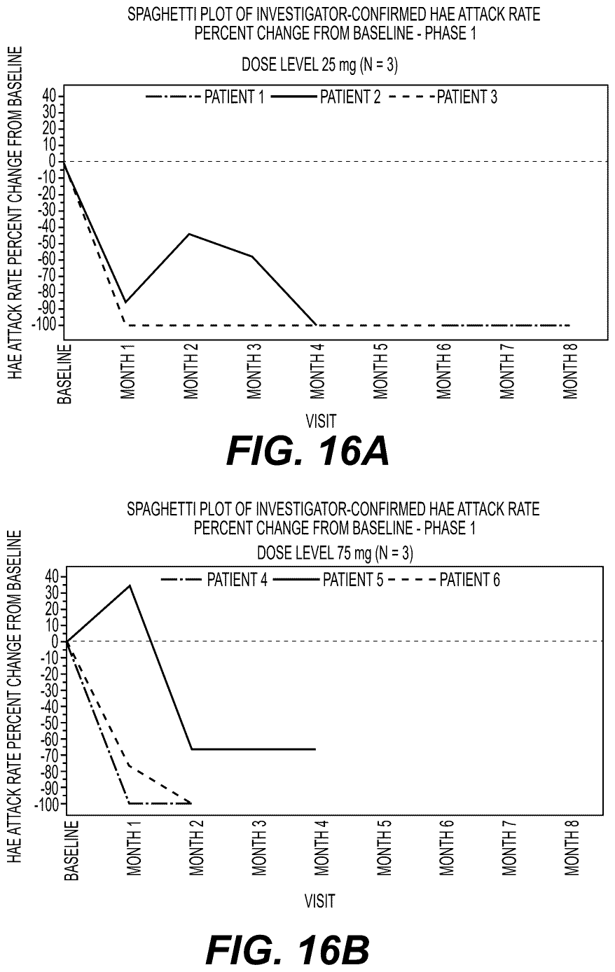

Data are reported for three subjects treated with 25 mg of NTLA-2002 in Cohort 1 of the phase 1 study described in Example 9. Cohort 1 demographics and HAE attack characteristics are described in Table 13. The subjects completed a 16-week primary observation period. The subjects had no observed DLTs. The subjects had no clinically significant laboratory abnormalities. TEAEs were non-serious and spontaneously resolving, with the most common being infusion-related reactions (n=2; CTCAE G1). All infusion-related reactions were considered mild, resolving without clinical sequelae. No treatment emergent SAEs or ≥Grade 3 AEs were observed. The subjects demonstrated a reduction in HAE attack frequency from baseline, with two subjects remaining attack-free since infusion (Tables 11 and 12 and FIG. 15 and FIG. 16A). Compared to the screening period, in weeks 1-16 following infusion, subjects in cohort 1 had a >90% mean reduction in attack frequency (mean −91±16% SD. Analysis including the 90-day patient reported historical period resulted in a mean percent change of −94% in monthly attack rate. The subjects also demonstrated a reduction, e.g., clinically significant, durable reduction, in plasma kallikrein levels (mean 62±27% at week 16) (see, e.g., FIG. 12). FIG. 13 depicts mean absolute protein reduction after infusion. FIG. 14 depicts updated results of mean absolute protein reduction after infusion. The single 25 mg dose of NTLA-2002 has been well-tolerated to date and meets pre-specified criteria for advancement to phase 2, with reduction in plasma kallikrein levels and HAE attack rate maintained throughout the 16-week period following infusion.

TABLE 11
Cohort 1 (25 mg) Reduction in Attack Rate

| | |
|---|---|
| | 25 mg |
| | n = 3 |
| Parameter | Events |

90-Day Historical Period (Patient-Reported)

| | |
|---|---|
| Mean (SD) | 6.0 (6.9) |
| Median (Min, Max) | 2.2 (1.9, 14) |

Screening Period (Investigator-Confirmed)

| | |
|---|---|
| Mean (SD) | 3.7 (3.1) |
| Median (Min, Max) | 2.9 (1.1, 7.2) |

Weeks 1-16 (Investigate Confirmed)

| | |
|---|---|
| Mean (SD) | 0.7 (1.2) |
| Median (Min, Max) | 0 (0, 2) |

TABLE 12
Cohort 1 (25 mg) Individual Reduction in Attack Rate

Patient

| Monthly Attack Rate | 1 | 2 | 3 |
|---|---|---|---|
| 90-Day Historical Period (Patient-Reported) | 1.9 | 14.0 | 2.2 |
| Screening Period (Investigator-Confirmed) | 1.1 | 7.2 | 2.9 |
| Weeks 1-16 (Investigator Confirmed) | 0 | 2 | 0 |

TABLE 13
Cohort 1 (25 mg) Demographic Data and Attack Characterization

| | Patient 1 | Patient 2 | Patient 3 |
|---|---|---|---|
| Age | 30 | 52 | 26 |
| HAE Type I or | Type II, family | Type I, family | Type I |
| Type II | history of HAE | history of HAE | |
| Attack rate | 1.1 | 7.2 | 2.9 |
| during screening | | | |
| per month | | | |
| Prophylaxis | no prophylaxis | long term danazol | berotralstat |
| | | prophylaxis | |
| Attack | Typically moderate | Breakthrough | Breakthrough |
| characteristics | in severity, | attacks, typically | attacks involved |
| | involving swelling | severe, involving | laryngeal swelling, |
| | in extremities | abdominal swelling, | cutaneous swelling |
| | | pain and peripheral | in the genitourinary |
| | | edema | regions and |
| | | | extremities, and |
| | | | abdomen |

### Cohort 2:

Three subjects have been treated with 75 mg of NTLA-2002 in Cohort 2 of the phase 1 study described in Example 9.

In a follow up analysis with Cohort 2, the subjects had no clinically significant laboratory abnormalities. TEAEs were non-serious and spontaneously resolving, with the most common being infusion-related reactions (n=2; CTCAE G1 and n=2; CTCAE G2). All infusion-related reactions were considered mild, resolving without clinical sequelae. No grade 3 or higher TEAEs were reported. FIG. 13 depicts mean absolute protein reduction after infusion. FIG. 14 depicts updated results of mean absolute protein reduction after infusion. With % reductions from baseline to nadir of 65% (25 mg) and 92% (75 mg), both dose levels demonstrated pharmacodynamic responses expected to be associated with effective prevention of HAE attacks.

Patient demographics and characteristics are provided in Table 14. Patient HAE attack history and prophylaxis use for Cohorts 1 and 2 individually and in aggregate are presented in Table 15.

TABLE 14
Patient Demographics and Characteristics

| | 25 mg | 75 mg | All patients |
|---|---|---|---|
| Parameter | (n = 3) | (n = 3 ) | (n = 6) |
| Median age, years | 30 (26, 52) | 45 (27, 49) | 38 (26, 52) |
| (Min, Max) | | | |
| Sex, n (%) | | | |
| Male | 3 (100%) | 2 (67%) | 5 (83%) |
| Female | — | 1 (33%) | 1 (17%) |
| Median weight, kg | 83 (78, 135) | 72 (64, 84) | 81 (64, 135) |
| (Min, Max) | | | |
| HAE Type, n (%) | | | |
| Type I | 2 (67%) | 2 (67%) | 4 (67%) |
| Type II | 1 (33%) | 1 (33%) | 2 (33%) |

TABLE 15
HAE Attack History and Prophylaxis

| | 25 mg | | 75 mg | | All patients | |
|---|---|---|---|---|---|---|
| Parameter | n = 3 | | n = 3 | | n = 6 | |
| Prior Use of | | | | | | |
| Prophylaxis, n (%) | | | | | | |
| Yes | 3 | (100%) | 3 | (100%) | 6 | (100%) |
| No | — | | — | | — | |
| Historical Monthly Attack | | | | | | |
| Rate (Patient-reported) | | | | | | |
| Mean (SD) | 6.0 | (6.9) | 7.7 | (8.0) | 6.8 | (6.8) |
| Typical Attack | | | | | | |
| Severity, n (%) | | | | | | |
| Mild | 1 | (33%) | 1 | (33%) | 2 | (33%) |
| Moderate | 1 | (33%) | 1 | (33%) | 2 | (33%) |
| Severe | 1 | (33%) | 1 | (33%) | 2 | (33%) |

NTLA-2002 was generally well tolerated across both dose levels. Across both dose levels, the most frequent adverse events were fatigue and infusion-related reactions. The majority of treatment emergent adverse events were mild in severity with 67% (n=4) and 3300 (n=2) of patients reporting a maximal adverse event severity of Grade 1 or 2, respectively. All infusion-related reactions were considered mild (n=4) or moderate (n=1), resolving without clinical sequelae. All patients received a complete study dose of NTLA-2002.

No clinically significant laboratory findings observed. Transient Grade 1 elevations in AST (n=3) and ALT (n=2) were observed. No increases in activated partial thromboplastin time were observed. No treatment emergent SAEs or ≥Grade 3 AEs were observed. Adverse events are summarized in Table 16 below.

TABLE 16
Adverse Events

| AE | Cohort 1 | | Cohort 2 | | All patients | |
|---|---|---|---|---|---|---|
| occurring | (25 mg) n = 3 | | (75 mg) n = 3 | | n = 6 | |
| in >2 | Grade | Grade | Grade | Grade | Grade | Grade |
| patients | 1 | 2 | 1 | 2 | 1 | 2 |
| Infusion | 2 | — | 2 | 1 | 4 | 1 |
| related | | | | | | |
| reaction | | | | | | |
| Fatigue | 1 | — | 2 | — | 3 | — |
| Headache | — | — | 2 | — | 2 | — |
| COVID- | 2 | — | — | — | 2 | — |
| 19 | | | | | | |
| Upper | 1 | — | 1 | — | 2 | — |
| respiratory | | | | | | |
| tract | | | | | | |
| infection | | | | | | |

All other adverse events (abdominal pain, chest injury, soft tissue injury, disease prodromal stage, rhinitis, diarrhea, vomiting, somnolence, myalgia, insomnia, oropharyngeal pain, viral upper respiratory tract infection) were reported in no more than one patient.

A single dose of NTLA-2002 led to robust, dose-dependent and durable reductions in total plasma kallikrein levels. Mean reductions of 65% (25 mg) and 92% (75 mg) achieved at week 8. Mean >90% reduction in HAE attacks in the 25 mg cohort through week 16. All patients in the 25 mg cohort achieved complete attack control. Patients on prior prophylactic therapy were able to discontinue and remain attack free.

NTLA-2002 was generally well-tolerated across both dose levels; all AEs were of mild or moderate severity. A 50 mg cohort has been enrolled and treated.

### Cohort 3:

Four subjects have been treated with 50 mg of NTLA-2002 in Cohort 3 of the phase 1 study described in Example 9.

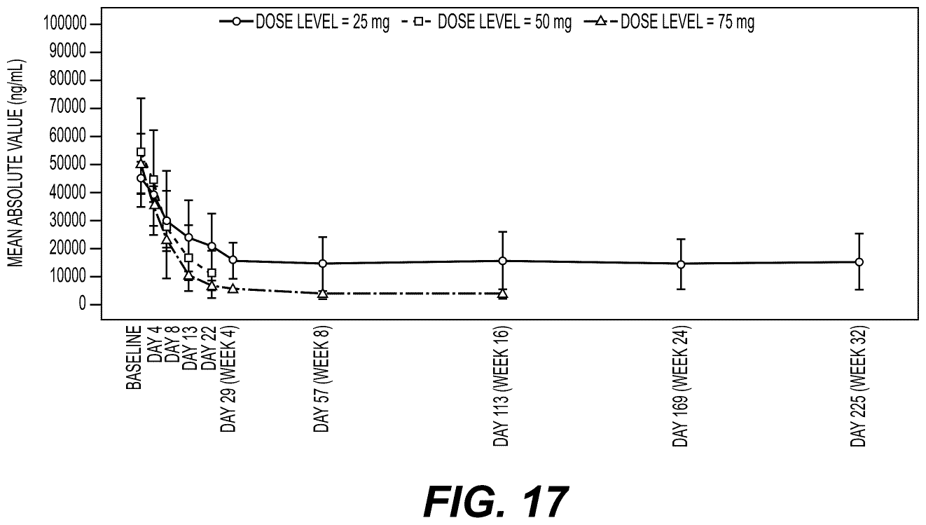

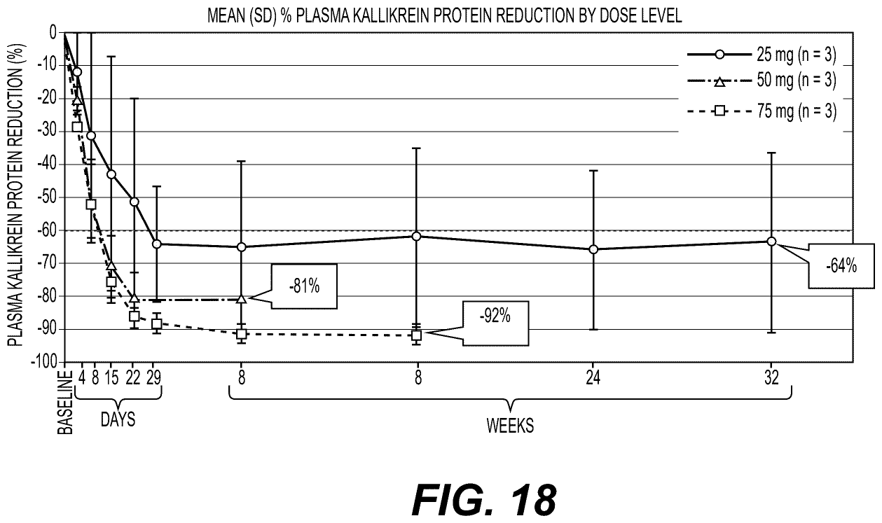

In a follow up analysis with Cohort 3, the subjects had no clinically significant laboratory abnormalities. Across all dose levels, TEAEs were mild (n=5) to moderate (n=4), resolving without clinical sequelae. No increases in activated partial thromboplastin time were observed. No treatment emergent SAEs or grade 3 or higher TEAEs were reported. FIG. 17 depicts updated results of the mean absolute protein reduction after infusion. FIG. 18 depicts updated results of mean percent kallikrein protein reduction relative to baseline after infusion. All three dose levels demonstrated pharmacodynamic responses expected to be associated with effective prevention of HAE attacks.

Patient demographics and characteristics are provided in Table 17. Patient HAE attack history and prophylaxis use for Cohorts 1-3 are presented in Table 18.

TABLE 17
Patient Demographics and Characteristics

| | | | | | | | All | |
|---|---|---|---|---|---|---|---|---|
| | 25 mg | | 50 mg | | 75 mg | | patients | |
| Parameter | (n = 3) | | (n = 4) | | (n = 3) | | (n = 10) | |
| Median age, | 30 | (26, 52) | 65 | (52, 73) | 45 | (27, 49) | 51 | (26, 73) |
| years | | | | | | | | |
| (Min, Max) | | | | | | | | |
| Sex, n (%) | | | | | | | | |
| Male | 3 | (100%) | 1 | (25%) | 2 | (67%) | 6 | (60%) |
| Female | — | | 3 | (75%) | 1 | (33%) | 4 | (40%) |
| Median weight, | 83 | (78, 135) | 86 | (74, 107) | 72 | (64, 84) | 83 | (64, 135) |
| kg (Min, Max) | | | | | | | | |
| HAE Type, | | | | | | | | |
| n (%) | | | | | | | | |
| Type I | 2 | (67%) | 1 | (25%) | 2 | (67%) | 5 | (50%) |
| Type II | 1 | (33%) | 2 | (50%) | 1 | (33%) | 4 | (40%) |

| | | | | | | |
|---|---|---|---|---|---|---|
| Unknown | — | 1 | (25%) | — | 1 | (10%) |

TABLE 18
HAE Attack History and Prophylaxis

| | | | | | | | All | |
|---|---|---|---|---|---|---|---|---|
| | 25 mg | | 50 mg | | 75 mg | | patients | |
| Parameter | n = 3 | | n = 4 | | n = 3 | | n = 10 | |
| Prior Use of | | | | | | | | |
| Long-Term | | | | | | | | |
| Prophylaxis, | | | | | | | | |
| n (%) | | | | | | | | |
| Yes | 2 | (67%) | 4 | (100%) | 3 | (100%) | 9 | (90%) |
| No | 1 | (33%) | | | | | 1 | (10%) |
| Concomitant | | | | | | | | |
| Long-Term | | | | | | | | |
| Prophylaxis, | | | | | | | | |
| n (%) | | | | | | | | |
| Yes | 2 | (67%) | 3 | (75%) | 1 | (33%) | 6 | (60%) |
| No | 1 | (33%) | 1 | (25%) | 2 | (67%) | 4 | (40%) |
| Historical | | | | | | | | |
| Monthly Attack | | | | | | | | |
| Rate | | | | | | | | |
| (Patient- | | | | | | | | |
| reported) | | | | | | | | |
| Mean (SD) | 6.0 | (6.9) | 1.2 | (0.47) | 7.7 | (8.00) | 4.6 | (5.83) |
| Typical | | | | | | | | |
| Attack | | | | | | | | |
| Severity, | | | | | | | | |
| n (%) | | | | | | | | |
| Mild | 1 | (33%) | 2 | (50%) | 1 | (33%) | 4 | (40%) |
| Moderate | 1 | (33%) | 2 | (50%) | 1 | (33%) | 4 | (40%) |

| | | | | | | | |
|---|---|---|---|---|---|---|---|
| Severe | 1 | (33%) | 0 | 1 | (33%) | 2 | (20%) |

NTLA-2002 was generally well tolerated across all three dose levels. Across all three dose levels, the most frequent adverse events were fatigue and infusion-related reactions. The majority of treatment emergent adverse events were mild in severity with 50% (n=5) and 40% (n=4) of patients reporting a maximal adverse event severity of Grade 1 or 2, respectively. All infusion-related reactions were considered mild (n=5) or moderate (n=2), resolving without clinical sequelae. All patients received a complete study dose of NTLA-2002.

No clinically significant laboratory findings observed. Adverse events are summarized in Table 19 below.

TABLE 19
Adverse Events

| | Cohort 1 | | Cohort 3 | | Cohort 2 | | All patients | |
|---|---|---|---|---|---|---|---|---|
| | (25 mg) n = 3 | | (50 mg) n = 4 | | (75 mg) n = 3 | | n = 10 | |
| AE occurring in | Grade | Grade | Grade | Grade | Grade | Grade | Grade | Grade |
| >2 patients | 1 | 2 | 1 | 2 | 1 | 2 | 1 | 2 |
| Any TEAE (max | 2 | 1 | 2 | 1 | 1 | 2 | 5 | 4 |
| grade) | | | | | | | | |
| Infusion related | 2 | — | 1 | 1 | 2 | 1 | 5 | 2 |
| reaction | | | | | | | | |
| Fatigue | 1 | — | 2 | 1 | 2 | — | 5 | 1 |
| COVID-19 | 2 | — | 1 | — | 1 | — | 4 | — |
| Oropharyngeal | 2 | — | — | — | 1 | — | 3 | — |
| pain | | | | | | | | |
| Headache | — | — | — | — | 2 | — | 2 | — |
| Upper respiratory | 1 | — | — | — | 1 | — | 2 | — |
| tract infection | | | | | | | | |
| Viral upper | — | — | — | — | 2 | — | 2 | — |
| respiratory tract | | | | | | | | |
| infection | | | | | | | | |

All other adverse events AEs (abdominal discomfort, abdominal pain, abdominal pain upper, arthralgia, asthenia, chest injury, depressed mood, diarrhea, disease prodromal stage, flank pain, insomnia, myalgia, rhinitis, sinusitis, soft tissue injury, somnolence, vomiting) were reported in no more than one patient.

A single dose of NTLA-2002 led to robust, dose-dependent and durable reductions in total plasma kallikrein levels. Mean plasma reductions of between 65% (25 mg) and 92% (75 mg) were observed at nadir, with responses persisting for the duration of follow up.

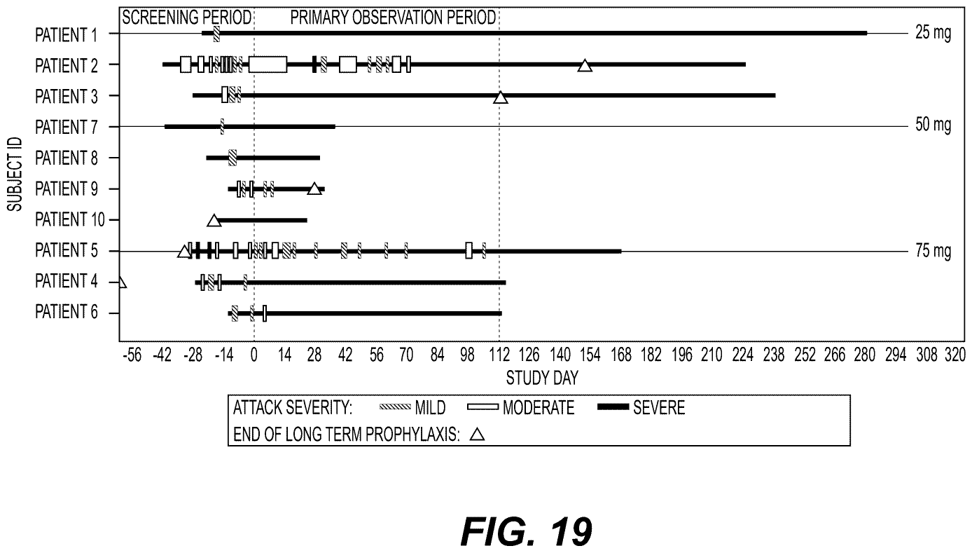

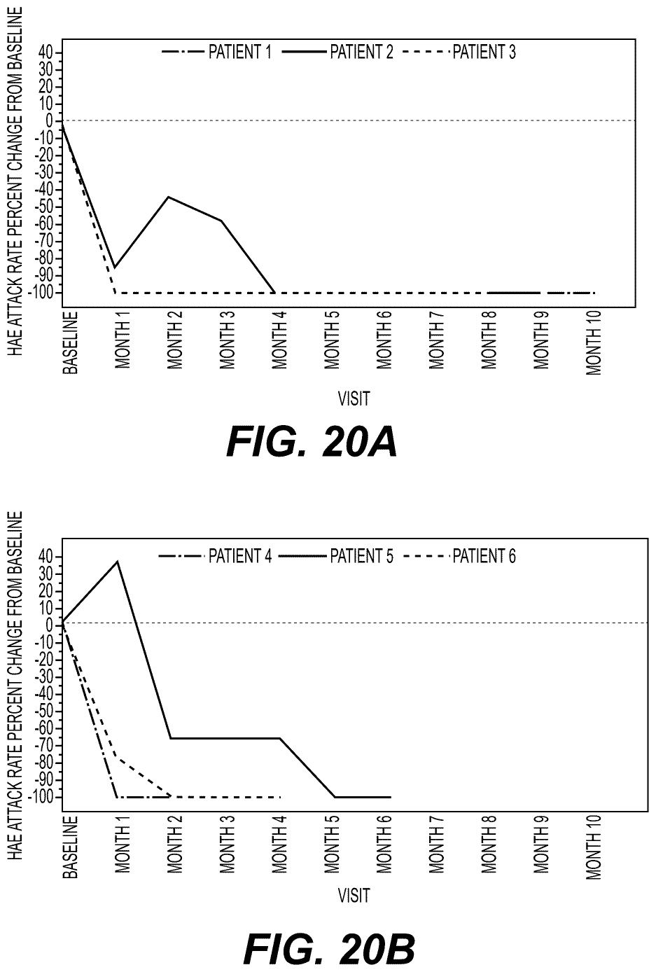

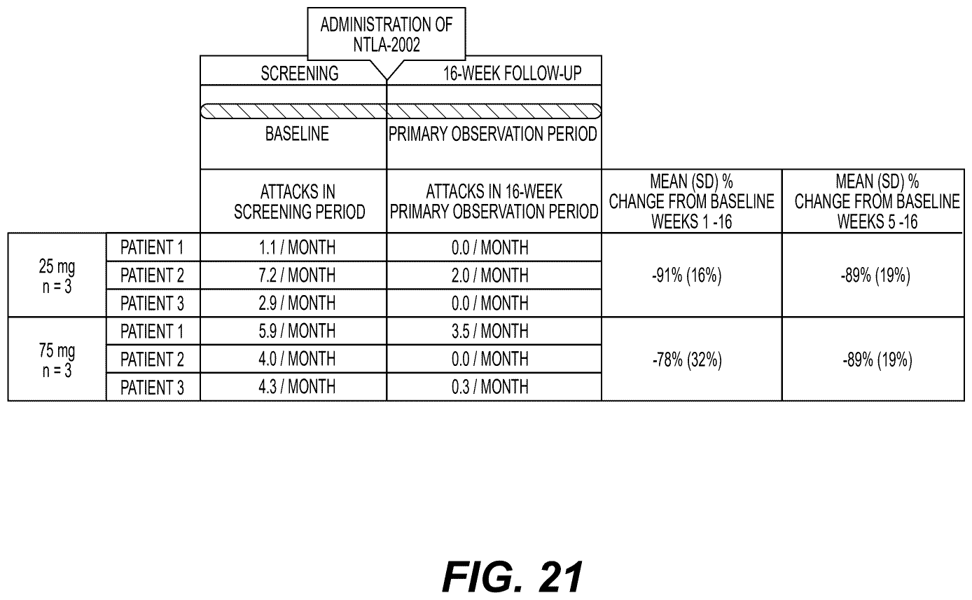

Clinically meaningful reductions in HAL attack rate were observed in all patients with at least 16 weeks of follow-up. 100% of patients in Cohort 1 (25 mg) and Cohort 2 (75 mg) have an ongoing attack-free interval of 2.3 to 10.6 months; the first three patients treated have been attack free for 5.5-10.6 months (FIGS. 19, 20A, and 20B). The mean percentage change from baseline in attacks was 91% for Cohort 1 and 78% in Cohort 2 in weeks 1-16 following administration of NTLA-2002. The mean percentage change from baseline in attacks was 89% for Cohort 1 and 89% in Cohort 2 in weeks 5-16 following administration of NTLA-2002 (FIG. 21). Patients who discontinued prophylactic therapy after NTLA-2002 infusion remained attack free.

Consistent with earlier analyses, NTLA-2002 was generally well-tolerated across both dose levels; all AEs were of mild or moderate severity.

### Example 11. In Vivo CRISPR/-Cas9 Editing of KLKB1 in Patients with Hereditary Angioedema: Updated Clinical Data

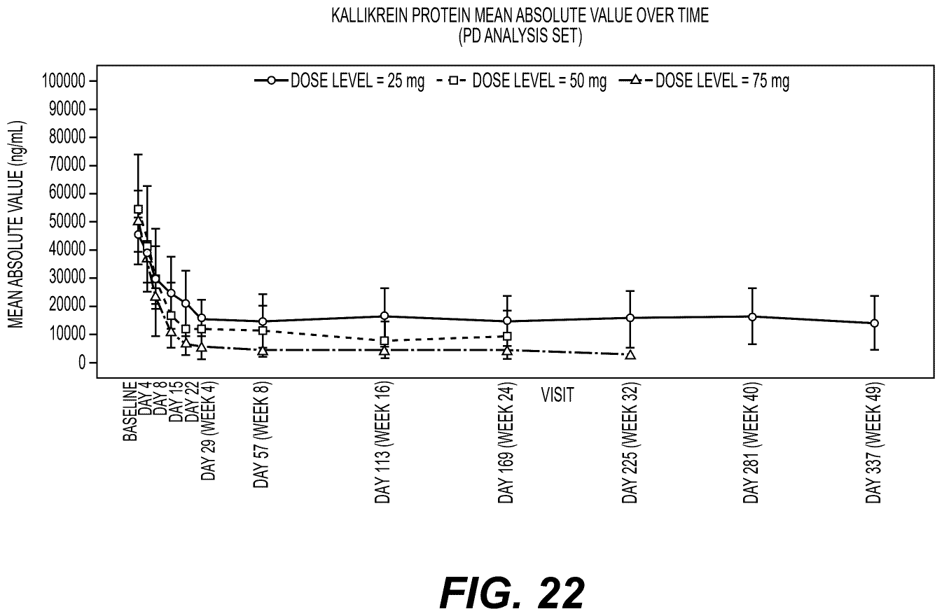

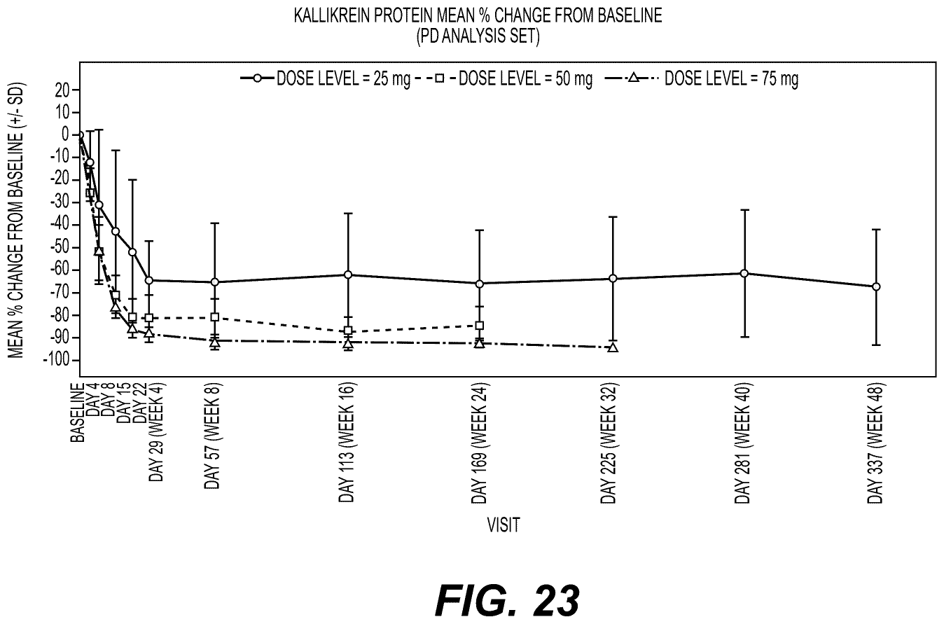

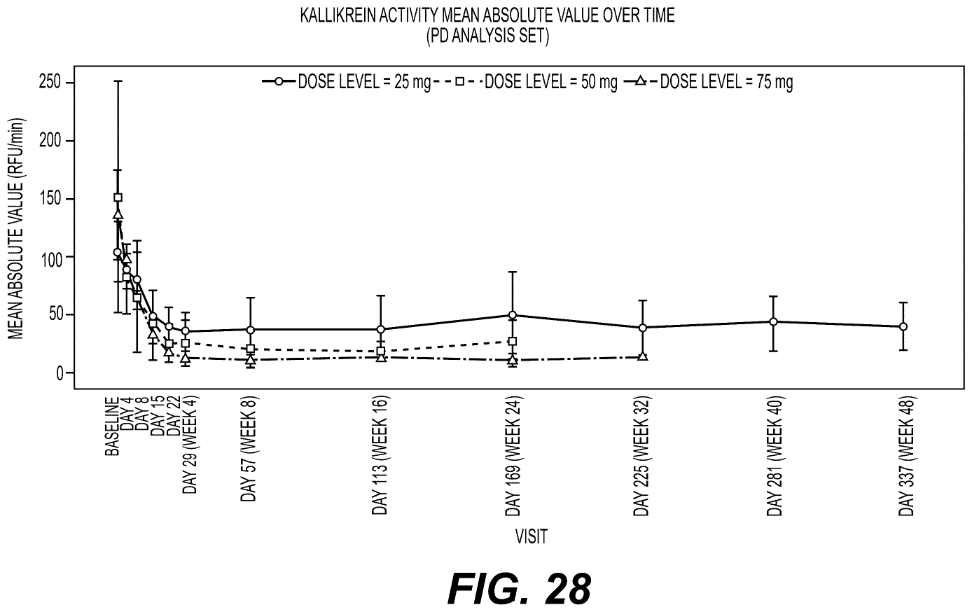

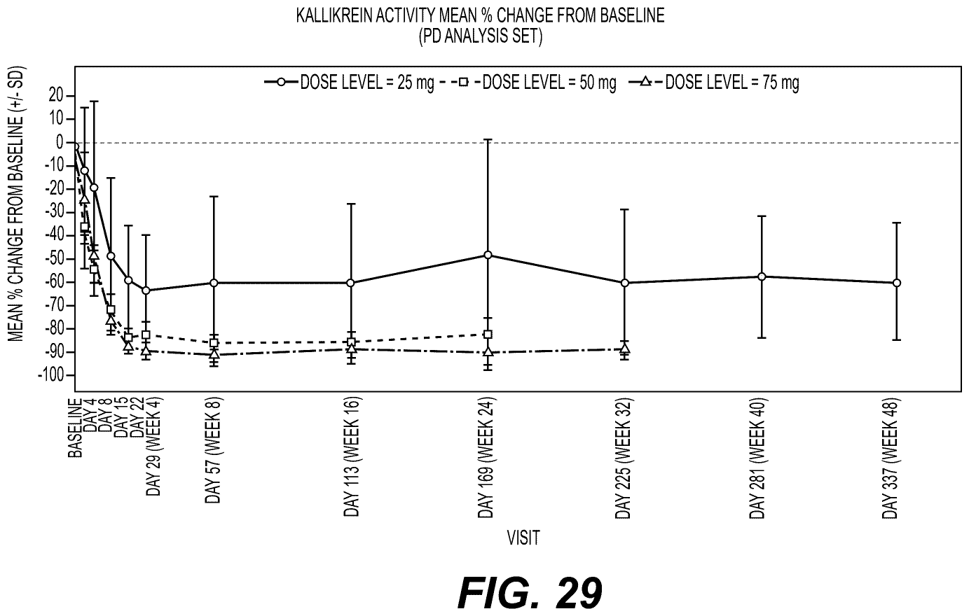

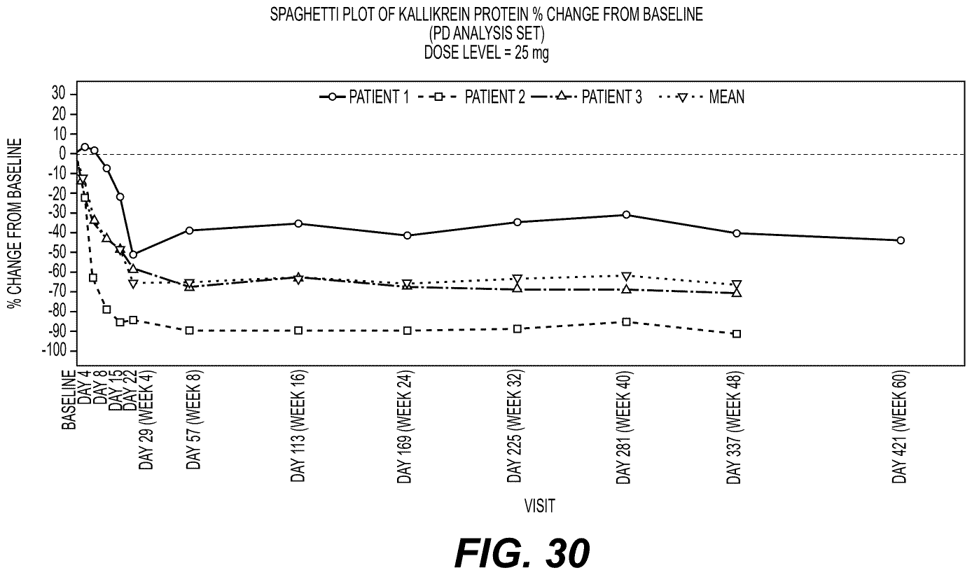

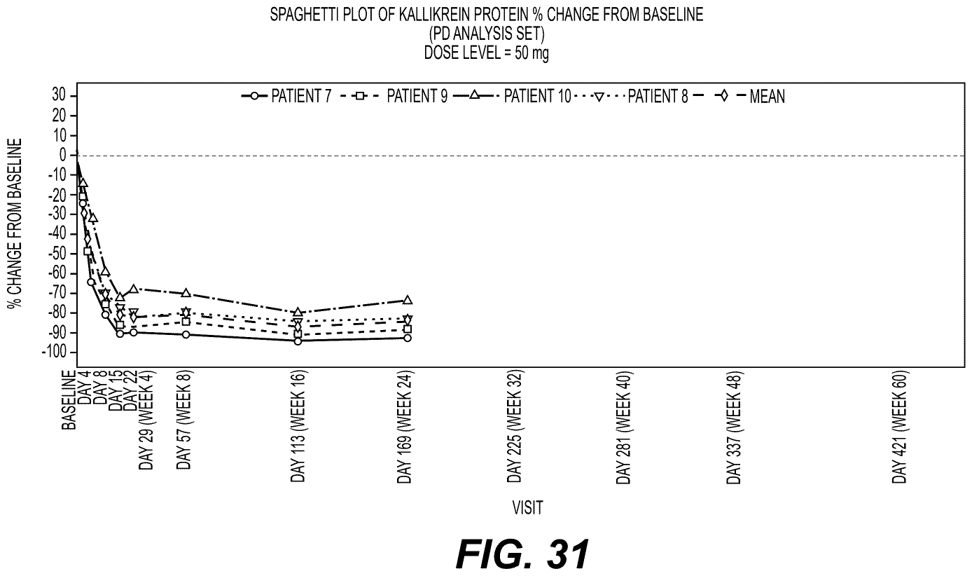

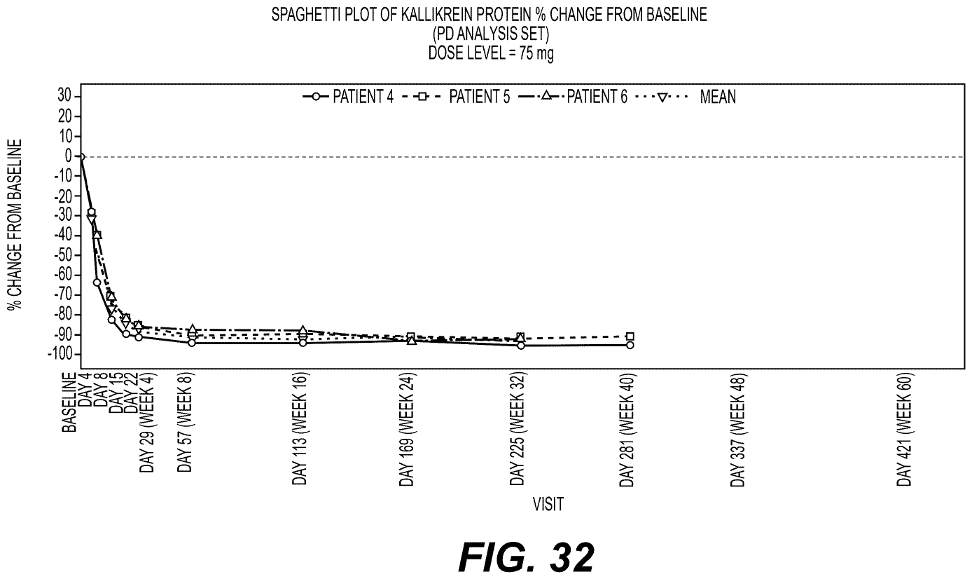

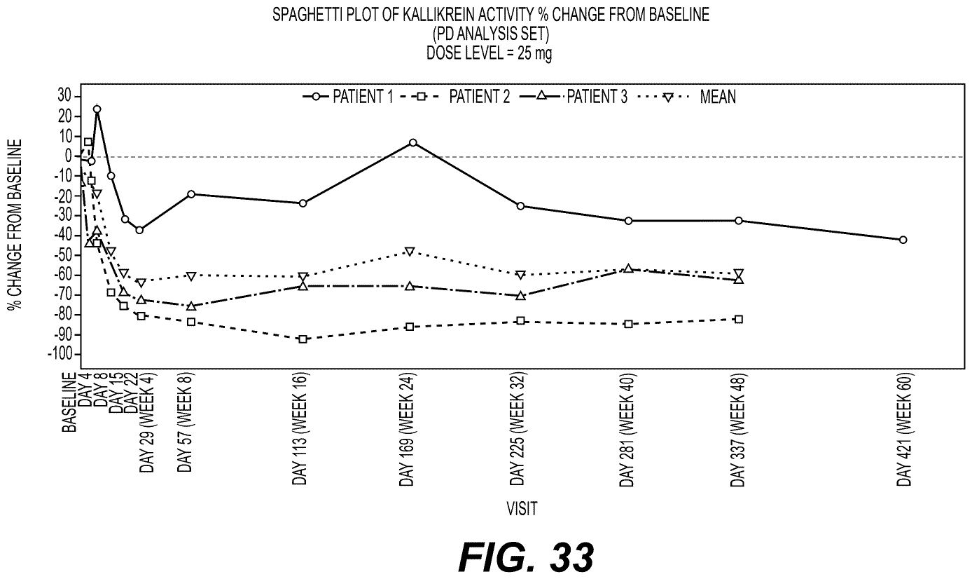

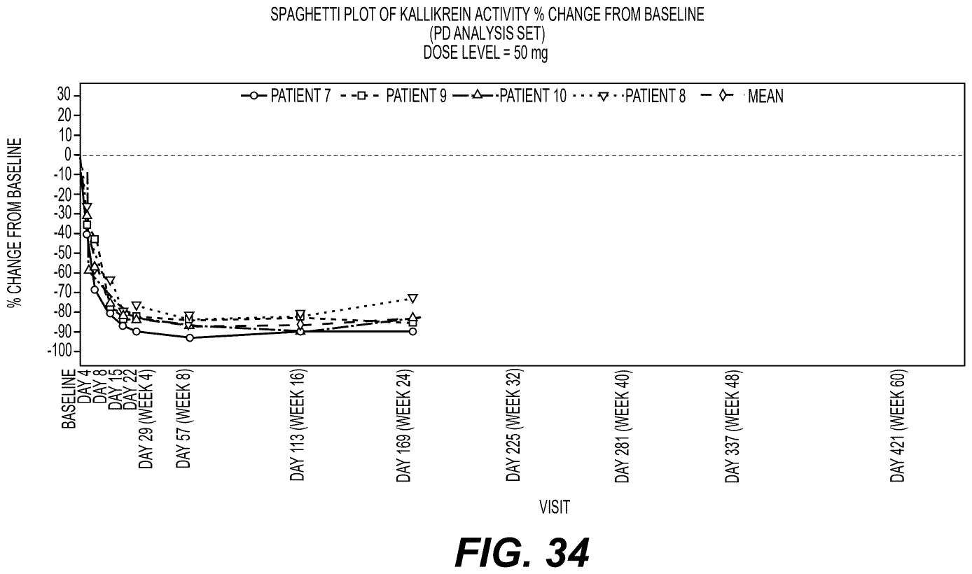

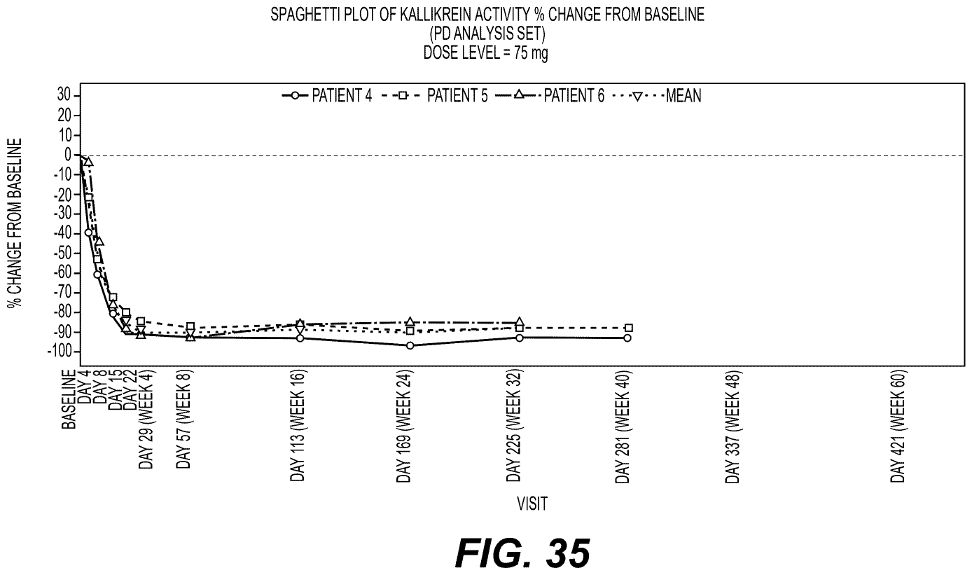

A follow up analysis of Cohorts 1-3 from Example 10 is provided. FIG. 22 depicts updated results of the mean absolute protein reduction after infusion. FIG. 23 depicts updated results of mean percent kallikrein protein reduction relative to baseline after infusion. Plasma kallikrein activity mean absolute values after infusion for each Cohort are depicted in FIG. 28. Plasma kallikrein activity mean percentage change from baseline after infusion for each Cohort are depicted in FIG. 29. Spaghetti plots of individual patient data of kallikrein protein levels as a percentage change from baseline are depicted in FIGS. 30-32. Spaghetti plots of individual patient data of kallikrein activity levels as a percentage change from baseline are depicted in FIGS. 33-35. Consistent with earlier reported findings, all three dose levels demonstrated pharmacodynamic responses expected to be associated with effective prevention of HAE attacks.

A single dose of NTLA-2002 led to robust, dose-dependent and durable reductions in total plasma kallikrein levels. Mean plasma reductions of 65% (25 mg), 81% (50 mg), and 92% (75 mg) were observed at nadir, with responses persisting for the duration of follow up.

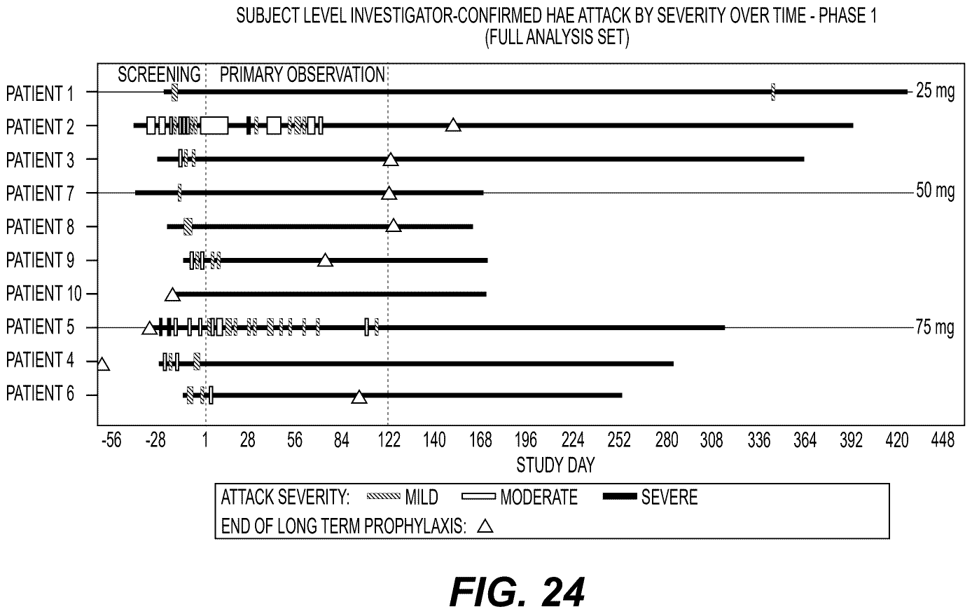

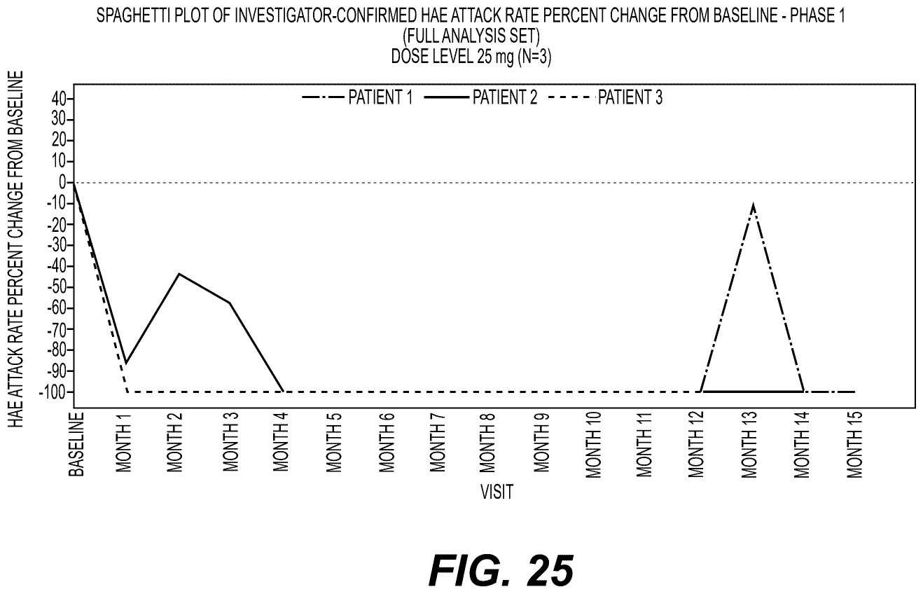

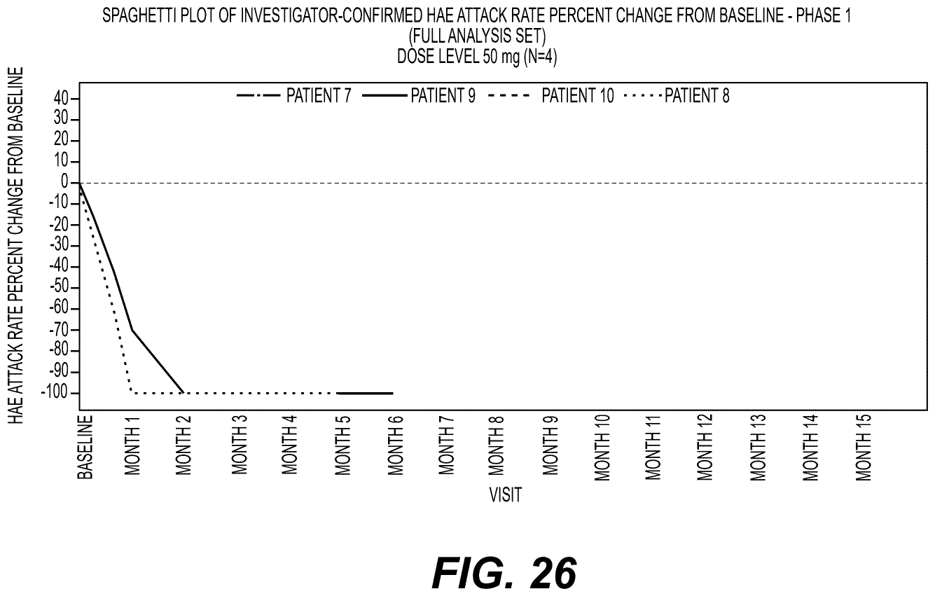

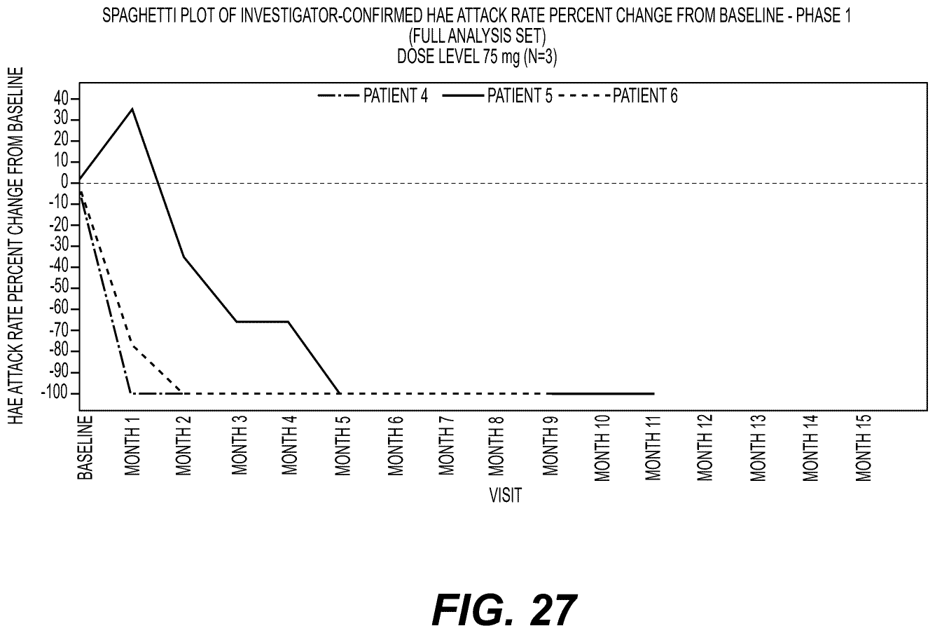

Clinically meaningful reductions in HAE attack rate were observed in all patients with at least 24 weeks of follow-up. With the exception of a patient in Cohort 1 (25 mg), all patients have an ongoing attack-free interval of 3.7 to 12 months (FIGS. 24-27). A patient in Cohort 1 (25 mg) experienced a single, mild attack. The mean percentage change from baseline in attacks was 93.8% for Cohort 1 (25 mg), 83.7% in Cohort 2 (75 mg), and 98.3% in Cohort 3 (50 mg) in weeks 1-24 following administration of NTLA-2002. The mean percentage change from baseline in attacks was 95.4% for Cohort 1, 91.2% in Cohort 2, and 98.3% in Cohort 3 in the on-study period following administration of NTLA-2002 (Table 20, 21). Among the 6 patients who received concomitant long-term prophylaxis, (Table 18) 100% were able to discontinue prophylactic therapy and remain attack free (FIG. 24). Patient HAE attack history for Cohorts 1-3 are presented in Table 20 (investigator-confirmed HAE attacks) and Table 21 (investigator-confirmed HAE attacks requiring acute therapy).

Consistent with earlier analyses, NTLA-2002 was generally well-tolerated across all three dose levels; all AEs were of mild or moderate severity. All treatment-related TEAEs were grade 2 or lower. All patients received a complete study dose of NTLA-2002. Adverse events are summarized in Table 22 below.

TABLE 20
Descriptive Summary of Investigator-confirmed HAE Attacks

| | | Dose Level 25 mg | | Dose Level 50 mg | | Dose Level 75 mg | | Total | |
|---|---|---|---|---|---|---|---|---|---|
| | | N = 3 | | N = 4 | | N = 3 | | N = 10 | |
| | | | % change | | % change | | % change | | % change |
| | | | from | | from | | from | | from |
| | | Observed | Baseline | Observed | Baseline | Observed | Baseline | Observed | Baseline |
| Baseline | N (%) | 3 | | 4 | | 3 | | 10 | |
| | | (100) | | (100) | | (100) | | (100) | |
| | Mean | 3.7 | | 2.1 | | 4.7 | | 3.4 | |
| | (SD) | (3.11) | | (2.96) | | (1.04) | | (2.59) | |
| | Median | 2.9 | | 0.9 | | 4.3 | | 3.4 | |
| | Min, | 1.1, | | 0.0, | | 4.0, | | 0.0, | |
| | Max | 7.2 | | 6.5 | | 5.9 | | 7.2 | |
| Week 1-16 | N (%) | 3 | 3 | 4 | 3 | 3 | 3 | 10 | 9 |
| | | (100) | (100) | (100) | (75.0) | (100) | (100) | (100) | (90.0) |
| | Mean | 0.7 | −90.7 | 0.1 | −97.4 | 1.4 | −75.6 | 0.7 | −87.9 |
| | (SD) | (1.15) | (16.12) | (0.25) | (4.47) | (2.24) | (37.32) | (1.32) | (22.62) |
| | Median | 0.0 | −100.0 | 0.0 | −100.0 | 0.3 | −94.2 | 0.0 | −100.0 |
| | Min, | 0.0, | −100.0, | 0.0, | −100.0, | 0.0, | −100.0, | 0.0, | −100.0, |
| | Max | 2.0 | −72.1 | 0.5 | −92.3 | 4.0 | −32.7 | 4.0 | −32.7 |
| Week 5-16 | N (%) | 3 | 3 | 4 | 3 | 3 | 3 | 10 | 9 |
| | | (100) | (100) | (100) | (75.0) | (100) | (100) | (100) | (90.0) |
| | Mean | 0.8 | −89.1 | 0.0 | −100.0 | 0.9 | −85.0 | 0.5 | −91.4 |
| | (SD) | (1.35) | (18.81) | (0.00) | (0.00) | (1.54) | (25.92) | (1.06) | (17.36) |
| | Median | 0.0 | −100.0 | 0.0 | −100.0 | 0.0 | −100.0 | 0.0 | −100.0 |
| | Min, | 0.0, | −100.0, | 0.0, | −100.0, | 0.0, | −100.0, | 0.0, | −100.0, |
| | Max | 2.2 | −67.4 | 0.0 | −100.0 | 2.7 | −55.1 | 2.7 | −55.1 |
| Week 1-24 | N (%) | 3 | 3 | 4 | 3 | 3 | 3 | 10 | 9 |
| | | (100) | (100) | (100) | (75.0) | (100) | (100) | (100) | (90.0) |
| | Mean | 0.4 | −93.8 | 0.1 | −98.3 | 0.9 | −83.7 | 0.5 | −91.9 |
| | (SD) | (0.77) | (10.75) | (0.17) | (2.98) | (1.49) | (24.88) | (0.88) | (15.08) |
| | Median | 0.0 | −100.0 | 0.0 | −100.0 | 0.2 | −96.1 | 0.0 | −100.0 |
| | Min, | 0.0, | −100.0, | 0.0, | −100.0, | 0.0, | −100.0, | 0.0, | −100.0, |
| | Max | 1.3 | −81.4 | 0.3 | −94.8 | 2.7 | −55.1 | 2.7 | −55.1 |
| On-study | N (%) | 3 | 3 | 4 | 3 | 3 | 3 | 10 | 9 |
| Period | | (100) | (100) | (100) | (75.0) | (100) | (100) | (100) | (90.0) |
| | Mean | 0.2 | −95.4 | 0.1 | −98.3 | 0.5 | −91.2 | 0.2 | −95.0 |
| | (SD) | (0.31) | (4.12) | (0.16) | (2.91) | (0.79) | (13.15) | (0.45) | (7.70) |
| | Median | 0.1 | −94.2 | 0.0 | −100.0 | 0.1 | −97.4 | 0.0 | −97.4 |
| | Min, | 0.0, | −100.0, | 0.0, | −100.0, | 0.0, | −100.0, | 0.0, | −100.0, |
| | Max | 0.6 | −92.0 | 0.3 | −95.0 | 1.4 | −76.1 | 1.4 | −76.1 |

Baseline is defined as up to 42 days screening period prior to the administration of NTLA-2002. The HAE attacks are based on “PI Attack Assessment” CRF. Percent change from baseline is calculated only for subjects with HAE attacks during baseline. On-study period is defined as the time from the date of study drug infusion through the last HAE attack assessment as of the data cutoff date. Number of HAE attacks per month is calculated as the number of HAE attacks occurring during the Baseline/specified observation period divided by number of days the subject contributed to the Baseline/specified observation period multiplied by 28 days.

TABLE 21
Descriptive Summary of Investigator-confirmed HAE Attacks requiring Acute Therapy

| | | Dose Level 25 mg | | Dose Level 50 mg | | Dose Level 75 mg | | Total | |
|---|---|---|---|---|---|---|---|---|---|
| | | N = 3 | | N = 4 | | N = 3 | | N = 10 | |
| | | | % change | | % change | | % change | | % change |
| | | | from | | from | | from | | from |
| | | Observed | Baseline | Observed | Baseline | Observed | Baseline | Observed | Baseline |
| Baseline | N (%) | 3 | | 4 | | 3 | | 10 | |
| | | (100) | | (100) | | (100) | | (100) | |
| | Mean | 2.1 | | 0.7 | | 4.4 | | 2.2 | |
| | (SD) | (1.79) | | (1.02) | | (1.47) | | (2.04) | |
| | Median | 2.9 | | 0.3 | | 4.3 | | 2.5 | |
| | Min, | 0.0, | | 0.0, | | 3.0, | | 0.0, | |
| | Max | 3.3 | | 2.2 | | 5.9 | | 5.9 | |
| Week 1-16 | N (%) | 3 | 2 | 4 | 2 | 3 | 3 | 10 | 7 |
| | | (100) | (66.7) | (100) | (50.0) | (100) | (100) | (100) | (70.0) |
| | Mean | 0.6 | −73.1 | 0.1 | −88.4 | 1.3 | −78.4 | 0.6 | −79.8 |
| | (SD) | (1.01) | (38.01) | (0.25) | (16.41) | (1.95) | (32.48) | (1.16) | (26.03) |
| | Median | 0.0 | −73.1 | 0.0 | −88.4 | 0.3 | −94.2 | 0.0 | −94.2 |
| | Min, | 0.0, | −100, | 0.0, | −100.0, | 0.0, | −100.0, | 0.0, | −100.0, |
| | Max | 1.8 | −46.3 | 0.5 | −76.8 | 3.5 | −41.1 | 3.5 | −41.1 |
| Week 5-16 | N (%) | 3 | 2 | 4 | 2 | 3 | 3 | 10 | 7 |
| | | (100) | (66.7) | (100) | (50.0) | (100) | (100) | (100) | (70.0) |
| | Mean | 0.8 | −64.2 | 0.0 | −100.0 | 0.7 | −88.8 | 0.4 | −85.0 |
| | (SD) | (1.35) | (50.68) | (0) | (0) | (1.15) | (19.44) | (0.92) | (27.94) |
| | Median | 0.0 | −64.2 | 0.0 | −100.0 | 0.0 | −100.0 | 0.0 | −100.0 |
| | Min, | 0.0, | −100.0, | 0.0, | −100.0, | 0.0, | −100.0, | 0.0, | −100.0, |
| | Max | 2.3 | −28.3 | 0.0 | −100.0 | 0.2 | −66.3 | 2.3 | −28.3 |
| Week 1-24 | N (%) | 3 | 2 | 4 | 2 | 3 | 3 | 10 | 7 |
| | | (100) | (66.7) | (100) | (50.0) | (100) | (100) | (100) | (70.0) |
| | Mean | 0.4 | −82.1 | 0.1 | −92.3 | 0.8 | −85.6 | 0.4 | −86.5 |
| | (SD) | (0.67) | (25.34) | (0.17) | (10.94) | (1.30) | (21.65) | (0.77) | (17.35) |
| | Median | 0.0 | −82.1 | 0.0 | −92.3 | 0.2 | −96.1 | 0.0 | −96.1 |
| | Min, | 0.0, | −100.0, | 0.0, | −100.0, | 0.0, | −100.0, | 0.0, | −100.0, |
| | Max | 1.2 | −64.2 | 0.3 | −84.5 | 2.3 | −60.7 | 2.3 | −60.7 |
| On-study | N (%) | 3 | 2 | 4 | 2 | 3 | 3 | 10 | 7 |
| Period | | (100) | (66.7) | (100) | (50.0) | (100) | (100) | (100) | (70.0) |
| | Mean | 0.2 | −92.3 | 0.1 | −92.4 | 0.5 | −92.2 | 0.2 | −92.3 |
| | (SD) | (0.29) | (10.83) | (0.16) | (10.69) | (0.69) | (11.43) | (0.40) | (9.06) |
| | Median | 0.0 | −92.3 | 0.0 | −92.4 | 0.1 | −97.4 | 0.0 | −97.4 |
| | Min, | 0.0, | −100.0, | 0.0, | −100.0, | 0.0, | −100.0, | 0.0, | −100.0, |
| | Max | 0.5 | −84.7 | 0.3 | −84.9 | 1.2 | −79.0 | 1.2 | −79.0 |

Baseline is defined as up to 42 days screening period prior to the administration of NTLA-2002. The HAE attacks are based on “PI Attack Assessment” CRF. Percent change from baseline is calculated only for subjects with HAE attacks requiring acute therapy during baseline. On-study period is defined as the time from the date of study drug infusion through the last HAE attack assessment as of the data cutoff date. Number of HAE attacks per month is calculated as the number of HAE attacks occurring during the Baseline/specified observation period divided by number of days the subject contributed to the Baseline/specified observation period multiplied by 28 days.

TABLE 22
Adverse Events

| | Cohort 1 | Cohort 3 | Cohort 2 | All |
|---|---|---|---|---|
| | (25 mg ) | (50 mg) | (75 mg) | patients |
| | n = 3 | n = 4 | n = 3 | n = 10 |
| Any TEAEs | 3 (100) | 3 (75.0) | 3 (100) | 9 (90.0) |
| Dose-Limiting Toxicities | 0 | 0 | 0 | 0 |
| Treatment-related TEAEs | 2 (66.7) | 3 (75.0) | 3 (100) | 8 (80.0) |
| Serious TEAEs | 0 | 0 | 0 | 0 |
| Treatment-related serious TEAEs | 0 | 0 | 0 | 0 |
| TEAEs with grade ≥ 3 | 0 | 0 | 0 | 0 |
| Grade 3 | 0 | 0 | 0 | 0 |
| Grade 4 | 0 | 0 | 0 | 0 |
| Grade 5 | 0 | 0 | 0 | 0 |
| Treatment-related TEAEs with grade ≥ 3 | 0 | 0 | 0 | 0 |
| Grade 3 | 0 | 0 | 0 | 0 |
| Grade 4 | 0 | 0 | 0 | 0 |
| Grade 5 | 0 | 0 | 0 | 0 |
| TEAEs leading to study drug infusion | 0 | 1(25.0) | 0 | 1 (10.0) |
| interruption | | | | |
| Treatment-related TEAEs leading to study | 0 | 1(25.0) | 0 | 1 (10.0) |
| drug infusion interruption | | | | |
| TEAEs leading to study drug infusion | 0 | 0 | 0 | 0 |
| discontinuation | | | | |
| Treatment-related TEAEs leading to study | 0 | 0 | 0 | 0 |
| drug infusion discontinuation | | | | |
| Treatment-emergent AESIs | 2 (66.7) | 2 (50.0) | 3 (100) | 7 (70.0) |
| Treatment-related treatment-emergent | 2 (66.7) | 2 (50.0) | 3 (100) | 7 (70.0) |
| AESIs | | | | |
| TEAEs leading to death | 0 | 0 | 0 | 0 |
| Treatment-related TEAEs leading to death | 0 | 0 | 0 | 0 |

TEAE = treatment-emergent adverse event; AESI = adverse event of special interest.
TEAEs are AEs that started on or after the date of study drug infusion or worsened after study drug infusion through the end of the study. An AE is considered treatment related if the relationship to NTLA-2002 = “Possible” or “Probable” on the “Adverse Events” eCRF page, or if relationship is missing. Each subject is counted only once with the maximum grade if the subject experienced multiple events in the given AE category.

### Example 12. In vivo CRISPR/-Cas9 Editing of KLKB1 in Patients with Hereditary Angioedema: Updated Clinical Data

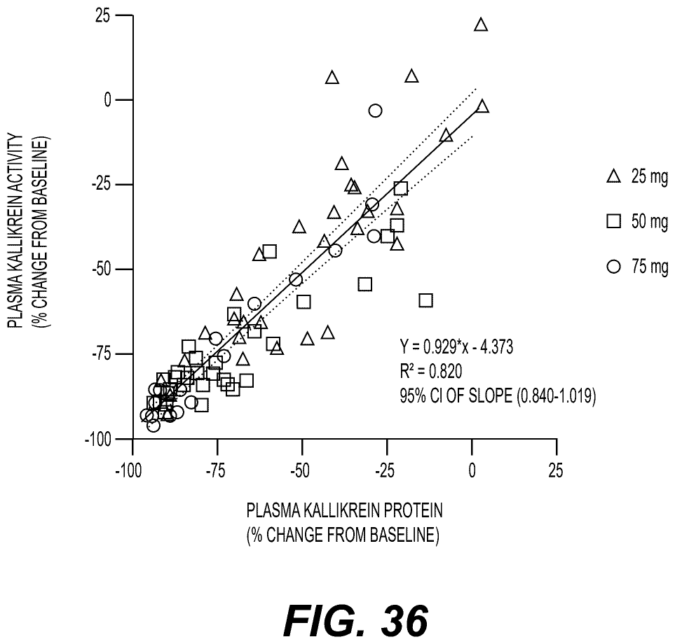

The data in Example 11 was used to analyze the correlation plot between plasma kallikrein protein concentration (% change from baseline) and kallikrein activity (% change from baseline) in HAE patients following administration of a single dose (25, 50 or 75 mg) of NTLA-2002 (FIG. 36). The analysis demonstrates a strong linear correlation (R²=0.820) between % change in kallikrein protein and kallikrein activity from baseline with a slope of 0.929 (95% CI: 0.840-1.019).

### Example 13. In vivo CRISPR/-Cas9 Editing of KLKB1 in Patients with Hereditary Angioedema: Updated Clinical Data, Pharmacokinetic Analysis

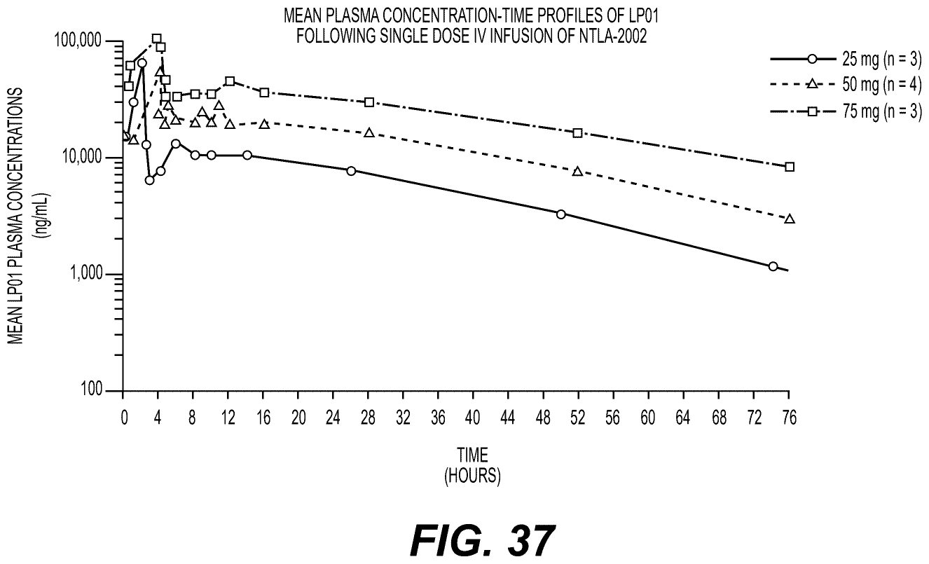

Blood samples were collected to assess plasma concentration time profiles pre-infusion, during the infusion period, and at the end of the infusion period. Post infusion samples were collected at predefined time points as indicated in the graph (FIG. 37). LP01 (Lipid A) was quantified using liquid chromatography with tandem mass spectrometry (LC-MS/MS) methods. Plasma samples were evaluated using validated methods following regulatory guidelines. LPO1 demonstrated dose-dependent exposure and rapid decline below limit of quantification by day 15 with mean t1/2 ranging between 16.8 and 21.3 h.

### Example 14. In vivo CRISPR/-Cas9 Editing of KLKB1 in Patients with Hereditary Angioedema: Updated Clinical Data, Pharmacokinetic Analysis

A follow up analysis of Cohorts 1-3 from Example 10 is provided.

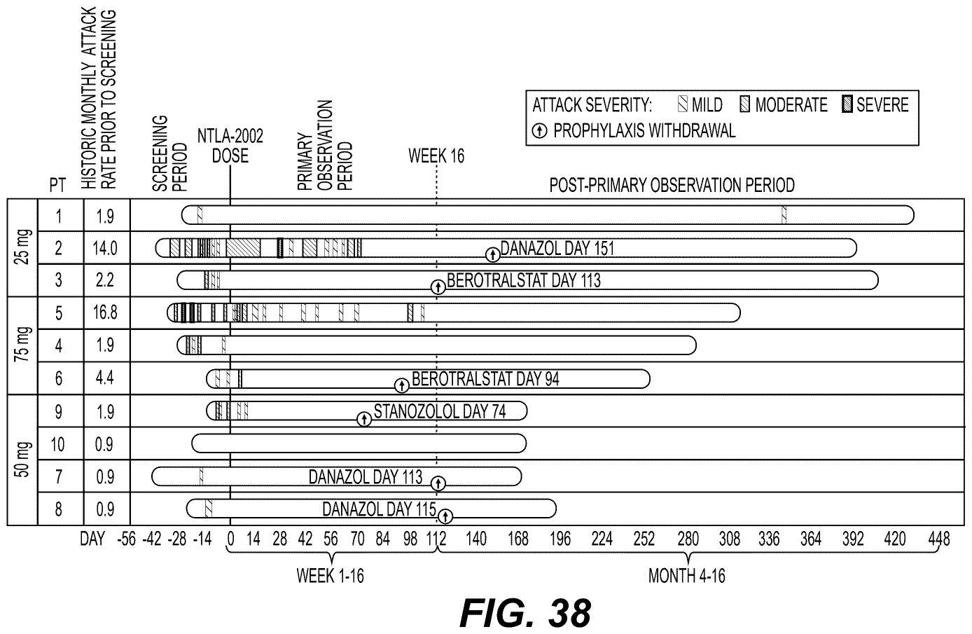

Clinically meaningful reductions in HAE attack rate were observed in all patients with at least 24 weeks of follow-up. With the exception of one patient in Cohort 1 (25 mg), all patients had an ongoing attack-free interval of 3.7 to 12 months (FIG. 38). Each row shows individual patient angioedema attack data, with the historic monthly attack rate prior to screening indicated on the left. Patients are presented in the order of NTLA-2002 treatment date. The length of the colored bars indicates the duration of the attack. Arrows indicate the time of withdrawal of any concomitant long-term prophylaxis (LTP). Patients without arrows were not taking long-term prophylaxis concomitant to study drug.

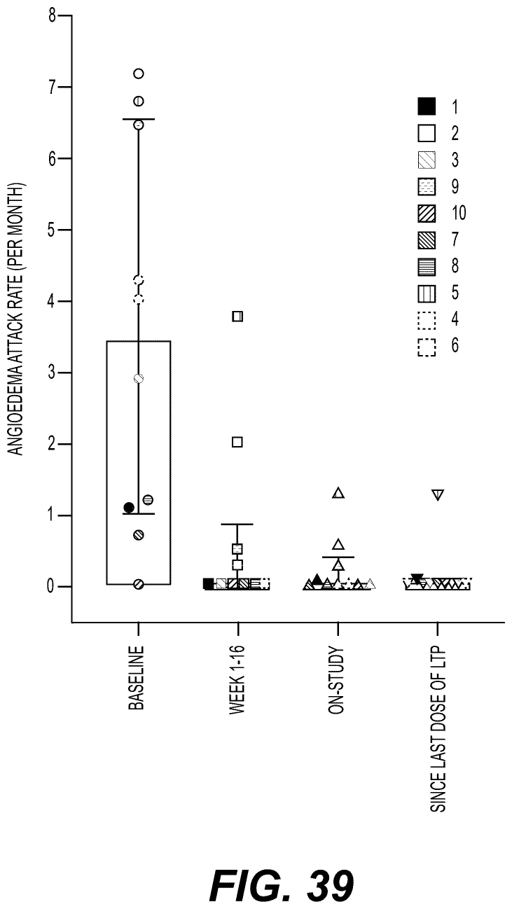

Six patients who were taking concomitant long-term prophylaxis upon study entry with a historic attack rate ranging from 0.9-14.0 attacks per month were able to withdraw their prophylaxis between 2.6-5.4 months after NTLA-2002 administration (FIG. 37). These patients did not report any attacks after withdrawal of long-term prophylaxis. Across all patients, in the absence of background concomitant LTP, the mean monthly attack rate was 0.14 (SD, 0.45) compared to 3.5 (SD, 2.7) at baseline (FIG. 39). The rate of investigator-confirmed angioedema attacks per month at four time periods over the course of the study. Columns indicate median; bars indicate interquartile range; each color indicates an individual patient. Baseline is defined as up to 42-day screening period prior to the administration of NTLA-2002. The on-study period is defined as the time from the administration of NTLA-2002 through the last angioedema attack assessment as of the data cutoff date. Long-term prophylaxis is defined as any type of long-term prophylaxis for the treatment of hereditary angioedema, including NTLA-2002. Only angioedema attacks that occurred after NTLA-2002 administration are included.

All patients demonstrated a high degree of angioedema attack control. Patient 1 experienced mild swelling of the hand precipitated by a sports injury on day 343, but no medical intervention or acute therapy was required, and swelling resolved within two days.

The mean percentage change from baseline in investigator-confirmed attacks per month assessing all patients in all cohorts for various intervals are presented in Table 26 below.

TABLE 26
Percent Change from Baseline in Number of
Investigator-confirmed Attacks per Month

| | NTLA-2002 | NTLA-2002 | NTLA-2002 | All |
|---|---|---|---|---|
| | 25 mg | 50 mg | 75 mg | Patients |
| | (n = 3) | (n = 4) | (n = 3) | (N = 10) |
| Week 1-16 | −91 (16) | −97 (5) | −80 (30) | −89 (19) |
| Week 5-16 | −89 (19) | −100 (0) | −87 (23) | −92 (16) |
| Week 1-24 | −94 (11) | −98 (3) | −86 (20) | −93 (13) |
| On-study | −95 (4) | −98 (3) | −93 (11) | −95 (6) |
| period | | | | |

Data are mean % change from baseline (standard deviation). Baseline is defined as up to 42-day screening period prior to the administration of NTLA-2002. As no minimum number of attacks were required during screening, percent change from baseline in monthly attack rate was calculated only for patients who had attacks at baseline. One patient in the 50 mg cohort experienced no attacks during screening therefore a percent change from baseline could not be determined. The on-study period is defined as the time from the administration of NTLA-2002 through the last angioedema attack assessment as of the data cutoff date.

Consistent with earlier analyses, NTLA-2002 was generally well-tolerated across all three dose levels; all AEs were of mild or moderate severity. All treatment-related TEAEs were grade 2 or lower. All patients received a complete study dose of NTLA-2002. Adverse events are summarized in Table 27 below.

TABLE 27
Treatment-emergent Adverse Events

| Adverse events | NTLA-2002 | NTLA-2002 | NTLA-2002 | All |
|---|---|---|---|---|
| occurring in ≥ 2 | 25 mg | 50 mg | 75 mg | Patients |
| patients | (n = 3) | (n = 4) | (n = 3) | (N = 10) |
| Any TEAE | 3 (100) | 3 (75) | 3 (100) | 9 (90) |
| Any TEAE ≥ | 0 | 0 | 0 | 0 |
| Grade 3 | | | | |
| Infusion-related | 2 (67) | 2 (50) | 3 (100) | 7 (70) |
| reaction | | | | |
| Fatigue | 1 (33) | 3 (75) | 2 (67) | 6 (60) |
| COVID-19 | 3 (100) | 1 (25) | 1 (33) | 5 (50) |
| Upper respiratory | 1 (33) | 1 (25) | 2 (67) | 4 (40) |
| tract infection | | | | |
| Oropharyngeal | 2 (67) | 0 | 1 (33) | 3 (30) |
| pain | | | | |
| Abdominal pain | 1 (33) | 0 | 1 (33) | 2 (20) |
| Headache | 0 | 0 | 2 (67) | 2 (20) |
| Viral upper | 0 | 0 | 2 (67) | 2 (20) |
| respiratory | | | | |
| tract infection | | | | |

Data are n (%). COVID-19, coronavirus disease 2019; mg, milligram; TEAE, treatment-emergent adverse event.

Among 10 patients with hereditary angioedema, a single dose of NTLA-2002 was well-tolerated. A robust, dose-dependent, and durable reduction of plasma kallikrein of 65-95%, and a reduction in monthly angioedema attack rate of 95% was observed across all patients for the duration of follow-up. Patients who discontinued concomitant LTP continued to have well-controlled disease. Based on these data, 25 mg and 50 mg doses were selected for investigation in the randomized, double-blind, placebo-controlled phase 2 portion of the study. These findings provide further clinical validation for targeting kallikrein protein expression as a therapeutic modality for hereditary angioedema.

The patients enrolled in phase 1 had varying historic monthly angioedema attack rates prior to screening. Some patients had more severe baseline disease, and these patients had a longer time to attack control. Additionally, the time needed to achieve steady-state reduction in plasma kallikrein (~4 weeks) was associated with continued attacks early in the primary observation period in some patients. This may be inferred based on the improved attack rate reductions, ranging from 93-98% when evaluated over the full duration of follow-up, vs 80-97% from Week 1-16.

As treatment with NTLA-2002 resulted in a significant reduction of plasma kallikrein, a comparison may be drawn to individuals with congenital plasma prekallikrein deficiency, or “Fletcher factor syndrome,” a rare, autosomal-recessive disorder. These individuals have been reported to be overtly healthy, with the only well-established hallmark being prolonged aPTT without apparent clinical consequence. Although plasma kallikrein is involved in coagulation, the prevalence of major bleeding events was found to be very low in these individuals and was comparable to the rate seen in the general population. None of the patients in the current study experienced prolonged aPTT or any thromboembolic or bleeding events.

The study was a single-arm study and permitted concomitant use of LTP after NTLA-2002, making it difficult to isolate the treatment effect of NTLA-2002. Once LTP was withdrawn, nearly all patients achieved a monthly attack rate of 0, suggesting that clinical benefit was being derived from NTLA-2002 alone. The ongoing randomized phase 2 study requires washout of LTP prior to treatment with NTLA-2002 and will more precisely define its treatment effect.

TABLE 23
Sequence Table

| SEQ ID | | |
|---|---|---|
| NO: | Description | Sequence |
| 15 | KLKB1 targeting | GGAUUGCGUAUGGGACACAA |
| | sequence: | |
| | G012267 | |
| 387 | Mod6 scaffold | GUUUUAGAGCUAGAAAUAGCAAGUUAAAA |
| | sequence | UAAGGCUAGUCCGUUAUCAACUUGAAAAA |
| | (unmodified) of | GUGGCACCGAGUCGGUGCUUUU |
| | G012267 | |
| 388 | Full length | GGAUUGCGUAUGGGACACAAGUUUUAGAG |
| | G012267 | CUAGAAAUAGCAAGUUAAAAUAAGGCUAG |
| | sequence | UCCGUUAUCAACUUGAAAAAGUGGCACCG |
| | | AGUCGGUGCUUUU |
| 389 | 90 mer of | GUUUUAGAGCUAGAAAUAGCAAGUUAAAAU |
| | G012267 | AAGGCUAGUCCGUUAUCACGAAAGGGCACCG |
| | sequence | AGUCGGUGC |
| 390 | 88 mer of | GUUUUAGAGCUAGAAAUAGCAAGUUAAAAUA |
| | G012267 | AGGCUAGUCCGUUAUCAAAAUGGCACCGAGUCGGUGC |
| | sequence | |
| 391 | G012267 | mG*mG*mA*UUGCGUAUGGGACACAAGUUUUAGAmGmCmU |
| | modified | mAmGmAmAmAmUmAmGmCAAGUUAAAAUAAGGCUAGUCCG |
| | | UUAUCAmAmCmUmUmGmAmAmAmAmAmGmUmGmGmCmAmC |
| | | mCmGmAmGmUmCmGmGmUmGmCmU*mU*mU*mU |
| 393 | ORF encoding | AUGGAUAAGAAGUACUCAAUCGGGCUGGAUAUCGGAACUAAUUC |
| | Sp. Cas9 | CGUGGGUUGGGCAGUGAUCACGGAUGAAUACAAAGUGCCGUCCA |
| | | AGAAGUUCAAGGUCCUGGGGAACACCGAUAGACACAGCAUCAAG |
| | | AAAAAUCUCAUCGGAGCCCUGCUGUUUGACUCCGGCGAAACCGC |
| | | AGAAGCGACCCGGCUCAAACGUACCGCGAGGCGACGCUACACCC |
| | | GGCGGAAGAAUCGCAUCUGCUAUCUGCAAGAGAUCUUUUCGAAC |
| | | GAAAUGGCAAAGGUCGACGACAGCUUCUUCCACCGCCUGGAAGA |
| | | AUCUUUCCUGGUGGAGGAGGACAAGAAGCAUGAACGGCAUCCUA |
| | | UCUUUGGAAACAUCGUCGACGAAGUGGCGUACCACGAAAAGUAC |
| | | CCGACCAUCUACCAUCUGCGGAAGAAGUUGGUUGACUCAACUGA |
| | | CAAGGCCGACCUCAGAUUGAUCUACUUGGCCCUCGCCCAUAUGA |
| | | UCAAAUUCCGCGGACACUUCCUGAUCGAAGGCGAUCUGAACCCU |
| | | GAUAACUCCGACGUGGAUAAGCUUUUCAUUCAACUGGUGCAGAC |
| | | CUACAACCAACUGUUCGAAGAAAACCCAAUCAAUGCUAGCGGCG |
| | | UCGAUGCCAAGGCCAUCCUGUCCGCCCGGCUGUCGAAGUCGCGG |
| | | CGCCUCGAAAACCUGAUCGCACAGCUGCCGGGAGAGAAAAAGAA |
| | | CGGACUUUUCGGCAACUUGAUCGCUCUCUCACUGGGACUCACUC |
| | | CCAAUUUCAAGUCCAAUUUUGACCUGGCCGAGGACGCGAAGCUG |
| | | CAACUCUCAAAGGACACCUACGACGACGACUUGGACAAUUUGCU |
| | | GGCACAAAUUGGCGAUCAGUACGCGGAUCUGUUCCUUGCCGCUA |
| | | AGAACCUUUCGGACGCAAUCUUGCUGUCCGAUAUCCUGCGCGUG |
| | | AACACCGAAAUAACCAAAGCGCCGCUUAGCGCCUCGAUGAUUAA |
| | | GCGGUACGACGAGCAUCACCAGGAUCUCACGCUGCUCAAAGCGC |
| | | UCGUGAGACAGCAACUGCCUGAAAAGUACAAGGAGAUCUUCUUC |
| | | GACCAGUCCAAGAAUGGGUACGCAGGGUACAUCGAUGGAGGCGC |
| | | UAGCCAGGAAGAGUUCUAUAAGUUCAUCAAGCCAAUCCUGGAAA |
| | | AGAUGGACGGAACCGAAGAACUGCUGGUCAAGCUGAACAGGGAG |
| | | GAUCUGCUCCGGAAACAGAGAACCUUUGACAACGGAUCCAUUCC |
| | | CCACCAGAUCCAUCUGGGUGAGCUGCACGCCAUCUUGCGGCGCC |
| | | AGGAGGACUUUUACCCAUUCCUCAAGGACAACCGGGAAAAGAUC |
| | | GAGAAAAUUCUGACGUUCCGCAUCCCGUAUUACGUGGGCCCACU |
| | | GGCGCGCGGCAAUUCGCGCUUCGCGUGGAUGACUAGAAAAUCAG |
| | | AGGAAACCAUCACUCCUUGGAAUUUCGAGGAAGUUGUGGAUAAG |
| | | GGAGCUUCGGCACAAAGCUUCAUCGAACGAAUGACCAACUUCGA |
| | | CAAGAAUCUCCCAAACGAGAAGGUGCUUCCUAAGCACAGCCUCC |
| | | UUUACGAAUACUUCACUGUCUACAACGAACUGACUAAAGUGAAA |
| | | UACGUUACUGAAGGAAUGAGGAAGCCGGCCUUUCUGUCCGGAGA |
| | | ACAGAAGAAAGCAAUUGUCGAUCUGCUGUUCAAGACCAACCGCA |
| | | AGGUGACCGUCAAGCAGCUUAAAGAGGACUACUUCAAGAAGAUC |
| | | GAGUGUUUCGACUCAGUGGAAAUCAGCGGGGUGGAGGACAGAUU |
| | | CAACGCUUCGCUGGGAACCUAUCAUGAUCUCCUGAAGAUCAUCA |
| | | AGGACAAGGACUUCCUUGACAACGAGGAGAACGAGGACAUCCUG |
| | | GAAGAUAUCGUCCUGACCUUGACCCUUUUCGAGGAUCGCGAGAU |
| | | GAUCGAGGAGAGGCUUAAGACCUACGCUCAUCUCUUCGACGAUA |
| | | AGGUCAUGAAACAACUCAAGCGCCGCCGGUACACUGGUUGGGGC |
| | | CGCCUCUCCCGCAAGCUGAUCAACGGUAUUCGCGAUAAACAGAG |
| | | CGGUAAAACUAUCCUGGAUUUCCUCAAAUCGGAUGGCUUCGCUA |
| | | AUCGUAACUUCAUGCAAUUGAUCCACGACGACAGCCUGACCUUU |
| | | AAGGAGGACAUCCAAAAAGCACAAGUGUCCGGACAGGGAGACUC |
| | | ACUCCAUGAACACAUCGCGAAUCUGGCCGGUUCGCCGGCGAUUA |
| | | AGAAGGGAAUUCUGCAAACUGUGAAGGUGGUCGACGAGCUGGUG |
| | | AAGGUCAUGGGACGGCACAAACCGGAGAAUAUCGUGAUUGAAAU |
| | | GGCCCGAGAAAACCAGACUACCCAGAAGGGCCAGAAAAACUCCC |
| | | GCGAAAGGAUGAAGCGGAUCGAAGAAGGAAUCAAGGAGCUGGGC |
| | | AGCCAGAUCCUGAAAGAGCACCCGGUGGAAAACACGCAGCUGCA |
| | | GAACGAGAAGCUCUACCUGUACUAUUUGCAAAAUGGACGGGACA |
| | | UGUACGUGGACCAAGAGCUGGACAUCAAUCGGUUGUCUGAUUAC |
| | | GACGUGGACCACAUCGUUCCACAGUCCUUUCUGAAGGAUGACUC |
| | | GAUCGAUAACAAGGUGUUGACUCGCAGCGACAAGAACAGAGGGA |
| | | AGUCAGAUAAUGUGCCAUCGGAGGAGGUCGUGAAGAAGAUGAAG |
| | | AAUUACUGGCGGCAGCUCCUGAAUGCGAAGCUGAUUACCCAGAG |
| | | AAAGUUUGACAAUCUCACUAAAGCCGAGCGCGGCGGACUCUCAG |
| | | AGCUGGAUAAGGCUGGAUUCAUCAAACGGCAGCUGGUCGAGACU |
| | | CGGCAGAUUACCAAGCACGUGGCGCAGAUCUUGGACUCCCGCAU |
| | | GAACACUAAAUACGACGAGAACGAUAAGCUCAUCCGGGAAGUGA |
| | | AGGUGAUUACCCUGAAAAGCAAACUUGUGUCGGACUUUCGGAAG |
| | | GACUUUCAGUUUUACAAAGUGAGAGAAAUCAACAACUACCAUCA |
| | | CGCGCAUGACGCAUACCUCAACGCUGUGGUCGGUACCGCCCUGA |
| | | UCAAAAAGUACCCUAAACUUGAAUCGGAGUUUGUGUACGGAGAC |
| | | UACAAGGUCUACGACGUGAGGAAGAUGAUAGCCAAGUCCGAACA |
| | | GGAAAUCGGGAAAGCAACUGCGAAAUACUUCUUUUACUCAAACA |
| | | UCAUGAACUUUUUCAAGACUGAAAUUACGCUGGCCAAUGGAGAA |
| | | AUCAGGAAGAGGCCACUGAUCGAAACUAACGGAGAAACGGGCGA |
| | | AAUCGUGUGGGACAAGGGCAGGGACUUCGCAACUGUUCGCAAAG |
| | | UGCUCUCUAUGCCGCAAGUCAAUAUUGUGAAGAAAACCGAAGUG |
| | | CAAACCGGCGGAUUUUCAAAGGAAUCGAUCCUCCCAAAGAGAAA |
| | | UAGCGACAAGCUCAUUGCACGCAAGAAAGACUGGGACCCGAAGA |
| | | AGUACGGAGGAUUCGAUUCGCCGACUGUCGCAUACUCCGUCCUC |
| | | GUGGUGGCCAAGGUGGAGAAGGGAAAGAGCAAAAAGCUCAAAUC |
| | | CGUCAAAGAGCUGCUGGGGAUUACCAUCAUGGAACGAUCCUCGU |
| | | UCGAGAAGAACCCGAUUGAUUUCCUCGAGGCGAAGGGUUACAAG |
| | | GAGGUGAAGAAGGAUCUGAUCAUCAAACUCCCCAAGUACUCACU |
| | | GUUCGAACUGGAAAAUGGUCGGAAGCGCAUGCUGGCUUCGGCCG |
| | | GAGAACUCCAAAAAGGAAAUGAGCUGGCCUUGCCUAGCAAGUAC |
| | | GUCAACUUCCUCUAUCUUGCUUCGCACUACGAAAAACUCAAAGG |
| | | GUCACCGGAAGAUAACGAACAGAAGCAGCUUUUCGUGGAGCAGC |
| | | ACAAGCAUUAUCUGGAUGAAAUCAUCGAACAAAUCUCCGAGUUU |
| | | UCAAAGCGCGUGAUCCUCGCCGACGCCAACCUCGACAAAGUCCU |
| | | GUCGGCCUACAAUAAGCAUAGAGAUAAGCCGAUCAGAGAACAGG |
| | | CCGAGAACAUUAUCCACUUGUUCACCCUGACUAACCUGGGAGCC |
| | | CCAGCCGCCUUCAAGUACUUCGAUACUACUAUCGAUCGCAAAAG |
| | | AUACACGUCCACCAAGGAAGUUCUGGACGCGACCCUGAUCCACC |
| | | AAAGCAUCACUGGACUCUACGAAACUAGGAUCGAUCUGUCGCAG |
| | | CUGGGUGGCGAUGGCGGUGGAUCUCCGAAAAAGAAGAGAAAGGU |
| | | GUAAUGA |
| 394 | ORF encoding | ATGGACAAGAAGTACAGCATCGGACTGGACATCGGAACAAACAG |
| | Sp. Cas9 | CGTCGGATGGGCAGTCATCACAGACGAATACAAGGTCCCGAGCA |
| | | AGAAGTTCAAGGTCCTGGGAAACACAGACAGACACAGCATCAAG |
| | | AAGAACCTGATCGGAGCACTGCTGTTCGACAGCGGAGAAACAGC |
| | | AGAAGCAACAAGACTGAAGAGAACAGCAAGAAGAAGATACACAA |
| | | GAAGAAAGAACAGAATCTGCTACCTGCAGGAAATCTTCAGCAAC |
| | | GAAATGGCAAAGGTCGACGACAGCTTCTTCCACAGACTGGAAGA |
| | | AAGCTTCCTGGTCGAAGAAGACAAGAAGCACGAAAGACACCCGA |
| | | TCTTCGGAAACATCGTCGACGAAGTCGCATACCACGAAAAGTAC |
| | | CCGACAATCTACCACCTGAGAAAGAAGCTGGTCGACAGCACAGA |
| | | CAAGGCAGACCTGAGACTGATCTACCTGGCACTGGCACACATGA |
| | | TCAAGTTCAGAGGACACTTCCTGATCGAAGGAGACCTGAACCCG |
| | | GACAACAGCGACGTCGACAAGCTGTTCATCCAGCTGGTCCAGAC |
| | | ATACAACCAGCTGTTCGAAGAAAACCCGATCAACGCAAGCGGAG |
| | | TCGACGCAAAGGCAATCCTGAGCGCAAGACTGAGCAAGAGCAGA |
| | | AGACTGGAAAACCTGATCGCACAGCTGCCGGGAGAAAAGAAGAA |
| | | CGGACTGTTCGGAAACCTGATCGCACTGAGCCTGGGACTGACAC |
| | | CGAACTTCAAGAGCAACTTCGACCTGGCAGAAGACGCAAAGCTG |
| | | CAGCTGAGCAAGGACACATACGACGACGACCTGGACAACCTGCT |
| | | GGCACAGATCGGAGACCAGTACGCAGACCTGTTCCTGGCAGCAA |
| | | AGAACCTGAGCGACGCAATCCTGCTGAGCGACATCCTGAGAGTC |
| | | AACACAGAAATCACAAAGGCACCGCTGAGCGCAAGCATGATCAA |
| | | GAGATACGACGAACACCACCAGGACCTGACACTGCTGAAGGCAC |
| | | TGGTCAGACAGCAGCTGCCGGAAAAGTACAAGGAAATCTTCTTC |
| | | GACCAGAGCAAGAACGGATACGCAGGATACATCGACGGAGGAGC |
| | | AAGCCAGGAAGAATTCTACAAGTTCATCAAGCCGATCCTGGAAA |
| | | AGATGGACGGAACAGAAGAACTGCTGGTCAAGCTGAACAGAGAA |
| | | GACCTGCTGAGAAAGCAGAGAACATTCGACAACGGAAGCATCCC |
| | | GCACCAGATCCACCTGGGAGAACTGCACGCAATCCTGAGAAGAC |
| | | AGGAAGACTTCTACCCGTTCCTGAAGGACAACAGAGAAAAGATC |
| | | GAAAAGATCCTGACATTCAGAATCCCGTACTACGTCGGACCGCT |
| | | GGCAAGAGGAAACAGCAGATTCGCATGGATGACAAGAAAGAGCG |
| | | AAGAAACAATCACACCGTGGAACTTCGAAGAAGTCGTCGACAAG |
| | | GGAGCAAGCGCACAGAGCTTCATCGAAAGAATGACAAACTTCGA |
| | | CAAGAACCTGCCGAACGAAAAGGTCCTGCCGAAGCACAGCCTGC |
| | | TGTACGAATACTTCACAGTCTACAACGAACTGACAAAGGTCAAG |
| | | TACGTCACAGAAGGAATGAGAAAGCCGGCATTCCTGAGCGGAGA |
| | | ACAGAAGAAGGCAATCGTCGACCTGCTGTTCAAGACAAACAGAA |
| | | AGGTCACAGTCAAGCAGCTGAAGGAAGACTACTTCAAGAAGATC |
| | | GAATGCTTCGACAGCGTCGAAATCAGCGGAGTCGAAGACAGATT |
| | | CAACGCAAGCCTGGGAACATACCACGACCTGCTGAAGATCATCA |
| | | AGGACAAGGACTTCCTGGACAACGAAGAAAACGAAGACATCCTG |
| | | GAAGACATCGTCCTGACACTGACACTGTTCGAAGACAGAGAAAT |
| | | GATCGAAGAAAGACTGAAGACATACGCACACCTGTTCGACGACA |
| | | AGGTCATGAAGCAGCTGAAGAGAAGAAGATACACAGGATGGGGA |
| | | AGACTGAGCAGAAAGCTGATCAACGGAATCAGAGACAAGCAGAG |
| | | CGGAAAGACAATCCTGGACTTCCTGAAGAGCGACGGATTCGCAA |
| | | ACAGAAACTTCATGCAGCTGATCCACGACGACAGCCTGACATTC |
| | | AAGGAAGACATCCAGAAGGCACAGGTCAGCGGACAGGGAGACAG |
| | | CCTGCACGAACACATCGCAAACCTGGCAGGAAGCCCGGCAATCA |
| | | AGAAGGGAATCCTGCAGACAGTCAAGGTCGTCGACGAACTGGTC |
| | | AAGGTCATGGGAAGACACAAGCCGGAAAACATCGTCATCGAAAT |
| | | GGCAAGAGAAAACCAGACAACACAGAAGGGACAGAAGAACAGCA |
| | | GAGAAAGAATGAAGAGAATCGAAGAAGGAATCAAGGAACTGGGA |
| | | AGCCAGATCCTGAAGGAACACCCGGTCGAAAACACACAGCTGCA |
| | | GAACGAAAAGCTGTACCTGTACTACCTGCAGAACGGAAGAGACA |
| | | TGTACGTCGACCAGGAACTGGACATCAACAGACTGAGCGACTAC |
| | | GACGTCGACCACATCGTCCCGCAGAGCTTCCTGAAGGACGACAG |
| | | CATCGACAACAAGGTCCTGACAAGAAGCGACAAGAACAGAGGAA |
| | | AGAGCGACAACGTCCCGAGCGAAGAAGTCGTCAAGAAGATGAAG |
| | | AACTACTGGAGACAGCTGCTGAACGCAAAGCTGATCACACAGAG |
| | | AAAGTTCGACAACCTGACAAAGGCAGAGAGAGGAGGACTGAGCG |
| | | AACTGGACAAGGCAGGATTCATCAAGAGACAGCTGGTCGAAACA |
| | | AGACAGATCACAAAGCACGTCGCACAGATCCTGGACAGCAGAAT |
| | | GAACACAAAGTACGACGAAAACGACAAGCTGATCAGAGAAGTCA |
| | | AGGTCATCACACTGAAGAGCAAGCTGGTCAGCGACTTCAGAAAG |
| | | GACTTCCAGTTCTACAAGGTCAGAGAAATCAACAACTACCACCA |
| | | CGCACACGACGCATACCTGAACGCAGTCGTCGGAACAGCACTGA |
| | | TCAAGAAGTACCCGAAGCTGGAAAGCGAATTCGTCTACGGAGAC |
| | | TACAAGGTCTACGACGTCAGAAAGATGATCGCAAAGAGCGAACA |
| | | GGAAATCGGAAAGGCAACAGCAAAGTACTTCTTCTACAGCAACA |
| | | TCATGAACTTCTTCAAGACAGAAATCACACTGGCAAACGGAGAA |
| | | ATCAGAAAGAGACCGCTGATCGAAACAAACGGAGAAACAGGAGA |
| | | AATCGTCTGGGACAAGGGAAGAGACTTCGCAACAGTCAGAAAGG |
| | | TCCTGAGCATGCCGCAGGTCAACATCGTCAAGAAGACAGAAGTC |
| | | CAGACAGGAGGATTCAGCAAGGAAAGCATCCTGCCGAAGAGAAA |
| | | CAGCGACAAGCTGATCGCAAGAAAGAAGGACTGGGACCCGAAGA |
| | | AGTACGGAGGATTCGACAGCCCGACAGTCGCATACAGCGTCCTG |
| | | GTCGTCGCAAAGGTCGAAAAGGGAAAGAGCAAGAAGCTGAAGAG |
| | | CGTCAAGGAACTGCTGGGAATCACAATCATGGAAAGAAGCAGCT |
| | | TCGAAAAGAACCCGATCGACTTCCTGGAAGCAAAGGGATACAAG |
| | | GAAGTCAAGAAGGACCTGATCATCAAGCTGCCGAAGTACAGCCT |
| | | GTTCGAACTGGAAAACGGAAGAAAGAGAATGCTGGCAAGCGCAG |
| | | GAGAACTGCAGAAGGGAAACGAACTGGCACTGCCGAGCAAGTAC |
| | | GTCAACTTCCTGTACCTGGCAAGCCACTACGAAAAGCTGAAGGG |
| | | AAGCCCGGAAGACAACGAACAGAAGCAGCTGTTCGTCGAACAGC |
| | | ACAAGCACTACCTGGACGAAATCATCGAACAGATCAGCGAATTC |
| | | AGCAAGAGAGTCATCCTGGCAGACGCAAACCTGGACAAGGTCCT |
| | | GAGCGCATACAACAAGCACAGAGACAAGCCGATCAGAGAACAGG |
| | | CAGAAAACATCATCCACCTGTTCACACTGACAAACCTGGGAGCA |
| | | CCGGCAGCATTCAAGTACTTCGACACAACAATCGACAGAAAGAG |
| | | ATACACAAGCACAAAGGAAGTCCTGGACGCAACACTGATCCACC |
| | | AGAGCATCACAGGACTGTACGAAACAAGAATCGACCTGAGCCAG |
| | | CTGGGAGGAGACGGAGGAGGAAGCCCGAAGAAGAAGAGAAAGGT |
| | | CTAG |
| 395 | ORF encoding | ATGGACAAGAAGTACTCCATCGGCCTGGACATCGGCACCAACTC |
| | Sp. Cas9 | CGTGGGCTGGGCCGTGATCACCGACGAGTACAAGGTGCCCTCCA |
| | | AGAAGTTCAAGGTGCTGGGCAACACCGACCGGCACTCCATCAAG |
| | | AAGAACCTGATCGGCGCCCTGCTGTTCGACTCCGGCGAGACCGC |
| | | CGAGGCCACCCGGCTGAAGCGGACCGCCCGGCGGCGGTACACCC |
| | | GGCGGAAGAACCGGATCTGCTACCTGCAGGAGATCTTCTCCAAC |
| | | GAGATGGCCAAGGTGGACGACTCCTTCTTCCACCGGCTGGAGGA |
| | | GTCCTTCCTGGTGGAGGAGGACAAGAAGCACGAGCGGCACCCCA |
| | | TCTTCGGCAACATCGTGGACGAGGTGGCCTACCACGAGAAGTAC |
| | | CCCACCATCTACCACCTGCGGAAGAAGCTGGTGGACTCCACCGA |
| | | CAAGGCCGACCTGCGGCTGATCTACCTGGCCCTGGCCCACATGA |
| | | TCAAGTTCCGGGGCCACTTCCTGATCGAGGGCGACCTGAACCCC |
| | | GACAACTCCGACGTGGACAAGCTGTTCATCCAGCTGGTGCAGAC |
| | | CTACAACCAGCTGTTCGAGGAGAACCCCATCAACGCCTCCGGCG |
| | | TGGACGCCAAGGCCATCCTGTCCGCCCGGCTGTCCAAGTCCCGG |
| | | CGGCTGGAGAACCTGATCGCCCAGCTGCCCGGCGAGAAGAAGAA |
| | | CGGCCTGTTCGGCAACCTGATCGCCCTGTCCCTGGGCCTGACCC |
| | | CCAACTTCAAGTCCAACTTCGACCTGGCCGAGGACGCCAAGCTG |
| | | CAGCTGTCCAAGGACACCTACGACGACGACCTGGACAACCTGCT |
| | | GGCCCAGATCGGCGACCAGTACGCCGACCTGTTCCTGGCCGCCA |
| | | AGAACCTGTCCGACGCCATCCTGCTGTCCGACATCCTGCGGGTG |
| | | AACACCGAGATCACCAAGGCCCCCCTGTCCGCCTCCATGATCAA |
| | | GCGGTACGACGAGCACCACCAGGACCTGACCCTGCTGAAGGCCC |
| | | TGGTGCGGCAGCAGCTGCCCGAGAAGTACAAGGAGATCTTCTTC |
| | | GACCAGTCCAAGAACGGCTACGCCGGCTACATCGACGGCGGCGC |
| | | CTCCCAGGAGGAGTTCTACAAGTTCATCAAGCCCATCCTGGAGA |
| | | AGATGGACGGCACCGAGGAGCTGCTGGTGAAGCTGAACCGGGAG |
| | | GACCTGCTGCGGAAGCAGCGGACCTTCGACAACGGCTCCATCCC |
| | | CCACCAGATCCACCTGGGCGAGCTGCACGCCATCCTGCGGCGGC |
| | | AGGAGGACTTCTACCCCTTCCTGAAGGACAACCGGGAGAAGATC |
| | | GAGAAGATCCTGACCTTCCGGATCCCCTACTACGTGGGCCCCCT |
| | | GGCCCGGGGCAACTCCCGGTTCGCCTGGATGACCCGGAAGTCCG |
| | | AGGAGACCATCACCCCCTGGAACTTCGAGGAGGTGGTGGACAAG |
| | | GGCGCCTCCGCCCAGTCCTTCATCGAGCGGATGACCAACTTCGA |
| | | CAAGAACCTGCCCAACGAGAAGGTGCTGCCCAAGCACTCCCTGC |
| | | TGTACGAGTACTTCACCGTGTACAACGAGCTGACCAAGGTGAAG |
| | | TACGTGACCGAGGGCATGCGGAAGCCCGCCTTCCTGTCCGGCGA |
| | | GCAGAAGAAGGCCATCGTGGACCTGCTGTTCAAGACCAACCGGA |
| | | AGGTGACCGTGAAGCAGCTGAAGGAGGACTACTTCAAGAAGATC |
| | | GAGTGCTTCGACTCCGTGGAGATCTCCGGCGTGGAGGACCGGTT |
| | | CAACGCCTCCCTGGGCACCTACCACGACCTGCTGAAGATCATCA |
| | | AGGACAAGGACTTCCTGGACAACGAGGAGAACGAGGACATCCTG |
| | | GAGGACATCGTGCTGACCCTGACCCTGTTCGAGGACCGGGAGAT |
| | | GATCGAGGAGCGGCTGAAGACCTACGCCCACCTGTTCGACGACA |
| | | AGGTGATGAAGCAGCTGAAGCGGCGGCGGTACACCGGCTGGGGC |
| | | CGGCTGTCCCGGAAGCTGATCAACGGCATCCGGGACAAGCAGTC |
| | | CGGCAAGACCATCCTGGACTTCCTGAAGTCCGACGGCTTCGCCA |
| | | ACCGGAACTTCATGCAGCTGATCCACGACGACTCCCTGACCTTC |
| | | AAGGAGGACATCCAGAAGGCCCAGGTGTCCGGCCAGGGCGACTC |
| | | CCTGCACGAGCACATCGCCAACCTGGCCGGCTCCCCCGCCATCA |
| | | AGAAGGGCATCCTGCAGACCGTGAAGGTGGTGGACGAGCTGGTG |
| | | AAGGTGATGGGCCGGCACAAGCCCGAGAACATCGTGATCGAGAT |
| | | GGCCCGGGAGAACCAGACCACCCAGAAGGGCCAGAAGAACTCCC |
| | | GGGAGCGGATGAAGCGGATCGAGGAGGGCATCAAGGAGCTGGGC |
| | | TCCCAGATCCTGAAGGAGCACCCCGTGGAGAACACCCAGCTGCA |
| | | GAACGAGAAGCTGTACCTGTACTACCTGCAGAACGGCCGGGACA |
| | | TGTACGTGGACCAGGAGCTGGACATCAACCGGCTGTCCGACTAC |
| | | GACGTGGACCACATCGTGCCCCAGTCCTTCCTGAAGGACGACTC |
| | | CATCGACAACAAGGTGCTGACCCGGTCCGACAAGAACCGGGGCA |
| | | AGTCCGACAACGTGCCCTCCGAGGAGGTGGTGAAGAAGATGAAG |
| | | AACTACTGGCGGCAGCTGCTGAACGCCAAGCTGATCACCCAGCG |
| | | GAAGTTCGACAACCTGACCAAGGCCGAGCGGGGCGGCCTGTCCG |
| | | AGCTGGACAAGGCCGGCTTCATCAAGCGGCAGCTGGTGGAGACC |
| | | CGGCAGATCACCAAGCACGTGGCCCAGATCCTGGACTCCCGGAT |
| | | GAACACCAAGTACGACGAGAACGACAAGCTGATCCGGGAGGTGA |
| | | AGGTGATCACCCTGAAGTCCAAGCTGGTGTCCGACTTCCGGAAG |
| | | GACTTCCAGTTCTACAAGGTGCGGGAGATCAACAACTACCACCA |
| | | CGCCCACGACGCCTACCTGAACGCCGTGGTGGGCACCGCCCTGA |
| | | TCAAGAAGTACCCCAAGCTGGAGTCCGAGTTCGTGTACGGCGAC |
| | | TACAAGGTGTACGACGTGCGGAAGATGATCGCCAAGTCCGAGCA |
| | | GGAGATCGGCAAGGCCACCGCCAAGTACTTCTTCTACTCCAACA |
| | | TCATGAACTTCTTCAAGACCGAGATCACCCTGGCCAACGGCGAG |
| | | ATCCGGAAGCGGCCCCTGATCGAGACCAACGGCGAGACCGGCGA |
| | | GATCGTGTGGGACAAGGGCCGGGACTTCGCCACCGTGCGGAAGG |
| | | TGCTGTCCATGCCCCAGGTGAACATCGTGAAGAAGACCGAGGTG |
| | | CAGACCGGCGGCTTCTCCAAGGAGTCCATCCTGCCCAAGCGGAA |
| | | CTCCGACAAGCTGATCGCCCGGAAGAAGGACTGGGACCCCAAGA |
| | | AGTACGGCGGCTTCGACTCCCCCACCGTGGCCTACTCCGTGCTG |
| | | GTGGTGGCCAAGGTGGAGAAGGGCAAGTCCAAGAAGCTGAAGTC |
| | | CGTGAAGGAGCTGCTGGGCATCACCATCATGGAGCGGTCCTCCT |
| | | TCGAGAAGAACCCCATCGACTTCCTGGAGGCCAAGGGCTACAAG |
| | | GAGGTGAAGAAGGACCTGATCATCAAGCTGCCCAAGTACTCCCT |
| | | GTTCGAGCTGGAGAACGGCCGGAAGCGGATGCTGGCCTCCGCCG |
| | | GCGAGCTGCAGAAGGGCAACGAGCTGGCCCTGCCCTCCAAGTAC |
| | | GTGAACTTCCTGTACCTGGCCTCCCACTACGAGAAGCTGAAGGG |
| | | CTCCCCCGAGGACAACGAGCAGAAGCAGCTGTTCGTGGAGCAGC |
| | | ACAAGCACTACCTGGACGAGATCATCGAGCAGATCTCCGAGTTC |
| | | TCCAAGCGGGTGATCCTGGCCGACGCCAACCTGGACAAGGTGCT |
| | | GTCCGCCTACAACAAGCACCGGGACAAGCCCATCCGGGAGCAGG |
| | | CCGAGAACATCATCCACCTGTTCACCCTGACCAACCTGGGCGCC |
| | | CCCGCCGCCTTCAAGTACTTCGACACCACCATCGACCGGAAGCG |
| | | GTACACCTCCACCAAGGAGGTGCTGGACGCCACCCTGATCCACC |
| | | AGTCCATCACCGGCCTGTACGAGACCCGGATCGACCTGTCCCAG |
| | | CTGGGCGGCGACGGCGGCGGCTCCCCCAAGAAGAAGCGGAAGGT |
| | | GTGA |
| 396 | ORF encoding | AUGGACAAGAAGUACAGCAUCGGCCUGGACAUCGGCACGAACAG |
| | Sp. Cas9 | CGUUGGCUGGGCUGUGAUCACGGACGAGUACAAGGUUCCCUCAA |
| | | AGAAGUUCAAGGUGCUGGGCAACACGGACCGGCACAGCAUCAAG |
| | | AAGAAUCUCAUCGGUGCACUGCUGUUCGACAGCGGUGAGACGGC |
| | | CGAAGCCACGCGGCUGAAGCGGACGGCCCGCCGGCGGUACACGC |
| | | GGCGGAAGAACCGGAUCUGCUACCUGCAGGAGAUCUUCAGCAAC |
| | | GAGAUGGCCAAGGUGGACGACAGCUUCUUCCACCGGCUGGAGGA |
| | | GAGCUUCCUGGUGGAGGAGGACAAGAAGCACGAGCGGCACCCCA |
| | | UCUUCGGCAACAUCGUGGACGAAGUCGCCUACCACGAGAAGUAC |
| | | CCCACCAUCUACCACCUGCGGAAGAAGCUGGUGGACUCGACUGA |
| | | CAAGGCCGACCUGCGGCUGAUCUACCUGGCACUGGCCCACAUGA |
| | | UAAAGUUCCGGGGCCACUUCCUGAUCGAGGGCGACCUGAACCCU |
| | | GACAACAGCGACGUGGACAAGCUGUUCAUCCAGCUGGUGCAGAC |
| | | CUACAACCAGCUGUUCGAGGAGAACCCCAUCAACGCCAGCGGCG |
| | | UGGACGCCAAGGCCAUCCUCAGCGCCCGCCUCAGCAAGAGCCGG |
| | | CGGCUGGAGAAUCUCAUCGCCCAGCUUCCAGGUGAGAAGAAGAA |
| | | UGGGCUGUUCGGCAAUCUCAUCGCACUCAGCCUGGGCCUGACUC |
| | | CCAACUUCAAGAGCAACUUCGACCUGGCCGAGGACGCCAAGCUG |
| | | CAGCUCAGCAAGGACACCUACGACGACGACCUGGACAAUCUCCU |
| | | GGCCCAGAUCGGCGACCAGUACGCCGACCUGUUCCUGGCUGCCA |
| | | AGAAUCUCAGCGACGCCAUCCUGCUCAGCGACAUCCUGCGGGUG |
| | | AACACAGAGAUCACGAAGGCCCCCCUCAGCGCCAGCAUGAUAAA |
| | | GCGGUACGACGAGCACCACCAGGACCUGACGCUGCUGAAGGCAC |
| | | UGGUGCGGCAGCAGCUUCCAGAGAAGUACAAGGAGAUCUUCUUC |
| | | GACCAGAGCAAGAAUGGGUACGCCGGGUACAUCGACGGUGGUGC |
| | | CAGCCAGGAGGAGUUCUACAAGUUCAUCAAGCCCAUCCUGGAGA |
| | | AGAUGGACGGCACAGAGGAGCUGCUGGUGAAGCUGAACAGGGAG |
| | | GACCUGCUGCGGAAGCAGCGGACGUUCGACAAUGGGAGCAUCCC |
| | | CCACCAGAUCCACCUGGGUGAGCUGCACGCCAUCCUGCGGCGGC |
| | | AGGAGGACUUCUACCCCUUCCUGAAGGACAACAGGGAGAAGAUC |
| | | GAGAAGAUCCUGACGUUCCGGAUCCCCUACUACGUUGGCCCCCU |
| | | GGCCCGCGGCAACAGCCGGUUCGCCUGGAUGACGCGGAAGAGCG |
| | | AGGAGACGAUCACUCCCUGGAACUUCGAGGAAGUCGUGGACAAG |
| | | GGUGCCAGCGCCCAGAGCUUCAUCGAGCGGAUGACGAACUUCGA |
| | | CAAGAAUCUUCCAAACGAGAAGGUGCUUCCAAAGCACAGCCUGC |
| | | UGUACGAGUACUUCACGGUGUACAACGAGCUGACGAAGGUGAAG |
| | | UACGUGACAGAGGGCAUGCGGAAGCCCGCCUUCCUCAGCGGUGA |
| | | GCAGAAGAAGGCCAUCGUGGACCUGCUGUUCAAGACGAACCGGA |
| | | AGGUGACGGUGAAGCAGCUGAAGGAGGACUACUUCAAGAAGAUC |
| | | GAGUGCUUCGACAGCGUGGAGAUCAGCGGCGUGGAGGACCGGUU |
| | | CAACGCCAGCCUGGGCACCUACCACGACCUGCUGAAGAUCAUCA |
| | | AGGACAAGGACUUCCUGGACAACGAGGAGAACGAGGACAUCCUG |
| | | GAGGACAUCGUGCUGACGCUGACGCUGUUCGAGGACAGGGAGAU |
| | | GAUAGAGGAGCGGCUGAAGACCUACGCCCACCUGUUCGACGACA |
| | | AGGUGAUGAAGCAGCUGAAGCGGCGGCGGUACACGGGCUGGGGC |
| | | CGGCUCAGCCGGAAGCUGAUCAAUGGGAUCCGAGACAAGCAGAG |
| | | CGGCAAGACGAUCCUGGACUUCCUGAAGAGCGACGGCUUCGCCA |
| | | ACCGGAACUUCAUGCAGCUGAUCCACGACGACAGCCUGACGUUC |
| | | AAGGAGGACAUCCAGAAGGCCCAGGUCAGCGGCCAGGGCGACAG |
| | | CCUGCACGAGCACAUCGCCAAUCUCGCCGGGAGCCCCGCCAUCA |
| | | AGAAGGGGAUCCUGCAGACGGUGAAGGUGGUGGACGAGCUGGUG |
| | | AAGGUGAUGGGCCGGCACAAGCCAGAGAACAUCGUGAUCGAGAU |
| | | GGCCAGGGAGAACCAGACGACUCAAAAGGGGCAGAAGAACAGCA |
| | | GGGAGCGGAUGAAGCGGAUCGAGGAGGGCAUCAAGGAGCUGGGC |
| | | AGCCAGAUCCUGAAGGAGCACCCCGUGGAGAACACUCAACUGCA |
| | | GAACGAGAAGCUGUACCUGUACUACCUGCAGAAUGGGCGAGACA |
| | | UGUACGUGGACCAGGAGCUGGACAUCAACCGGCUCAGCGACUAC |
| | | GACGUGGACCACAUCGUUCCCCAGAGCUUCCUGAAGGACGACAG |
| | | CAUCGACAACAAGGUGCUGACGCGGAGCGACAAGAACCGGGGCA |
| | | AGAGCGACAACGUUCCCUCAGAGGAAGUCGUGAAGAAGAUGAAG |
| | | AACUACUGGCGGCAGCUGCUGAACGCCAAGCUGAUCACUCAACG |
| | | GAAGUUCGACAAUCUCACGAAGGCCGAGCGGGGUGGCCUCAGCG |
| | | AGCUGGACAAGGCCGGGUUCAUCAAGCGGCAGCUGGUGGAGACG |
| | | CGGCAGAUCACGAAGCACGUGGCCCAGAUCCUGGACAGCCGGAU |
| | | GAACACGAAGUACGACGAGAACGACAAGCUGAUCAGGGAAGUCA |
| | | AGGUGAUCACGCUGAAGAGCAAGCUGGUCAGCGACUUCCGGAAG |
| | | GACUUCCAGUUCUACAAGGUGAGGGAGAUCAACAACUACCACCA |
| | | CGCCCACGACGCCUACCUGAACGCUGUGGUUGGCACGGCACUGA |
| | | UCAAGAAGUACCCCAAGCUGGAGAGCGAGUUCGUGUACGGCGAC |
| | | UACAAGGUGUACGACGUGCGGAAGAUGAUAGCCAAGAGCGAGCA |
| | | GGAGAUCGGCAAGGCCACGGCCAAGUACUUCUUCUACAGCAACA |
| | | UCAUGAACUUCUUCAAGACAGAGAUCACGCUGGCCAAUGGUGAG |
| | | AUCCGGAAGCGGCCCCUGAUCGAGACGAAUGGUGAGACGGGUGA |
| | | GAUCGUGUGGGACAAGGGGCGAGACUUCGCCACGGUGCGGAAGG |
| | | UGCUCAGCAUGCCCCAGGUGAACAUCGUGAAGAAGACAGAAGUC |
| | | CAGACGGGUGGCUUCAGCAAGGAGAGCAUCCUUCCAAAGCGGAA |
| | | CAGCGACAAGCUGAUCGCCCGCAAGAAGGACUGGGACCCCAAGA |
| | | AGUACGGUGGCUUCGACAGCCCCACCGUGGCCUACAGCGUGCUG |
| | | GUGGUGGCCAAGGUGGAGAAGGGGAAGAGCAAGAAGCUGAAGAG |
| | | CGUGAAGGAGCUGCUGGGCAUCACGAUCAUGGAGCGGAGCAGCU |
| | | UCGAGAAGAACCCCAUCGACUUCCUGGAAGCCAAGGGGUACAAG |
| | | GAAGUCAAGAAGGACCUGAUCAUCAAGCUUCCAAAGUACAGCCU |
| | | GUUCGAGCUGGAGAAUGGGCGGAAGCGGAUGCUGGCCAGCGCCG |
| | | GUGAGCUGCAGAAGGGGAACGAGCUGGCACUUCCCUCAAAGUAC |
| | | GUGAACUUCCUGUACCUGGCCAGCCACUACGAGAAGCUGAAGGG |
| | | GAGCCCAGAGGACAACGAGCAGAAGCAGCUGUUCGUGGAGCAGC |
| | | ACAAGCACUACCUGGACGAGAUCAUCGAGCAGAUCAGCGAGUUC |
| | | AGCAAGCGGGUGAUCCUGGCCGACGCCAAUCUCGACAAGGUGCU |
| | | CAGCGCCUACAACAAGCACCGAGACAAGCCCAUCAGGGAGCAGG |
| | | CCGAGAACAUCAUCCACCUGUUCACGCUGACGAAUCUCGGUGCC |
| | | CCCGCUGCCUUCAAGUACUUCGACACGACGAUCGACCGGAAGCG |
| | | GUACACGUCGACUAAGGAAGUCCUGGACGCCACGCUGAUCCACC |
| | | AGAGCAUCACGGGCCUGUACGAGACGCGGAUCGACCUCAGCCAG |
| | | CUGGGUGGCGACGGUGGUGGCAGCCCCAAGAAGAAGCGGAAGGU |
| | | GUAG |
| 397 | ORF encoding | AUGGACAAGAAGUACAGCAUCGGCCUCGACAUCGGCACCAACAG |
| | Sp. Cas9 | CGUCGGCUGGGCCGUCAUCACCGACGAGUACAAGGUCCCCAGCA |
| | | AGAAGUUCAAGGUCCUCGGCAACACCGACCGCCACAGCAUCAAG |
| | | AAGAACCUCAUCGGCGCCCUCCUCUUCGACAGCGGCGAGACCGC |
| | | CGAGGCCACCCGCCUCAAGCGCACCGCCCGCCGCCGCUACACCC |
| | | GCCGCAAGAACCGCAUCUGCUACCUCCAGGAGAUCUUCAGCAAC |
| | | GAGAUGGCCAAGGUCGACGACAGCUUCUUCCACCGCCUCGAGGA |
| | | GAGCUUCCUCGUCGAGGAGGACAAGAAGCACGAGCGCCACCCCA |
| | | UCUUCGGCAACAUCGUCGACGAGGUCGCCUACCACGAGAAGUAC |
| | | CCCACCAUCUACCACCUCCGCAAGAAGCUCGUCGACAGCACCGA |
| | | CAAGGCCGACCUCCGCCUCAUCUACCUCGCCCUCGCCCACAUGA |
| | | UCAAGUUCCGCGGCCACUUCCUCAUCGAGGGCGACCUCAACCCC |
| | | GACAACAGCGACGUCGACAAGCUCUUCAUCCAGCUCGUCCAGAC |
| | | CUACAACCAGCUCUUCGAGGAGAACCCCAUCAACGCCAGCGGCG |
| | | UCGACGCCAAGGCCAUCCUCAGCGCCCGCCUCAGCAAGAGCCGC |
| | | CGCCUCGAGAACCUCAUCGCCCAGCUCCCCGGCGAGAAGAAGAA |
| | | CGGCCUCUUCGGCAACCUCAUCGCCCUCAGCCUCGGCCUCACCC |
| | | CCAACUUCAAGAGCAACUUCGACCUCGCCGAGGACGCCAAGCUC |
| | | CAGCUCAGCAAGGACACCUACGACGACGACCUCGACAACCUCCU |
| | | CGCCCAGAUCGGCGACCAGUACGCCGACCUCUUCCUCGCCGCCA |
| | | AGAACCUCAGCGACGCCAUCCUCCUCAGCGACAUCCUCCGCGUC |
| | | AACACCGAGAUCACCAAGGCCCCCCUCAGCGCCAGCAUGAUCAA |
| | | GCGCUACGACGAGCACCACCAGGACCUCACCCUCCUCAAGGCCC |
| | | UCGUCCGCCAGCAGCUCCCCGAGAAGUACAAGGAGAUCUUCUUC |
| | | GACCAGAGCAAGAACGGCUACGCCGGCUACAUCGACGGCGGCGC |
| | | CAGCCAGGAGGAGUUCUACAAGUUCAUCAAGCCCAUCCUCGAGA |
| | | AGAUGGACGGCACCGAGGAGCUCCUCGUCAAGCUCAACCGCGAG |
| | | GACCUCCUCCGCAAGCAGCGCACCUUCGACAACGGCAGCAUCCC |
| | | CCACCAGAUCCACCUCGGCGAGCUCCACGCCAUCCUCCGCCGCC |
| | | AGGAGGACUUCUACCCCUUCCUCAAGGACAACCGCGAGAAGAUC |
| | | GAGAAGAUCCUCACCUUCCGCAUCCCCUACUACGUCGGCCCCCU |
| | | CGCCCGCGGCAACAGCCGCUUCGCCUGGAUGACCCGCAAGAGCG |
| | | AGGAGACCAUCACCCCCUGGAACUUCGAGGAGGUCGUCGACAAG |
| | | GGCGCCAGCGCCCAGAGCUUCAUCGAGCGCAUGACCAACUUCGA |
| | | CAAGAACCUCCCCAACGAGAAGGUCCUCCCCAAGCACAGCCUCC |
| | | UCUACGAGUACUUCACCGUCUACAACGAGCUCACCAAGGUCAAG |
| | | UACGUCACCGAGGGCAUGCGCAAGCCCGCCUUCCUCAGCGGCGA |
| | | GCAGAAGAAGGCCAUCGUCGACCUCCUCUUCAAGACCAACCGCA |
| | | AGGUCACCGUCAAGCAGCUCAAGGAGGACUACUUCAAGAAGAUC |
| | | GAGUGCUUCGACAGCGUCGAGAUCAGCGGCGUCGAGGACCGCUU |
| | | CAACGCCAGCCUCGGCACCUACCACGACCUCCUCAAGAUCAUCA |
| | | AGGACAAGGACUUCCUCGACAACGAGGAGAACGAGGACAUCCUC |
| | | GAGGACAUCGUCCUCACCCUCACCCUCUUCGAGGACCGCGAGAU |
| | | GAUCGAGGAGCGCCUCAAGACCUACGCCCACCUCUUCGACGACA |
| | | AGGUCAUGAAGCAGCUCAAGCGCCGCCGCUACACCGGCUGGGGC |
| | | CGCCUCAGCCGCAAGCUCAUCAACGGCAUCCGCGACAAGCAGAG |
| | | CGGCAAGACCAUCCUCGACUUCCUCAAGAGCGACGGCUUCGCCA |
| | | ACCGCAACUUCAUGCAGCUCAUCCACGACGACAGCCUCACCUUC |
| | | AAGGAGGACAUCCAGAAGGCCCAGGUCAGCGGCCAGGGCGACAG |
| | | CCUCCACGAGCACAUCGCCAACCUCGCCGGCAGCCCCGCCAUCA |
| | | AGAAGGGCAUCCUCCAGACCGUCAAGGUCGUCGACGAGCUCGUC |
| | | AAGGUCAUGGGCCGCCACAAGCCCGAGAACAUCGUCAUCGAGAU |
| | | GGCCCGCGAGAACCAGACCACCCAGAAGGGCCAGAAGAACAGCC |
| | | GCGAGCGCAUGAAGCGCAUCGAGGAGGGCAUCAAGGAGCUCGGC |
| | | AGCCAGAUCCUCAAGGAGCACCCCGUCGAGAACACCCAGCUCCA |
| | | GAACGAGAAGCUCUACCUCUACUACCUCCAGAACGGCCGCGACA |
| | | UGUACGUCGACCAGGAGCUCGACAUCAACCGCCUCAGCGACUAC |
| | | GACGUCGACCACAUCGUCCCCCAGAGCUUCCUCAAGGACGACAG |
| | | CAUCGACAACAAGGUCCUCACCCGCAGCGACAAGAACCGCGGCA |
| | | AGAGCGACAACGUCCCCAGCGAGGAGGUCGUCAAGAAGAUGAAG |
| | | AACUACUGGCGCCAGCUCCUCAACGCCAAGCUCAUCACCCAGCG |
| | | CAAGUUCGACAACCUCACCAAGGCCGAGCGCGGCGGCCUCAGCG |
| | | AGCUCGACAAGGCCGGCUUCAUCAAGCGCCAGCUCGUCGAGACC |
| | | CGCCAGAUCACCAAGCACGUCGCCCAGAUCCUCGACAGCCGCAU |
| | | GAACACCAAGUACGACGAGAACGACAAGCUCAUCCGCGAGGUCA |
| | | AGGUCAUCACCCUCAAGAGCAAGCUCGUCAGCGACUUCCGCAAG |
| | | GACUUCCAGUUCUACAAGGUCCGCGAGAUCAACAACUACCACCA |
| | | CGCCCACGACGCCUACCUCAACGCCGUCGUCGGCACCGCCCUCA |
| | | UCAAGAAGUACCCCAAGCUCGAGAGCGAGUUCGUCUACGGCGAC |
| | | UACAAGGUCUACGACGUCCGCAAGAUGAUCGCCAAGAGCGAGCA |
| | | GGAGAUCGGCAAGGCCACCGCCAAGUACUUCUUCUACAGCAACA |
| | | UCAUGAACUUCUUCAAGACCGAGAUCACCCUCGCCAACGGCGAG |
| | | AUCCGCAAGCGCCCCCUCAUCGAGACCAACGGCGAGACCGGCGA |
| | | GAUCGUCUGGGACAAGGGCCGCGACUUCGCCACCGUCCGCAAGG |
| | | UCCUCAGCAUGCCCCAGGUCAACAUCGUCAAGAAGACCGAGGUC |
| | | CAGACCGGCGGCUUCAGCAAGGAGAGCAUCCUCCCCAAGCGCAA |
| | | CAGCGACAAGCUCAUCGCCCGCAAGAAGGACUGGGACCCCAAGA |
| | | AGUACGGCGGCUUCGACAGCCCCACCGUCGCCUACAGCGUCCUC |
| | | GUCGUCGCCAAGGUCGAGAAGGGCAAGAGCAAGAAGCUCAAGAG |
| | | CGUCAAGGAGCUCCUCGGCAUCACCAUCAUGGAGCGCAGCAGCU |
| | | UCGAGAAGAACCCCAUCGACUUCCUCGAGGCCAAGGGCUACAAG |
| | | GAGGUCAAGAAGGACCUCAUCAUCAAGCUCCCCAAGUACAGCCU |
| | | CUUCGAGCUCGAGAACGGCCGCAAGCGCAUGCUCGCCAGCGCCG |
| | | GCGAGCUCCAGAAGGGCAACGAGCUCGCCCUCCCCAGCAAGUAC |
| | | GUCAACUUCCUCUACCUCGCCAGCCACUACGAGAAGCUCAAGGG |
| | | CAGCCCCGAGGACAACGAGCAGAAGCAGCUCUUCGUCGAGCAGC |
| | | ACAAGCACUACCUCGACGAGAUCAUCGAGCAGAUCAGCGAGUUC |
| | | AGCAAGCGCGUCAUCCUCGCCGACGCCAACCUCGACAAGGUCCU |
| | | CAGCGCCUACAACAAGCACCGCGACAAGCCCAUCCGCGAGCAGG |
| | | CCGAGAACAUCAUCCACCUCUUCACCCUCACCAACCUCGGCGCC |
| | | CCCGCCGCCUUCAAGUACUUCGACACCACCAUCGACCGCAAGCG |
| | | CUACACCAGCACCAAGGAGGUCCUCGACGCCACCCUCAUCCACC |
| | | AGAGCAUCACCGGCCUCUACGAGACCCGCAUCGACCUCAGCCAG |
| | | CUCGGCGGCGACGGCGGCGGCAGCCCCAAGAAGAAGCGCAAGGU |
| | | CUAG |
| 398 | ORF encoding | ATGGACAAGAAGTACAGCATCGGCCTGGACATCGGCACCAACAG |
| | Sp. Cas9 | CGTGGGCTGGGCCGTGATCACCGACGAGTACAAGGTGCCCAGCA |
| | | AGAAGTTCAAGGTGCTGGGCAACACCGACCGGCACAGCATCAAG |
| | | AAGAACCTGATCGGCGCCCTGCTGTTCGACAGCGGCGAGACCGC |
| | | CGAGGCCACCCGGCTGAAGCGGACCGCCCGGCGGCGGTACACCC |
| | | GGCGGAAGAACCGGATCTGCTACCTGCAGGAGATCTTCAGCAAC |
| | | GAGATGGCCAAGGTGGACGACAGCTTCTTCCACCGGCTGGAGGA |
| | | GAGCTTCCTGGTGGAGGAGGACAAGAAGCACGAGCGGCACCCCA |
| | | TCTTCGGCAACATCGTGGACGAGGTGGCCTACCACGAGAAGTAC |
| | | CCCACCATCTACCACCTGCGGAAGAAGCTGGTGGACAGCACCGA |
| | | CAAGGCCGACCTGCGGCTGATCTACCTGGCCCTGGCCCACATGA |
| | | TCAAGTTCCGGGGCCACTTCCTGATCGAGGGCGACCTGAACCCC |
| | | GACAACAGCGACGTGGACAAGCTGTTCATCCAGCTGGTGCAGAC |
| | | CTACAACCAGCTGTTCGAGGAGAACCCCATCAACGCCAGCGGCG |
| | | TGGACGCCAAGGCCATCCTGAGCGCCCGGCTGAGCAAGAGCCGG |
| | | CGGCTGGAGAACCTGATCGCCCAGCTGCCCGGCGAGAAGAAGAA |
| | | CGGCCTGTTCGGCAACCTGATCGCCCTGAGCCTGGGCCTGACCC |
| | | CCAACTTCAAGAGCAACTTCGACCTGGCCGAGGACGCCAAGCTG |
| | | CAGCTGAGCAAGGACACCTACGACGACGACCTGGACAACCTGCT |
| | | GGCCCAGATCGGCGACCAGTACGCCGACCTGTTCCTGGCCGCCA |
| | | AGAACCTGAGCGACGCCATCCTGCTGAGCGACATCCTGCGGGTG |
| | | AACACCGAGATCACCAAGGCCCCCCTGAGCGCCAGCATGATCAA |
| | | GCGGTACGACGAGCACCACCAGGACCTGACCCTGCTGAAGGCCC |
| | | TGGTGCGGCAGCAGCTGCCCGAGAAGTACAAGGAGATCTTCTTC |
| | | GACCAGAGCAAGAACGGCTACGCCGGCTACATCGACGGCGGCGC |
| | | CAGCCAGGAGGAGTTCTACAAGTTCATCAAGCCCATCCTGGAGA |
| | | AGATGGACGGCACCGAGGAGCTGCTGGTGAAGCTGAACCGGGAG |
| | | GACCTGCTGCGGAAGCAGCGGACCTTCGACAACGGCAGCATCCC |
| | | CCACCAGATCCACCTGGGCGAGCTGCACGCCATCCTGCGGCGGC |
| | | AGGAGGACTTCTACCCCTTCCTGAAGGACAACCGGGAGAAGATC |
| | | GAGAAGATCCTGACCTTCCGGATCCCCTACTACGTGGGCCCCCT |
| | | GGCCCGGGGCAACAGCCGGTTCGCCTGGATGACCCGGAAGAGCG |
| | | AGGAGACCATCACCCCCTGGAACTTCGAGGAGGTGGTGGACAAG |
| | | GGCGCCAGCGCCCAGAGCTTCATCGAGCGGATGACCAACTTCGA |
| | | CAAGAACCTGCCCAACGAGAAGGTGCTGCCCAAGCACAGCCTGC |
| | | TGTACGAGTACTTCACCGTGTACAACGAGCTGACCAAGGTGAAG |
| | | TACGTGACCGAGGGCATGCGGAAGCCCGCCTTCCTGAGCGGCGA |
| | | GCAGAAGAAGGCCATCGTGGACCTGCTGTTCAAGACCAACCGGA |
| | | AGGTGACCGTGAAGCAGCTGAAGGAGGACTACTTCAAGAAGATC |
| | | GAGTGCTTCGACAGCGTGGAGATCAGCGGCGTGGAGGACCGGTT |
| | | CAACGCCAGCCTGGGCACCTACCACGACCTGCTGAAGATCATCA |
| | | AGGACAAGGACTTCCTGGACAACGAGGAGAACGAGGACATCCTG |
| | | GAGGACATCGTGCTGACCCTGACCCTGTTCGAGGACCGGGAGAT |
| | | GATCGAGGAGCGGCTGAAGACCTACGCCCACCTGTTCGACGACA |
| | | AGGTGATGAAGCAGCTGAAGCGGCGGCGGTACACCGGCTGGGGC |
| | | CGGCTGAGCCGGAAGCTGATCAACGGCATCCGGGACAAGCAGAG |
| | | CGGCAAGACCATCCTGGACTTCCTGAAGAGCGACGGCTTCGCCA |
| | | ACCGGAACTTCATGCAGCTGATCCACGACGACAGCCTGACCTTC |
| | | AAGGAGGACATCCAGAAGGCCCAGGTGAGCGGCCAGGGCGACAG |
| | | CCTGCACGAGCACATCGCCAACCTGGCCGGCAGCCCCGCCATCA |
| | | AGAAGGGCATCCTGCAGACCGTGAAGGTGGTGGACGAGCTGGTG |
| | | AAGGTGATGGGCCGGCACAAGCCCGAGAACATCGTGATCGAGAT |
| | | GGCCCGGGAGAACCAGACCACCCAGAAGGGCCAGAAGAACAGCC |
| | | GGGAGCGGATGAAGCGGATCGAGGAGGGCATCAAGGAGCTGGGC |
| | | AGCCAGATCCTGAAGGAGCACCCCGTGGAGAACACCCAGCTGCA |
| | | GAACGAGAAGCTGTACCTGTACTACCTGCAGAACGGCCGGGACA |
| | | TGTACGTGGACCAGGAGCTGGACATCAACCGGCTGAGCGACTAC |
| | | GACGTGGACCACATCGTGCCCCAGAGCTTCCTGAAGGACGACAG |
| | | CATCGACAACAAGGTGCTGACCCGGAGCGACAAGAACCGGGGCA |
| | | AGAGCGACAACGTGCCCAGCGAGGAGGTGGTGAAGAAGATGAAG |
| | | AACTACTGGCGGCAGCTGCTGAACGCCAAGCTGATCACCCAGCG |
| | | GAAGTTCGACAACCTGACCAAGGCCGAGCGGGGCGGCCTGAGCG |
| | | AGCTGGACAAGGCCGGCTTCATCAAGCGGCAGCTGGTGGAGACC |
| | | CGGCAGATCACCAAGCACGTGGCCCAGATCCTGGACAGCCGGAT |
| | | GAACACCAAGTACGACGAGAACGACAAGCTGATCCGGGAGGTGA |
| | | AGGTGATCACCCTGAAGAGCAAGCTGGTGAGCGACTTCCGGAAG |
| | | GACTTCCAGTTCTACAAGGTGCGGGAGATCAACAACTACCACCA |
| | | CGCCCACGACGCCTACCTGAACGCCGTGGTGGGCACCGCCCTGA |
| | | TCAAGAAGTACCCCAAGCTGGAGAGCGAGTTCGTGTACGGCGAC |
| | | TACAAGGTGTACGACGTGCGGAAGATGATCGCCAAGAGCGAGCA |
| | | GGAGATCGGCAAGGCCACCGCCAAGTACTTCTTCTACAGCAACA |
| | | TCATGAACTTCTTCAAGACCGAGATCACCCTGGCCAACGGCGAG |
| | | ATCCGGAAGCGGCCCCTGATCGAGACCAACGGCGAGACCGGCGA |
| | | GATCGTGTGGGACAAGGGCCGGGACTTCGCCACCGTGCGGAAGG |
| | | TGCTGAGCATGCCCCAGGTGAACATCGTGAAGAAGACCGAGGTG |
| | | CAGACCGGCGGCTTCAGCAAGGAGAGCATCCTGCCCAAGCGGAA |
| | | CAGCGACAAGCTGATCGCCCGGAAGAAGGACTGGGACCCCAAGA |
| | | AGTACGGCGGCTTCGACAGCCCCACCGTGGCCTACAGCGTGCTG |
| | | GTGGTGGCCAAGGTGGAGAAGGGCAAGAGCAAGAAGCTGAAGAG |
| | | CGTGAAGGAGCTGCTGGGCATCACCATCATGGAGCGGAGCAGCT |
| | | TCGAGAAGAACCCCATCGACTTCCTGGAGGCCAAGGGCTACAAG |
| | | GAGGTGAAGAAGGACCTGATCATCAAGCTGCCCAAGTACAGCCT |
| | | GTTCGAGCTGGAGAACGGCCGGAAGCGGATGCTGGCCAGCGCCG |
| | | GCGAGCTGCAGAAGGGCAACGAGCTGGCCCTGCCCAGCAAGTAC |
| | | GTGAACTTCCTGTACCTGGCCAGCCACTACGAGAAGCTGAAGGG |
| | | CAGCCCCGAGGACAACGAGCAGAAGCAGCTGTTCGTGGAGCAGC |
| | | ACAAGCACTACCTGGACGAGATCATCGAGCAGATCAGCGAGTTC |
| | | AGCAAGCGGGTGATCCTGGCCGACGCCAACCTGGACAAGGTGCT |
| | | GAGCGCCTACAACAAGCACCGGGACAAGCCCATCCGGGAGCAGG |
| | | CCGAGAACATCATCCACCTGTTCACCCTGACCAACCTGGGCGCC |
| | | CCCGCCGCCTTCAAGTACTTCGACACCACCATCGACCGGAAGCG |
| | | GTACACCAGCACCAAGGAGGTGCTGGACGCCACCCTGATCCACC |
| | | AGAGCATCACCGGCCTGTACGAGACCCGGATCGACCTGAGCCAG |
| | | CTGGGCGGCGACGGCGGCGGCAGCCCCAAGAAGAAGCGGAAGGT |
| | | GTGA |
| 399 | ORF encoding | ATGGACAAGAAGTACTCCATCGGCCTGGCCATCGGCACCAACTC |
| | Sp. Cas9 nickase | CGTGGGCTGGGCCGTGATCACCGACGAGTACAAGGTGCCCTCCA |
| | | AGAAGTTCAAGGTGCTGGGCAACACCGACCGGCACTCCATCAAG |
| | | AAGAACCTGATCGGCGCCCTGCTGTTCGACTCCGGCGAGACCGC |
| | | CGAGGCCACCCGGCTGAAGCGGACCGCCCGGCGGCGGTACACCC |
| | | GGCGGAAGAACCGGATCTGCTACCTGCAGGAGATCTTCTCCAAC |
| | | GAGATGGCCAAGGTGGACGACTCCTTCTTCCACCGGCTGGAGGA |
| | | GTCCTTCCTGGTGGAGGAGGACAAGAAGCACGAGCGGCACCCCA |
| | | TCTTCGGCAACATCGTGGACGAGGTGGCCTACCACGAGAAGTAC |
| | | CCCACCATCTACCACCTGCGGAAGAAGCTGGTGGACTCCACCGA |
| | | CAAGGCCGACCTGCGGCTGATCTACCTGGCCCTGGCCCACATGA |
| | | TCAAGTTCCGGGGCCACTTCCTGATCGAGGGCGACCTGAACCCC |
| | | GACAACTCCGACGTGGACAAGCTGTTCATCCAGCTGGTGCAGAC |
| | | CTACAACCAGCTGTTCGAGGAGAACCCCATCAACGCCTCCGGCG |
| | | TGGACGCCAAGGCCATCCTGTCCGCCCGGCTGTCCAAGTCCCGG |
| | | CGGCTGGAGAACCTGATCGCCCAGCTGCCCGGCGAGAAGAAGAA |
| | | CGGCCTGTTCGGCAACCTGATCGCCCTGTCCCTGGGCCTGACCC |
| | | CCAACTTCAAGTCCAACTTCGACCTGGCCGAGGACGCCAAGCTG |
| | | CAGCTGTCCAAGGACACCTACGACGACGACCTGGACAACCTGCT |
| | | GGCCCAGATCGGCGACCAGTACGCCGACCTGTTCCTGGCCGCCA |
| | | AGAACCTGTCCGACGCCATCCTGCTGTCCGACATCCTGCGGGTG |
| | | AACACCGAGATCACCAAGGCCCCCCTGTCCGCCTCCATGATCAA |
| | | GCGGTACGACGAGCACCACCAGGACCTGACCCTGCTGAAGGCCC |
| | | TGGTGCGGCAGCAGCTGCCCGAGAAGTACAAGGAGATCTTCTTC |
| | | GACCAGTCCAAGAACGGCTACGCCGGCTACATCGACGGCGGCGC |
| | | CTCCCAGGAGGAGTTCTACAAGTTCATCAAGCCCATCCTGGAGA |
| | | AGATGGACGGCACCGAGGAGCTGCTGGTGAAGCTGAACCGGGAG |
| | | GACCTGCTGCGGAAGCAGCGGACCTTCGACAACGGCTCCATCCC |
| | | CCACCAGATCCACCTGGGCGAGCTGCACGCCATCCTGCGGCGGC |
| | | AGGAGGACTTCTACCCCTTCCTGAAGGACAACCGGGAGAAGATC |
| | | GAGAAGATCCTGACCTTCCGGATCCCCTACTACGTGGGCCCCCT |
| | | GGCCCGGGGCAACTCCCGGTTCGCCTGGATGACCCGGAAGTCCG |
| | | AGGAGACCATCACCCCCTGGAACTTCGAGGAGGTGGTGGACAAG |
| | | GGCGCCTCCGCCCAGTCCTTCATCGAGCGGATGACCAACTTCGA |
| | | CAAGAACCTGCCCAACGAGAAGGTGCTGCCCAAGCACTCCCTGC |
| | | TGTACGAGTACTTCACCGTGTACAACGAGCTGACCAAGGTGAAG |
| | | TACGTGACCGAGGGCATGCGGAAGCCCGCCTTCCTGTCCGGCGA |
| | | GCAGAAGAAGGCCATCGTGGACCTGCTGTTCAAGACCAACCGGA |
| | | AGGTGACCGTGAAGCAGCTGAAGGAGGACTACTTCAAGAAGATC |
| | | GAGTGCTTCGACTCCGTGGAGATCTCCGGCGTGGAGGACCGGTT |
| | | CAACGCCTCCCTGGGCACCTACCACGACCTGCTGAAGATCATCA |
| | | AGGACAAGGACTTCCTGGACAACGAGGAGAACGAGGACATCCTG |
| | | GAGGACATCGTGCTGACCCTGACCCTGTTCGAGGACCGGGAGAT |
| | | GATCGAGGAGCGGCTGAAGACCTACGCCCACCTGTTCGACGACA |
| | | AGGTGATGAAGCAGCTGAAGCGGCGGCGGTACACCGGCTGGGGC |
| | | CGGCTGTCCCGGAAGCTGATCAACGGCATCCGGGACAAGCAGTC |
| | | CGGCAAGACCATCCTGGACTTCCTGAAGTCCGACGGCTTCGCCA |
| | | ACCGGAACTTCATGCAGCTGATCCACGACGACTCCCTGACCTTC |
| | | AAGGAGGACATCCAGAAGGCCCAGGTGTCCGGCCAGGGCGACTC |
| | | CCTGCACGAGCACATCGCCAACCTGGCCGGCTCCCCCGCCATCA |
| | | AGAAGGGCATCCTGCAGACCGTGAAGGTGGTGGACGAGCTGGTG |
| | | AAGGTGATGGGCCGGCACAAGCCCGAGAACATCGTGATCGAGAT |
| | | GGCCCGGGAGAACCAGACCACCCAGAAGGGCCAGAAGAACTCCC |
| | | GGGAGCGGATGAAGCGGATCGAGGAGGGCATCAAGGAGCTGGGC |
| | | TCCCAGATCCTGAAGGAGCACCCCGTGGAGAACACCCAGCTGCA |
| | | GAACGAGAAGCTGTACCTGTACTACCTGCAGAACGGCCGGGACA |
| | | TGTACGTGGACCAGGAGCTGGACATCAACCGGCTGTCCGACTAC |
| | | GACGTGGACCACATCGTGCCCCAGTCCTTCCTGAAGGACGACTC |
| | | CATCGACAACAAGGTGCTGACCCGGTCCGACAAGAACCGGGGCA |
| | | AGTCCGACAACGTGCCCTCCGAGGAGGTGGTGAAGAAGATGAAG |
| | | AACTACTGGCGGCAGCTGCTGAACGCCAAGCTGATCACCCAGCG |
| | | GAAGTTCGACAACCTGACCAAGGCCGAGCGGGGCGGCCTGTCCG |
| | | AGCTGGACAAGGCCGGCTTCATCAAGCGGCAGCTGGTGGAGACC |
| | | CGGCAGATCACCAAGCACGTGGCCCAGATCCTGGACTCCCGGAT |
| | | GAACACCAAGTACGACGAGAACGACAAGCTGATCCGGGAGGTGA |
| | | AGGTGATCACCCTGAAGTCCAAGCTGGTGTCCGACTTCCGGAAG |
| | | GACTTCCAGTTCTACAAGGTGCGGGAGATCAACAACTACCACCA |
| | | CGCCCACGACGCCTACCTGAACGCCGTGGTGGGCACCGCCCTGA |
| | | TCAAGAAGTACCCCAAGCTGGAGTCCGAGTTCGTGTACGGCGAC |
| | | TACAAGGTGTACGACGTGCGGAAGATGATCGCCAAGTCCGAGCA |
| | | GGAGATCGGCAAGGCCACCGCCAAGTACTTCTTCTACTCCAACA |
| | | TCATGAACTTCTTCAAGACCGAGATCACCCTGGCCAACGGCGAG |
| | | ATCCGGAAGCGGCCCCTGATCGAGACCAACGGCGAGACCGGCGA |
| | | GATCGTGTGGGACAAGGGCCGGGACTTCGCCACCGTGCGGAAGG |
| | | TGCTGTCCATGCCCCAGGTGAACATCGTGAAGAAGACCGAGGTG |
| | | CAGACCGGCGGCTTCTCCAAGGAGTCCATCCTGCCCAAGCGGAA |
| | | CTCCGACAAGCTGATCGCCCGGAAGAAGGACTGGGACCCCAAGA |
| | | AGTACGGCGGCTTCGACTCCCCCACCGTGGCCTACTCCGTGCTG |
| | | GTGGTGGCCAAGGTGGAGAAGGGCAAGTCCAAGAAGCTGAAGTC |
| | | CGTGAAGGAGCTGCTGGGCATCACCATCATGGAGCGGTCCTCCT |
| | | TCGAGAAGAACCCCATCGACTTCCTGGAGGCCAAGGGCTACAAG |
| | | GAGGTGAAGAAGGACCTGATCATCAAGCTGCCCAAGTACTCCCT |
| | | GTTCGAGCTGGAGAACGGCCGGAAGCGGATGCTGGCCTCCGCCG |
| | | GCGAGCTGCAGAAGGGCAACGAGCTGGCCCTGCCCTCCAAGTAC |
| | | GTGAACTTCCTGTACCTGGCCTCCCACTACGAGAAGCTGAAGGG |
| | | CTCCCCCGAGGACAACGAGCAGAAGCAGCTGTTCGTGGAGCAGC |
| | | ACAAGCACTACCTGGACGAGATCATCGAGCAGATCTCCGAGTTC |
| | | TCCAAGCGGGTGATCCTGGCCGACGCCAACCTGGACAAGGTGCT |
| | | GTCCGCCTACAACAAGCACCGGGACAAGCCCATCCGGGAGCAGG |
| | | CCGAGAACATCATCCACCTGTTCACCCTGACCAACCTGGGCGCC |
| | | CCCGCCGCCTTCAAGTACTTCGACACCACCATCGACCGGAAGCG |
| | | GTACACCTCCACCAAGGAGGTGCTGGACGCCACCCTGATCCACC |
| | | AGTCCATCACCGGCCTGTACGAGACCCGGATCGACCTGTCCCAG |
| | | CTGGGCGGCGACGGCGGCGGCTCCCCCAAGAAGAAGCGGAAGGT |
| | | GTGA |
| 400 | ORF encoding | ATGGACAAGAAGTACAGCATCGGCCTGGCCATCGGCACCAACAG |
| | Sp. Cas9 nickase | CGTGGGCTGGGCCGTGATCACCGACGAGTACAAGGTGCCCAGCA |
| | | AGAAGTTCAAGGTGCTGGGCAACACCGACCGGCACAGCATCAAG |
| | | AAGAACCTGATCGGCGCCCTGCTGTTCGACAGCGGCGAGACCGC |
| | | CGAGGCCACCCGGCTGAAGCGGACCGCCCGGCGGCGGTACACCC |
| | | GGCGGAAGAACCGGATCTGCTACCTGCAGGAGATCTTCAGCAAC |
| | | GAGATGGCCAAGGTGGACGACAGCTTCTTCCACCGGCTGGAGGA |
| | | GAGCTTCCTGGTGGAGGAGGACAAGAAGCACGAGCGGCACCCCA |
| | | TCTTCGGCAACATCGTGGACGAGGTGGCCTACCACGAGAAGTAC |
| | | CCCACCATCTACCACCTGCGGAAGAAGCTGGTGGACAGCACCGA |
| | | CAAGGCCGACCTGCGGCTGATCTACCTGGCCCTGGCCCACATGA |
| | | TCAAGTTCCGGGGCCACTTCCTGATCGAGGGCGACCTGAACCCC |
| | | GACAACAGCGACGTGGACAAGCTGTTCATCCAGCTGGTGCAGAC |
| | | CTACAACCAGCTGTTCGAGGAGAACCCCATCAACGCCAGCGGCG |
| | | TGGACGCCAAGGCCATCCTGAGCGCCCGGCTGAGCAAGAGCCGG |
| | | CGGCTGGAGAACCTGATCGCCCAGCTGCCCGGCGAGAAGAAGAA |
| | | CGGCCTGTTCGGCAACCTGATCGCCCTGAGCCTGGGCCTGACCC |
| | | CCAACTTCAAGAGCAACTTCGACCTGGCCGAGGACGCCAAGCTG |
| | | CAGCTGAGCAAGGACACCTACGACGACGACCTGGACAACCTGCT |
| | | GGCCCAGATCGGCGACCAGTACGCCGACCTGTTCCTGGCCGCCA |
| | | AGAACCTGAGCGACGCCATCCTGCTGAGCGACATCCTGCGGGTG |
| | | AACACCGAGATCACCAAGGCCCCCCTGAGCGCCAGCATGATCAA |
| | | GCGGTACGACGAGCACCACCAGGACCTGACCCTGCTGAAGGCCC |
| | | TGGTGCGGCAGCAGCTGCCCGAGAAGTACAAGGAGATCTTCTTC |
| | | GACCAGAGCAAGAACGGCTACGCCGGCTACATCGACGGCGGCGC |
| | | CAGCCAGGAGGAGTTCTACAAGTTCATCAAGCCCATCCTGGAGA |
| | | AGATGGACGGCACCGAGGAGCTGCTGGTGAAGCTGAACCGGGAG |
| | | GACCTGCTGCGGAAGCAGCGGACCTTCGACAACGGCAGCATCCC |
| | | CCACCAGATCCACCTGGGCGAGCTGCACGCCATCCTGCGGCGGC |
| | | AGGAGGACTTCTACCCCTTCCTGAAGGACAACCGGGAGAAGATC |
| | | GAGAAGATCCTGACCTTCCGGATCCCCTACTACGTGGGCCCCCT |
| | | GGCCCGGGGCAACAGCCGGTTCGCCTGGATGACCCGGAAGAGCG |
| | | AGGAGACCATCACCCCCTGGAACTTCGAGGAGGTGGTGGACAAG |
| | | GGCGCCAGCGCCCAGAGCTTCATCGAGCGGATGACCAACTTCGA |
| | | CAAGAACCTGCCCAACGAGAAGGTGCTGCCCAAGCACAGCCTGC |
| | | TGTACGAGTACTTCACCGTGTACAACGAGCTGACCAAGGTGAAG |
| | | TACGTGACCGAGGGCATGCGGAAGCCCGCCTTCCTGAGCGGCGA |
| | | GCAGAAGAAGGCCATCGTGGACCTGCTGTTCAAGACCAACCGGA |
| | | AGGTGACCGTGAAGCAGCTGAAGGAGGACTACTTCAAGAAGATC |
| | | GAGTGCTTCGACAGCGTGGAGATCAGCGGCGTGGAGGACCGGTT |
| | | CAACGCCAGCCTGGGCACCTACCACGACCTGCTGAAGATCATCA |
| | | AGGACAAGGACTTCCTGGACAACGAGGAGAACGAGGACATCCTG |
| | | GAGGACATCGTGCTGACCCTGACCCTGTTCGAGGACCGGGAGAT |
| | | GATCGAGGAGCGGCTGAAGACCTACGCCCACCTGTTCGACGACA |
| | | AGGTGATGAAGCAGCTGAAGCGGCGGCGGTACACCGGCTGGGGC |
| | | CGGCTGAGCCGGAAGCTGATCAACGGCATCCGGGACAAGCAGAG |
| | | CGGCAAGACCATCCTGGACTTCCTGAAGAGCGACGGCTTCGCCA |
| | | ACCGGAACTTCATGCAGCTGATCCACGACGACAGCCTGACCTTC |
| | | AAGGAGGACATCCAGAAGGCCCAGGTGAGCGGCCAGGGCGACAG |
| | | CCTGCACGAGCACATCGCCAACCTGGCCGGCAGCCCCGCCATCA |
| | | AGAAGGGCATCCTGCAGACCGTGAAGGTGGTGGACGAGCTGGTG |
| | | AAGGTGATGGGCCGGCACAAGCCCGAGAACATCGTGATCGAGAT |
| | | GGCCCGGGAGAACCAGACCACCCAGAAGGGCCAGAAGAACAGCC |
| | | GGGAGCGGATGAAGCGGATCGAGGAGGGCATCAAGGAGCTGGGC |
| | | AGCCAGATCCTGAAGGAGCACCCCGTGGAGAACACCCAGCTGCA |
| | | GAACGAGAAGCTGTACCTGTACTACCTGCAGAACGGCCGGGACA |
| | | TGTACGTGGACCAGGAGCTGGACATCAACCGGCTGAGCGACTAC |
| | | GACGTGGACCACATCGTGCCCCAGAGCTTCCTGAAGGACGACAG |
| | | CATCGACAACAAGGTGCTGACCCGGAGCGACAAGAACCGGGGCA |
| | | AGAGCGACAACGTGCCCAGCGAGGAGGTGGTGAAGAAGATGAAG |
| | | AACTACTGGCGGCAGCTGCTGAACGCCAAGCTGATCACCCAGCG |
| | | GAAGTTCGACAACCTGACCAAGGCCGAGCGGGGCGGCCTGAGCG |
| | | AGCTGGACAAGGCCGGCTTCATCAAGCGGCAGCTGGTGGAGACC |
| | | CGGCAGATCACCAAGCACGTGGCCCAGATCCTGGACAGCCGGAT |
| | | GAACACCAAGTACGACGAGAACGACAAGCTGATCCGGGAGGTGA |
| | | AGGTGATCACCCTGAAGAGCAAGCTGGTGAGCGACTTCCGGAAG |
| | | GACTTCCAGTTCTACAAGGTGCGGGAGATCAACAACTACCACCA |
| | | CGCCCACGACGCCTACCTGAACGCCGTGGTGGGCACCGCCCTGA |
| | | TCAAGAAGTACCCCAAGCTGGAGAGCGAGTTCGTGTACGGCGAC |
| | | TACAAGGTGTACGACGTGCGGAAGATGATCGCCAAGAGCGAGCA |
| | | GGAGATCGGCAAGGCCACCGCCAAGTACTTCTTCTACAGCAACA |
| | | TCATGAACTTCTTCAAGACCGAGATCACCCTGGCCAACGGCGAG |
| | | ATCCGGAAGCGGCCCCTGATCGAGACCAACGGCGAGACCGGCGA |
| | | GATCGTGTGGGACAAGGGCCGGGACTTCGCCACCGTGCGGAAGG |
| | | TGCTGAGCATGCCCCAGGTGAACATCGTGAAGAAGACCGAGGTG |
| | | CAGACCGGCGGCTTCAGCAAGGAGAGCATCCTGCCCAAGCGGAA |
| | | CAGCGACAAGCTGATCGCCCGGAAGAAGGACTGGGACCCCAAGA |
| | | AGTACGGCGGCTTCGACAGCCCCACCGTGGCCTACAGCGTGCTG |
| | | GTGGTGGCCAAGGTGGAGAAGGGCAAGAGCAAGAAGCTGAAGAG |
| | | CGTGAAGGAGCTGCTGGGCATCACCATCATGGAGCGGAGCAGCT |
| | | TCGAGAAGAACCCCATCGACTTCCTGGAGGCCAAGGGCTACAAG |
| | | GAGGTGAAGAAGGACCTGATCATCAAGCTGCCCAAGTACAGCCT |
| | | GTTCGAGCTGGAGAACGGCCGGAAGCGGATGCTGGCCAGCGCCG |
| | | GCGAGCTGCAGAAGGGCAACGAGCTGGCCCTGCCCAGCAAGTAC |
| | | GTGAACTTCCTGTACCTGGCCAGCCACTACGAGAAGCTGAAGGG |
| | | CAGCCCCGAGGACAACGAGCAGAAGCAGCTGTTCGTGGAGCAGC |
| | | ACAAGCACTACCTGGACGAGATCATCGAGCAGATCAGCGAGTTC |
| | | AGCAAGCGGGTGATCCTGGCCGACGCCAACCTGGACAAGGTGCT |
| | | GAGCGCCTACAACAAGCACCGGGACAAGCCCATCCGGGAGCAGG |
| | | CCGAGAACATCATCCACCTGTTCACCCTGACCAACCTGGGCGCC |
| | | CCCGCCGCCTTCAAGTACTTCGACACCACCATCGACCGGAAGCG |
| | | GTACACCAGCACCAAGGAGGTGCTGGACGCCACCCTGATCCACC |
| | | AGAGCATCACCGGCCTGTACGAGACCCGGATCGACCTGAGCCAG |
| | | CTGGGCGGCGACGGCGGCGGCAGCCCCAAGAAGAAGCGGAAGGT |
| | | GTGA |
| 401 | mRNA encoding | GGGUCCCGCAGUCGGCGUCCAGCGGCUCUGCUUGUUCGUGUGUG |
| | Sp. Cas9 | UGUCGUUGCAGGCCUUAUUCGGAUCCGCCACCAUGGACAAGAAG |
| | | UACAGCAUCGGACUGGACAUCGGAACAAACAGCGUCGGAUGGGC |
| | | AGUCAUCACAGACGAAUACAAGGUCCCGAGCAAGAAGUUCAAGG |
| | | UCCUGGGAAACACAGACAGACACAGCAUCAAGAAGAACCUGAUC |
| | | GGAGCACUGCUGUUCGACAGCGGAGAAACAGCAGAAGCAACAAG |
| | | ACUGAAGAGAACAGCAAGAAGAAGAUACACAAGAAGAAAGAACA |
| | | GAAUCUGCUACCUGCAGGAAAUCUUCAGCAACGAAAUGGCAAAG |
| | | GUCGACGACAGCUUCUUCCACAGACUGGAAGAAAGCUUCCUGGU |
| | | CGAAGAAGACAAGAAGCACGAAAGACACCCGAUCUUCGGAAACA |
| | | UCGUCGACGAAGUCGCAUACCACGAAAAGUACCCGACAAUCUAC |
| | | CACCUGAGAAAGAAGCUGGUCGACAGCACAGACAAGGCAGACCU |
| | | GAGACUGAUCUACCUGGCACUGGCACACAUGAUCAAGUUCAGAG |
| | | GACACUUCCUGAUCGAAGGAGACCUGAACCCGGACAACAGCGAC |
| | | GUCGACAAGCUGUUCAUCCAGCUGGUCCAGACAUACAACCAGCU |
| | | GUUCGAAGAAAACCCGAUCAACGCAAGCGGAGUCGACGCAAAGG |
| | | CAAUCCUGAGCGCAAGACUGAGCAAGAGCAGAAGACUGGAAAAC |
| | | CUGAUCGCACAGCUGCCGGGAGAAAAGAAGAACGGACUGUUCGG |
| | | AAACCUGAUCGCACUGAGCCUGGGACUGACACCGAACUUCAAGA |
| | | GCAACUUCGACCUGGCAGAAGACGCAAAGCUGCAGCUGAGCAAG |
| | | GACACAUACGACGACGACCUGGACAACCUGCUGGCACAGAUCGG |
| | | AGACCAGUACGCAGACCUGUUCCUGGCAGCAAAGAACCUGAGCG |
| | | ACGCAAUCCUGCUGAGCGACAUCCUGAGAGUCAACACAGAAAUC |
| | | ACAAAGGCACCGCUGAGCGCAAGCAUGAUCAAGAGAUACGACGA |
| | | ACACCACCAGGACCUGACACUGCUGAAGGCACUGGUCAGACAGC |
| | | AGCUGCCGGAAAAGUACAAGGAAAUCUUCUUCGACCAGAGCAAG |
| | | AACGGAUACGCAGGAUACAUCGACGGAGGAGCAAGCCAGGAAGA |
| | | AUUCUACAAGUUCAUCAAGCCGAUCCUGGAAAAGAUGGACGGAA |
| | | CAGAAGAACUGCUGGUCAAGCUGAACAGAGAAGACCUGCUGAGA |
| | | AAGCAGAGAACAUUCGACAACGGAAGCAUCCCGCACCAGAUCCA |
| | | CCUGGGAGAACUGCACGCAAUCCUGAGAAGACAGGAAGACUUCU |
| | | ACCCGUUCCUGAAGGACAACAGAGAAAAGAUCGAAAAGAUCCUG |
| | | ACAUUCAGAAUCCCGUACUACGUCGGACCGCUGGCAAGAGGAAA |
| | | CAGCAGAUUCGCAUGGAUGACAAGAAAGAGCGAAGAAACAAUCA |
| | | CACCGUGGAACUUCGAAGAAGUCGUCGACAAGGGAGCAAGCGCA |
| | | CAGAGCUUCAUCGAAAGAAUGACAAACUUCGACAAGAACCUGCC |
| | | GAACGAAAAGGUCCUGCCGAAGCACAGCCUGCUGUACGAAUACU |
| | | UCACAGUCUACAACGAACUGACAAAGGUCAAGUACGUCACAGAA |
| | | GGAAUGAGAAAGCCGGCAUUCCUGAGCGGAGAACAGAAGAAGGC |
| | | AAUCGUCGACCUGCUGUUCAAGACAAACAGAAAGGUCACAGUCA |
| | | AGCAGCUGAAGGAAGACUACUUCAAGAAGAUCGAAUGCUUCGAC |
| | | AGCGUCGAAAUCAGCGGAGUCGAAGACAGAUUCAACGCAAGCCU |
| | | GGGAACAUACCACGACCUGCUGAAGAUCAUCAAGGACAAGGACU |
| | | UCCUGGACAACGAAGAAAACGAAGACAUCCUGGAAGACAUCGUC |
| | | CUGACACUGACACUGUUCGAAGACAGAGAAAUGAUCGAAGAAAG |
| | | ACUGAAGACAUACGCACACCUGUUCGACGACAAGGUCAUGAAGC |
| | | AGCUGAAGAGAAGAAGAUACACAGGAUGGGGAAGACUGAGCAGA |
| | | AAGCUGAUCAACGGAAUCAGAGACAAGCAGAGCGGAAAGACAAU |
| | | CCUGGACUUCCUGAAGAGCGACGGAUUCGCAAACAGAAACUUCA |
| | | UGCAGCUGAUCCACGACGACAGCCUGACAUUCAAGGAAGACAUC |
| | | CAGAAGGCACAGGUCAGCGGACAGGGAGACAGCCUGCACGAACA |
| | | CAUCGCAAACCUGGCAGGAAGCCCGGCAAUCAAGAAGGGAAUCC |
| | | UGCAGACAGUCAAGGUCGUCGACGAACUGGUCAAGGUCAUGGGA |
| | | AGACACAAGCCGGAAAACAUCGUCAUCGAAAUGGCAAGAGAAAA |
| | | CCAGACAACACAGAAGGGACAGAAGAACAGCAGAGAAAGAAUGA |
| | | AGAGAAUCGAAGAAGGAAUCAAGGAACUGGGAAGCCAGAUCCUG |
| | | AAGGAACACCCGGUCGAAAACACACAGCUGCAGAACGAAAAGCU |
| | | GUACCUGUACUACCUGCAGAACGGAAGAGACAUGUACGUCGACC |
| | | AGGAACUGGACAUCAACAGACUGAGCGACUACGACGUCGACCAC |
| | | AUCGUCCCGCAGAGCUUCCUGAAGGACGACAGCAUCGACAACAA |
| | | GGUCCUGACAAGAAGCGACAAGAACAGAGGAAAGAGCGACAACG |
| | | UCCCGAGCGAAGAAGUCGUCAAGAAGAUGAAGAACUACUGGAGA |
| | | CAGCUGCUGAACGCAAAGCUGAUCACACAGAGAAAGUUCGACAA |
| | | CCUGACAAAGGCAGAGAGAGGAGGACUGAGCGAACUGGACAAGG |
| | | CAGGAUUCAUCAAGAGACAGCUGGUCGAAACAAGACAGAUCACA |
| | | AAGCACGUCGCACAGAUCCUGGACAGCAGAAUGAACACAAAGUA |
| | | CGACGAAAACGACAAGCUGAUCAGAGAAGUCAAGGUCAUCACAC |
| | | UGAAGAGCAAGCUGGUCAGCGACUUCAGAAAGGACUUCCAGUUC |
| | | UACAAGGUCAGAGAAAUCAACAACUACCACCACGCACACGACGC |
| | | AUACCUGAACGCAGUCGUCGGAACAGCACUGAUCAAGAAGUACC |
| | | CGAAGCUGGAAAGCGAAUUCGUCUACGGAGACUACAAGGUCUAC |
| | | GACGUCAGAAAGAUGAUCGCAAAGAGCGAACAGGAAAUCGGAAA |
| | | GGCAACAGCAAAGUACUUCUUCUACAGCAACAUCAUGAACUUCU |
| | | UCAAGACAGAAAUCACACUGGCAAACGGAGAAAUCAGAAAGAGA |
| | | CCGCUGAUCGAAACAAACGGAGAAACAGGAGAAAUCGUCUGGGA |
| | | CAAGGGAAGAGACUUCGCAACAGUCAGAAAGGUCCUGAGCAUGC |
| | | CGCAGGUCAACAUCGUCAAGAAGACAGAAGUCCAGACAGGAGGA |
| | | UUCAGCAAGGAAAGCAUCCUGCCGAAGAGAAACAGCGACAAGCU |
| | | GAUCGCAAGAAAGAAGGACUGGGACCCGAAGAAGUACGGAGGAU |
| | | UCGACAGCCCGACAGUCGCAUACAGCGUCCUGGUCGUCGCAAAG |
| | | GUCGAAAAGGGAAAGAGCAAGAAGCUGAAGAGCGUCAAGGAACU |
| | | GCUGGGAAUCACAAUCAUGGAAAGAAGCAGCUUCGAAAAGAACC |
| | | CGAUCGACUUCCUGGAAGCAAAGGGAUACAAGGAAGUCAAGAAG |
| | | GACCUGAUCAUCAAGCUGCCGAAGUACAGCCUGUUCGAACUGGA |
| | | AAACGGAAGAAAGAGAAUGCUGGCAAGCGCAGGAGAACUGCAGA |
| | | AGGGAAACGAACUGGCACUGCCGAGCAAGUACGUCAACUUCCUG |
| | | UACCUGGCAAGCCACUACGAAAAGCUGAAGGGAAGCCCGGAAGA |
| | | CAACGAACAGAAGCAGCUGUUCGUCGAACAGCACAAGCACUACC |
| | | UGGACGAAAUCAUCGAACAGAUCAGCGAAUUCAGCAAGAGAGUC |
| | | AUCCUGGCAGACGCAAACCUGGACAAGGUCCUGAGCGCAUACAA |
| | | CAAGCACAGAGACAAGCCGAUCAGAGAACAGGCAGAAAACAUCA |
| | | UCCACCUGUUCACACUGACAAACCUGGGAGCACCGGCAGCAUUC |
| | | AAGUACUUCGACACAACAAUCGACAGAAAGAGAUACACAAGCAC |
| | | AAAGGAAGUCCUGGACGCAACACUGAUCCACCAGAGCAUCACAG |
| | | GACUGUACGAAACAAGAAUCGACCUGAGCCAGCUGGGAGGAGAC |
| | | GGAGGAGGAAGCCCGAAGAAGAAGAGAAAGGUCUAGCUAGCCAU |
| | | CACAUUUAAAAGCAUCUCAGCCUACCAUGAGAAUAAGAGAAAGA |
| | | AAAUGAAGAUCAAUAGCUUAUUCAUCUCUUUUUCUUUUUCGUUG |
| | | GUGUAAAGCCAACACCCUGUCUAAAAAACAUAAAUUUCUUUAAU |
| | | CAUUUUGCCUCUUUUCUCUGUGCUUCAAUUAAUAAAAAAUGGAA |
| | | AGAACCUCGAGAAAAAAAAAAAAAAAAAAAAAAAAAAAAAAAAA |
| | | AAAAAAAAAAAAAAAAAAAAAAAAAAAAAAAAAAAAAAAAAAAA |
| | | AAAAAAAAAAAAAAAAAAAAAAA |
| 402 | mRNA encoding | GGGAAGCUCAGAAUAAACGCUCAACUUUGGCCGGAUCUGCCACC |
| | Sp. Cas9 | AUGGACAAGAAGUACUCCAUCGGCCUGGACAUCGGCACCAACUC |
| | | CGUGGGCUGGGCCGUGAUCACCGACGAGUACAAGGUGCCCUCCA |
| | | AGAAGUUCAAGGUGCUGGGCAACACCGACCGGCACUCCAUCAAG |
| | | AAGAACCUGAUCGGCGCCCUGCUGUUCGACUCCGGCGAGACCGC |
| | | CGAGGCCACCCGGCUGAAGCGGACCGCCCGGCGGCGGUACACCC |
| | | GGCGGAAGAACCGGAUCUGCUACCUGCAGGAGAUCUUCUCCAAC |
| | | GAGAUGGCCAAGGUGGACGACUCCUUCUUCCACCGGCUGGAGGA |
| | | GUCCUUCCUGGUGGAGGAGGACAAGAAGCACGAGCGGCACCCCA |
| | | UCUUCGGCAACAUCGUGGACGAGGUGGCCUACCACGAGAAGUAC |
| | | CCCACCAUCUACCACCUGCGGAAGAAGCUGGUGGACUCCACCGA |
| | | CAAGGCCGACCUGCGGCUGAUCUACCUGGCCCUGGCCCACAUGA |
| | | UCAAGUUCCGGGGCCACUUCCUGAUCGAGGGCGACCUGAACCCC |
| | | GACAACUCCGACGUGGACAAGCUGUUCAUCCAGCUGGUGCAGAC |
| | | CUACAACCAGCUGUUCGAGGAGAACCCCAUCAACGCCUCCGGCG |
| | | UGGACGCCAAGGCCAUCCUGUCCGCCCGGCUGUCCAAGUCCCGG |
| | | CGGCUGGAGAACCUGAUCGCCCAGCUGCCCGGCGAGAAGAAGAA |
| | | CGGCCUGUUCGGCAACCUGAUCGCCCUGUCCCUGGGCCUGACCC |
| | | CCAACUUCAAGUCCAACUUCGACCUGGCCGAGGACGCCAAGCUG |
| | | CAGCUGUCCAAGGACACCUACGACGACGACCUGGACAACCUGCU |
| | | GGCCCAGAUCGGCGACCAGUACGCCGACCUGUUCCUGGCCGCCA |
| | | AGAACCUGUCCGACGCCAUCCUGCUGUCCGACAUCCUGCGGGUG |
| | | AACACCGAGAUCACCAAGGCCCCCCUGUCCGCCUCCAUGAUCAA |
| | | GCGGUACGACGAGCACCACCAGGACCUGACCCUGCUGAAGGCCC |
| | | UGGUGCGGCAGCAGCUGCCCGAGAAGUACAAGGAGAUCUUCUUC |
| | | GACCAGUCCAAGAACGGCUACGCCGGCUACAUCGACGGCGGCGC |
| | | CUCCCAGGAGGAGUUCUACAAGUUCAUCAAGCCCAUCCUGGAGA |
| | | AGAUGGACGGCACCGAGGAGCUGCUGGUGAAGCUGAACCGGGAG |
| | | GACCUGCUGCGGAAGCAGCGGACCUUCGACAACGGCUCCAUCCC |
| | | CCACCAGAUCCACCUGGGCGAGCUGCACGCCAUCCUGCGGCGGC |
| | | AGGAGGACUUCUACCCCUUCCUGAAGGACAACCGGGAGAAGAUC |
| | | GAGAAGAUCCUGACCUUCCGGAUCCCCUACUACGUGGGCCCCCU |
| | | GGCCCGGGGCAACUCCCGGUUCGCCUGGAUGACCCGGAAGUCCG |
| | | AGGAGACCAUCACCCCCUGGAACUUCGAGGAGGUGGUGGACAAG |
| | | GGCGCCUCCGCCCAGUCCUUCAUCGAGCGGAUGACCAACUUCGA |
| | | CAAGAACCUGCCCAACGAGAAGGUGCUGCCCAAGCACUCCCUGC |
| | | UGUACGAGUACUUCACCGUGUACAACGAGCUGACCAAGGUGAAG |
| | | UACGUGACCGAGGGCAUGCGGAAGCCCGCCUUCCUGUCCGGCGA |
| | | GCAGAAGAAGGCCAUCGUGGACCUGCUGUUCAAGACCAACCGGA |
| | | AGGUGACCGUGAAGCAGCUGAAGGAGGACUACUUCAAGAAGAUC |
| | | GAGUGCUUCGACUCCGUGGAGAUCUCCGGCGUGGAGGACCGGUU |
| | | CAACGCCUCCCUGGGCACCUACCACGACCUGCUGAAGAUCAUCA |
| | | AGGACAAGGACUUCCUGGACAACGAGGAGAACGAGGACAUCCUG |
| | | GAGGACAUCGUGCUGACCCUGACCCUGUUCGAGGACCGGGAGAU |
| | | GAUCGAGGAGCGGCUGAAGACCUACGCCCACCUGUUCGACGACA |
| | | AGGUGAUGAAGCAGCUGAAGCGGCGGCGGUACACCGGCUGGGGC |
| | | CGGCUGUCCCGGAAGCUGAUCAACGGCAUCCGGGACAAGCAGUC |
| | | CGGCAAGACCAUCCUGGACUUCCUGAAGUCCGACGGCUUCGCCA |
| | | ACCGGAACUUCAUGCAGCUGAUCCACGACGACUCCCUGACCUUC |
| | | AAGGAGGACAUCCAGAAGGCCCAGGUGUCCGGCCAGGGCGACUC |
| | | CCUGCACGAGCACAUCGCCAACCUGGCCGGCUCCCCCGCCAUCA |
| | | AGAAGGGCAUCCUGCAGACCGUGAAGGUGGUGGACGAGCUGGUG |
| | | AAGGUGAUGGGCCGGCACAAGCCCGAGAACAUCGUGAUCGAGAU |
| | | GGCCCGGGAGAACCAGACCACCCAGAAGGGCCAGAAGAACUCCC |
| | | GGGAGCGGAUGAAGCGGAUCGAGGAGGGCAUCAAGGAGCUGGGC |
| | | UCCCAGAUCCUGAAGGAGCACCCCGUGGAGAACACCCAGCUGCA |
| | | GAACGAGAAGCUGUACCUGUACUACCUGCAGAACGGCCGGGACA |
| | | UGUACGUGGACCAGGAGCUGGACAUCAACCGGCUGUCCGACUAC |
| | | GACGUGGACCACAUCGUGCCCCAGUCCUUCCUGAAGGACGACUC |
| | | CAUCGACAACAAGGUGCUGACCCGGUCCGACAAGAACCGGGGCA |
| | | AGUCCGACAACGUGCCCUCCGAGGAGGUGGUGAAGAAGAUGAAG |
| | | AACUACUGGCGGCAGCUGCUGAACGCCAAGCUGAUCACCCAGCG |
| | | GAAGUUCGACAACCUGACCAAGGCCGAGCGGGGCGGCCUGUCCG |
| | | AGCUGGACAAGGCCGGCUUCAUCAAGCGGCAGCUGGUGGAGACC |
| | | CGGCAGAUCACCAAGCACGUGGCCCAGAUCCUGGACUCCCGGAU |
| | | GAACACCAAGUACGACGAGAACGACAAGCUGAUCCGGGAGGUGA |
| | | AGGUGAUCACCCUGAAGUCCAAGCUGGUGUCCGACUUCCGGAAG |
| | | GACUUCCAGUUCUACAAGGUGCGGGAGAUCAACAACUACCACCA |
| | | CGCCCACGACGCCUACCUGAACGCCGUGGUGGGCACCGCCCUGA |
| | | UCAAGAAGUACCCCAAGCUGGAGUCCGAGUUCGUGUACGGCGAC |
| | | UACAAGGUGUACGACGUGCGGAAGAUGAUCGCCAAGUCCGAGCA |
| | | GGAGAUCGGCAAGGCCACCGCCAAGUACUUCUUCUACUCCAACA |
| | | UCAUGAACUUCUUCAAGACCGAGAUCACCCUGGCCAACGGCGAG |
| | | AUCCGGAAGCGGCCCCUGAUCGAGACCAACGGCGAGACCGGCGA |
| | | GAUCGUGUGGGACAAGGGCCGGGACUUCGCCACCGUGCGGAAGG |
| | | UGCUGUCCAUGCCCCAGGUGAACAUCGUGAAGAAGACCGAGGUG |
| | | CAGACCGGCGGCUUCUCCAAGGAGUCCAUCCUGCCCAAGCGGAA |
| | | CUCCGACAAGCUGAUCGCCCGGAAGAAGGACUGGGACCCCAAGA |
| | | AGUACGGCGGCUUCGACUCCCCCACCGUGGCCUACUCCGUGCUG |
| | | GUGGUGGCCAAGGUGGAGAAGGGCAAGUCCAAGAAGCUGAAGUC |
| | | CGUGAAGGAGCUGCUGGGCAUCACCAUCAUGGAGCGGUCCUCCU |
| | | UCGAGAAGAACCCCAUCGACUUCCUGGAGGCCAAGGGCUACAAG |
| | | GAGGUGAAGAAGGACCUGAUCAUCAAGCUGCCCAAGUACUCCCU |
| | | GUUCGAGCUGGAGAACGGCCGGAAGCGGAUGCUGGCCUCCGCCG |
| | | GCGAGCUGCAGAAGGGCAACGAGCUGGCCCUGCCCUCCAAGUAC |
| | | GUGAACUUCCUGUACCUGGCCUCCCACUACGAGAAGCUGAAGGG |
| | | CUCCCCCGAGGACAACGAGCAGAAGCAGCUGUUCGUGGAGCAGC |
| | | ACAAGCACUACCUGGACGAGAUCAUCGAGCAGAUCUCCGAGUUC |
| | | UCCAAGCGGGUGAUCCUGGCCGACGCCAACCUGGACAAGGUGCU |
| | | GUCCGCCUACAACAAGCACCGGGACAAGCCCAUCCGGGAGCAGG |
| | | CCGAGAACAUCAUCCACCUGUUCACCCUGACCAACCUGGGCGCC |
| | | CCCGCCGCCUUCAAGUACUUCGACACCACCAUCGACCGGAAGCG |
| | | GUACACCUCCACCAAGGAGGUGCUGGACGCCACCCUGAUCCACC |
| | | AGUCCAUCACCGGCCUGUACGAGACCCGGAUCGACCUGUCCCAG |
| | | CUGGGCGGCGACGGCGGCGGCUCCCCCAAGAAGAAGCGGAAGGU |
| | | GUGACUAGCACCAGCCUCAAGAACACCCGAAUGGAGUCUCUAAG |
| | | CUACAUAAUACCAACUUACACUUUACAAAAUGUUGUCCCCCAAA |
| | | AUGUAGCCAUUCGUAUCUGCUCCUAAUAAAAAGAAAGUUUCUUC |
| | | ACAUUCUCUCGAGAAAAAAAAAAAAAAAAAAAAAAAAAAAAAAA |
| | | AAAAAAAAAAAAAAAAAAAAAAAAAAAAAAAAAAAAAAAAAAAA |
| | | AAAAAAAAAAAAAAAAAA |
| 403 | mRNA encoding | GGGAAGCUCAGAAUAAACGCUCAACUUUGGCCGGAUCUGCCACC |
| | Sp. Cas9 | AUGGACAAGAAGUACAGCAUCGGCCUGGACAUCGGCACCAACAG |
| | | CGUGGGCUGGGCCGUGAUCACCGACGAGUACAAGGUGCCCAGCA |
| | | AGAAGUUCAAGGUGCUGGGCAACACCGACCGGCACAGCAUCAAG |
| | | AAGAACCUGAUCGGCGCCCUGCUGUUCGACAGCGGCGAGACCGC |
| | | CGAGGCCACCCGGCUGAAGCGGACCGCCCGGCGGCGGUACACCC |
| | | GGCGGAAGAACCGGAUCUGCUACCUGCAGGAGAUCUUCAGCAAC |
| | | GAGAUGGCCAAGGUGGACGACAGCUUCUUCCACCGGCUGGAGGA |
| | | GAGCUUCCUGGUGGAGGAGGACAAGAAGCACGAGCGGCACCCCA |
| | | UCUUCGGCAACAUCGUGGACGAGGUGGCCUACCACGAGAAGUAC |
| | | CCCACCAUCUACCACCUGCGGAAGAAGCUGGUGGACAGCACCGA |
| | | CAAGGCCGACCUGCGGCUGAUCUACCUGGCCCUGGCCCACAUGA |
| | | UCAAGUUCCGGGGCCACUUCCUGAUCGAGGGCGACCUGAACCCC |
| | | GACAACAGCGACGUGGACAAGCUGUUCAUCCAGCUGGUGCAGAC |
| | | CUACAACCAGCUGUUCGAGGAGAACCCCAUCAACGCCAGCGGCG |
| | | UGGACGCCAAGGCCAUCCUGAGCGCCCGGCUGAGCAAGAGCCGG |
| | | CGGCUGGAGAACCUGAUCGCCCAGCUGCCCGGCGAGAAGAAGAA |
| | | CGGCCUGUUCGGCAACCUGAUCGCCCUGAGCCUGGGCCUGACCC |
| | | CCAACUUCAAGAGCAACUUCGACCUGGCCGAGGACGCCAAGCUG |
| | | CAGCUGAGCAAGGACACCUACGACGACGACCUGGACAACCUGCU |
| | | GGCCCAGAUCGGCGACCAGUACGCCGACCUGUUCCUGGCCGCCA |
| | | AGAACCUGAGCGACGCCAUCCUGCUGAGCGACAUCCUGCGGGUG |
| | | AACACCGAGAUCACCAAGGCCCCCCUGAGCGCCAGCAUGAUCAA |
| | | GCGGUACGACGAGCACCACCAGGACCUGACCCUGCUGAAGGCCC |
| | | UGGUGCGGCAGCAGCUGCCCGAGAAGUACAAGGAGAUCUUCUUC |
| | | GACCAGAGCAAGAACGGCUACGCCGGCUACAUCGACGGCGGCGC |
| | | CAGCCAGGAGGAGUUCUACAAGUUCAUCAAGCCCAUCCUGGAGA |
| | | AGAUGGACGGCACCGAGGAGCUGCUGGUGAAGCUGAACCGGGAG |
| | | GACCUGCUGCGGAAGCAGCGGACCUUCGACAACGGCAGCAUCCC |
| | | CCACCAGAUCCACCUGGGCGAGCUGCACGCCAUCCUGCGGCGGC |
| | | AGGAGGACUUCUACCCCUUCCUGAAGGACAACCGGGAGAAGAUC |
| | | GAGAAGAUCCUGACCUUCCGGAUCCCCUACUACGUGGGCCCCCU |
| | | GGCCCGGGGCAACAGCCGGUUCGCCUGGAUGACCCGGAAGAGCG |
| | | AGGAGACCAUCACCCCCUGGAACUUCGAGGAGGUGGUGGACAAG |
| | | GGCGCCAGCGCCCAGAGCUUCAUCGAGCGGAUGACCAACUUCGA |
| | | CAAGAACCUGCCCAACGAGAAGGUGCUGCCCAAGCACAGCCUGC |
| | | UGUACGAGUACUUCACCGUGUACAACGAGCUGACCAAGGUGAAG |
| | | UACGUGACCGAGGGCAUGCGGAAGCCCGCCUUCCUGAGCGGCGA |
| | | GCAGAAGAAGGCCAUCGUGGACCUGCUGUUCAAGACCAACCGGA |
| | | AGGUGACCGUGAAGCAGCUGAAGGAGGACUACUUCAAGAAGAUC |
| | | GAGUGCUUCGACAGCGUGGAGAUCAGCGGCGUGGAGGACCGGUU |
| | | CAACGCCAGCCUGGGCACCUACCACGACCUGCUGAAGAUCAUCA |
| | | AGGACAAGGACUUCCUGGACAACGAGGAGAACGAGGACAUCCUG |
| | | GAGGACAUCGUGCUGACCCUGACCCUGUUCGAGGACCGGGAGAU |
| | | GAUCGAGGAGCGGCUGAAGACCUACGCCCACCUGUUCGACGACA |
| | | AGGUGAUGAAGCAGCUGAAGCGGCGGCGGUACACCGGCUGGGGC |
| | | CGGCUGAGCCGGAAGCUGAUCAACGGCAUCCGGGACAAGCAGAG |
| | | CGGCAAGACCAUCCUGGACUUCCUGAAGAGCGACGGCUUCGCCA |
| | | ACCGGAACUUCAUGCAGCUGAUCCACGACGACAGCCUGACCUUC |
| | | AAGGAGGACAUCCAGAAGGCCCAGGUGAGCGGCCAGGGCGACAG |
| | | CCUGCACGAGCACAUCGCCAACCUGGCCGGCAGCCCCGCCAUCA |
| | | AGAAGGGCAUCCUGCAGACCGUGAAGGUGGUGGACGAGCUGGUG |
| | | AAGGUGAUGGGCCGGCACAAGCCCGAGAACAUCGUGAUCGAGAU |
| | | GGCCCGGGAGAACCAGACCACCCAGAAGGGCCAGAAGAACAGCC |
| | | GGGAGCGGAUGAAGCGGAUCGAGGAGGGCAUCAAGGAGCUGGGC |
| | | AGCCAGAUCCUGAAGGAGCACCCCGUGGAGAACACCCAGCUGCA |
| | | GAACGAGAAGCUGUACCUGUACUACCUGCAGAACGGCCGGGACA |
| | | UGUACGUGGACCAGGAGCUGGACAUCAACCGGCUGAGCGACUAC |
| | | GACGUGGACCACAUCGUGCCCCAGAGCUUCCUGAAGGACGACAG |
| | | CAUCGACAACAAGGUGCUGACCCGGAGCGACAAGAACCGGGGCA |
| | | AGAGCGACAACGUGCCCAGCGAGGAGGUGGUGAAGAAGAUGAAG |
| | | AACUACUGGCGGCAGCUGCUGAACGCCAAGCUGAUCACCCAGCG |
| | | GAAGUUCGACAACCUGACCAAGGCCGAGCGGGGCGGCCUGAGCG |
| | | AGCUGGACAAGGCCGGCUUCAUCAAGCGGCAGCUGGUGGAGACC |
| | | CGGCAGAUCACCAAGCACGUGGCCCAGAUCCUGGACAGCCGGAU |
| | | GAACACCAAGUACGACGAGAACGACAAGCUGAUCCGGGAGGUGA |
| | | AGGUGAUCACCCUGAAGAGCAAGCUGGUGAGCGACUUCCGGAAG |
| | | GACUUCCAGUUCUACAAGGUGCGGGAGAUCAACAACUACCACCA |
| | | CGCCCACGACGCCUACCUGAACGCCGUGGUGGGCACCGCCCUGA |
| | | UCAAGAAGUACCCCAAGCUGGAGAGCGAGUUCGUGUACGGCGAC |
| | | UACAAGGUGUACGACGUGCGGAAGAUGAUCGCCAAGAGCGAGCA |
| | | GGAGAUCGGCAAGGCCACCGCCAAGUACUUCUUCUACAGCAACA |
| | | UCAUGAACUUCUUCAAGACCGAGAUCACCCUGGCCAACGGCGAG |
| | | AUCCGGAAGCGGCCCCUGAUCGAGACCAACGGCGAGACCGGCGA |
| | | GAUCGUGUGGGACAAGGGCCGGGACUUCGCCACCGUGCGGAAGG |
| | | UGCUGAGCAUGCCCCAGGUGAACAUCGUGAAGAAGACCGAGGUG |
| | | CAGACCGGCGGCUUCAGCAAGGAGAGCAUCCUGCCCAAGCGGAA |
| | | CAGCGACAAGCUGAUCGCCCGGAAGAAGGACUGGGACCCCAAGA |
| | | AGUACGGCGGCUUCGACAGCCCCACCGUGGCCUACAGCGUGCUG |
| | | GUGGUGGCCAAGGUGGAGAAGGGCAAGAGCAAGAAGCUGAAGAG |
| | | CGUGAAGGAGCUGCUGGGCAUCACCAUCAUGGAGCGGAGCAGCU |
| | | UCGAGAAGAACCCCAUCGACUUCCUGGAGGCCAAGGGCUACAAG |
| | | GAGGUGAAGAAGGACCUGAUCAUCAAGCUGCCCAAGUACAGCCU |
| | | GUUCGAGCUGGAGAACGGCCGGAAGCGGAUGCUGGCCAGCGCCG |
| | | GCGAGCUGCAGAAGGGCAACGAGCUGGCCCUGCCCAGCAAGUAC |
| | | GUGAACUUCCUGUACCUGGCCAGCCACUACGAGAAGCUGAAGGG |
| | | CAGCCCCGAGGACAACGAGCAGAAGCAGCUGUUCGUGGAGCAGC |
| | | ACAAGCACUACCUGGACGAGAUCAUCGAGCAGAUCAGCGAGUUC |
| | | AGCAAGCGGGUGAUCCUGGCCGACGCCAACCUGGACAAGGUGCU |
| | | GAGCGCCUACAACAAGCACCGGGACAAGCCCAUCCGGGAGCAGG |
| | | CCGAGAACAUCAUCCACCUGUUCACCCUGACCAACCUGGGCGCC |
| | | CCCGCCGCCUUCAAGUACUUCGACACCACCAUCGACCGGAAGCG |
| | | GUACACCAGCACCAAGGAGGUGCUGGACGCCACCCUGAUCCACC |
| | | AGAGCAUCACCGGCCUGUACGAGACCCGGAUCGACCUGAGCCAG |
| | | CUGGGCGGCGACGGCGGCGGCAGCCCCAAGAAGAAGCGGAAGGU |
| | | GUGACUAGCACCAGCCUCAAGAACACCCGAAUGGAGUCUCUAAG |
| | | CUACAUAAUACCAACUUACACUUUACAAAAUGUUGUCCCCCAAA |
| | | AUGUAGCCAUUCGUAUCUGCUCCUAAUAAAAAGAAAGUUUCUUC |
| | | ACAUUCUCUCGAGAAAAAAAAAAAAAAAAAAAAAAAAAAAAAAA |
| | | AAAAAAAAAAAAAAAAAAAAAAAAAAAAAAAAAAAAAAAAAAAA |
| | | AAAAAAAAAAAAAAAA |
| 404 | mRNA encoding | GGGAAGCUCAGAAUAAACGCUCAACUUUGGCCGGAUCUGCCACC |
| | Sp. Cas9 | AUGGACAAGAAGUACAGCAUCGGCCUGGACAUCGGCACGAACAG |
| | | CGUUGGCUGGGCUGUGAUCACGGACGAGUACAAGGUUCCCUCAA |
| | | AGAAGUUCAAGGUGCUGGGCAACACGGACCGGCACAGCAUCAAG |
| | | AAGAAUCUCAUCGGUGCACUGCUGUUCGACAGCGGUGAGACGGC |
| | | CGAAGCCACGCGGCUGAAGCGGACGGCCCGCCGGCGGUACACGC |
| | | GGCGGAAGAACCGGAUCUGCUACCUGCAGGAGAUCUUCAGCAAC |
| | | GAGAUGGCCAAGGUGGACGACAGCUUCUUCCACCGGCUGGAGGA |
| | | GAGCUUCCUGGUGGAGGAGGACAAGAAGCACGAGCGGCACCCCA |
| | | UCUUCGGCAACAUCGUGGACGAAGUCGCCUACCACGAGAAGUAC |
| | | CCCACCAUCUACCACCUGCGGAAGAAGCUGGUGGACUCGACUGA |
| | | CAAGGCCGACCUGCGGCUGAUCUACCUGGCACUGGCCCACAUGA |
| | | UAAAGUUCCGGGGCCACUUCCUGAUCGAGGGCGACCUGAACCCU |
| | | GACAACAGCGACGUGGACAAGCUGUUCAUCCAGCUGGUGCAGAC |
| | | CUACAACCAGCUGUUCGAGGAGAACCCCAUCAACGCCAGCGGCG |
| | | UGGACGCCAAGGCCAUCCUCAGCGCCCGCCUCAGCAAGAGCCGG |
| | | CGGCUGGAGAAUCUCAUCGCCCAGCUUCCAGGUGAGAAGAAGAA |
| | | UGGGCUGUUCGGCAAUCUCAUCGCACUCAGCCUGGGCCUGACUC |
| | | CCAACUUCAAGAGCAACUUCGACCUGGCCGAGGACGCCAAGCUG |
| | | CAGCUCAGCAAGGACACCUACGACGACGACCUGGACAAUCUCCU |
| | | GGCCCAGAUCGGCGACCAGUACGCCGACCUGUUCCUGGCUGCCA |
| | | AGAAUCUCAGCGACGCCAUCCUGCUCAGCGACAUCCUGCGGGUG |
| | | AACACAGAGAUCACGAAGGCCCCCCUCAGCGCCAGCAUGAUAAA |
| | | GCGGUACGACGAGCACCACCAGGACCUGACGCUGCUGAAGGCAC |
| | | UGGUGCGGCAGCAGCUUCCAGAGAAGUACAAGGAGAUCUUCUUC |
| | | GACCAGAGCAAGAAUGGGUACGCCGGGUACAUCGACGGUGGUGC |
| | | CAGCCAGGAGGAGUUCUACAAGUUCAUCAAGCCCAUCCUGGAGA |
| | | AGAUGGACGGCACAGAGGAGCUGCUGGUGAAGCUGAACAGGGAG |
| | | GACCUGCUGCGGAAGCAGCGGACGUUCGACAAUGGGAGCAUCCC |
| | | CCACCAGAUCCACCUGGGUGAGCUGCACGCCAUCCUGCGGCGGC |
| | | AGGAGGACUUCUACCCCUUCCUGAAGGACAACAGGGAGAAGAUC |
| | | GAGAAGAUCCUGACGUUCCGGAUCCCCUACUACGUUGGCCCCCU |
| | | GGCCCGCGGCAACAGCCGGUUCGCCUGGAUGACGCGGAAGAGCG |
| | | AGGAGACGAUCACUCCCUGGAACUUCGAGGAAGUCGUGGACAAG |
| | | GGUGCCAGCGCCCAGAGCUUCAUCGAGCGGAUGACGAACUUCGA |
| | | CAAGAAUCUUCCAAACGAGAAGGUGCUUCCAAAGCACAGCCUGC |
| | | UGUACGAGUACUUCACGGUGUACAACGAGCUGACGAAGGUGAAG |
| | | UACGUGACAGAGGGCAUGCGGAAGCCCGCCUUCCUCAGCGGUGA |
| | | GCAGAAGAAGGCCAUCGUGGACCUGCUGUUCAAGACGAACCGGA |
| | | AGGUGACGGUGAAGCAGCUGAAGGAGGACUACUUCAAGAAGAUC |
| | | GAGUGCUUCGACAGCGUGGAGAUCAGCGGCGUGGAGGACCGGUU |
| | | CAACGCCAGCCUGGGCACCUACCACGACCUGCUGAAGAUCAUCA |
| | | AGGACAAGGACUUCCUGGACAACGAGGAGAACGAGGACAUCCUG |
| | | GAGGACAUCGUGCUGACGCUGACGCUGUUCGAGGACAGGGAGAU |
| | | GAUAGAGGAGCGGCUGAAGACCUACGCCCACCUGUUCGACGACA |
| | | AGGUGAUGAAGCAGCUGAAGCGGCGGCGGUACACGGGCUGGGGC |
| | | CGGCUCAGCCGGAAGCUGAUCAAUGGGAUCCGAGACAAGCAGAG |
| | | CGGCAAGACGAUCCUGGACUUCCUGAAGAGCGACGGCUUCGCCA |
| | | ACCGGAACUUCAUGCAGCUGAUCCACGACGACAGCCUGACGUUC |
| | | AAGGAGGACAUCCAGAAGGCCCAGGUCAGCGGCCAGGGCGACAG |
| | | CCUGCACGAGCACAUCGCCAAUCUCGCCGGGAGCCCCGCCAUCA |
| | | AGAAGGGGAUCCUGCAGACGGUGAAGGUGGUGGACGAGCUGGUG |
| | | AAGGUGAUGGGCCGGCACAAGCCAGAGAACAUCGUGAUCGAGAU |
| | | GGCCAGGGAGAACCAGACGACUCAAAAGGGGCAGAAGAACAGCA |
| | | GGGAGCGGAUGAAGCGGAUCGAGGAGGGCAUCAAGGAGCUGGGC |
| | | AGCCAGAUCCUGAAGGAGCACCCCGUGGAGAACACUCAACUGCA |
| | | GAACGAGAAGCUGUACCUGUACUACCUGCAGAAUGGGCGAGACA |
| | | UGUACGUGGACCAGGAGCUGGACAUCAACCGGCUCAGCGACUAC |
| | | GACGUGGACCACAUCGUUCCCCAGAGCUUCCUGAAGGACGACAG |
| | | CAUCGACAACAAGGUGCUGACGCGGAGCGACAAGAACCGGGGCA |
| | | AGAGCGACAACGUUCCCUCAGAGGAAGUCGUGAAGAAGAUGAAG |
| | | AACUACUGGCGGCAGCUGCUGAACGCCAAGCUGAUCACUCAACG |
| | | GAAGUUCGACAAUCUCACGAAGGCCGAGCGGGGUGGCCUCAGCG |
| | | AGCUGGACAAGGCCGGGUUCAUCAAGCGGCAGCUGGUGGAGACG |
| | | CGGCAGAUCACGAAGCACGUGGCCCAGAUCCUGGACAGCCGGAU |
| | | GAACACGAAGUACGACGAGAACGACAAGCUGAUCAGGGAAGUCA |
| | | AGGUGAUCACGCUGAAGAGCAAGCUGGUCAGCGACUUCCGGAAG |
| | | GACUUCCAGUUCUACAAGGUGAGGGAGAUCAACAACUACCACCA |
| | | CGCCCACGACGCCUACCUGAACGCUGUGGUUGGCACGGCACUGA |
| | | UCAAGAAGUACCCCAAGCUGGAGAGCGAGUUCGUGUACGGCGAC |
| | | UACAAGGUGUACGACGUGCGGAAGAUGAUAGCCAAGAGCGAGCA |
| | | GGAGAUCGGCAAGGCCACGGCCAAGUACUUCUUCUACAGCAACA |
| | | UCAUGAACUUCUUCAAGACAGAGAUCACGCUGGCCAAUGGUGAG |
| | | AUCCGGAAGCGGCCCCUGAUCGAGACGAAUGGUGAGACGGGUGA |
| | | GAUCGUGUGGGACAAGGGGCGAGACUUCGCCACGGUGCGGAAGG |
| | | UGCUCAGCAUGCCCCAGGUGAACAUCGUGAAGAAGACAGAAGUC |
| | | CAGACGGGUGGCUUCAGCAAGGAGAGCAUCCUUCCAAAGCGGAA |
| | | CAGCGACAAGCUGAUCGCCCGCAAGAAGGACUGGGACCCCAAGA |
| | | AGUACGGUGGCUUCGACAGCCCCACCGUGGCCUACAGCGUGCUG |
| | | GUGGUGGCCAAGGUGGAGAAGGGGAAGAGCAAGAAGCUGAAGAG |
| | | CGUGAAGGAGCUGCUGGGCAUCACGAUCAUGGAGCGGAGCAGCU |
| | | UCGAGAAGAACCCCAUCGACUUCCUGGAAGCCAAGGGGUACAAG |
| | | GAAGUCAAGAAGGACCUGAUCAUCAAGCUUCCAAAGUACAGCCU |
| | | GUUCGAGCUGGAGAAUGGGCGGAAGCGGAUGCUGGCCAGCGCCG |
| | | GUGAGCUGCAGAAGGGGAACGAGCUGGCACUUCCCUCAAAGUAC |
| | | GUGAACUUCCUGUACCUGGCCAGCCACUACGAGAAGCUGAAGGG |
| | | GAGCCCAGAGGACAACGAGCAGAAGCAGCUGUUCGUGGAGCAGC |
| | | ACAAGCACUACCUGGACGAGAUCAUCGAGCAGAUCAGCGAGUUC |
| | | AGCAAGCGGGUGAUCCUGGCCGACGCCAAUCUCGACAAGGUGCU |
| | | CAGCGCCUACAACAAGCACCGAGACAAGCCCAUCAGGGAGCAGG |
| | | CCGAGAACAUCAUCCACCUGUUCACGCUGACGAAUCUCGGUGCC |
| | | CCCGCUGCCUUCAAGUACUUCGACACGACGAUCGACCGGAAGCG |
| | | GUACACGUCGACUAAGGAAGUCCUGGACGCCACGCUGAUCCACC |
| | | AGAGCAUCACGGGCCUGUACGAGACGCGGAUCGACCUCAGCCAG |
| | | CUGGGUGGCGACGGUGGUGGCAGCCCCAAGAAGAAGCGGAAGGU |
| | | GUAGCUAGCACCAGCCUCAAGAACACCCGAAUGGAGUCUCUAAG |
| | | CUACAUAAUACCAACUUACACUUUACAAAAUGUUGUCCCCCAAA |
| | | AUGUAGCCAUUCGUAUCUGCUCCUAAUAAAAAGAAAGUUUCUUC |
| | | ACAUUCUCUCGAGAAAAAAAAAAAAAAAAAAAAAAAAAAAAAAA |
| | | AAAAAAAAAAAAAAAAAAAAAAAAAAAAAAAAAAAAAAAAAAAA |
| | | AAAAAAAAAAAAAAAAAAAA |
| 405 | mRNA encoding | GGGAAGCUCAGAAUAAACGCUCAACUUUGGCCGGAUCUGCCACC |
| | Sp. Cas9 | AUGGACAAGAAGUACAGCAUCGGCCUCGACAUCGGCACCAACAG |
| | | CGUCGGCUGGGCCGUCAUCACCGACGAGUACAAGGUCCCCAGCA |
| | | AGAAGUUCAAGGUCCUCGGCAACACCGACCGCCACAGCAUCAAG |
| | | AAGAACCUCAUCGGCGCCCUCCUCUUCGACAGCGGCGAGACCGC |
| | | CGAGGCCACCCGCCUCAAGCGCACCGCCCGCCGCCGCUACACCC |
| | | GCCGCAAGAACCGCAUCUGCUACCUCCAGGAGAUCUUCAGCAAC |
| | | GAGAUGGCCAAGGUCGACGACAGCUUCUUCCACCGCCUCGAGGA |
| | | GAGCUUCCUCGUCGAGGAGGACAAGAAGCACGAGCGCCACCCCA |
| | | UCUUCGGCAACAUCGUCGACGAGGUCGCCUACCACGAGAAGUAC |
| | | CCCACCAUCUACCACCUCCGCAAGAAGCUCGUCGACAGCACCGA |
| | | CAAGGCCGACCUCCGCCUCAUCUACCUCGCCCUCGCCCACAUGA |
| | | UCAAGUUCCGCGGCCACUUCCUCAUCGAGGGCGACCUCAACCCC |
| | | GACAACAGCGACGUCGACAAGCUCUUCAUCCAGCUCGUCCAGAC |
| | | CUACAACCAGCUCUUCGAGGAGAACCCCAUCAACGCCAGCGGCG |
| | | UCGACGCCAAGGCCAUCCUCAGCGCCCGCCUCAGCAAGAGCCGC |
| | | CGCCUCGAGAACCUCAUCGCCCAGCUCCCCGGCGAGAAGAAGAA |
| | | CGGCCUCUUCGGCAACCUCAUCGCCCUCAGCCUCGGCCUCACCC |
| | | CCAACUUCAAGAGCAACUUCGACCUCGCCGAGGACGCCAAGCUC |
| | | CAGCUCAGCAAGGACACCUACGACGACGACCUCGACAACCUCCU |
| | | CGCCCAGAUCGGCGACCAGUACGCCGACCUCUUCCUCGCCGCCA |
| | | AGAACCUCAGCGACGCCAUCCUCCUCAGCGACAUCCUCCGCGUC |
| | | AACACCGAGAUCACCAAGGCCCCCCUCAGCGCCAGCAUGAUCAA |
| | | GCGCUACGACGAGCACCACCAGGACCUCACCCUCCUCAAGGCCC |
| | | UCGUCCGCCAGCAGCUCCCCGAGAAGUACAAGGAGAUCUUCUUC |
| | | GACCAGAGCAAGAACGGCUACGCCGGCUACAUCGACGGCGGCGC |
| | | CAGCCAGGAGGAGUUCUACAAGUUCAUCAAGCCCAUCCUCGAGA |
| | | AGAUGGACGGCACCGAGGAGCUCCUCGUCAAGCUCAACCGCGAG |
| | | GACCUCCUCCGCAAGCAGCGCACCUUCGACAACGGCAGCAUCCC |
| | | CCACCAGAUCCACCUCGGCGAGCUCCACGCCAUCCUCCGCCGCC |
| | | AGGAGGACUUCUACCCCUUCCUCAAGGACAACCGCGAGAAGAUC |
| | | GAGAAGAUCCUCACCUUCCGCAUCCCCUACUACGUCGGCCCCCU |
| | | CGCCCGCGGCAACAGCCGCUUCGCCUGGAUGACCCGCAAGAGCG |
| | | AGGAGACCAUCACCCCCUGGAACUUCGAGGAGGUCGUCGACAAG |
| | | GGCGCCAGCGCCCAGAGCUUCAUCGAGCGCAUGACCAACUUCGA |
| | | CAAGAACCUCCCCAACGAGAAGGUCCUCCCCAAGCACAGCCUCC |
| | | UCUACGAGUACUUCACCGUCUACAACGAGCUCACCAAGGUCAAG |
| | | UACGUCACCGAGGGCAUGCGCAAGCCCGCCUUCCUCAGCGGCGA |
| | | GCAGAAGAAGGCCAUCGUCGACCUCCUCUUCAAGACCAACCGCA |
| | | AGGUCACCGUCAAGCAGCUCAAGGAGGACUACUUCAAGAAGAUC |
| | | GAGUGCUUCGACAGCGUCGAGAUCAGCGGCGUCGAGGACCGCUU |
| | | CAACGCCAGCCUCGGCACCUACCACGACCUCCUCAAGAUCAUCA |
| | | AGGACAAGGACUUCCUCGACAACGAGGAGAACGAGGACAUCCUC |
| | | GAGGACAUCGUCCUCACCCUCACCCUCUUCGAGGACCGCGAGAU |
| | | GAUCGAGGAGCGCCUCAAGACCUACGCCCACCUCUUCGACGACA |
| | | AGGUCAUGAAGCAGCUCAAGCGCCGCCGCUACACCGGCUGGGGC |
| | | CGCCUCAGCCGCAAGCUCAUCAACGGCAUCCGCGACAAGCAGAG |
| | | CGGCAAGACCAUCCUCGACUUCCUCAAGAGCGACGGCUUCGCCA |
| | | ACCGCAACUUCAUGCAGCUCAUCCACGACGACAGCCUCACCUUC |
| | | AAGGAGGACAUCCAGAAGGCCCAGGUCAGCGGCCAGGGCGACAG |
| | | CCUCCACGAGCACAUCGCCAACCUCGCCGGCAGCCCCGCCAUCA |
| | | AGAAGGGCAUCCUCCAGACCGUCAAGGUCGUCGACGAGCUCGUC |
| | | AAGGUCAUGGGCCGCCACAAGCCCGAGAACAUCGUCAUCGAGAU |
| | | GGCCCGCGAGAACCAGACCACCCAGAAGGGCCAGAAGAACAGCC |
| | | GCGAGCGCAUGAAGCGCAUCGAGGAGGGCAUCAAGGAGCUCGGC |
| | | AGCCAGAUCCUCAAGGAGCACCCCGUCGAGAACACCCAGCUCCA |
| | | GAACGAGAAGCUCUACCUCUACUACCUCCAGAACGGCCGCGACA |
| | | UGUACGUCGACCAGGAGCUCGACAUCAACCGCCUCAGCGACUAC |
| | | GACGUCGACCACAUCGUCCCCCAGAGCUUCCUCAAGGACGACAG |
| | | CAUCGACAACAAGGUCCUCACCCGCAGCGACAAGAACCGCGGCA |
| | | AGAGCGACAACGUCCCCAGCGAGGAGGUCGUCAAGAAGAUGAAG |
| | | AACUACUGGCGCCAGCUCCUCAACGCCAAGCUCAUCACCCAGCG |
| | | CAAGUUCGACAACCUCACCAAGGCCGAGCGCGGCGGCCUCAGCG |
| | | AGCUCGACAAGGCCGGCUUCAUCAAGCGCCAGCUCGUCGAGACC |
| | | CGCCAGAUCACCAAGCACGUCGCCCAGAUCCUCGACAGCCGCAU |
| | | GAACACCAAGUACGACGAGAACGACAAGCUCAUCCGCGAGGUCA |
| | | AGGUCAUCACCCUCAAGAGCAAGCUCGUCAGCGACUUCCGCAAG |
| | | GACUUCCAGUUCUACAAGGUCCGCGAGAUCAACAACUACCACCA |
| | | CGCCCACGACGCCUACCUCAACGCCGUCGUCGGCACCGCCCUCA |
| | | UCAAGAAGUACCCCAAGCUCGAGAGCGAGUUCGUCUACGGCGAC |
| | | UACAAGGUCUACGACGUCCGCAAGAUGAUCGCCAAGAGCGAGCA |
| | | GGAGAUCGGCAAGGCCACCGCCAAGUACUUCUUCUACAGCAACA |
| | | UCAUGAACUUCUUCAAGACCGAGAUCACCCUCGCCAACGGCGAG |
| | | AUCCGCAAGCGCCCCCUCAUCGAGACCAACGGCGAGACCGGCGA |
| | | GAUCGUCUGGGACAAGGGCCGCGACUUCGCCACCGUCCGCAAGG |
| | | UCCUCAGCAUGCCCCAGGUCAACAUCGUCAAGAAGACCGAGGUC |
| | | CAGACCGGCGGCUUCAGCAAGGAGAGCAUCCUCCCCAAGCGCAA |
| | | CAGCGACAAGCUCAUCGCCCGCAAGAAGGACUGGGACCCCAAGA |
| | | AGUACGGCGGCUUCGACAGCCCCACCGUCGCCUACAGCGUCCUC |
| | | GUCGUCGCCAAGGUCGAGAAGGGCAAGAGCAAGAAGCUCAAGAG |
| | | CGUCAAGGAGCUCCUCGGCAUCACCAUCAUGGAGCGCAGCAGCU |
| | | UCGAGAAGAACCCCAUCGACUUCCUCGAGGCCAAGGGCUACAAG |
| | | GAGGUCAAGAAGGACCUCAUCAUCAAGCUCCCCAAGUACAGCCU |
| | | CUUCGAGCUCGAGAACGGCCGCAAGCGCAUGCUCGCCAGCGCCG |
| | | GCGAGCUCCAGAAGGGCAACGAGCUCGCCCUCCCCAGCAAGUAC |
| | | GUCAACUUCCUCUACCUCGCCAGCCACUACGAGAAGCUCAAGGG |
| | | CAGCCCCGAGGACAACGAGCAGAAGCAGCUCUUCGUCGAGCAGC |
| | | ACAAGCACUACCUCGACGAGAUCAUCGAGCAGAUCAGCGAGUUC |
| | | AGCAAGCGCGUCAUCCUCGCCGACGCCAACCUCGACAAGGUCCU |
| | | CAGCGCCUACAACAAGCACCGCGACAAGCCCAUCCGCGAGCAGG |
| | | CCGAGAACAUCAUCCACCUCUUCACCCUCACCAACCUCGGCGCC |
| | | CCCGCCGCCUUCAAGUACUUCGACACCACCAUCGACCGCAAGCG |
| | | CUACACCAGCACCAAGGAGGUCCUCGACGCCACCCUCAUCCACC |
| | | AGAGCAUCACCGGCCUCUACGAGACCCGCAUCGACCUCAGCCAG |
| | | CUCGGCGGCGACGGCGGCGGCAGCCCCAAGAAGAAGCGCAAGGU |
| | | CUAGCUAGCACCAGCCUCAAGAACACCCGAAUGGAGUCUCUAAG |
| | | CUACAUAAUACCAACUUACACUUUACAAAAUGUUGUCCCCCAAA |
| | | AUGUAGCCAUUCGUAUCUGCUCCUAAUAAAAAGAAAGUUUCUUC |
| | | ACAUUCUCUCGAGAAAAAAAAAAAAAAAAAAAAAAAAAAAAAAA |
| | | AAAAAAAAAAAAAAAAAAAAAAAAAAAAAAAAAAAAAAAAAAAA |
| | | AAAAAAAAAAAAAAAAAAAAAAAAAAAAA |
| 406 | amino acid | MDKKYSIGLDIGTNSVGWAVITDEYKVPSKKFKVLGNTDRHSIK |
| | sequence for Sp. | KNLIGALLFDSGETAEATRLKRTARRRYTRRKNRICYLQEIFSN |
| | Cas9 | EMAKVDDSFFHRLEESFLVEEDKKHERHPIFGNIVDEVAYHEKY |
| | | PTIYHLRKKLVDSTDKADLRLIYLALAHMIKFRGHFLIEGDLNP |
| | | DNSDVDKLFIQLVQTYNQLFEENPINASGVDAKAILSARLSKSR |
| | | RLENLIAQLPGEKKNGLFGNLIALSLGLTPNFKSNFDLAEDAKL |
| | | QLSKDTYDDDLDNLLAQIGDQYADLFLAAKNLSDAILLSDILRV |
| | | NTEITKAPLSASMIKRYDEHHQDLTLLKALVRQQLPEKYKEIFF |
| | | DQSKNGYAGYIDGGASQEEFYKFIKPILEKMDGTEELLVKLNRE |
| | | DLLRKQRTFDNGSIPHQIHLGELHAILRRQEDFYPFLKDNREKI |
| | | EKILTFRIPYYVGPLARGNSRFAWMTRKSEETITPWNFEEVVDK |
| | | GASAQSFIERMTNEDKNLPNEKVLPKHSLLYEYFTVYNELTKVK |
| | | YVTEGMRKPAFLSGEQKKAIVDLLFKTNRKVTVKQLKEDYFKKI |
| | | ECFDSVEISGVEDRFNASLGTYHDLLKIIKDKDFLDNEENEDIL |
| | | EDIVLTLTLFEDREMIEERLKTYAHLFDDKVMKQLKRRRYTGWG |
| | | RLSRKLINGIRDKQSGKTILDFLKSDGFANRNFMQLIHDDSLTF |
| | | KEDIQKAQVSGQGDSLHEHIANLAGSPAIKKGILQTVKVVDELV |
| | | KVMGRHKPENIVIEMARENQTTQKGQKNSRERMKRIEEGIKELG |
| | | SQILKEHPVENTQLQNEKLYLYYLONGRDMYVDQELDINRLSDY |
| | | DVDHIVPQSFLKDDSIDNKVLTRSDKNRGKSDNVPSEEVVKKMK |
| | | NYWRQLLNAKLITQRKFDNLTKAERGGLSELDKAGFIKRQLVET |
| | | RQITKHVAQILDSRMNTKYDENDKLIREVKVITLKSKLVSDFRK |
| | | DFQFYKVREINNYHHAHDAYLNAVVGTALIKKYPKLESEFVYGD |
| | | YKVYDVRKMIAKSEQEIGKATAKYFFYSNIMNFFKTEITLANGE |
| | | IRKRPLIETNGETGEIVWDKGRDFATVRKVLSMPQVNIVKKTEV |
| | | QTGGFSKESILPKRNSDKLIARKKDWDPKKYGGFDSPTVAYSVL |
| | | VVAKVEKGKSKKLKSVKELLGITIMERSSFEKNPIDFLEAKGYK |
| | | EVKKDLIIKLPKYSLFELENGRKRMLASAGELQKGNELALPSKY |
| | | VNFLYLASHYEKLKGSPEDNEQKQLFVEQHKHYLDEIIEQISEF |
| | | SKRVILADANLDKVLSAYNKHRDKPIREQAENIIHLFTLTNLGA |
| | | PAAFKYFDTTIDRKRYTSTKEVLDATLIHQSITGLYETRIDLSQ |
| | | LGGDGGGSPKKKRKV |
| 407 | amino acid | MDKKYSIGLAIGTNSVGWAVITDEYKVPSKKFKVLGNTDRHSIK |
| | sequence Sp. | KNLIGALLFDSGETAEATRLKRTARRRYTRRKNRICYLQEIFSN |
| | Cas9 nickase | EMAKVDDSFFHRLEESFLVEEDKKHERHPIFGNIVDEVAYHEKY |
| | | PTIYHLRKKLVDSTDKADLRLIYLALAHMIKFRGHFLIEGDLNP |
| | | DNSDVDKLFIQLVQTYNQLFEENPINASGVDAKAILSARLSKSR |
| | | RLENLIAQLPGEKKNGLFGNLIALSLGLTPNFKSNFDLAEDAKL |
| | | QLSKDTYDDDLDNLLAQIGDQYADLFLAAKNLSDAILLSDILRV |
| | | NTEITKAPLSASMIKRYDEHHQDLTLLKALVRQQLPEKYKEIFF |
| | | DQSKNGYAGYIDGGASQEEFYKFIKPILEKMDGTEELLVKLNRE |
| | | DLLRKQRTFDNGSIPHQIHLGELHAILRRQEDFYPFLKDNREKI |
| | | EKILTFRIPYYVGPLARGNSRFAWMTRKSEETITPWNFEEVVDK |
| | | GASAQSFIERMTNFDKNLPNEKVLPKHSLLYEYFTVYNELTKVK |
| | | YVTEGMRKPAFLSGEQKKAIVDLLFKTNRKVTVKQLKEDYFKKI |
| | | ECFDSVEISGVEDRFNASLGTYHDLLKIIKDKDFLDNEENEDIL |
| | | EDIVLTLTLFEDREMIEERLKTYAHLFDDKVMKOLKRRRYTGWG |
| | | RLSRKLINGIRDKQSGKTILDFLKSDGFANRNFMQLIHDDSLTF |
| | | KEDIQKAQVSGQGDSLHEHIANLAGSPAIKKGILQTVKVVDELV |
| | | KVMGRHKPENIVIEMARENQTTQKGQKNSRERMKRIEEGIKELG |
| | | SQILKEHPVENTQLQNEKLYLYYLONGRDMYVDQELDINRLSDY |
| | | DVDHIVPQSFLKDDSIDNKVLTRSDKNRGKSDNVPSEEVVKKMK |
| | | NYWRQLLNAKLITQRKFDNLTKAERGGLSELDKAGFIKRQLVET |
| | | RQITKHVAQILDSRMNTKYDENDKLIREVKVITLKSKLVSDFRK |
| | | DFQFYKVREINNYHHAHDAYLNAVVGTALIKKYPKLESEFVYGD |
| | | YKVYDVRKMIAKSEQEIGKATAKYFFYSNIMNFFKTEITLANGE |
| | | IRKRPLIETNGETGEIVWDKGRDFATVRKVLSMPQVNIVKKTEV |
| | | QTGGFSKESILPKRNSDKLIARKKDWDPKKYGGFDSPTVAYSVL |
| | | VVAKVEKGKSKKLKSVKELLGITIMERSSFEKNPIDFLEAKGYK |
| | | EVKKDLIIKLPKYSLFELENGRKRMLASAGELQKGNELALPSKY |
| | | VNFLYLASHYEKLKGSPEDNEQKQLFVEQHKHYLDEIIEQISEF |
| | | SKRVILADANLDKVLSAYNKHRDKPIREQAENIIHLFTLTNLGA |
| | | PAAFKYFDTTIDRKRYTSTKEVLDATLIHQSITGLYETRIDLSQ |
| | | LGGDGGGSPKKKRKV |
| 392 | exemplary Kozak | GCCGCCRCCAUGG |
| | sequence | |
| 408 | Cas9 ORF | GGGAAGCUCAGAAUAAACGCUCAACUUUGGCCGGAUC |
| | | UGCCACCAUGGACAAGAAGUACUCCAUCGGCCUGGAC |
| | | AUCGGCACCAACUCCGUGGGCUGGGCCGUGAUCACCG |
| | | ACGAGUACAAGGUGCCCUCCAAGAAGUUCAAGGUGCU |
| | | GGGCAACACCGACCGGCACUCCAUCAAGAAGAACCUG |
| | | AUCGGCGCCCUGCUGUUCGACUCCGGCGAGACCGCCG |
| | | AGGCCACCCGGCUGAAGCGGACCGCCCGGCGGCGGUA |
| | | CACCCGGCGGAAGAACCGGAUCUGCUACCUGCAGGAG |
| | | AUCUUCUCCAACGAGAUGGCCAAGGUGGACGACUCCU |
| | | UCUUCCACCGGCUGGAGGAGUCCUUCCUGGUGGAGGA |
| | | GGACAAGAAGCACGAGCGGCACCCCAUCUUCGGCAAC |
| | | AUCGUGGACGAGGUGGCCUACCACGAGAAGUACCCCA |
| | | CCAUCUACCACCUGCGGAAGAAGCUGGUGGACUCCAC |
| | | CGACAAGGCCGACCUGCGGCUGAUCUACCUGGCCCUG |
| | | GCCCACAUGAUCAAGUUCCGGGGCCACUUCCUGAUCG |
| | | AGGGCGACCUGAACCCCGACAACUCCGACGUGGACAA |
| | | GCUGUUCAUCCAGCUGGUGCAGACCUACAACCAGCUG |
| | | UUCGAGGAGAACCCCAUCAACGCCUCCGGCGUGGACG |
| | | CCAAGGCCAUCCUGUCCGCCCGGCUGUCCAAGUCCCG |
| | | GCGGCUGGAGAACCUGAUCGCCCAGCUGCCCGGCGAG |
| | | AAGAAGAACGGCCUGUUCGGCAACCUGAUCGCCCUGU |
| | | CCCUGGGCCUGACCCCCAACUUCAAGUCCAACUUCGA |
| | | CCUGGCCGAGGACGCCAAGCUGCAGCUGUCCAAGGAC |
| | | ACCUACGACGACGACCUGGACAACCUGCUGGCCCAGA |
| | | UCGGCGACCAGUACGCCGACCUGUUCCUGGCCGCCAA |
| | | GAACCUGUCCGACGCCAUCCUGCUGUCCGACAUCCUG |
| | | CGGGUGAACACCGAGAUCACCAAGGCCCCCCUGUCCG |
| | | CCUCCAUGAUCAAGCGGUACGACGAGCACCACCAGGA |
| | | CCUGACCCUGCUGAAGGCCCUGGUGCGGCAGCAGCUG |
| | | CCCGAGAAGUACAAGGAGAUCUUCUUCGACCAGUCCA |
| | | AGAACGGCUACGCCGGCUACAUCGACGGCGGCGCCUC |
| | | CCAGGAGGAGUUCUACAAGUUCAUCAAGCCCAUCCUG |
| | | GAGAAGAUGGACGGCACCGAGGAGCUGCUGGUGAAGC |
| | | UGAACCGGGAGGACCUGCUGCGGAAGCAGCGGACCUU |
| | | CGACAACGGCUCCAUCCCCCACCAGAUCCACCUGGGC |
| | | GAGCUGCACGCCAUCCUGCGGCGGCAGGAGGACUUCU |
| | | ACCCCUUCCUGAAGGACAACCGGGAGAAGAUCGAGAA |
| | | GAUCCUGACCUUCCGGAUCCCCUACUACGUGGGCCCC |
| | | CUGGCCCGGGGCAACUCCCGGUUCGCCUGGAUGACCC |
| | | GGAAGUCCGAGGAGACCAUCACCCCCUGGAACUUCGA |
| | | GGAGGUGGUGGACAAGGGCGCCUCCGCCCAGUCCUUC |
| | | AUCGAGCGGAUGACCAACUUCGACAAGAACCUGCCCA |
| | | ACGAGAAGGUGCUGCCCAAGCACUCCCUGCUGUACGA |
| | | GUACUUCACCGUGUACAACGAGCUGACCAAGGUGAAG |
| | | UACGUGACCGAGGGCAUGCGGAAGCCCGCCUUCCUGU |
| | | CCGGCGAGCAGAAGAAGGCCAUCGUGGACCUGCUGUU |
| | | CAAGACCAACCGGAAGGUGACCGUGAAGCAGCUGAAG |
| | | GAGGACUACUUCAAGAAGAUCGAGUGCUUCGACUCCG |
| | | UGGAGAUCUCCGGCGUGGAGGACCGGUUCAACGCCUC |
| | | CCUGGGCACCUACCACGACCUGCUGAAGAUCAUCAAG |
| | | GACAAGGACUUCCUGGACAACGAGGAGAACGAGGACA |
| | | UCCUGGAGGACAUCGUGCUGACCCUGACCCUGUUCGA |
| | | GGACCGGGAGAUGAUCGAGGAGCGGCUGAAGACCUAC |
| | | GCCCACCUGUUCGACGACAAGGUGAUGAAGCAGCUGA |
| | | AGCGGCGGCGGUACACCGGCUGGGGCCGGCUGUCCCG |
| | | GAAGCUGAUCAACGGCAUCCGGGACAAGCAGUCCGGC |
| | | AAGACCAUCCUGGACUUCCUGAAGUCCGACGGCUUCG |
| | | CCAACCGGAACUUCAUGCAGCUGAUCCACGACGACUC |
| | | CCUGACCUUCAAGGAGGACAUCCAGAAGGCCCAGGUG |
| | | UCCGGCCAGGGCGACUCCCUGCACGAGCACAUCGCCA |
| | | ACCUGGCCGGCUCCCCCGCCAUCAAGAAGGGCAUCCU |
| | | GCAGACCGUGAAGGUGGUGGACGAGCUGGUGAAGGU |
| | | GAUGGGCCGGCACAAGCCCGAGAACAUCGUGAUCGAG |
| | | AUGGCCCGGGAGAACCAGACCACCCAGAAGGGCCAGA |
| | | AGAACUCCCGGGAGCGGAUGAAGCGGAUCGAGGAGGG |
| | | CAUCAAGGAGCUGGGCUCCCAGAUCCUGAAGGAGCAC |
| | | CCCGUGGAGAACACCCAGCUGCAGAACGAGAAGCUGU |
| | | ACCUGUACUACCUGCAGAACGGCCGGGACAUGUACGU |
| | | GGACCAGGAGCUGGACAUCAACCGGCUGUCCGACUAC |
| | | GACGUGGACCACAUCGUGCCCCAGUCCUUCCUGAAGG |
| | | ACGACUCCAUCGACAACAAGGUGCUGACCCGGUCCGA |
| | | CAAGAACCGGGGCAAGUCCGACAACGUGCCCUCCGAG |
| | | GAGGUGGUGAAGAAGAUGAAGAACUACUGGCGGCAG |
| | | CUGCUGAACGCCAAGCUGAUCACCCAGCGGAAGUUCG |
| | | ACAACCUGACCAAGGCCGAGCGGGGCGGCCUGUCCGA |
| | | GCUGGACAAGGCCGGCUUCAUCAAGCGGCAGCUGGUG |
| | | GAGACCCGGCAGAUCACCAAGCACGUGGCCCAGAUCC |
| | | UGGACUCCCGGAUGAACACCAAGUACGACGAGAACGA |
| | | CAAGCUGAUCCGGGAGGUGAAGGUGAUCACCCUGAAG |
| | | UCCAAGCUGGUGUCCGACUUCCGGAAGGACUUCCAGU |
| | | UCUACAAGGUGCGGGAGAUCAACAACUACCACCACGC |
| | | CCACGACGCCUACCUGAACGCCGUGGUGGGCACCGCC |
| | | CUGAUCAAGAAGUACCCCAAGCUGGAGUCCGAGUUCG |
| | | UGUACGGCGACUACAAGGUGUACGACGUGCGGAAGAU |
| | | GAUCGCCAAGUCCGAGCAGGAGAUCGGCAAGGCCACC |
| | | GCCAAGUACUUCUUCUACUCCAACAUCAUGAACUUCU |
| | | UCAAGACCGAGAUCACCCUGGCCAACGGCGAGAUCCG |
| | | GAAGCGGCCCCUGAUCGAGACCAACGGCGAGACCGGC |
| | | GAGAUCGUGUGGGACAAGGGCCGGGACUUCGCCACCG |
| | | UGCGGAAGGUGCUGUCCAUGCCCCAGGUGAACAUCGU |
| | | GAAGAAGACCGAGGUGCAGACCGGCGGCUUCUCCAAG |
| | | GAGUCCAUCCUGCCCAAGCGGAACUCCGACAAGCUGA |
| | | UCGCCCGGAAGAAGGACUGGGACCCCAAGAAGUACGG |
| | | CGGCUUCGACUCCCCCACCGUGGCCUACUCCGUGCUG |
| | | GUGGUGGCCAAGGUGGAGAAGGGCAAGUCCAAGAAGC |
| | | UGAAGUCCGUGAAGGAGCUGCUGGGCAUCACCAUCAU |
| | | GGAGCGGUCCUCCUUCGAGAAGAACCCCAUCGACUUC |
| | | CUGGAGGCCAAGGGCUACAAGGAGGUGAAGAAGGACC |
| | | UGAUCAUCAAGCUGCCCAAGUACUCCCUGUUCGAGCU |
| | | GGAGAACGGCCGGAAGCGGAUGCUGGCCUCCGCCGGC |
| | | GAGCUGCAGAAGGGCAACGAGCUGGCCCUGCCCUCCA |
| | | AGUACGUGAACUUCCUGUACCUGGCCUCCCACUACGA |
| | | GAAGCUGAAGGGCUCCCCCGAGGACAACGAGCAGAAG |
| | | CAGCUGUUCGUGGAGCAGCACAAGCACUACCUGGACG |
| | | AGAUCAUCGAGCAGAUCUCCGAGUUCUCCAAGCGGGU |
| | | GAUCCUGGCCGACGCCAACCUGGACAAGGUGCUGUCC |
| | | GCCUACAACAAGCACCGGGACAAGCCCAUCCGGGAGC |
| | | AGGCCGAGAACAUCAUCCACCUGUUCACCCUGACCAA |
| | | CCUGGGCGCCCCCGCCGCCUUCAAGUACUUCGACACC |
| | | ACCAUCGACCGGAAGCGGUACACCUCCACCAAGGAGG |
| | | UGCUGGACGCCACCCUGAUCCACCAGUCCAUCACCGG |
| | | CCUGUACGAGACCCGGAUCGACCUGUCCCAGCUGGGC |
| | | GGCGACGGCGGCGGCUCCCCCAAGAAGAAGCGGAAGG |
| | | UGUGACUAGCACCAGCCUCAAGAACACCCGAAUGGAG |
| | | UCUCUAAGCUACAUAAUACCAACUUACACUUUACAAA |
| | | AUGUUGUCCCCCAAAAUGUAGCCAUUCGUAUCUGCUC |
| | | CUAAUAAAAAGAAAGUUUCUUCACAUUCUCUCGAG |

* = PS linkage; ‘m’ = 2′-O-Me nucleotide, ‘f’ = 2′-F nucleotide

TABLE 24
Human KLKB1 targeted guide sequence, chromosomal
coordinates, and human single guide RNAs and dual guide RNAs, and
surrogate cynomolgus (cyno) monkey single guides

| | Exemplary | | | | | Cyno |
|---|---|---|---|---|---|---|
| SEQ ID | Genomic | | | | | guide |
| NO: | Coordinates | human guide | human | human | cyno | SEQ |
| (human) | (hg38) | sequence | sgRNA | dgRNA | sgRNA | ID NO: |
| 1 | chr4:186228230- | ACAGGAAACUGUAGCAAACA | G012253 | CR005916 | NA | NA |
| | 186228252 | | | | | |
| 2 | chr4:186228248- | AUAGAUAAUUCACUUACCAC | G012254 | CR005917 | NA | NA |
| | 186228270 | | | | | |
| 3 | chr4:186232154- | UACAUCCCCACCUCUGAAGA | G012255 | CR005918 | NA | NA |
| | 186232176 | | | | | |
| 4 | chr4:186251256- | UCUUGAGGAGUAGAGGAACU | G012256 | CR005922 | NA | NA |
| | 186251278 | | | | | |
| 5 | chr4:186251308- | ACCAGGUAAAGUUCUUUUGC | G012257 | CR005924 | NA | NA |
| | 186251330 | | | | | |
| 6 | chr4:186251489- | GGGUAAAUUUUAGAAUGGCA | G012258 | CR005925 | NA | NA |
| | 186251511 | | | | | |
| 7 | chr4:186251504- | AUUUACCCGGGAGUUGACUU | G012259 | CR005928 | NA | NA |
| | 186251526 | | | | | |
| 8 | chr4:186251507- | UACCCGGGAGUUGACUUUGG | G012260 | CR005929 | NA | NA |
| | 186251529 | | | | | |
| 9 | chr4:186251828- | UCUUUGAGAUUGUGUAACAC | G012261 | CR005931 | NA | NA |
| | 186251850 | | | | | |
| 10 | chr4:186251829- | CUUUGAGAUUGUGUAACACU | G012262 | CR005932 | NA | NA |
| | 186251851 | | | | | |
| 11 | chr4:186251830- | UUUGAGAUUGUGUAACACUG | G012263 | CR005933 | NA | NA |
| | 186251852 | | | | | |
| 12 | chr4:186254748- | UACAUACCAGUGUAAUUCAA | G012264 | CR005943 | NA | NA |
| | 186254770 | | | | | |
| 13 | chr4:186251784- | CUCCAACUAGGAUUGCGUAU | G012265 | CR005949 | G013933 | 373 |
| | 186251806 | | | | | |
| 14 | chr4:186251792- | AGGAUUGCGUAUGGGACACA | G012266 | CR005951 | G013904 | 344 |
| | 186251814 | | | | | |
| 15 | chr4:186251793- | GGAUUGCGUAUGGGACACAA | G012267 | CR005952 | G013901 | 341 |
| | 186251815 | | | | | |
| 16 | chr4:186238297- | GUUACUCAGCACCUUUAUAG | G012268 | CR005956 | G013945 | 385 |
| | 186238319 | | | | | |
| 17 | chr4:186238263- | UGCCUAUUAAAGUACAGUCC | G012269 | CR005959 | NA | NA |
| | 186238285 | | | | | |
| 18 | chr4:186251772- | CUAUGGAUGGUUCUCCAACU | G012270 | CR005960 | G013922 | 362 |
| | 186251794 | | | | | |
| 19 | chr4:186254601- | GAUGUUUGGCGCAUCUAUAG | G012271 | CR005963 | G013921 | 361 |
| | 186254623 | | | | | |
| 20 | chr4:186254592- | AUGCGCCAAACAUCCUGCAG | G012272 | CR005970 | G013885 | 325 |
| | 186254614 | | | | | |
| 21 | chr4:186236785- | CUCCUUUAUAAAUGUCUCGA | G012273 | CR005979 | G013905 | 345 |
| | 186236807 | | | | | |
| 22 | chr4:186236863- | UGUUACUGGUGCACCUUUUU | G012274 | CR005982 | NA | NA |
| | 186236885 | | | | | |
| 23 | chr4:186254593- | GAUGCGCCAAACAUCCUGCA | G012275 | CR005983 | G013876 | 316 |
| | 186254615 | | | | | |
| 24 | chr4:186232192- | AUCUGGCAGUAUUGGGCAUU | G012276 | CR005992 | G013915 | 355 |
| | 186232214 | | | | | |
| 25 | chr4:186236893- | GCGUGGCAUAUGAAAAAAAC | G012277 | CR005994 | NA | NA |
| | 186236915 | | | | | |
| 26 | chr4:186236798- | UAUAAAGGAGUUGAUAUGAG | G012278 | CR005995 | G013913 | 353 |
| | 186236820 | | | | | |
| 27 | chr4:186236938- | ACACCUUGAAUUGUACUCAC | G012279 | CR005998 | NA | NA |
| | 186236960 | | | | | |
| 28 | chr4:186232214- | UGAGGUGCACAUUCCACCCA | G012280 | NA | NA | NA |
| | 186232236 | | | | | |
| 29 | chr4:186232190- | CUGGCAGUAUUGGGCAUUUG | G012281 | NA | NA | NA |
| | 186232212 | | | | | |
| 30 | chr4:186232148- | AAAACGCCUUCUUCAGAGGU | G012282 | NA | NA | NA |
| | 186232170 | | | | | |
| 31 | chr4:186232227- | UGAAUAGCAAACACCUUGGG | G012283 | NA | NA | NA |
| | 186232249 | | | | | |
| 32 | chr4:186236821- | AGUCAAUUUUAAUGUGUCUA | G012284 | NA | NA | NA |
| | 186236843 | | | | | |
| 33 | chr4:186236850- | GUGUUGAAGAAUGCCAAAAA | G012285 | NA | NA | NA |
| | 186236872 | | | | | |
| 34 | chr4:186236910- | UGCCUUGUGAAAUGUUUGCG | G012286 | NA | NA | NA |
| | 186236932 | | | | | |
| 35 | chr4:186250265- | GCAUCUUGCGUUCUCAGAUG | G012287 | NA | G013927 | 367 |
| | 186250287 | | | | | |
| 36 | chr4:186250276- | UCUCAGAUGUGGAUGUUGCC | G012288 | NA | NA | NA |
| | 186250298 | | | | | |
| 37 | chr4:186250306- | CUCCAGAUGCUUUUGUGUGU | G012289 | NA | NA | NA |
| | 186250328 | | | | | |
| 38 | chr4:186251325- | UAUUAUCAAAUCACAUUACC | G012290 | NA | NA | NA |
| | 186251347 | | | | | |
| 39 | chr4:186251271- | CCAGAUAUGGUGUUUUCUUG | G012291 | NA | NA | NA |
| | 186251293 | | | | | |
| 40 | chr4:186251300- | AAGUUCUUUUGCAGGUUAAA | G012292 | NA | NA | NA |
| | 186251322 | | | | | |
| 41 | chr4:186251620- | UUUACUCCCAGAAGACUGUA | G012293 | NA | NA | NA |
| | 186251642 | | | | | |
| 42 | chr4:186251492- | UGCCAUUCUAAAAUUUACCC | G012294 | NA | NA | NA |
| | 186251514 | | | | | |
| 43 | chr4:186251572- | UCAUCUUUGUGCAAGUCUCU | G012295 | NA | NA | NA |
| | 186251594 | | | | | |
| 44 | chr4:186251510- | UCUCCUCCAAAGUCAACUCC | G012296 | NA | NA | NA |
| | 186251532 | | | | | |
| 45 | chr4:186252049- | GGAGGAACAAACUCUUCUUG | G012297 | NA | NA | NA |
| | 186252071 | | | | | |
| 46 | chr4:186252098- | AGGUGAAGCUGACAGCUCAG | G012298 | NA | NA | NA |
| | 186252120 | | | | | |
| 47 | chr4:186256046- | CCAUCCGGUUACCCAACAGU | G012299 | NA | G013931 | 371 |
| | 186256068 | | | | | |
| 48 | chr4:186256042- | UAUACCAACUGUUGGGUAAC | G012300 | NA | G012300 | NA |
| | 186256064 | | | | | |
| 49 | chr4:186256034- | GCACAAUUUAUACCAACUGU | G012301 | NA | NA | NA |
| | 186256056 | | | | | |
| 50 | chr4:186256059- | AACCGGAUGGGGCUUCUCGA | G012302 | NA | G013932 | 372 |
| | 186256081 | | | | | |
| 51 | chr4:186256047- | CAACUGUUGGGUAACCGGAU | G012303 | NA | G013882 | 322 |
| | 186256069 | | | | | |
| 52 | chr4:186256035- | CACAAUUUAUACCAACUGUU | G012304 | NA | G012304 | NA |
| | 186256057 | | | | | |
| 53 | chr4:186256046- | CCAACUGUUGGGUAACCGGA | G012305 | NA | G013924 | 364 |
| | 186256068 | | | | | |
| 54 | chr4:186256061- | CUCCUUCGAGAAGCCCCAUC | G012306 | NA | NA | NA |
| | 186256083 | | | | | |
| 55 | chr4:186256048- | AACUGUUGGGUAACCGGAUG | G012307 | NA | G013914 | 354 |
| | 186256070 | | | | | |
| 56 | chr4:186256003- | CCAAUAUGCCUACCUUCCAA | G012308 | NA | NA | NA |
| | 186256025 | | | | | |
| 57 | chr4:186256015- | GUGCUUGUGUCACCUUUGGA | G012309 | NA | G013900 | 340 |
| | 186256037 | | | | | |
| 58 | chr4:186256011- | UUGUGUCACCUUUGGAAGGU | G012310 | NA | NA | NA |
| | 186256033 | | | | | |
| 59 | chr4:186256019- | AAUUGUGCUUGUGUCACCUU | G012311 | NA | NA | NA |
| | 186256041 | | | | | |
| 60 | chr4:186255996- | AAGGUAGGCAUAUUGGUUUU | G012312 | NA | NA | NA |
| | 186256018 | | | | | |
| 61 | chr4:186257312- | ACCCAACGGAUGGUCUGUGC | G012313 | NA | NA | NA |
| | 186257334 | | | | | |
| 62 | chr4:186257314- | AGCCAGCACAGACCAUCCGU | G012314 | NA | NA | NA |
| | 186257336 | | | | | |
| 63 | chr4:186257302- | UUAUAAAAUAACCCAACGGA | G012315 | NA | G012315 | NA |
| | 186257324 | | | | | |
| 64 | chr4:186257326- | CUGUGCUGGCUAUAAAGAAG | G012316 | NA | NA | NA |
| | 186257348 | | | | | |
| 65 | chr4:186257261- | CAUUCUUCAUUUGUUACCAA | G012317 | NA | NA | NA |
| | 186257283 | | | | | |
| 66 | chr4:186257284- | UAUAAUCUUGAUAUCUUUUC | G012318 | NA | NA | NA |
| | 186257306 | | | | | |
| 67 | chr4:186257313- | GCCAGCACAGACCAUCCGUU | G012319 | NA | NA | NA |
| | 186257335 | | | | | |
| 68 | chr4:186257324- | GUCUGUGCUGGCUAUAAAGA | G012320 | NA | G012320 | NA |
| | 186257346 | | | | | |
| 69 | chr4:186257325- | UCUGUGCUGGCUAUAAAGAA | G012321 | NA | NA | NA |
| | 186257347 | | | | | |
| 70 | chr4:186258130- | GUCCAUGUACUCAGCGACUU | G012322 | NA | G012322 | NA |
| | 186258152 | | | | | |
| 71 | chr4:186258128- | CACCAAAGUCGCUGAGUACA | G012323 | NA | G012323 | NA |
| | 186258150 | | | | | |
| 72 | chr4:186258050- | ACACAAUGGAAUGUGGCGUU | G012324 | NA | G012324 | NA |
| | 186258072 | | | | | |
| 73 | chr4:186258068- | UUUGGUGGGCAUCACCAGCU | G012325 | NA | G012325 | NA |
| | 186258090 | | | | | |
| 74 | chr4:186258204- | CUCUGGACUGCUUCUCAUGC | G012326 | NA | NA | NA |
| | 186258226 | | | | | |
| 75 | chr4:186258133- | AAGUCGCUGAGUACAUGGAC | G012327 | NA | G012327 | NA |
| | 186258155 | | | | | |
| 76 | chr4:186258089- | GGGUGAAGGCUGUGCCCGCA | G012328 | NA | G013895 | 335 |
| | 186258111 | | | | | |
| 77 | chr4:186258054- | AAUGGAAUGUGGCGUUUGGU | G012329 | NA | G012329 | NA |
| | 186258076 | | | | | |
| 78 | chr4:186258037- | UCCAUUGUGUUUGCAAACUA | G012330 | NA | G013942 | 382 |
| | 186258059 | | | | | |
| 79 | chr4:186258067- | GUUUGGUGGGCAUCACCAGC | G012331 | NA | NA | NA |
| | 186258089 | | | | | |
| 80 | chr4:186258043- | UUUGCAAACACAAUGGAAUG | G012332 | NA | G013916 | 356 |
| | 186258065 | | | | | |
| 81 | chr4:186258103- | GACACCAGGUUGCUCCCUGC | G012333 | NA | NA | NA |
| | 186258125 | | | | | |
| 82 | chr4:186258009- | ACUGUGACUCAGGGAGAUUC | G012334 | NA | G013943 | 383 |
| | 186258031 | | | | | |
| 83 | chr4:186258099- | UGUGCCCGCAGGGAGCAACC | G012335 | NA | NA | NA |
| | 186258121 | | | | | |
| 84 | chr4:186258036- | CCAUUGUGUUUGCAAACUAA | G012336 | NA | G013929 | 369 |
| | 186258058 | | | | | |
| 85 | chr4:186258088- | GGGGUGAAGGCUGUGCCCGC | G012337 | NA | NA | NA |
| | 186258110 | | | | | |
| 86 | chr4:186258117- | GCGACUUUGGUGUAGACACC | G012338 | NA | NA | NA |
| | 186258139 | | | | | |
| 87 | chr4:186258036- | CCCUUAGUUUGCAAACACAA | G012339 | NA | NA | NA |
| | 186258058 | | | | | |
| 88 | chr4:186258053- | CAAUGGAAUGUGGCGUUUGG | G012340 | NA | G012340 | NA |
| | 186258075 | | | | | |
| 89 | chr4:186232230- | AACUGAAUAGCAAACACCUU | NA | CR005919 | NA | NA |
| | 186232252 | | | | | |
| 90 | chr4:186238351- | ACAAUUACCAAUUUCUGAAA | NA | CR005920 | NA | NA |
| | 186238373 | | | | | |
| 91 | chr4:186238352- | UACAAUUACCAAUUUCUGAA | NA | CR005921 | NA | NA |
| | 186238374 | | | | | |
| 92 | chr4:186251263- | GGUGUUUUCUUGAGGAGUAG | NA | CR005923 | NA | NA |
| | 186251285 | | | | | |
| 93 | chr4:186251490- | CGGGUAAAUUUUAGAAUGGC | NA | CR005926 | G013884 | 324 |
| | 186251512 | | | | | |
| 94 | chr4:186251494- | CUCCCGGGUAAAUUUUAGAA | NA | CR005927 | G013925 | 365 |
| | 186251516 | | | | | |
| 95 | chr4:186251801- | UAUGGGACACAAGGGAGCUC | NA | CR005930 | NA | NA |
| | 186251823 | | | | | |
| 96 | chr4:186252047- | UUGGAGGAACAAACUCUUCU | NA | CR005934 | G013912 | 352 |
| | 186252069 | | | | | |
| 97 | chr4:186252048- | UGGAGGAACAAACUCUUCUU | NA | CR005935 | NA | NA |
| | 186252070 | | | | | |
| 98 | chr4:186252056- | CAAACUCUUCUUGGGGAGAG | NA | CR005936 | NA | NA |
| | 186252078 | | | | | |
| 99 | chr4:186252123- | CUAUGAGUGACCCUCCACAC | NA | CR005937 | G013886 | 326 |
| | 186252145 | | | | | |
| 100 | chr4:186252124- | CUGUGUGGAGGGUCACUCAU | NA | CR005938 | G013938 | 378 |
| | 186252146 | | | | | |
| 101 | chr4:186252134- | GGUCACUCAUAGGACACCAG | NA | CR005939 | G013946 | 386 |
| | 186252156 | | | | | |
| 102 | chr4:186252135- | GUCACUCAUAGGACACCAGU | NA | CR005940 | G013896 | 336 |
| | 186252157 | | | | | |
| 103 | chr4:186252163- | ACUGCUGCCCACUGCUUUGA | NA | CR005941 | NA | NA |
| | 186252185 | | | | | |
| 104 | chr4:186252171- | ACACUUACCCAUCAAAGCAG | NA | CR005942 | G013902 | 342 |
| | 186252193 | | | | | |
| 105 | chr4:186238286- | AGGAACACCUACCGCUAUAA | NA | CR005944 | G013871 | 311 |
| | 186238308 | | | | | |
| 106 | chr4:186238265- | CUCCGGGACUGUACUUUAAU | NA | CR005945 | G013889 | 329 |
| | 186238287 | | | | | |
| 107 | chr4:186251786- | GUCCCAUACGCAAUCCUAGU | NA | CR005946 | G013890 | 330 |
| | 186251808 | | | | | |
| 108 | chr4:186238293- | CUCAGCACCUUUAUAGCGGU | NA | CR005947 | G013892 | 332 |
| | 186238315 | | | | | |
| 109 | chr4:186238282- | UAUAGCGGUAGGUGUUCCUC | NA | CR005948 | G013874 | 314 |
| | 186238304 | | | | | |
| 110 | chr4:186238266- | CUAUUAAAGUACAGUCCCGG | NA | CR005950 | G013875 | 315 |
| | 186238288 | | | | | |
| 111 | chr4:186238308- | GUGCUGAGUAACGUGGAAUC | NA | CR005953 | G013883 | 323 |
| | 186238330 | | | | | |
| 112 | chr4:186238301- | UAUAAAGGUGCUGAGUAACG | NA | CR005954 | G013878 | 318 |
| | 186238323 | | | | | |
| 113 | chr4:186251783- | UCUCCAACUAGGAUUGCGUA | NA | CR005955 | G013908 | 348 |
| | 186251805 | | | | | |
| 114 | chr4:186238281- | AUAGCGGUAGGUGUUCCUCC | NA | CR005957 | G013873 | 313 |
| | 186238303 | | | | | |
| 115 | chr4:186233989- | CUGCCAAAAGUACAUCGAAC | NA | CR005958 | G013877 | 317 |
| | 186234011 | | | | | |
| 116 | chr4:186238345- | ACCAAUUUCUGAAAGGGCAC | NA | CR005961 | NA | NA |
| | 186238367 | | | | | |
| 117 | chr4:186251755- | GUGUUUCUUAAGAUUAUCUA | NA | CR005962 | NA | NA |
| | 186251777 | | | | | |
| 118 | chr4:186238344- | CCAAUUUCUGAAAGGGCACA | NA | CR005964 | NA | NA |
| | 186238366 | | | | | |
| 119 | chr4:186251759- | UUCUUAAGAUUAUCUAUGGA | NA | CR005965 | G013940 | 380 |
| | 186251781 | | | | | |
| 120 | chr4:186233988- | CUGUUCGAUGUACUUUUGGC | NA | CR005966 | NA | NA |
| | 186234010 | | | | | |
| 121 | chr4:186233987- | UGUUCGAUGUACUUUUGGCA | NA | CR005967 | G013880 | 320 |
| | 186234009 | | | | | |
| 122 | chr4:186232209- | GGUGGAAUGUGCACCUCAUC | NA | CR005968 | G013939 | 379 |
| | 186232231 | | | | | |
| 123 | chr4:186250308- | GUCCGACACACAAAAGCAUC | NA | CR005969 | G013894 | 334 |
| | 186250330 | | | | | |
| 124 | chr4:186236877- | AAACUGGCAGCGAAUGUUAC | NA | CR005971 | G013930 | 370 |
| | 186236899 | | | | | |
| 125 | chr4:186236908- | UGCCACGCAAACAUUUCACA | NA | CR005972 | NA | NA |
| | 186236930 | | | | | |
| 126 | chr4:186233992- | GCACCUGUUCGAUGUACUUU | NA | CR005973 | G013870 | 310 |
| | 186234014 | | | | | |
| 127 | chr4:186254594- | AGAUGCGCCAAACAUCCUGC | NA | CR005974 | NA | NA |
| | 186254616 | | | | | |
| 128 | chr4:186232199- | GCACCUCAUCUGGCAGUAUU | NA | CR005975 | NA | NA |
| | 186232221 | | | | | |
| 129 | chr4:186250262- | CAUCUGAGAACGCAAGAUGC | NA | CR005976 | G013934 | 374 |
| | 186250284 | | | | | |
| 130 | chr4:186232196- | AUGCCCAAUACUGCCAGAUG | NA | CR005977 | NA | NA |
| | 186232218 | | | | | |
| 131 | chr4:186232200- | UGCACCUCAUCUGGCAGUAU | NA | CR005978 | G013944 | 384 |
| | 186232222 | | | | | |
| 132 | chr4:186232258- | AUGUCAUUGAUUGAACUUGC | NA | CR005980 | G013936 | 376 |
| | 186232280 | | | | | |
| 133 | chr4:186252031- | ACAAGCACACGCAUUGUUGG | NA | CR005981 | G013893 | 333 |
| | 186252053 | | | | | |
| 134 | chr4:186254723- | UAUCGCCUUGAUAAAACUCC | NA | CR005984 | G013926 | 366 |
| | 186254745 | | | | | |
| 135 | chr4:186251271- | CCUCAAGAAAACACCAUAUC | NA | CR005985 | G013906 | 346 |
| | 186251293 | | | | | |
| 136 | chr4:186232149- | AAACGCCUUCUUCAGAGGUG | NA | CR005986 | NA | NA |
| | 186232171 | | | | | |
| 137 | chr4:186252028- | AAAACAAGCACACGCAUUGU | NA | CR005987 | G013891 | 331 |
| | 186252050 | | | | | |
| 138 | chr4:186234001- | CAUCGAACAGGUGCAGUUUC | NA | CR005988 | G013879 | 319 |
| | 186234023 | | | | | |
| 139 | chr4:186254587- | GGCUUCCCCUGCAGGAUGUU | NA | CR005989 | G013881 | 321 |
| | 186254609 | | | | | |
| 140 | chr4:186234029- | UUGAUGACCACAUUGCUUCA | NA | CR005990 | G013937 | 377 |
| | 186234051 | | | | | |
| 141 | chr4:186254728- | AGGAGCCUGGAGUUUUAUCA | NA | CR005991 | NA | NA |
| | 186254750 | | | | | |
| 142 | chr4:186236783- | UGCCAUCGAGACAUUUAUAA | NA | CR005993 | G013899 | 339 |
| | 186236805 | | | | | |
| 143 | chr4:186232260- | AGCAAGUUCAAUCAAUGACA | NA | CR005996 | G013897 | 337 |
| | 186232282 | | | | | |
| 144 | chr4:186234022- | GGACAUUCCUUGAAGCAAUG | NA | CR005997 | NA | NA |
| | 186234044 | | | | | |
| 145 | chr4:186250330- | GUUGGGGUGAUAGGUGCAGA | NA | CR005999 | NA | NA |
| | 186250352 | | | | | |
| 146 | chr4:186232147- | GAAAACGCCUUCUUCAGAGG | NA | CR006000 | NA | NA |
| | 186232169 | | | | | |
| 147 | chr4:186232144- | UAUGAAAACGCCUUCUUCAG | NA | CR006001 | NA | NA |
| | 186232166 | | | | | |
| 148 | chr4:186250277- | CUCAGAUGUGGAUGUUGCCA | NA | CR006002 | NA | NA |
| | 186250299 | | | | | |
| 149 | chr4:186254579- | CUCUCCUAGGCUUCCCCUGC | NA | CR006003 | NA | NA |
| | 186254601 | | | | | |

TABLE 25
Cyno KLKB1 targeted single guide sequences,
chromosomal coordinates, and guide sequence homology to human

| | Cyno | | | Percent |
|---|---|---|---|---|
| | SEQ | | | homology |
| Cyno | ID | Exemplary Genomic | cyno guide | to human |
| sgRNA | NO | Coordinates (mf5) | sequence | guide |
| G013870 | 310 | chr5:185648888-185648908 | GCACCUGCUCGACGUACUUU | 90 |
| G013871 | 311 | chr5:185652966-185652986 | AGGAACGCCUACCACUAUAA | 90 |
| G013872 | 312 | chr5:185688465-185688485 | UGAUGGAAACGCUCGGAUGC | NA |
| G013873 | 313 | chr5:185652964-185652984 | AUAGUGGUAGGCGUUCCUCC | 90 |
| G013874 | 314 | chr5:185652965-185652985 | UAUAGUGGUAGGCGUUCCUC | 90 |
| G013875 | 315 | chr5:185652946-185652966 | CUCUUAAAGCACAGUCCCGG | 90 |
| G013876 | 316 | chr5:185684512-185684532 | AAUGCGCCAAACAUCCGGUA | 100 |
| G013877 | 317 | chr5:185648882-185648902 | UUGCCAAAAGUACGUCGAGC | 85 |
| G013878 | 318 | chr5:185652981-185653001 | UAUAAAGGUGCUGAAUAACG | 95 |
| G013879 | 319 | chr5:185648894-185648914 | CGUCGAGCAGGUGCAAUUUC | 85 |
| G013880 | 320 | chr5:185648883-185648903 | UGCUCGACGUACUUUUGGCA | 90 |
| G013881 | 321 | chr5:185684503-185684523 | GGCUUCCCUUACCGGAUGUU | 85 |
| G013882 | 322 | chr4:186256046-186256066 | CAACUGUUGGGUAACUGGAU | 100 |
| G013883 | 323 | chr5:185652988-185653008 | GUGCUGAAUAACGUGGAAUC | 95 |
| G013884 | 324 | chr5:185680852-185680872 | CGGGUAAAUUUUAGAAUGGC | 100 |
| G013885 | 325 | chr5:185684511-185684531 | AUGCGCCAAACAUCCGGUAA | 100 |
| G013886 | 326 | chr5:185681472-185681492 | CUAUGAGUGACCCUCCACAC | 100 |
| G013887 | 327 | chr5:185679339-185679359 | GGCAACAUCCACAUCCGAGA | NA |
| G013888 | 328 | chr5:185679426-185679446 | UUACGUUCUAUACGAAUGCA | 85 |
| G013889 | 329 | chr5:185652948-185652968 | CUCCGGGACUGUGCUUUAAG | 90 |
| G013890 | 330 | chr5:185681135-185681155 | GUCCCAUAUGUAAUCCUAGU | 90 |
| G013891 | 331 | chr5:185681374-185681394 | AAAACAAGCUCACGCAUUGU | 95 |
| G013892 | 332 | chr5:185652976-185652996 | UUCAGCACCUUUAUAGUGGU | 90 |
| G013893 | 333 | chr5:185681377-185681397 | ACAAGCUCACGCAUUGUUGG | 95 |
| G013894 | 334 | chr5:185679374-185679394 | GUUCGACACACAAAAGCAUC | 95 |
| G013895 | 335 | chr4:186258088-186258108 | GGGCGAAGGCUGUGCCCGCA | 100 |
| G013896 | 336 | chr5:185681481-185681501 | GUCACUCAUAGGACACCAGU | 100 |
| G013897 | 337 | chr5:185647160-185647180 | AGCAAGUUCCAUCAAUGACA | 95 |
| G013898 | 338 | chr5:185679413-185679433 | AACGUAAAGAAGAGGCAGCU | 100 |
| G013899 | 339 | chr5:185651465-185651485 | UGCCACCGAGACAUUUAUAA | 95 |
| G013900 | 340 | chr4:186256017-186256037 | GUGUUUGUGUCACCUUUGGA | 100 |
| G013901 | 341 | chr4:186251792-186251812 | GGAUUACAUAUGGGACACAA | 100 |
| G013902 | 342 | chr5:185681520-185681540 | ACACUUACCCAUCAAAGCAG | 100 |
| G013903 | 343 | chr5:185684660-185684680 | CAGUGUAAUUCAAAGGAGCC | 100 |
| G013904 | 344 | chr5:185681138-185681158 | AGGAUUACAUAUGGGACACA | 100 |
| G013905 | 345 | chr5:185651470-185651490 | UUCCUUUAUAAAUGUCUCGG | 100 |
| G013906 | 346 | chr5:185680632-185680652 | CCUCAAGAAAACACCACAUC | 95 |
| G013907 | 347 | chr5:185688458-185688478 | AGAGCAGUGAUGGAAACGCU | NA |
| G013908 | 348 | chr5:185681129-185681149 | UCUCCAACUAGGAUUACAUA | 90 |
| G013909 | 349 | chr5:185680982-185681002 | ACUCCCAGAAGACUGUAAGG | NA |
| G013910 | 350 | chr5:185679360-185679380 | AGCAUCUGGGGCGAGAACUC | 100 |
| G013911 | 351 | chr5:185679372-185679392 | UCGACACACAAAAGCAUCUG | NA |
| G013912 | 352 | chr5:185681393-185681413 | UUGGAGGAACAAACUCUUCU | 100 |
| G013913 | 353 | chr4:186236797-186236817 | UAUAAAGGAAUUGAUAUGAG | 100 |
| G013914 | 354 | chr4:186256047-186256067 | AACUGUUGGGUAACUGGAUG | 100 |
| G013915 | 355 | chr5:185647095-185647115 | AUCUGGCAGUGCUGGGCGUU | 100 |
| G013916 | 356 | chr4:186258042-186258062 | CUUGCAAACACAAUGGAAUG | 100 |
| G013917 | 357 | chr4:186251628-186251648 | UCUCCUCCUUACAGUCUUCU | |
| G013918 | 358 | chr5:185688296-185688316 | CUGUGACUCAGGGAGAUUCA | NA |
| G013918 | 358 | chr5:185688296-185688316 | CUGUGACUCAGGGAGAUUCA | 100 |
| G013919 | 359 | chr4:186258084-186258104 | GGCACAGCCUUCGCCCCAGC | 100 |
| G013920 | 360 | chr5:185647084-185647104 | CUGGGCGUUCGGGGUGUACA | 100 |
| G013921 | 361 | chr4:186254600-186254620 | GAUGUUUGGCGCAUUUAUAG | 100 |
| G013922 | 362 | chr4:186251771-186251791 | CUUCGGAUGGUUCUCCAACU | |
| G013923 | 363 | chr5:185684517-185684537 | CUAUAAAUGCGCCAAACAUC | NA |
| G013923 | 363 | chr5:185684517-185684537 | CUAUAAAUGCGCCAAACAUC | 100 |
| G013924 | 364 | chr4:186256045-186256065 | CCAACUGUUGGGUAACUGGA | |
| G013925 | 365 | chr5:185680856-185680876 | CUCCCGGGUAAAUUUUAGAA | 100 |
| G013926 | 366 | chr5:185684639-185684659 | UAUCGCCUUAAUAAAACUCC | 95 |
| G013926 | 366 | chr4:186254722-186254742 | UAUCGCCUUAAUAAAACUCC | 100 |
| G013927 | 367 | chr4:186250264-186250284 | GCAUCUUGCCUUCUCGGAUG | |
| G013928 | 368 | chr5:185679421-185679441 | UCGUAUAGAACGUAAAGAAG | 90 |
| G013929 | 369 | chr4:186258038-186258058 | CCAUUGUGUUUGCAAGCUAA | 100 |
| G013930 | 370 | chr5:185651562-185651582 | AAAUUGGCAGCGAAUGUUAU | 90 |
| G013930 | 370 | chr4:186236879-186236899 | AAAUUGGCAGCGAAUGUUAU | 100 |
| G013931 | 371 | chr4:186256048-186256068 | CCAUCCAGUUACCCAACAGU | 100 |
| G013932 | 372 | chr4:186256058-186256078 | AACUGGAUGGGGCUUCUCGA | 100 |
| G013933 | 373 | chr5:185681130-185681150 | CUCCAACUAGGAUUACAUAU | 100 |
| G013934 | 374 | chr5:185679328-185679348 | CAUCCGAGAAGGCAAGAUGC | 90 |
| G013935 | 375 | chr4:186251536-186251556 | UUGAAUGUGACUUUCGUUAA | 100 |
| G013936 | 376 | chr5:185647161-185647181 | AUGUCAUUGAUGGAACUUGC | 95 |
| G013937 | 377 | chr5:185648925-185648945 | UUGAUGACCACACUGCUUUA | 90 |
| G013938 | 378 | chr5:185681470-185681490 | CUGUGUGGAGGGUCACUCAU | 100 |
| G013939 | 379 | chr5:185647112-185647132 | GGUGGAAUGUGCACAUCAUC | 95 |
| G013940 | 380 | chr5:185681105-185681125 | UUCUUAAGAUUAUCUUCGGA | 90 |
| G013941 | 381 | chr5:185685921-185685941 | CCUUUGGAAGGUAGGCAUAU | 100 |
| G013942 | 382 | chr4:186258039-186258059 | UCCAUUGUGUUUGCAAGCUA | 100 |
| G013943 | 383 | chr4:186258008-186258028 | CCUGUGACUCAGGGAGAUUC | 100 |
| G013944 | 384 | chr5:185647103-185647123 | UGCACAUCAUCUGGCAGUGC | 85 |
| G013945 | 385 | chr4:186238299-186238319 | GUUAUUCAGCACCUUUAUAG | 100 |
| G013946 | 386 | chr5:185681480-185681500 | GGUCACUCAUAGGACACCAG | 100 |

1. A method of treating hereditary angioedema (HAE) in a human subject comprising systemically administering to the human subject a LNP composition comprising a therapeutically effective amount of:
i. an mRNA encoding a Cas nuclease, and
ii. a guide RNA that targets the KLKB1 gene in the liver.

2. A method of preventing HAE attacks in a human subject comprising systemically administering to the human subject a LNP composition comprising a therapeutically effective amount of:
i. an mRNA encoding a Cas nuclease, and
ii. a guide RNA that targets the KLKB1 gene in the liver.

3. A method of reducing the frequency of angioedema attacks in a human subject with HAE comprising systemically administering to the human subject a LNP composition comprising a therapeutically effective amount of:
i. an mRNA encoding a Cas nuclease, and
ii. a guide RNA that targets the KLKB1 gene in the liver.

4. A method for in vivo editing of the kallikrein (KLKB1) gene in a human subject having HAE, comprising:
a. systemically administering to the human subject a LNP composition comprising an therapeutically effective amount of:
i. an mRNA encoding a Cas nuclease, and
ii. a guide RNA that targets the KLKB1 gene; and
b. editing the KLKB1 gene at the site targeted by the guide RNA in a hepatocyte of the subject;
wherein the administration of the composition results in a clinically significant improvement in a level of a clinical metric in the subject as compared to a baseline level.

5. A method for treating hereditary angioedema (HAE) in a human subject, comprising:
a. systemically administering to the human subject a LNP composition comprising a therapeutically effective amount of:
i. an mRNA encoding a Cas nuclease, and
ii. a guide RNA that targets the KLKB1 wherein the guide RNA comprises a targeting sequence comprising the nucleotide sequence GGAUUGCGUAUGGGACACAA (SEQ ID NO: 15),
wherein the administration of the composition reduces total plasma kallikrein protein level relative to baseline total plasma kallikrein protein level.

6. A method of treating hereditary angioedema (HAE) in a human subject comprising:
a. systemically administering to the human subject a LNP composition comprising a therapeutically effective amount of:
i. an mRNA encoding a Cas nuclease, and
ii. a guide RNA that targets the KLKB1 gene in the liver;
b. determining a first level of a clinical metric in the subject prior to administration;
c. determining a second level of the clinical metric in the subject a period of time after administration; and
d. assessing the change between the first and the second levels of the clinical metric, wherein the administration of the composition results in a change in the level of the clinical metric in the subject that is improved as compared to a baseline level, thereby treating HAE.

7. A method of treating hereditary angioedema (HAE) in a human subject comprising:
a. systemically administering to the human subject a LNP composition comprising a therapeutically effective amount of:
i. an mRNA encoding a Cas nuclease, and
ii. a guide RNA that targets the KLKB1 gene in the liver;
b. determining a first level of a biosafety metric in the subject prior to administration;
c. determining a second level of the biosafety metric in the subject a period of time after administration; and
d. assessing the change between the first and the second levels of the biosafety metric, wherein the administration of the composition results in a change in a level of a biosafety metric in the subject that is acceptable as compared to a baseline level.

8. A method of treating hereditary angioedema (HAE) in a human subject comprising:
a. systemically administering to the human subject a LNP composition comprising an therapeutically effective amount of:
i. an mRNA encoding a Cas nuclease, and
ii. a guide RNA that targets the KLKB1 gene,
wherein the administration of the composition results in a clinically significant improvement in a level of a clinical metric in the subject as compared to a baseline level of the clinical metric.

9. A method for treating HAE in a human subject, comprising:
a. systemically administering to the human subject a LNP composition comprising a therapeutically effective amount of:
i. an mRNA encoding a Cas nuclease, and
ii. a guide RNA that targets the KLKB1 gene,
wherein the mRNA encoding a Cas nuclease and the guide RNA that targets the KLKB1 gene are administered at a combined dose of about 25 mg to about 75 mg.

10. A method for treating HAE in a human subject, comprising:
a. systemically administering to the human subject a LNP composition comprising a therapeutically effective amount of:
i. an mRNA encoding a Cas nuclease, and
ii. a guide RNA that targets the KLKB1 gene,
wherein the mRNA encoding a Cas nuclease and the guide RNA that targets the KLKB1 gene are administered at a combined dose of about 50 mg to about 75 mg.

11. The method of any one of claims 1-10, wherein kallikrein protein level is reduced by at least 60% following administration of the composition.

12. The method of any one of claims 1-11, further comprising reducing kallikrein activity level by at least 60% following administration of the composition.

13. The method of any one of claims 1-4 or 6-12, wherein the guide RNA comprises a targeting sequence comprising the nucleotide sequence GGAUUGCGUAUGGGACACAA (SEQ ID NO: 15).

14. The method of any one of claims 1-13, wherein the guide RNA further comprises a scaffold sequence comprising the nucleotide sequence
(SEQ ID NO: 387)
GUUUUAGAGCUAGAAAUAGCAAGUUAAAAUAAGGCUAGUCCGU
UAUCAACUUGAAAAAGUGGCACCGAGUCGGUGCUUUU.

15. The method of any one of claims 1-14, wherein the guide RNA is a modified guide RNA comprising or consisting of the nucleotide sequence mG*mG*mA*UUGCGUAUGGGACACAAGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 391), wherein m indicates a 2′-O-methyl modified nucleotide and * indicates a phosphorthioate linkage between nucleotides.

16. The method of any one of claims 1-15, wherein the subject is concurrently treated with HAE prophylaxis at the time of systemically administering to the human subject the LNP composition.

17. The method of claim 16, wherein the HAE prophylaxis comprises an agent selected from a C1 esterase inhibitor (C1-INH) replacement (recombinant or plasma-derived), an inhibitor of the B2 bradykinin receptor (B2R) (icatibant), a kallikrein inhibitor (ecallantide, lanadelumab, berotralstat), an attenuated androgen (danazol, oxandrolone, stanozolol), or an anti-fibrinolytic agent.

18. The method of claim 17, wherein the HAE prophylaxis comprises an agent selected from berotralstat and danazol.

19. The method of any one of claims 1-18, wherein the subject has an attack frequency of at least 2 confirmed HAE attacks in 90 days immediately prior to systemically administering to the human subject the LNP composition.

20. The method of any one of claims 1-18, wherein the subject has an attack frequency of at least 3 confirmed HAE attacks per 90 days immediately prior to systemically administering to the human subject the LNP composition.

21. The method of any one of claims 1-18, wherein the subject has an average attack frequency of at least 6 confirmed HAE attacks per 90 days immediately prior to systemically administering to the human subject the LNP composition.

22. The method of any one of claims 19-21, wherein the subject confirmed attack frequency is reduced by at least 50% for at least 90 days immediately after systemically administering to the human subject the LNP composition, optionally for at least 6 months immediately after systemically administering to the human subject the LNP composition.

23. The method of any one of claims 19-21, wherein the subject confirmed attack frequency is reduced by at least 80% for at least 90 days immediately after systemically administering to the human subject the LNP composition, optionally for 6 months immediately after systemically administering to the human subject the LNP composition.

24. The method of any one of claims 19-21, wherein the subject has no confirmed attacks for 90 days immediately after systemically administering to the human subject the LNP composition, optionally for 6 months immediately after systemically administering to the human subject the LNP composition.

25. The method of any one of claims 19-21, wherein the subject confirmed attack frequency is reduced by at least 80% on days 29-112 after systemically administering to the human subject the LNP composition, optionally on days 29-216 after systemically administering to the human subject the LNP composition.

26. The method of any one of claims 19-21, wherein the subject confirmed attack frequency is reduced by at least 90% on days 29-112 after systemically administering to the human subject the LNP composition, optionally on days 29-216 after systemically administering to the human subject the LNP composition.

27. The method of any one of claims 19-21, wherein the subject has no confirmed attacks on days 29-112 after systemically administering to the human subject the LNP composition, optionally on days 29-216 after systemically administering to the human subject the LNP composition.

28. The method of any one of claims 16-27, wherein prophylaxis is withdrawn concurrently with systemically administering to the human subject the LNP composition.

29. The method of any one of claims 16-28, wherein prophylaxis is withdrawn on or after day 36 after systemically administering to the human subject the LNP composition.

30. The method of any one of claims 1-29, wherein the subject has a reduced frequency of use of acute therapies for treatment of an HAE attack.

31. The method of any one of claims 1-30, wherein the subject has a reduced frequency of use of hospitalization related to an HAE attack as compared to control, e.g., the subject prior to treatment with the composition.

32. The method of any one of claims 1-31, wherein the subject has a reduced severity of confirmed HAE attacks.

33. The method of any one of claims 1-32, wherein the subject has a reduced frequency of confirmed severe HAE attacks.

34. The method of any one of claims 1-32, wherein the subject has a reduced frequency of confirmed HAE attacks with laryngeal edema.

35. The method of any one of claims 1-34, wherein the subject has an improved quality of life, optionally as determined by a suitable Quality of Life (QoL) assessment.

36. The method of any one of claims 1-35, wherein the subject does not tolerate long term treatment with an attenuated androgen or has an average attack frequency of at least 1 per 90 days while on treatment with an attenuated androgen.

37. The method of any one of claims 1-36, wherein the subject is a child, a pregnant woman, a subject with liver disease, a subject with breast cancer, a subject with prostate cancer, a subject with cardiovascular risk factors, and a subject with hepatocellular carcinoma.

38. The method of claim 37, wherein the subject is a pregnant woman.

39. The method of any one of claims 1-38, wherein the subject is a woman of child-bearing potential.

40. The method of any one of claims 1-39, wherein the subject has had at least one confirmed HAE attack with laryngeal edema.

41. The method of any one of claims 1-40 wherein the LNP comprises (9Z, 12Z)-3-((4,4-bis(octyloxy)butanoyl)oxy)-2-((((3-(diethylamino)propoxy)carbonyl)oxy)methyl)propyl octadeca-9, 12-dienoate.

42. The method of any one of claims 1-41, wherein the LNP comprises a PEG lipid.

43. The method of claim 42, wherein the PEG lipid comprises 1,2-dimyristoyl-rac-glycero-3-methylpolyoxyethylene glycol 2000.

44. The method of any one of claims 1-43, wherein the LNP comprises a neutral lipid.

45. The method of claim 44, wherein the neutral lipid comprises 1,2-distearoyl-sn-glycero-3-phosphocholine (DSPC).

46. The method of any one of claims 1-45, wherein the LNP composition has an N/P ratio of about 5-7.

47. The method of any one of claims 1-46, wherein the guide RNA and the mRNA encoding the Cas nuclease are present in a ratio ranging from about 5:1 to about 1:5 by weight.

48. The method of any one of claims 1-47, wherein the mRNA encodes a Cas9 nuclease.

49. The method of any one of claims 1-48, wherein the mRNA encodes S. pyogenes Cas9.

50. The method of any one of claims 1-49, wherein the mRNA encoding the Cas nuclease is codon-optimized.

51. The method of any one of claims 1-50, wherein the effective amount of mRNA encoding a Cas nuclease and the guide RNA that targets the KLKB1 gene is a combined dose of about 25-75 mg total RNA.

52. The method of any one of claims 1-51, wherein the effective amount of mRNA encoding a Cas nuclease and the guide RNA that targets the KLKB1 gene is a combined dose of about 25 mg total RNA.

53. The method of any one of claims 1-51, wherein the effective amount of mRNA encoding a Cas nuclease and the guide RNA that targets the KLKB1 gene is a combined dose of about 50 mg total RNA.

54. The method of any one of claims 1-51, wherein the effective amount of mRNA encoding a Cas nuclease and the guide RNA that targets the KLKB1 gene is a combined dose of about 75 mg total RNA.

55. The method of any one of claims 1-54, wherein administration of the composition reduces total plasma kallikrein protein level by 60%-95%, 60%-90%, 70%-95%, 70%-90%, or 80%-95% as compared to baseline total plasma kallikrein level before administration of the composition.

56. The method of any one of claims 1-55, wherein administration of the composition reduces plasma kallikrein activity by 60%-95%, 60%-90%, 70%-95%, 70%-90%, or 80%-95% as compared to baseline plasma kallikrein activity level before administration of the composition.

57. The method of any one of claims 55-56, wherein the plasma kallikrein level is determined at least 28 days after administration of the LNP composition.

58. The method of any one of claims 55-57, wherein the plasma kallikrein level is determined at 56 days after administration of the LNP composition.

59. The method of any one of claims 1-58, wherein administration of the composition results in a change in a level of a biosafety metric in the subject that is acceptable as compared to a baseline level of the biosafety metric.

60. The method of claim 59, wherein the biosafety metric is activated partial thromboplastin time (aPTT).

61. The method of claim 59 or 60, wherein the biosafety metric is alanine aminotransferase (ALT).

62. The method of any one of claims 59-61, wherein the biosafety metric is aspartate aminotransferase (AST).

63. The method of any one of claims 59-62, wherein the change in the biosafety metric resolves within 14 days.

64. The method of any one of claims 59-63, wherein the biosafety metric is grade 3 or less.

65. The method of any one of claims 1-64, wherein the composition is administered with a second therapeutic for treatment of HAE.

66. The method of claim 65, where in the second therapeutic is an HAE prophylaxis treatment.


SEQUENCE LISTING (410 sequences)

SEQ ID NO: 1 - RNA, 20 nt, Homo sapiens
  source 1..20: mol_type = genomic RNA
acaggaaactgtagcaaaca

SEQ ID NO: 2 - RNA, 20 nt, Homo sapiens
  source 1..20: mol_type = genomic RNA
atagataattcacttaccac

SEQ ID NO: 3 - RNA, 20 nt, Homo sapiens
  source 1..20: mol_type = genomic RNA
tacatccccacctctgaaga

SEQ ID NO: 4 - RNA, 20 nt, Homo sapiens
  source 1..20: mol_type = genomic RNA
tcttgaggagtagaggaact

SEQ ID NO: 5 - RNA, 20 nt, Homo sapiens
  source 1..20: mol_type = genomic RNA
accaggtaaagttcttttgc

SEQ ID NO: 6 - RNA, 20 nt, Homo sapiens
  source 1..20: mol_type = genomic RNA
gggtaaattttagaatggca

SEQ ID NO: 7 - RNA, 20 nt, Homo sapiens
  source 1..20: mol_type = genomic RNA
atttacccgggagttgactt

SEQ ID NO: 8 - RNA, 20 nt, Homo sapiens
  source 1..20: mol_type = genomic RNA
tacccgggagttgactttgg

SEQ ID NO: 9 - RNA, 20 nt, Homo sapiens
  source 1..20: mol_type = genomic RNA
tctttgagattgtgtaacac

SEQ ID NO: 10 - RNA, 20 nt, Homo sapiens
  source 1..20: mol_type = genomic RNA
ctttgagattgtgtaacact

SEQ ID NO: 11 - RNA, 20 nt, Homo sapiens
  source 1..20: mol_type = genomic RNA
tttgagattgtgtaacactg

SEQ ID NO: 12 - RNA, 20 nt, Homo sapiens
  source 1..20: mol_type = genomic RNA
tacataccagtgtaattcaa

SEQ ID NO: 13 - RNA, 20 nt, Homo sapiens
  source 1..20: mol_type = genomic RNA
ctccaactaggattgcgtat

SEQ ID NO: 14 - RNA, 20 nt, Homo sapiens
  source 1..20: mol_type = genomic RNA
aggattgcgtatgggacaca

SEQ ID NO: 15 - RNA, 20 nt, Homo sapiens
  source 1..20: mol_type = genomic RNA
ggattgcgtatgggacacaa

SEQ ID NO: 16 - RNA, 20 nt, Homo sapiens
  source 1..20: mol_type = genomic RNA
gttactcagcacctttatag

SEQ ID NO: 17 - RNA, 20 nt, Homo sapiens
  source 1..20: mol_type = genomic RNA
tgcctattaaagtacagtcc

SEQ ID NO: 18 - RNA, 20 nt, Homo sapiens
  source 1..20: mol_type = genomic RNA
ctatggatggttctccaact

SEQ ID NO: 19 - RNA, 20 nt, Homo sapiens
  source 1..20: mol_type = genomic RNA
gatgtttggcgcatctatag

SEQ ID NO: 20 - RNA, 20 nt, Homo sapiens
  source 1..20: mol_type = genomic RNA
atgcgccaaacatcctgcag

SEQ ID NO: 21 - RNA, 20 nt, Homo sapiens
  source 1..20: mol_type = genomic RNA
ctcctttataaatgtctcga

SEQ ID NO: 22 - RNA, 20 nt, Homo sapiens
  source 1..20: mol_type = genomic RNA
tgttactggtgcaccttttt

SEQ ID NO: 23 - RNA, 20 nt, Homo sapiens
  source 1..20: mol_type = genomic RNA
gatgcgccaaacatcctgca

SEQ ID NO: 24 - RNA, 20 nt, Homo sapiens
  source 1..20: mol_type = genomic RNA
atctggcagtattgggcatt

SEQ ID NO: 25 - RNA, 20 nt, Homo sapiens
  source 1..20: mol_type = genomic RNA
gcgtggcatatgaaaaaaac

SEQ ID NO: 26 - RNA, 20 nt, Homo sapiens
  source 1..20: mol_type = genomic RNA
tataaaggagttgatatgag

SEQ ID NO: 27 - RNA, 20 nt, Homo sapiens
  source 1..20: mol_type = genomic RNA
acaccttgaattgtactcac

SEQ ID NO: 28 - RNA, 20 nt, Homo sapiens
  source 1..20: mol_type = genomic RNA
tgaggtgcacattccaccca

SEQ ID NO: 29 - RNA, 20 nt, Homo sapiens
  source 1..20: mol_type = genomic RNA
ctggcagtattgggcatttg

SEQ ID NO: 30 - RNA, 20 nt, Homo sapiens
  source 1..20: mol_type = genomic RNA
aaaacgccttcttcagaggt

SEQ ID NO: 31 - RNA, 20 nt, Homo sapiens
  source 1..20: mol_type = genomic RNA
tgaatagcaaacaccttggg

SEQ ID NO: 32 - RNA, 20 nt, Homo sapiens
  source 1..20: mol_type = genomic RNA
agtcaattttaatgtgtcta

SEQ ID NO: 33 - RNA, 20 nt, Homo sapiens
  source 1..20: mol_type = genomic RNA
gtgttgaagaatgccaaaaa

SEQ ID NO: 34 - RNA, 20 nt, Homo sapiens
  source 1..20: mol_type = genomic RNA
tgccttgtgaaatgtttgcg

SEQ ID NO: 35 - RNA, 20 nt, Homo sapiens
  source 1..20: mol_type = genomic RNA
gcatcttgcgttctcagatg

SEQ ID NO: 36 - RNA, 20 nt, Homo sapiens
  source 1..20: mol_type = genomic RNA
tctcagatgtggatgttgcc

SEQ ID NO: 37 - RNA, 20 nt, Homo sapiens
  source 1..20: mol_type = genomic RNA
ctccagatgcttttgtgtgt

SEQ ID NO: 38 - RNA, 20 nt, Homo sapiens
  source 1..20: mol_type = genomic RNA
tattatcaaatcacattacc

SEQ ID NO: 39 - RNA, 20 nt, Homo sapiens
  source 1..20: mol_type = genomic RNA
ccagatatggtgttttcttg

SEQ ID NO: 40 - RNA, 20 nt, Homo sapiens
  source 1..20: mol_type = genomic RNA
aagttcttttgcaggttaaa

SEQ ID NO: 41 - RNA, 20 nt, Homo sapiens
  source 1..20: mol_type = genomic RNA
tttactcccagaagactgta

SEQ ID NO: 42 - RNA, 20 nt, Homo sapiens
  source 1..20: mol_type = genomic RNA
tgccattctaaaatttaccc

SEQ ID NO: 43 - RNA, 20 nt, Homo sapiens
  source 1..20: mol_type = genomic RNA
tcatctttgtgcaagtctct

SEQ ID NO: 44 - RNA, 20 nt, Homo sapiens
  source 1..20: mol_type = genomic RNA
tctcctccaaagtcaactcc

SEQ ID NO: 45 - RNA, 20 nt, Homo sapiens
  source 1..20: mol_type = genomic RNA
ggaggaacaaactcttcttg

SEQ ID NO: 46 - RNA, 20 nt, Homo sapiens
  source 1..20: mol_type = genomic RNA
aggtgaagctgacagctcag

SEQ ID NO: 47 - RNA, 20 nt, Homo sapiens
  source 1..20: mol_type = genomic RNA
ccatccggttacccaacagt

SEQ ID NO: 48 - RNA, 20 nt, Homo sapiens
  source 1..20: mol_type = genomic RNA
tataccaactgttgggtaac

SEQ ID NO: 49 - RNA, 20 nt, Homo sapiens
  source 1..20: mol_type = genomic RNA
gcacaatttataccaactgt

SEQ ID NO: 50 - RNA, 20 nt, Homo sapiens
  source 1..20: mol_type = genomic RNA
aaccggatggggcttctcga

SEQ ID NO: 51 - RNA, 20 nt, Homo sapiens
  source 1..20: mol_type = genomic RNA
caactgttgggtaaccggat

SEQ ID NO: 52 - RNA, 20 nt, Homo sapiens
  source 1..20: mol_type = genomic RNA
cacaatttataccaactgtt

SEQ ID NO: 53 - RNA, 20 nt, Homo sapiens
  source 1..20: mol_type = genomic RNA
ccaactgttgggtaaccgga

SEQ ID NO: 54 - RNA, 20 nt, Homo sapiens
  source 1..20: mol_type = genomic RNA
ctccttcgagaagccccatc

SEQ ID NO: 55 - RNA, 20 nt, Homo sapiens
  source 1..20: mol_type = genomic RNA
aactgttgggtaaccggatg

SEQ ID NO: 56 - RNA, 20 nt, Homo sapiens
  source 1..20: mol_type = genomic RNA
ccaatatgcctaccttccaa

SEQ ID NO: 57 - RNA, 20 nt, Homo sapiens
  source 1..20: mol_type = genomic RNA
gtgcttgtgtcacctttgga

SEQ ID NO: 58 - RNA, 20 nt, Homo sapiens
  source 1..20: mol_type = genomic RNA
ttgtgtcacctttggaaggt

SEQ ID NO: 59 - RNA, 20 nt, Homo sapiens
  source 1..20: mol_type = genomic RNA
aattgtgcttgtgtcacctt

SEQ ID NO: 60 - RNA, 20 nt, Homo sapiens
  source 1..20: mol_type = genomic RNA
aaggtaggcatattggtttt

SEQ ID NO: 61 - RNA, 20 nt, Homo sapiens
  source 1..20: mol_type = genomic RNA
acccaacggatggtctgtgc

SEQ ID NO: 62 - RNA, 20 nt, Homo sapiens
  source 1..20: mol_type = genomic RNA
agccagcacagaccatccgt

SEQ ID NO: 63 - RNA, 20 nt, Homo sapiens
  source 1..20: mol_type = genomic RNA
ttataaaataacccaacgga

SEQ ID NO: 64 - RNA, 20 nt, Homo sapiens
  source 1..20: mol_type = genomic RNA
ctgtgctggctataaagaag

SEQ ID NO: 65 - RNA, 20 nt, Homo sapiens
  source 1..20: mol_type = genomic RNA
cattcttcatttgttaccaa

SEQ ID NO: 66 - RNA, 20 nt, Homo sapiens
  source 1..20: mol_type = genomic RNA
tataatcttgatatcttttc

SEQ ID NO: 67 - RNA, 20 nt, Homo sapiens
  source 1..20: mol_type = genomic RNA
gccagcacagaccatccgtt

SEQ ID NO: 68 - RNA, 20 nt, Homo sapiens
  source 1..20: mol_type = genomic RNA
gtctgtgctggctataaaga

SEQ ID NO: 69 - RNA, 20 nt, Homo sapiens
  source 1..20: mol_type = genomic RNA
tctgtgctggctataaagaa

SEQ ID NO: 70 - RNA, 20 nt, Homo sapiens
  source 1..20: mol_type = genomic RNA
gtccatgtactcagcgactt

SEQ ID NO: 71 - RNA, 20 nt, Homo sapiens
  source 1..20: mol_type = genomic RNA
caccaaagtcgctgagtaca

SEQ ID NO: 72 - RNA, 20 nt, Homo sapiens
  source 1..20: mol_type = genomic RNA
acacaatggaatgtggcgtt

SEQ ID NO: 73 - RNA, 20 nt, Homo sapiens
  source 1..20: mol_type = genomic RNA
tttggtgggcatcaccagct

SEQ ID NO: 74 - RNA, 20 nt, Homo sapiens
  source 1..20: mol_type = genomic RNA
ctctggactgcttctcatgc

SEQ ID NO: 75 - RNA, 20 nt, Homo sapiens
  source 1..20: mol_type = genomic RNA
aagtcgctgagtacatggac

SEQ ID NO: 76 - RNA, 20 nt, Homo sapiens
  source 1..20: mol_type = genomic RNA
gggtgaaggctgtgcccgca

SEQ ID NO: 77 - RNA, 20 nt, Homo sapiens
  source 1..20: mol_type = genomic RNA
aatggaatgtggcgtttggt

SEQ ID NO: 78 - RNA, 20 nt, Homo sapiens
  source 1..20: mol_type = genomic RNA
tccattgtgtttgcaaacta

SEQ ID NO: 79 - RNA, 20 nt, Homo sapiens
  source 1..20: mol_type = genomic RNA
gtttggtgggcatcaccagc

SEQ ID NO: 80 - RNA, 20 nt, Homo sapiens
  source 1..20: mol_type = genomic RNA
tttgcaaacacaatggaatg

SEQ ID NO: 81 - RNA, 20 nt, Homo sapiens
  source 1..20: mol_type = genomic RNA
gacaccaggttgctccctgc

SEQ ID NO: 82 - RNA, 20 nt, Homo sapiens
  source 1..20: mol_type = genomic RNA
actgtgactcagggagattc

SEQ ID NO: 83 - RNA, 20 nt, Homo sapiens
  source 1..20: mol_type = genomic RNA
tgtgcccgcagggagcaacc

SEQ ID NO: 84 - RNA, 20 nt, Homo sapiens
  source 1..20: mol_type = genomic RNA
ccattgtgtttgcaaactaa

SEQ ID NO: 85 - RNA, 20 nt, Homo sapiens
  source 1..20: mol_type = genomic RNA
ggggtgaaggctgtgcccgc

SEQ ID NO: 86 - RNA, 20 nt, Homo sapiens
  source 1..20: mol_type = genomic RNA
gcgactttggtgtagacacc

SEQ ID NO: 87 - RNA, 20 nt, Homo sapiens
  source 1..20: mol_type = genomic RNA
cccttagtttgcaaacacaa

SEQ ID NO: 88 - RNA, 20 nt, Homo sapiens
  source 1..20: mol_type = genomic RNA
caatggaatgtggcgtttgg

SEQ ID NO: 89 - RNA, 20 nt, Homo sapiens
  source 1..20: mol_type = genomic RNA
aactgaatagcaaacacctt

SEQ ID NO: 90 - RNA, 20 nt, Homo sapiens
  source 1..20: mol_type = genomic RNA
acaattaccaatttctgaaa

SEQ ID NO: 91 - RNA, 20 nt, Homo sapiens
  source 1..20: mol_type = genomic RNA
tacaattaccaatttctgaa

SEQ ID NO: 92 - RNA, 20 nt, Homo sapiens
  source 1..20: mol_type = genomic RNA
ggtgttttcttgaggagtag

SEQ ID NO: 93 - RNA, 20 nt, Homo sapiens
  source 1..20: mol_type = genomic RNA
cgggtaaattttagaatggc

SEQ ID NO: 94 - RNA, 20 nt, Homo sapiens
  source 1..20: mol_type = genomic RNA
ctcccgggtaaattttagaa

SEQ ID NO: 95 - RNA, 20 nt, Homo sapiens
  source 1..20: mol_type = genomic RNA
tatgggacacaagggagctc

SEQ ID NO: 96 - RNA, 20 nt, Homo sapiens
  source 1..20: mol_type = genomic RNA
ttggaggaacaaactcttct

SEQ ID NO: 97 - RNA, 20 nt, Homo sapiens
  source 1..20: mol_type = genomic RNA
tggaggaacaaactcttctt

SEQ ID NO: 98 - RNA, 20 nt, Homo sapiens
  source 1..20: mol_type = genomic RNA
caaactcttcttggggagag

SEQ ID NO: 99 - RNA, 20 nt, Homo sapiens
  source 1..20: mol_type = genomic RNA
ctatgagtgaccctccacac

SEQ ID NO: 100 - RNA, 20 nt, Homo sapiens
  source 1..20: mol_type = genomic RNA
ctgtgtggagggtcactcat

SEQ ID NO: 101 - RNA, 20 nt, Homo sapiens
  source 1..20: mol_type = genomic RNA
ggtcactcataggacaccag

SEQ ID NO: 102 - RNA, 20 nt, Homo sapiens
  source 1..20: mol_type = genomic RNA
gtcactcataggacaccagt

SEQ ID NO: 103 - RNA, 20 nt, Homo sapiens
  source 1..20: mol_type = genomic RNA
actgctgcccactgctttga

SEQ ID NO: 104 - RNA, 20 nt, Homo sapiens
  source 1..20: mol_type = genomic RNA
acacttacccatcaaagcag

SEQ ID NO: 105 - RNA, 20 nt, Homo sapiens
  source 1..20: mol_type = genomic RNA
aggaacacctaccgctataa

SEQ ID NO: 106 - RNA, 20 nt, Homo sapiens
  source 1..20: mol_type = genomic RNA
ctccgggactgtactttaat

SEQ ID NO: 107 - RNA, 20 nt, Homo sapiens
  source 1..20: mol_type = genomic RNA
gtcccatacgcaatcctagt

SEQ ID NO: 108 - RNA, 20 nt, Homo sapiens
  source 1..20: mol_type = genomic RNA
ctcagcacctttatagcggt

SEQ ID NO: 109 - RNA, 20 nt, Homo sapiens
  source 1..20: mol_type = genomic RNA
tatagcggtaggtgttcctc

SEQ ID NO: 110 - RNA, 20 nt, Homo sapiens
  source 1..20: mol_type = genomic RNA
ctattaaagtacagtcccgg

SEQ ID NO: 111 - RNA, 20 nt, Homo sapiens
  source 1..20: mol_type = genomic RNA
gtgctgagtaacgtggaatc

SEQ ID NO: 112 - RNA, 20 nt, Homo sapiens
  source 1..20: mol_type = genomic RNA
tataaaggtgctgagtaacg

SEQ ID NO: 113 - RNA, 20 nt, Homo sapiens
  source 1..20: mol_type = genomic RNA
tctccaactaggattgcgta

SEQ ID NO: 114 - RNA, 20 nt, Homo sapiens
  source 1..20: mol_type = genomic RNA
atagcggtaggtgttcctcc

SEQ ID NO: 115 - RNA, 20 nt, Homo sapiens
  source 1..20: mol_type = genomic RNA
ctgccaaaagtacatcgaac

SEQ ID NO: 116 - RNA, 20 nt, Homo sapiens
  source 1..20: mol_type = genomic RNA
accaatttctgaaagggcac

SEQ ID NO: 117 - RNA, 20 nt, Homo sapiens
  source 1..20: mol_type = genomic RNA
gtgtttcttaagattatcta

SEQ ID NO: 118 - RNA, 20 nt, Homo sapiens
  source 1..20: mol_type = genomic RNA
ccaatttctgaaagggcaca

SEQ ID NO: 119 - RNA, 20 nt, Homo sapiens
  source 1..20: mol_type = genomic RNA
ttcttaagattatctatgga

SEQ ID NO: 120 - RNA, 20 nt, Homo sapiens
  source 1..20: mol_type = genomic RNA
ctgttcgatgtacttttggc

SEQ ID NO: 121 - RNA, 20 nt, Homo sapiens
  source 1..20: mol_type = genomic RNA
tgttcgatgtacttttggca

SEQ ID NO: 122 - RNA, 20 nt, Homo sapiens
  source 1..20: mol_type = genomic RNA
ggtggaatgtgcacctcatc

SEQ ID NO: 123 - RNA, 20 nt, Homo sapiens
  source 1..20: mol_type = genomic RNA
gtccgacacacaaaagcatc

SEQ ID NO: 124 - RNA, 20 nt, Homo sapiens
  source 1..20: mol_type = genomic RNA
aaactggcagcgaatgttac

SEQ ID NO: 125 - RNA, 20 nt, Homo sapiens
  source 1..20: mol_type = genomic RNA
tgccacgcaaacatttcaca

SEQ ID NO: 126 - RNA, 20 nt, Homo sapiens
  source 1..20: mol_type = genomic RNA
gcacctgttcgatgtacttt

SEQ ID NO: 127 - RNA, 20 nt, Homo sapiens
  source 1..20: mol_type = genomic RNA
agatgcgccaaacatcctgc

SEQ ID NO: 128 - RNA, 20 nt, Homo sapiens
  source 1..20: mol_type = genomic RNA
gcacctcatctggcagtatt

SEQ ID NO: 129 - RNA, 20 nt, Homo sapiens
  source 1..20: mol_type = genomic RNA
catctgagaacgcaagatgc

SEQ ID NO: 130 - RNA, 20 nt, Homo sapiens
  source 1..20: mol_type = genomic RNA
atgcccaatactgccagatg

SEQ ID NO: 131 - RNA, 20 nt, Homo sapiens
  source 1..20: mol_type = genomic RNA
tgcacctcatctggcagtat

SEQ ID NO: 132 - RNA, 20 nt, Homo sapiens
  source 1..20: mol_type = genomic RNA
atgtcattgattgaacttgc

SEQ ID NO: 133 - RNA, 20 nt, Homo sapiens
  source 1..20: mol_type = genomic RNA
acaagcacacgcattgttgg

SEQ ID NO: 134 - RNA, 20 nt, Homo sapiens
  source 1..20: mol_type = genomic RNA
tatcgccttgataaaactcc

SEQ ID NO: 135 - RNA, 20 nt, Homo sapiens
  source 1..20: mol_type = genomic RNA
cctcaagaaaacaccatatc

SEQ ID NO: 136 - RNA, 20 nt, Homo sapiens
  source 1..20: mol_type = genomic RNA
aaacgccttcttcagaggtg

SEQ ID NO: 137 - RNA, 20 nt, Homo sapiens
  source 1..20: mol_type = genomic RNA
aaaacaagcacacgcattgt

SEQ ID NO: 138 - RNA, 20 nt, Homo sapiens
  source 1..20: mol_type = genomic RNA
catcgaacaggtgcagtttc

SEQ ID NO: 139 - RNA, 20 nt, Homo sapiens
  source 1..20: mol_type = genomic RNA
ggcttcccctgcaggatgtt

SEQ ID NO: 140 - RNA, 20 nt, Homo sapiens
  source 1..20: mol_type = genomic RNA
ttgatgaccacattgcttca

SEQ ID NO: 141 - RNA, 20 nt, Homo sapiens
  source 1..20: mol_type = genomic RNA
aggagcctggagttttatca

SEQ ID NO: 142 - RNA, 20 nt, Homo sapiens
  source 1..20: mol_type = genomic RNA
tgccatcgagacatttataa

SEQ ID NO: 143 - RNA, 20 nt, Homo sapiens
  source 1..20: mol_type = genomic RNA
agcaagttcaatcaatgaca

SEQ ID NO: 144 - RNA, 20 nt, Homo sapiens
  source 1..20: mol_type = genomic RNA
ggacattccttgaagcaatg

SEQ ID NO: 145 - RNA, 20 nt, Homo sapiens
  source 1..20: mol_type = genomic RNA
gttggggtgataggtgcaga

SEQ ID NO: 146 - RNA, 20 nt, Homo sapiens
  source 1..20: mol_type = genomic RNA
gaaaacgccttcttcagagg

SEQ ID NO: 147 - RNA, 20 nt, Homo sapiens
  source 1..20: mol_type = genomic RNA
tatgaaaacgccttcttcag

SEQ ID NO: 148 - RNA, 20 nt, Homo sapiens
  source 1..20: mol_type = genomic RNA
ctcagatgtggatgttgcca

SEQ ID NO: 149 - RNA, 20 nt, Homo sapiens
  source 1..20: mol_type = genomic RNA
ctctcctaggcttcccctgc

SEQ ID NO: 150-309 - skipped (160 entries)

SEQ ID NO: 310 - RNA, 20 nt, Macaca fascicularis
  source 1..20: mol_type = genomic RNA
gcacctgctcgacgtacttt

SEQ ID NO: 311 - RNA, 20 nt, Macaca fascicularis
  source 1..20: mol_type = genomic RNA
aggaacgcctaccactataa

SEQ ID NO: 312 - RNA, 20 nt, Macaca fascicularis
  source 1..20: mol_type = genomic RNA
tgatggaaacgctcggatgc

SEQ ID NO: 313 - RNA, 20 nt, Macaca fascicularis
  source 1..20: mol_type = genomic RNA
atagtggtaggcgttcctcc

SEQ ID NO: 314 - RNA, 20 nt, Macaca fascicularis
  source 1..20: mol_type = genomic RNA
tatagtggtaggcgttcctc

SEQ ID NO: 315 - RNA, 20 nt, Macaca fascicularis
  source 1..20: mol_type = genomic RNA
ctcttaaagcacagtcccgg

SEQ ID NO: 316 - RNA, 20 nt, Macaca fascicularis
  source 1..20: mol_type = genomic RNA
aatgcgccaaacatccggta

SEQ ID NO: 317 - RNA, 20 nt, Macaca fascicularis
  source 1..20: mol_type = genomic RNA
ttgccaaaagtacgtcgagc

SEQ ID NO: 318 - RNA, 20 nt, Macaca fascicularis
  source 1..20: mol_type = genomic RNA
tataaaggtgctgaataacg

SEQ ID NO: 319 - RNA, 20 nt, Macaca fascicularis
  source 1..20: mol_type = genomic RNA
cgtcgagcaggtgcaatttc

SEQ ID NO: 320 - RNA, 20 nt, Macaca fascicularis
  source 1..20: mol_type = genomic RNA
tgctcgacgtacttttggca

SEQ ID NO: 321 - RNA, 20 nt, Macaca fascicularis
  source 1..20: mol_type = genomic RNA
ggcttcccttaccggatgtt

SEQ ID NO: 322 - RNA, 20 nt, Macaca fascicularis
  source 1..20: mol_type = genomic RNA
caactgttgggtaactggat

SEQ ID NO: 323 - RNA, 20 nt, Macaca fascicularis
  source 1..20: mol_type = genomic RNA
gtgctgaataacgtggaatc

SEQ ID NO: 324 - RNA, 20 nt, Macaca fascicularis
  source 1..20: mol_type = genomic RNA
cgggtaaattttagaatggc

SEQ ID NO: 325 - RNA, 20 nt, Macaca fascicularis
  source 1..20: mol_type = genomic RNA
atgcgccaaacatccggtaa

SEQ ID NO: 326 - RNA, 20 nt, Macaca fascicularis
  source 1..20: mol_type = genomic RNA
ctatgagtgaccctccacac

SEQ ID NO: 327 - RNA, 20 nt, Macaca fascicularis
  source 1..20: mol_type = genomic RNA
ggcaacatccacatccgaga

SEQ ID NO: 328 - RNA, 20 nt, Macaca fascicularis
  source 1..20: mol_type = genomic RNA
ttacgttctatacgaatgca

SEQ ID NO: 329 - RNA, 20 nt, Macaca fascicularis
  source 1..20: mol_type = genomic RNA
ctccgggactgtgctttaag

SEQ ID NO: 330 - RNA, 20 nt, Macaca fascicularis
  source 1..20: mol_type = genomic RNA
gtcccatatgtaatcctagt

SEQ ID NO: 331 - RNA, 20 nt, Macaca fascicularis
  source 1..20: mol_type = genomic RNA
aaaacaagctcacgcattgt

SEQ ID NO: 332 - RNA, 20 nt, Macaca fascicularis
  source 1..20: mol_type = genomic RNA
ttcagcacctttatagtggt

SEQ ID NO: 333 - RNA, 20 nt, Macaca fascicularis
  source 1..20: mol_type = genomic RNA
acaagctcacgcattgttgg

SEQ ID NO: 334 - RNA, 20 nt, Macaca fascicularis
  source 1..20: mol_type = genomic RNA
gttcgacacacaaaagcatc

SEQ ID NO: 335 - RNA, 20 nt, Macaca fascicularis
  source 1..20: mol_type = genomic RNA
gggcgaaggctgtgcccgca

SEQ ID NO: 336 - RNA, 20 nt, Macaca fascicularis
  source 1..20: mol_type = genomic RNA
gtcactcataggacaccagt

SEQ ID NO: 337 - RNA, 20 nt, Macaca fascicularis
  source 1..20: mol_type = genomic RNA
agcaagttccatcaatgaca

SEQ ID NO: 338 - RNA, 20 nt, Macaca fascicularis
  source 1..20: mol_type = genomic RNA
aacgtaaagaagaggcagct

SEQ ID NO: 339 - RNA, 20 nt, Macaca fascicularis
  source 1..20: mol_type = genomic RNA
tgccaccgagacatttataa

SEQ ID NO: 340 - RNA, 20 nt, Macaca fascicularis
  source 1..20: mol_type = genomic RNA
gtgtttgtgtcacctttgga

SEQ ID NO: 341 - RNA, 20 nt, Macaca fascicularis
  source 1..20: mol_type = genomic RNA
ggattacatatgggacacaa

SEQ ID NO: 342 - RNA, 20 nt, Macaca fascicularis
  source 1..20: mol_type = genomic RNA
acacttacccatcaaagcag

SEQ ID NO: 343 - RNA, 20 nt, Macaca fascicularis
  source 1..20: mol_type = genomic RNA
cagtgtaattcaaaggagcc

SEQ ID NO: 344 - RNA, 20 nt, Macaca fascicularis
  source 1..20: mol_type = genomic RNA
aggattacatatgggacaca

SEQ ID NO: 345 - RNA, 20 nt, Macaca fascicularis
  source 1..20: mol_type = genomic RNA
ttcctttataaatgtctcgg

SEQ ID NO: 346 - RNA, 20 nt, Macaca fascicularis
  source 1..20: mol_type = genomic RNA
cctcaagaaaacaccacatc

SEQ ID NO: 347 - RNA, 20 nt, Macaca fascicularis
  source 1..20: mol_type = genomic RNA
agagcagtgatggaaacgct

SEQ ID NO: 348 - RNA, 20 nt, Macaca fascicularis
  source 1..20: mol_type = genomic RNA
tctccaactaggattacata

SEQ ID NO: 349 - RNA, 20 nt, Macaca fascicularis
  source 1..20: mol_type = genomic RNA
actcccagaagactgtaagg

SEQ ID NO: 350 - RNA, 20 nt, Macaca fascicularis
  source 1..20: mol_type = genomic RNA
agcatctggggcgagaactc

SEQ ID NO: 351 - RNA, 20 nt, Macaca fascicularis
  source 1..20: mol_type = genomic RNA
tcgacacacaaaagcatctg

SEQ ID NO: 352 - RNA, 20 nt, Macaca fascicularis
  source 1..20: mol_type = genomic RNA
ttggaggaacaaactcttct

SEQ ID NO: 353 - RNA, 20 nt, Macaca fascicularis
  source 1..20: mol_type = genomic RNA
tataaaggaattgatatgag

SEQ ID NO: 354 - RNA, 20 nt, Macaca fascicularis
  source 1..20: mol_type = genomic RNA
aactgttgggtaactggatg

SEQ ID NO: 355 - RNA, 20 nt, Macaca fascicularis
  source 1..20: mol_type = genomic RNA
atctggcagtgctgggcgtt

SEQ ID NO: 356 - RNA, 20 nt, Macaca fascicularis
  source 1..20: mol_type = genomic RNA
cttgcaaacacaatggaatg

SEQ ID NO: 357 - RNA, 20 nt, Macaca fascicularis
  source 1..20: mol_type = genomic RNA
tctcctccttacagtcttct

SEQ ID NO: 358 - RNA, 20 nt, Macaca fascicularis
  source 1..20: mol_type = genomic RNA
ctgtgactcagggagattca

SEQ ID NO: 359 - RNA, 20 nt, Macaca fascicularis
  source 1..20: mol_type = genomic RNA
ggcacagccttcgccccagc

SEQ ID NO: 360 - RNA, 20 nt, Macaca fascicularis
  source 1..20: mol_type = genomic RNA
ctgggcgttcggggtgtaca

SEQ ID NO: 361 - RNA, 20 nt, Macaca fascicularis
  source 1..20: mol_type = genomic RNA
gatgtttggcgcatttatag

SEQ ID NO: 362 - RNA, 20 nt, Macaca fascicularis
  source 1..20: mol_type = genomic RNA
cttcggatggttctccaact

SEQ ID NO: 363 - RNA, 20 nt, Macaca fascicularis
  source 1..20: mol_type = genomic RNA
ctataaatgcgccaaacatc

SEQ ID NO: 364 - RNA, 20 nt, Macaca fascicularis
  source 1..20: mol_type = genomic RNA
ccaactgttgggtaactgga

SEQ ID NO: 365 - RNA, 20 nt, Macaca fascicularis
  source 1..20: mol_type = genomic RNA
ctcccgggtaaattttagaa

SEQ ID NO: 366 - RNA, 20 nt, Macaca fascicularis
  source 1..20: mol_type = genomic RNA
tatcgccttaataaaactcc

SEQ ID NO: 367 - RNA, 20 nt, Macaca fascicularis
  source 1..20: mol_type = genomic RNA
gcatcttgccttctcggatg

SEQ ID NO: 368 - RNA, 20 nt, Macaca fascicularis
  source 1..20: mol_type = genomic RNA
tcgtatagaacgtaaagaag

SEQ ID NO: 369 - RNA, 20 nt, Macaca fascicularis
  source 1..20: mol_type = genomic RNA
ccattgtgtttgcaagctaa

SEQ ID NO: 370 - RNA, 20 nt, Macaca fascicularis
  source 1..20: mol_type = genomic RNA
aaattggcagcgaatgttat

SEQ ID NO: 371 - RNA, 20 nt, Macaca fascicularis
  source 1..20: mol_type = genomic RNA
ccatccagttacccaacagt

SEQ ID NO: 372 - RNA, 20 nt, Macaca fascicularis
  source 1..20: mol_type = genomic RNA
aactggatggggcttctcga

SEQ ID NO: 373 - RNA, 20 nt, Macaca fascicularis
  source 1..20: mol_type = genomic RNA
ctccaactaggattacatat

SEQ ID NO: 374 - RNA, 20 nt, Macaca fascicularis
  source 1..20: mol_type = genomic RNA
catccgagaaggcaagatgc

SEQ ID NO: 375 - RNA, 20 nt, Macaca fascicularis
  source 1..20: mol_type = genomic RNA
ttgaatgtgactttcgttaa

SEQ ID NO: 376 - RNA, 20 nt, Macaca fascicularis
  source 1..20: mol_type = genomic RNA
atgtcattgatggaacttgc

SEQ ID NO: 377 - RNA, 20 nt, Macaca fascicularis
  source 1..20: mol_type = genomic RNA
ttgatgaccacactgcttta

SEQ ID NO: 378 - RNA, 20 nt, Macaca fascicularis
  source 1..20: mol_type = genomic RNA
ctgtgtggagggtcactcat

SEQ ID NO: 379 - RNA, 20 nt, Macaca fascicularis
  source 1..20: mol_type = genomic RNA
ggtggaatgtgcacatcatc

SEQ ID NO: 380 - RNA, 20 nt, Macaca fascicularis
  source 1..20: mol_type = genomic RNA
ttcttaagattatcttcgga

SEQ ID NO: 381 - RNA, 20 nt, Macaca fascicularis
  source 1..20: mol_type = genomic RNA
cctttggaaggtaggcatat

SEQ ID NO: 382 - RNA, 20 nt, Macaca fascicularis
  source 1..20: mol_type = genomic RNA
tccattgtgtttgcaagcta

SEQ ID NO: 383 - RNA, 20 nt, Macaca fascicularis
  source 1..20: mol_type = genomic RNA
cctgtgactcagggagattc

SEQ ID NO: 384 - RNA, 20 nt, Macaca fascicularis
  source 1..20: mol_type = genomic RNA
tgcacatcatctggcagtgc

SEQ ID NO: 385 - RNA, 20 nt, Macaca fascicularis
  source 1..20: mol_type = genomic RNA
gttattcagcacctttatag

SEQ ID NO: 386 - RNA, 20 nt, Macaca fascicularis
  source 1..20: mol_type = genomic RNA
ggtcactcataggacaccag

SEQ ID NO: 387 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 388 - RNA, 100 nt, synthetic construct
  source 1..100: mol_type = other RNA
ggattgcgtatgggacacaagttttagagctagaaatagcaagttaaaataaggctagtccgttatcaacttgaaaaagtggcaccgagtcggtgctttt

SEQ ID NO: 389 - RNA, 70 nt, synthetic construct
  source 1..70: mol_type = other RNA
gttttagagctagaaatagcaagttaaaataaggctagtccgttatcacgaaagggcaccgagtcggtgc

SEQ ID NO: 390 - RNA, 68 nt, synthetic construct
  source 1..68: mol_type = other RNA
gttttagagctagaaatagcaagttaaaataaggctagtccgttatcaaaatggcaccgagtcggtgc

SEQ ID NO: 391 - RNA, 100 nt, synthetic construct; identical to SEQ ID NO: 388
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 392 - RNA, 13 nt, synthetic construct
  source 1..13: mol_type = other RNA
gccgccrccatgg

SEQ ID NO: 393 - RNA, 4143 nt, synthetic construct
  source 1..4143: mol_type = other RNA
atggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatga

SEQ ID NO: 394 - DNA, 4140 bp, synthetic construct
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 395 - DNA, 4140 bp, synthetic construct
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 396 - RNA, 4140 nt, synthetic construct
  source 1..4140: mol_type = other RNA
atggacaagaagtacagcatcggcctggacatcggcacgaacagcgttggctgggctgtgatcacggacgagtacaaggttccctcaaagaagttcaaggtgctgggcaacacggaccggcacagcatcaagaagaatctcatcggtgcactgctgttcgacagcggtgagacggccgaagccacgcggctgaagcggacggcccgccggcggtacacgcggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaagtcgcctaccacgagaagtaccccaccatctaccacctgcggaagaagctggtggactcgactgacaaggccgacctgcggctgatctacctggcactggcccacatgataaagttccggggccacttcctgatcgagggcgacctgaaccctgacaacagcgacgtggacaagctgttcatccagctggtgcagacctacaaccagctgttcgaggagaaccccatcaacgccagcggcgtggacgccaaggccatcctcagcgcccgcctcagcaagagccggcggctggagaatctcatcgcccagcttccaggtgagaagaagaatgggctgttcggcaatctcatcgcactcagcctgggcctgactcccaacttcaagagcaacttcgacctggccgaggacgccaagctgcagctcagcaaggacacctacgacgacgacctggacaatctcctggcccagatcggcgaccagtacgccgacctgttcctggctgccaagaatctcagcgacgccatcctgctcagcgacatcctgcgggtgaacacagagatcacgaaggcccccctcagcgccagcatgataaagcggtacgacgagcaccaccaggacctgacgctgctgaaggcactggtgcggcagcagcttccagagaagtacaaggagatcttcttcgaccagagcaagaatgggtacgccgggtacatcgacggtggtgccagccaggaggagttctacaagttcatcaagcccatcctggagaagatggacggcacagaggagctgctggtgaagctgaacagggaggacctgctgcggaagcagcggacgttcgacaatgggagcatcccccaccagatccacctgggtgagctgcacgccatcctgcggcggcaggaggacttctaccccttcctgaaggacaacagggagaagatcgagaagatcctgacgttccggatcccctactacgttggccccctggcccgcggcaacagccggttcgcctggatgacgcggaagagcgaggagacgatcactccctggaacttcgaggaagtcgtggacaagggtgccagcgcccagagcttcatcgagcggatgacgaacttcgacaagaatcttccaaacgagaaggtgcttccaaagcacagcctgctgtacgagtacttcacggtgtacaacgagctgacgaaggtgaagtacgtgacagagggcatgcggaagcccgccttcctcagcggtgagcagaagaaggccatcgtggacctgctgttcaagacgaaccggaaggtgacggtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgacgctgacgctgttcgaggacagggagatgatagaggagcggctgaagacctacgcccacctgttcgacgacaaggtgatgaagcagctgaagcggcggcggtacacgggctggggccggctcagccggaagctgatcaatgggatccgagacaagcagagcggcaagacgatcctggacttcctgaagagcgacggcttcgccaaccggaacttcatgcagctgatccacgacgacagcctgacgttcaaggaggacatccagaaggcccaggtcagcggccagggcgacagcctgcacgagcacatcgccaatctcgccgggagccccgccatcaagaaggggatcctgcagacggtgaaggtggtggacgagctggtgaaggtgatgggccggcacaagccagagaacatcgtgatcgagatggccagggagaaccagacgactcaaaaggggcagaagaacagcagggagcggatgaagcggatcgaggagggcatcaaggagctgggcagccagatcctgaaggagcaccccgtggagaacactcaactgcagaacgagaagctgtacctgtactacctgcagaatgggcgagacatgtacgtggaccaggagctggacatcaaccggctcagcgactacgacgtggaccacatcgttccccagagcttcctgaaggacgacagcatcgacaacaaggtgctgacgcggagcgacaagaaccggggcaagagcgacaacgttccctcagaggaagtcgtgaagaagatgaagaactactggcggcagctgctgaacgccaagctgatcactcaacggaagttcgacaatctcacgaaggccgagcggggtggcctcagcgagctggacaaggccgggttcatcaagcggcagctggtggagacgcggcagatcacgaagcacgtggcccagatcctggacagccggatgaacacgaagtacgacgagaacgacaagctgatcagggaagtcaaggtgatcacgctgaagagcaagctggtcagcgacttccggaaggacttccagttctacaaggtgagggagatcaacaactaccaccacgcccacgacgcctacctgaacgctgtggttggcacggcactgatcaagaagtaccccaagctggagagcgagttcgtgtacggcgactacaaggtgtacgacgtgcggaagatgatagccaagagcgagcaggagatcggcaaggccacggccaagtacttcttctacagcaacatcatgaacttcttcaagacagagatcacgctggccaatggtgagatccggaagcggcccctgatcgagacgaatggtgagacgggtgagatcgtgtgggacaaggggcgagacttcgccacggtgcggaaggtgctcagcatgccccaggtgaacatcgtgaagaagacagaagtccagacgggtggcttcagcaaggagagcatccttccaaagcggaacagcgacaagctgatcgcccgcaagaaggactgggaccccaagaagtacggtggcttcgacagccccaccgtggcctacagcgtgctggtggtggccaaggtggagaaggggaagagcaagaagctgaagagcgtgaaggagctgctgggcatcacgatcatggagcggagcagcttcgagaagaaccccatcgacttcctggaagccaaggggtacaaggaagtcaagaaggacctgatcatcaagcttccaaagtacagcctgttcgagctggagaatgggcggaagcggatgctggccagcgccggtgagctgcagaaggggaacgagctggcacttccctcaaagtacgtgaacttcctgtacctggccagccactacgagaagctgaaggggagcccagaggacaacgagcagaagcagctgttcgtggagcagcacaagcactacctggacgagatcatcgagcagatcagcgagttcagcaagcgggtgatcctggccgacgccaatctcgacaaggtgctcagcgcctacaacaagcaccgagacaagcccatcagggagcaggccgagaacatcatccacctgttcacgctgacgaatctcggtgcccccgctgccttcaagtacttcgacacgacgatcgaccggaagcggtacacgtcgactaaggaagtcctggacgccacgctgatccaccagagcatcacgggcctgtacgagacgcggatcgacctcagccagctgggtggcgacggtggtggcagccccaagaagaagcggaaggtgtag

SEQ ID NO: 397 - RNA, 4140 nt, synthetic construct
  source 1..4140: mol_type = other RNA
atggacaagaagtacagcatcggcctcgacatcggcaccaacagcgtcggctgggccgtcatcaccgacgagtacaaggtccccagcaagaagttcaaggtcctcggcaacaccgaccgccacagcatcaagaagaacctcatcggcgccctcctcttcgacagcggcgagaccgccgaggccacccgcctcaagcgcaccgcccgccgccgctacacccgccgcaagaaccgcatctgctacctccaggagatcttcagcaacgagatggccaaggtcgacgacagcttcttccaccgcctcgaggagagcttcctcgtcgaggaggacaagaagcacgagcgccaccccatcttcggcaacatcgtcgacgaggtcgcctaccacgagaagtaccccaccatctaccacctccgcaagaagctcgtcgacagcaccgacaaggccgacctccgcctcatctacctcgccctcgcccacatgatcaagttccgcggccacttcctcatcgagggcgacctcaaccccgacaacagcgacgtcgacaagctcttcatccagctcgtccagacctacaaccagctcttcgaggagaaccccatcaacgccagcggcgtcgacgccaaggccatcctcagcgcccgcctcagcaagagccgccgcctcgagaacctcatcgcccagctccccggcgagaagaagaacggcctcttcggcaacctcatcgccctcagcctcggcctcacccccaacttcaagagcaacttcgacctcgccgaggacgccaagctccagctcagcaaggacacctacgacgacgacctcgacaacctcctcgcccagatcggcgaccagtacgccgacctcttcctcgccgccaagaacctcagcgacgccatcctcctcagcgacatcctccgcgtcaacaccgagatcaccaaggcccccctcagcgccagcatgatcaagcgctacgacgagcaccaccaggacctcaccctcctcaaggccctcgtccgccagcagctccccgagaagtacaaggagatcttcttcgaccagagcaagaacggctacgccggctacatcgacggcggcgccagccaggaggagttctacaagttcatcaagcccatcctcgagaagatggacggcaccgaggagctcctcgtcaagctcaaccgcgaggacctcctccgcaagcagcgcaccttcgacaacggcagcatcccccaccagatccacctcggcgagctccacgccatcctccgccgccaggaggacttctaccccttcctcaaggacaaccgcgagaagatcgagaagatcctcaccttccgcatcccctactacgtcggccccctcgcccgcggcaacagccgcttcgcctggatgacccgcaagagcgaggagaccatcaccccctggaacttcgaggaggtcgtcgacaagggcgccagcgcccagagcttcatcgagcgcatgaccaacttcgacaagaacctccccaacgagaaggtcctccccaagcacagcctcctctacgagtacttcaccgtctacaacgagctcaccaaggtcaagtacgtcaccgagggcatgcgcaagcccgccttcctcagcggcgagcagaagaaggccatcgtcgacctcctcttcaagaccaaccgcaaggtcaccgtcaagcagctcaaggaggactacttcaagaagatcgagtgcttcgacagcgtcgagatcagcggcgtcgaggaccgcttcaacgccagcctcggcacctaccacgacctcctcaagatcatcaaggacaaggacttcctcgacaacgaggagaacgaggacatcctcgaggacatcgtcctcaccctcaccctcttcgaggaccgcgagatgatcgaggagcgcctcaagacctacgcccacctcttcgacgacaaggtcatgaagcagctcaagcgccgccgctacaccggctggggccgcctcagccgcaagctcatcaacggcatccgcgacaagcagagcggcaagaccatcctcgacttcctcaagagcgacggcttcgccaaccgcaacttcatgcagctcatccacgacgacagcctcaccttcaaggaggacatccagaaggcccaggtcagcggccagggcgacagcctccacgagcacatcgccaacctcgccggcagccccgccatcaagaagggcatcctccagaccgtcaaggtcgtcgacgagctcgtcaaggtcatgggccgccacaagcccgagaacatcgtcatcgagatggcccgcgagaaccagaccacccagaagggccagaagaacagccgcgagcgcatgaagcgcatcgaggagggcatcaaggagctcggcagccagatcctcaaggagcaccccgtcgagaacacccagctccagaacgagaagctctacctctactacctccagaacggccgcgacatgtacgtcgaccaggagctcgacatcaaccgcctcagcgactacgacgtcgaccacatcgtcccccagagcttcctcaaggacgacagcatcgacaacaaggtcctcacccgcagcgacaagaaccgcggcaagagcgacaacgtccccagcgaggaggtcgtcaagaagatgaagaactactggcgccagctcctcaacgccaagctcatcacccagcgcaagttcgacaacctcaccaaggccgagcgcggcggcctcagcgagctcgacaaggccggcttcatcaagcgccagctcgtcgagacccgccagatcaccaagcacgtcgcccagatcctcgacagccgcatgaacaccaagtacgacgagaacgacaagctcatccgcgaggtcaaggtcatcaccctcaagagcaagctcgtcagcgacttccgcaaggacttccagttctacaaggtccgcgagatcaacaactaccaccacgcccacgacgcctacctcaacgccgtcgtcggcaccgccctcatcaagaagtaccccaagctcgagagcgagttcgtctacggcgactacaaggtctacgacgtccgcaagatgatcgccaagagcgagcaggagatcggcaaggccaccgccaagtacttcttctacagcaacatcatgaacttcttcaagaccgagatcaccctcgccaacggcgagatccgcaagcgccccctcatcgagaccaacggcgagaccggcgagatcgtctgggacaagggccgcgacttcgccaccgtccgcaaggtcctcagcatgccccaggtcaacatcgtcaagaagaccgaggtccagaccggcggcttcagcaaggagagcatcctccccaagcgcaacagcgacaagctcatcgcccgcaagaaggactgggaccccaagaagtacggcggcttcgacagccccaccgtcgcctacagcgtcctcgtcgtcgccaaggtcgagaagggcaagagcaagaagctcaagagcgtcaaggagctcctcggcatcaccatcatggagcgcagcagcttcgagaagaaccccatcgacttcctcgaggccaagggctacaaggaggtcaagaaggacctcatcatcaagctccccaagtacagcctcttcgagctcgagaacggccgcaagcgcatgctcgccagcgccggcgagctccagaagggcaacgagctcgccctccccagcaagtacgtcaacttcctctacctcgccagccactacgagaagctcaagggcagccccgaggacaacgagcagaagcagctcttcgtcgagcagcacaagcactacctcgacgagatcatcgagcagatcagcgagttcagcaagcgcgtcatcctcgccgacgccaacctcgacaaggtcctcagcgcctacaacaagcaccgcgacaagcccatccgcgagcaggccgagaacatcatccacctcttcaccctcaccaacctcggcgcccccgccgccttcaagtacttcgacaccaccatcgaccgcaagcgctacaccagcaccaaggaggtcctcgacgccaccctcatccaccagagcatcaccggcctctacgagacccgcatcgacctcagccagctcggcggcgacggcggcggcagccccaagaagaagcgcaaggtctag

SEQ ID NO: 398 - DNA, 4140 bp, synthetic construct
  source 1..4140: mol_type = other DNA
atggacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtga

SEQ ID NO: 399 - DNA, 4140 bp, synthetic construct
  source 1..4140: mol_type = other DNA
atggacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 400 - DNA, 4140 bp, synthetic construct
  source 1..4140: mol_type = other DNA
atggacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtga

SEQ ID NO: 401 - RNA, 4511 nt, synthetic construct
  source 1..4511: mol_type = other RNA
gggtcccgcagtcggcgtccagcggctctgcttgttcgtgtgtgtgtcgttgcaggccttattcggatccgccaccatggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 402 - RNA, 4418 nt, synthetic construct
  source 1..4418: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 403 - RNA, 4416 nt, synthetic construct
  source 1..4416: mol_type = other RNA
gggaagctcagaataaacgctcaactttggccggatctgccaccatggacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 404 - RNA, 4420 nt, synthetic construct
  source 1..4420: mol_type = other RNA
gggaagctcagaataaacgctcaactttggccggatctgccaccatggacaagaagtacagcatcggcctggacatcggcacgaacagcgttggctgggctgtgatcacggacgagtacaaggttccctcaaagaagttcaaggtgctgggcaacacggaccggcacagcatcaagaagaatctcatcggtgcactgctgttcgacagcggtgagacggccgaagccacgcggctgaagcggacggcccgccggcggtacacgcggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaagtcgcctaccacgagaagtaccccaccatctaccacctgcggaagaagctggtggactcgactgacaaggccgacctgcggctgatctacctggcactggcccacatgataaagttccggggccacttcctgatcgagggcgacctgaaccctgacaacagcgacgtggacaagctgttcatccagctggtgcagacctacaaccagctgttcgaggagaaccccatcaacgccagcggcgtggacgccaaggccatcctcagcgcccgcctcagcaagagccggcggctggagaatctcatcgcccagcttccaggtgagaagaagaatgggctgttcggcaatctcatcgcactcagcctgggcctgactcccaacttcaagagcaacttcgacctggccgaggacgccaagctgcagctcagcaaggacacctacgacgacgacctggacaatctcctggcccagatcggcgaccagtacgccgacctgttcctggctgccaagaatctcagcgacgccatcctgctcagcgacatcctgcgggtgaacacagagatcacgaaggcccccctcagcgccagcatgataaagcggtacgacgagcaccaccaggacctgacgctgctgaaggcactggtgcggcagcagcttccagagaagtacaaggagatcttcttcgaccagagcaagaatgggtacgccgggtacatcgacggtggtgccagccaggaggagttctacaagttcatcaagcccatcctggagaagatggacggcacagaggagctgctggtgaagctgaacagggaggacctgctgcggaagcagcggacgttcgacaatgggagcatcccccaccagatccacctgggtgagctgcacgccatcctgcggcggcaggaggacttctaccccttcctgaaggacaacagggagaagatcgagaagatcctgacgttccggatcccctactacgttggccccctggcccgcggcaacagccggttcgcctggatgacgcggaagagcgaggagacgatcactccctggaacttcgaggaagtcgtggacaagggtgccagcgcccagagcttcatcgagcggatgacgaacttcgacaagaatcttccaaacgagaaggtgcttccaaagcacagcctgctgtacgagtacttcacggtgtacaacgagctgacgaaggtgaagtacgtgacagagggcatgcggaagcccgccttcctcagcggtgagcagaagaaggccatcgtggacctgctgttcaagacgaaccggaaggtgacggtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgacgctgacgctgttcgaggacagggagatgatagaggagcggctgaagacctacgcccacctgttcgacgacaaggtgatgaagcagctgaagcggcggcggtacacgggctggggccggctcagccggaagctgatcaatgggatccgagacaagcagagcggcaagacgatcctggacttcctgaagagcgacggcttcgccaaccggaacttcatgcagctgatccacgacgacagcctgacgttcaaggaggacatccagaaggcccaggtcagcggccagggcgacagcctgcacgagcacatcgccaatctcgccgggagccccgccatcaagaaggggatcctgcagacggtgaaggtggtggacgagctggtgaaggtgatgggccggcacaagccagagaacatcgtgatcgagatggccagggagaaccagacgactcaaaaggggcagaagaacagcagggagcggatgaagcggatcgaggagggcatcaaggagctgggcagccagatcctgaaggagcaccccgtggagaacactcaactgcagaacgagaagctgtacctgtactacctgcagaatgggcgagacatgtacgtggaccaggagctggacatcaaccggctcagcgactacgacgtggaccacatcgttccccagagcttcctgaaggacgacagcatcgacaacaaggtgctgacgcggagcgacaagaaccggggcaagagcgacaacgttccctcagaggaagtcgtgaagaagatgaagaactactggcggcagctgctgaacgccaagctgatcactcaacggaagttcgacaatctcacgaaggccgagcggggtggcctcagcgagctggacaaggccgggttcatcaagcggcagctggtggagacgcggcagatcacgaagcacgtggcccagatcctggacagccggatgaacacgaagtacgacgagaacgacaagctgatcagggaagtcaaggtgatcacgctgaagagcaagctggtcagcgacttccggaaggacttccagttctacaaggtgagggagatcaacaactaccaccacgcccacgacgcctacctgaacgctgtggttggcacggcactgatcaagaagtaccccaagctggagagcgagttcgtgtacggcgactacaaggtgtacgacgtgcggaagatgatagccaagagcgagcaggagatcggcaaggccacggccaagtacttcttctacagcaacatcatgaacttcttcaagacagagatcacgctggccaatggtgagatccggaagcggcccctgatcgagacgaatggtgagacgggtgagatcgtgtgggacaaggggcgagacttcgccacggtgcggaaggtgctcagcatgccccaggtgaacatcgtgaagaagacagaagtccagacgggtggcttcagcaaggagagcatccttccaaagcggaacagcgacaagctgatcgcccgcaagaaggactgggaccccaagaagtacggtggcttcgacagccccaccgtggcctacagcgtgctggtggtggccaaggtggagaaggggaagagcaagaagctgaagagcgtgaaggagctgctgggcatcacgatcatggagcggagcagcttcgagaagaaccccatcgacttcctggaagccaaggggtacaaggaagtcaagaaggacctgatcatcaagcttccaaagtacagcctgttcgagctggagaatgggcggaagcggatgctggccagcgccggtgagctgcagaaggggaacgagctggcacttccctcaaagtacgtgaacttcctgtacctggccagccactacgagaagctgaaggggagcccagaggacaacgagcagaagcagctgttcgtggagcagcacaagcactacctggacgagatcatcgagcagatcagcgagttcagcaagcgggtgatcctggccgacgccaatctcgacaaggtgctcagcgcctacaacaagcaccgagacaagcccatcagggagcaggccgagaacatcatccacctgttcacgctgacgaatctcggtgcccccgctgccttcaagtacttcgacacgacgatcgaccggaagcggtacacgtcgactaaggaagtcctggacgccacgctgatccaccagagcatcacgggcctgtacgagacgcggatcgacctcagccagctgggtggcgacggtggtggcagccccaagaagaagcggaaggtgtagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 405 - RNA, 4429 nt, synthetic construct
  source 1..4429: mol_type = other RNA
gggaagctcagaataaacgctcaactttggccggatctgccaccatggacaagaagtacagcatcggcctcgacatcggcaccaacagcgtcggctgggccgtcatcaccgacgagtacaaggtccccagcaagaagttcaaggtcctcggcaacaccgaccgccacagcatcaagaagaacctcatcggcgccctcctcttcgacagcggcgagaccgccgaggccacccgcctcaagcgcaccgcccgccgccgctacacccgccgcaagaaccgcatctgctacctccaggagatcttcagcaacgagatggccaaggtcgacgacagcttcttccaccgcctcgaggagagcttcctcgtcgaggaggacaagaagcacgagcgccaccccatcttcggcaacatcgtcgacgaggtcgcctaccacgagaagtaccccaccatctaccacctccgcaagaagctcgtcgacagcaccgacaaggccgacctccgcctcatctacctcgccctcgcccacatgatcaagttccgcggccacttcctcatcgagggcgacctcaaccccgacaacagcgacgtcgacaagctcttcatccagctcgtccagacctacaaccagctcttcgaggagaaccccatcaacgccagcggcgtcgacgccaaggccatcctcagcgcccgcctcagcaagagccgccgcctcgagaacctcatcgcccagctccccggcgagaagaagaacggcctcttcggcaacctcatcgccctcagcctcggcctcacccccaacttcaagagcaacttcgacctcgccgaggacgccaagctccagctcagcaaggacacctacgacgacgacctcgacaacctcctcgcccagatcggcgaccagtacgccgacctcttcctcgccgccaagaacctcagcgacgccatcctcctcagcgacatcctccgcgtcaacaccgagatcaccaaggcccccctcagcgccagcatgatcaagcgctacgacgagcaccaccaggacctcaccctcctcaaggccctcgtccgccagcagctccccgagaagtacaaggagatcttcttcgaccagagcaagaacggctacgccggctacatcgacggcggcgccagccaggaggagttctacaagttcatcaagcccatcctcgagaagatggacggcaccgaggagctcctcgtcaagctcaaccgcgaggacctcctccgcaagcagcgcaccttcgacaacggcagcatcccccaccagatccacctcggcgagctccacgccatcctccgccgccaggaggacttctaccccttcctcaaggacaaccgcgagaagatcgagaagatcctcaccttccgcatcccctactacgtcggccccctcgcccgcggcaacagccgcttcgcctggatgacccgcaagagcgaggagaccatcaccccctggaacttcgaggaggtcgtcgacaagggcgccagcgcccagagcttcatcgagcgcatgaccaacttcgacaagaacctccccaacgagaaggtcctccccaagcacagcctcctctacgagtacttcaccgtctacaacgagctcaccaaggtcaagtacgtcaccgagggcatgcgcaagcccgccttcctcagcggcgagcagaagaaggccatcgtcgacctcctcttcaagaccaaccgcaaggtcaccgtcaagcagctcaaggaggactacttcaagaagatcgagtgcttcgacagcgtcgagatcagcggcgtcgaggaccgcttcaacgccagcctcggcacctaccacgacctcctcaagatcatcaaggacaaggacttcctcgacaacgaggagaacgaggacatcctcgaggacatcgtcctcaccctcaccctcttcgaggaccgcgagatgatcgaggagcgcctcaagacctacgcccacctcttcgacgacaaggtcatgaagcagctcaagcgccgccgctacaccggctggggccgcctcagccgcaagctcatcaacggcatccgcgacaagcagagcggcaagaccatcctcgacttcctcaagagcgacggcttcgccaaccgcaacttcatgcagctcatccacgacgacagcctcaccttcaaggaggacatccagaaggcccaggtcagcggccagggcgacagcctccacgagcacatcgccaacctcgccggcagccccgccatcaagaagggcatcctccagaccgtcaaggtcgtcgacgagctcgtcaaggtcatgggccgccacaagcccgagaacatcgtcatcgagatggcccgcgagaaccagaccacccagaagggccagaagaacagccgcgagcgcatgaagcgcatcgaggagggcatcaaggagctcggcagccagatcctcaaggagcaccccgtcgagaacacccagctccagaacgagaagctctacctctactacctccagaacggccgcgacatgtacgtcgaccaggagctcgacatcaaccgcctcagcgactacgacgtcgaccacatcgtcccccagagcttcctcaaggacgacagcatcgacaacaaggtcctcacccgcagcgacaagaaccgcggcaagagcgacaacgtccccagcgaggaggtcgtcaagaagatgaagaactactggcgccagctcctcaacgccaagctcatcacccagcgcaagttcgacaacctcaccaaggccgagcgcggcggcctcagcgagctcgacaaggccggcttcatcaagcgccagctcgtcgagacccgccagatcaccaagcacgtcgcccagatcctcgacagccgcatgaacaccaagtacgacgagaacgacaagctcatccgcgaggtcaaggtcatcaccctcaagagcaagctcgtcagcgacttccgcaaggacttccagttctacaaggtccgcgagatcaacaactaccaccacgcccacgacgcctacctcaacgccgtcgtcggcaccgccctcatcaagaagtaccccaagctcgagagcgagttcgtctacggcgactacaaggtctacgacgtccgcaagatgatcgccaagagcgagcaggagatcggcaaggccaccgccaagtacttcttctacagcaacatcatgaacttcttcaagaccgagatcaccctcgccaacggcgagatccgcaagcgccccctcatcgagaccaacggcgagaccggcgagatcgtctgggacaagggccgcgacttcgccaccgtccgcaaggtcctcagcatgccccaggtcaacatcgtcaagaagaccgaggtccagaccggcggcttcagcaaggagagcatcctccccaagcgcaacagcgacaagctcatcgcccgcaagaaggactgggaccccaagaagtacggcggcttcgacagccccaccgtcgcctacagcgtcctcgtcgtcgccaaggtcgagaagggcaagagcaagaagctcaagagcgtcaaggagctcctcggcatcaccatcatggagcgcagcagcttcgagaagaaccccatcgacttcctcgaggccaagggctacaaggaggtcaagaaggacctcatcatcaagctccccaagtacagcctcttcgagctcgagaacggccgcaagcgcatgctcgccagcgccggcgagctccagaagggcaacgagctcgccctccccagcaagtacgtcaacttcctctacctcgccagccactacgagaagctcaagggcagccccgaggacaacgagcagaagcagctcttcgtcgagcagcacaagcactacctcgacgagatcatcgagcagatcagcgagttcagcaagcgcgtcatcctcgccgacgccaacctcgacaaggtcctcagcgcctacaacaagcaccgcgacaagcccatccgcgagcaggccgagaacatcatccacctcttcaccctcaccaacctcggcgcccccgccgccttcaagtacttcgacaccaccatcgaccgcaagcgctacaccagcaccaaggaggtcctcgacgccaccctcatccaccagagcatcaccggcctctacgagacccgcatcgacctcagccagctcggcggcgacggcggcggcagccccaagaagaagcgcaaggtctagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 406 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 407 - protein, 1379 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 408 - RNA, 4325 nt, synthetic construct
  source 1..4325: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgag

SEQ ID NO: 409 - RNA, 100 nt, synthetic construct
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
ggattacatatgggacacaagttttagagctagaaatagcaagttaaaataaggctagtccgttatcaacttgaaaaagtggcaccgagtcggtgctttt

SEQ ID NO: 410 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa